(19) United States
(12) Patent Application Publication
(10) Pub. No.: US 2023/0331824 A1
(43) Pub. Date: Oct. 19, 2023

(54) SINGLE-DOMAIN ANTIBODIES THAT BIND SARS-COV-2

(71) Applicants: The Rockefeller University, New York, NY (US); New York University, New York, NY (US); Seattle Children's Hospital d/b/a Seattle Children's Research Institute, Seattle, WA (US)

(72) Inventors: Brian T. Chait, New York, NY (US); Michael P. Rout, New York, NY (US); John Aitchison, Seattle, WA (US); Fred David Mast, Seattle, WA (US); Jean Paul Olivier, Seattle, WA (US); David Fenyo, New York, NY (US)

(73) Assignees: The Rockefeller University, New York, NY (US); Seattle Children's Hospital d/b/a Seattle Children's Research Institute, Seattle, WA (US); New York University, New York, NY (US)

(21) Appl. No.: 18/042,448

(22) PCT Filed: Aug. 20, 2021
(86) PCT No.: PCT/US21/47019

§ 371 Date: Feb. 21, 2023

Related U.S. Application Data
(60) Provisional application No. 63/197,270, filed on Jun. 4, 2021.
(60) Provisional application No. 63/068,720, filed on Aug. 21, 2020.

(51) Int. Cl. C07K 16/10; A61K 47/68; A61K 51/10; C07K 14/725; A61K 39/00; A61P 31/14; C07K 16/28; C07K 16/18

(52) U.S. Cl. CPC C07K 16/1003; A61K 47/6829; A61K 47/6827; A61K 47/6825; A61K 47/6821; A61K 47/6819; A61K 47/68031; A61K 47/68033; A61K 47/6809; A61K 47/6805; A61K 51/1006; C07K 14/7051; A61K 39/4631; A61K 39/4611; A61K 39/464838; A61K 39/4633; A61P 31/14; C07K 16/2809; C07K 16/2818; C07K 16/2815; C07K 16/18; C07K 2317/569; C07K 2317/52; C07K 2317/92; C07K 2317/94; C07K 2317/31; C07K 2319/33; C07K 2319/03; C07K 2317/76

(57) ABSTRACT

Single-domain antibodies that bind the severe acute respiratory syndrome corona virus 2 (SARS-CoV-2) spike protein are disclosed. The single-domain antibodies include binding domains that bind epitopes of the Spike ectodomain inside and outside the receptor binding domain. The single-domain antibodies can be used for multiple purposes including in the research, diagnosis, and prophylactic or therapeutic treatment of COVID-19.

### CROSS-REFERENCE TO RELATED APPLICATION

This application is a U.S. National Phase Patent Application based on International Patent Application No. PCT/US2021/047019, filed on Aug. 20, 2021, which claims priority to U.S. Provisional Patent Application No. 63/197,270, filed Jun. 4, 2021 and U.S. Provisional Patent Application No. 63/068,720, filed Aug. 21, 2020, each of which is incorporated by reference herein in its entirety as if fully set forth herein.

### REFERENCE TO SEQUENCE LISTING

The Sequence Listing associated with this application is provided in text format in lieu of a paper copy and is hereby incorporated by reference into the specification. The name of the text file containing the Sequence Listing is 2UK1148_ST25.txt. The text file is 323 KB, was created on Feb. 17, 2023 and is being submitted electronically via Patent Center.

### FIELD OF THE DISCLOSURE

The current disclosure provides single-domain antibodies (nanobodies) that bind the severe acute respiratory syndrome corona virus 2 (SARS-CoV-2) spike protein. The single-domain antibodies can be used for multiple purposes including in the research, diagnosis, and treatment of COVID-19.

### BACKGROUND OF THE DISCLOSURE

SARS-CoV-2, the viral causative agent of COVID-19, has infected over 191 million people since it emerged in 2019, killing more than 4 million; despite the great promise of vaccines, the resulting pandemic is ongoing. Inequities in vaccine distribution, waning immunity, the biological and behavioral diversity of the human population, and the emergence of viral variants that challenge monoclonal therapies and vaccine efficacy indicate that future outbreaks are highly likely (Diamond et al., 2021, Res Sq. doi: 10.21203/rs.3.rs-228079/v1; Fraser et al., 2004, Proc Natl Acad Sci USA 101, 6146-6151; Lavine et al., 2021, Science 371, 741-745; Wang et al., 2021, doi: 10.1101/2021.01.25.428137; Wang et al., 2021, Nature 592(7855):616-622). As such, multi-pronged containment strategies will be required for many years to keep SARS-CoV-2, future variants and novel coronaviruses at bay (McKenna, 2021, Scientific American 324; Phillips, 2021, Nature 590, 382-384.; Steenhuysen and Kelland, 2021, In Reuters (New York: Thomson Reuters); Weisblum et al., 2020, Elife 9). Already, the initial virus isolate, Wuhan-Hu-1 (Wu et al., A new coronavirus associated with human respiratory disease in China. Nature, 2020), has several variants including the B.1.1.7 (Alpha), B.1.351 (Beta), B.1.617.2 (Delta), P.1 (Gamma), B.1.526 (Iota), B.1.427 and B.1.429 (both referred to as Epsilon), B.1.617.1 (Kappa), B.1.525 (Eta), and P.2 (Zeta).

Spike (S), the major surface envelope glycoprotein of the SARS-CoV-2 virion, is key for infection as it attaches the virion to its cognate host surface receptor, angiotensin-converting enzyme 2 (ACE2) protein, and triggers fusion between the host and viral membranes, leading to viral entry into the cytoplasm (Walls et al., 2020, Cell 183, 1735; Wrapp et al., 2020, Science 367, 1260-1263; Zhou et al., 2020, Nature 579, 270-273). The Spike protein monomer is 200 kDa, is extensively glycosylated to help evade immune system surveillance, and exists as a homotrimer on the viral surface. Spike is highly dynamic and is composed of two domains: S1, which contains the host receptor binding domain (RBD); and S2, which undergoes large conformational changes that enable fusion of the viral membrane with that of its host (Hsieh et al., 2020, Science 369, 1501-1505; Letko et al., 2020, Nat Microbiol 5, 562-569; Li, 2016, Annu Rev Virol 3, 237-261; Li et al., 2003, Nature 426, 450-454; Watanabe et al., 2020, Science 369, 330-333). Based on the requirement for attachment to ACE2 for entry, the major target of immunotherapeutics has been the RBD (Barnes et al., 2020, Nature 588, 682-687; Baum et al., 2020, Science 369, 1014-1018; Finkelstein et al., 2021, Viruses 13; Hartenian et al., 2020, J Biol Chem 295, 12910-12934; Korber et al., 2020, Cell; Trigueiro-Louro et al., 2020, Comput Struct Biotechnol J 18, 2117-2131; Wu et al., 2020, Science 368, 1274-1278).

Major immunotherapeutic strategies to date have focused on immune sera and human monoclonal antibodies; however, these therapies now face the emergence of variants, including RBD point mutants, that have evolved to bypass the most potent neutralizing human antibodies, which are the very basis of immunotherapies (Garcia-Beltran et al., 2021, Cell 184(9):2372-2383; Liu et al., 2021, Cell Host Microbe 29, 477-488 e474; Starr et al., 2021, Science 371, 850-854; Wang et al., 2021, Nature 592(7855):616-622; Weisblum et al., 2020, Elife 9).

### SUMMARY OF THE DISCLOSURE

The current disclosure provides a large library of high affinity single-domain antibodies (nanobodies) against the SARS-CoV-2 Spike protein. Nanobodies are the smallest naturally occurring single domain antigen binding proteins identified to date, possessing numerous properties advantageous to their production and use. The nanobodies disclosed herein include high affinity nanobodies against the SARS-CoV-2 Spike protein with excellent kinetic and viral neutralization properties, which can be strongly enhanced with oligomerization. This library samples the epitope landscape of the Spike ectodomain inside and outside the receptor binding domain (RBD), recognizing a multitude of distinct epitopes and revealing multiple neutralization targets of pseudoviruses and authentic SARS-CoV-2. Combinatorial nanobody mixtures show highly synergistic activities and are resistant to emerging viral variants of concern. These nanobodies establish an exceptional resource for superior COVID-19 prophylactics and therapeutics.

### BRIEF DESCRIPTION OF THE SEVERAL VIEWS OF THE DRAWINGS

Some of the drawings submitted herein may be better understood in color. Applicants consider the color versions of the drawings as part of the original submission and reserve the right to present color images of the drawings in later proceedings.

FIG. 1. Schematics of classic antibodies, heavy chain only antibodies (HcAb), and single-domain antibodies. Adapted from Tillib S. V. Molecular Biology 2020; 54(3):317-326.

FIGS. 2A-2C. Approach. (2A) Schematic of the strategy for generating, identifying, and characterizing large, diverse repertoires of nanobodies that bind the spike protein of SARS-CoV-2. The highest quality nanobodies were assayed for their ability to neutralize SARS-CoV-2 pseudo-virus, SARS-CoV-2 virus, and viral entry into primary human airway epithelial cells. The activities of homodimers/homotrimers and mixtures were also measured. (2B) A network visualization of 374 high confidence CDR3 sequences identified from the mass spectrometry workflow. Nodes (CDR3 sequences) were connected by edges defined by a Damerau-Levenshtein distance of no more than 3, forming 183 isolated components. A thicker edge indicates a smaller distance value, i.e. a closer relation. (2C) Dendrogram showing sequence relationships between the 116 selected nanobodies, demonstrating that the repertoire generally retains significant diversity in both anti-S1 and anti-S2 nanobodies, albeit with a few closely related members. Scale, 0.2 substitutions per residue.

FIGS. 3A-3C. Binding of nanobody candidates to immobilized antigen; related to FIGS. 7A-7G. All nanobody candidates identified by (3A) S1, (3B) S1-RBD, or (3C) S2 purification were expressed in bacterial periplasm, which was bound to the respective immobilized antigen protein. After washes, loaded input (L) and elution (E) samples were analyzed by Coomassie stained SDS-PAGE. Positive binders (boxed) displayed a nanobody band in the elution, while negative candidates (unboxed) had none.

FIGS. 4A-4C. Quantified antigen binding of nanobody candidates; related to FIGS. 7A-7G. All nanobody candidates were expressed in bacterial periplasm, which was bound to immobilized (4A) S1, (4B) S1-RBD, or (4C) S2 antigen protein. Bound nanobody was quantified by Coomassie staining after SDS-PAGE. Binding intensity against each antigen was normalized to the maximum observed binding among all nanobodies. Candidates with >20% maximum activity (dark grey bars) were selected for follow up, while others (candidates marked with * asterisks) were generally discarded.

FIG. 5. S1 nanobody characterization; related to FIGS. 7A-7G and FIGS. 10A-10K. Nanobodies against S1 were determined to bind RBD or non-RBD epitopes by their affinity for recombinant full-length S1 and/or S1 RBD protein. Binding kinetics against these two recombinant proteins were determined by SPR, with on rates, off rates, and KDs determined by Langmuir fits to binding sensorgrams unless otherwise noted. Nanobody melting temperatures were determined by DSF. Nanobodies were assayed for neutralization activity against a SARS-CoV-2 spike pseudotyped HIV-1 virus (PSV), with IC50s calculated from neutralization curves. Standard error of the mean (s.e.m.) is reported where replicates were available. “−”—Not determined. NA—No activity.

FIG. 6. S2 nanobody characterization; related to FIGS. 7A-7G and FIGS. 10A-10K. Binding kinetics of S2 nanobodies were determined by SPR using recombinant S2 protein, with on rates, off rates, and K_{D}s determined by Langmuir fits to binding sensorgrams unless otherwise noted. Nanobody melting temperatures were determined by DSF. Nanobodies were assayed for neutralization activity against a SARS-CoV-2 or SARS-CoV-1 spike pseudotyped HIV-1 virus (PSV), with IC50s calculated from neutralization curves. Standard error of the mean (s.e.m.) is reported where replicates were available. “−”—Not determined. NA—No activity.

FIGS. 7A-7G. Biophysical characterization of anti-SARS-CoV-2 spike nanobodies. (7A) Each nanobody plotted against their affinity (K_{D}) for their antigen separated into three groups based on their binding region on SARS-CoV-2 spike protein. The data points with a white 4-point star correspond to nanobodies that neutralize. The majority of nanobodies have high affinity for their antigen with K_{D}s below 1 nm. 10 nanobodies are not included in this plot as they were unable to be analyzed successfully using surface plasmon resonance (SPR). (7E) SPR sensorgrams for each of the three targets on SARS-CoV-2 spike protein of the nanobody repertoire, showing three representatives for each binding region. (7C) The association rate of each nanobody (kon) versus the corresponding dissociation rate (k_{off}). The majority of the nanobodies have fast association rates (≥10⁺⁵ M⁻¹s⁻¹), with many surpassing the kₒₙ of high performing monoclonal antibodies and nanobodies with a kₒₙ>M⁻¹s⁻¹. (7D) Each nanobody plotted against their Tₘ as measured by differential scanning fluorimetry (DSF), revealing all but two nanobodies fall within the optimal Tₘ range (between 50° C. and 80° C.), where the bulk of the nanobodies have a Tₘ≥60° C. No data could be collected for two nanobodies and 10 nanobodies exhibited two dominant peaks in the thermal shift assay and were not included in this plot (a full summary of this data can be seen in FIGS. 5, 6, and 9). The K_{D} (7E) and T_{M} (7F) of six nanobodies were assessed pre- and post-freeze-drying, revealing no significant change in affinity or Tₘ after freeze-drying. (7G) SPR sensorgrams comparing the kinetic and affinity analysis of seven nanobodies against wildtype spike S1 (Wuhan str.), spike 20I/S1 501Y.V1 (alpha variant) and 20H/spike S1 501Y.V2 beta variant). In 7A and 7E, the x-axis reads, from left to right: S1-2, S1-3, S1-7, S1-9, S1-10, S1-24, S1-25, S1-30, S1-32, S1-41, S1-49, S1-50, S1-58, S1-60, S1-64, S1-66, S1-1, S1-4, S1-5, S1-6, S1-12, S1-14, S1-19, S1-20, S1-21, S1-23, S1-27, S1-28, S1-29, S1-31, S1-35, S1-36, S1-37, S1-38, S1-39, S1-46, S1-48, S1-51, S1-52, S1-53, S1-54, S1-55, S1-56, S1-61, S1-62, S1-63, S1-65, S1-RBD-3, S1-RBD-4, S1-RBD-5, S1-RBD-6, S1-RBD-9, S1-RBD-11, S1-RBD-12, S1-RBD-14, S1-RBD-15, S1-RBD-16, S1-RBD-18, S1-RBD-20, S1-RBD-21, S1-RBD-22, S1-RBD-23, S1-RBD-24, S1-RBD-25, S1-RBD-26, S1-RBD-27, S1-RBD-28, S1-RBD-29, S1-RBD-30, S1-RBD-32, S1-RBD-34, S1-RBD-35, S1-RBD-37, S1-RBD-38, S1-RBD-39, S1-RBD-40, S1-RBD-41, S1-RBD-43, S1-RBD-44, S1-RBD-45, S1-RBD-46, S1-RBD-47, S1-RBD-48, S1-RBD-49, S1-RBD-51, S2-1, S2-2, S2-3, S2-4, S2-5, S2-7, S2-9, S2-10, S2-11, S2-13, S2-14, S2-18, S2-26, S2-33, S2-35, S2-36, S2-40, S2-42, S2-47, S2-57, and S2-62.

FIGS. 8A-8G. Epitope characterization of nanobodies against the S1-RBD of SARS-CoV-2 spike. (8A) Major epitope bins are revealed by a clustered heat map of Pearson's Correlation Coefficients computed from the response values of nanobodies binding to the spike RBD in pairwise cross-competition assays on a biolayer interferometer. Correlated values (red) indicate that the two nanobodies respond similarly when measured against a panel of nine RBD nanobodies that bind to distinct regions of the RBD. A strong correlation score indicates binding to a similar/overlapping region on the RBD. Anti-correlated values (blue) indicate that a nanobody pair responds divergently when measured against nanobodies in the representative panel and indicate binding to distinct or non-overlapping regions on the RBD. The axes are labeld (from top to bottom on right y-axis and from left to right on x-axis): S1-6, S1-39, S1-35, S1-4, S1-RBD-22, S1-1, S1-RBD-12, S1-RBD-14, S1-RBD-29, S1-RBD-4, S1-RBD-36, S1-RBD-9, S1-RBD-16, S1-RBD-10, S1-RBD-24, S1-RBD-30, S1-RBD-25, S1-RBD-32, S1-RBD-34, S1-RBD-19, S1-RBD-26, S1-RBD-44, S1-RBD-48, S1-RBD-51, S1-RBD-47, S1-RBD-49, S1-RBD-45, S1-RBD-46, S1-RBD-41, S1-RBD-37, S1-RBD-43, S1-RBD-21, S1-RBD-27, S1-RBD-20, S1-RBD-40, S1-RBD-15, S1-RBD-28, S1-RBD-3, S1-RBD-5, S1-RBD-6, S1-31, S1-27, S1-36, S1-23, S1-62, S1-RBD-18, S1-28, S1-20, S1-RBD-35, S1-27, S1-48, S1-RBD-23, S1-RBD-38, S1-RBD-11, S1-29, and S1-46. (8E) As in (8A), but for sixteen S1 non-RBD-binding nanobodies. (8C) As in (8A), but for nineteen S2-binding nanobodies. (8D) A network visualization of anti-S1-RBD nanobodies. Each node is a nanobody and each edge is a response value measured by biolayer interferometry from pairwise cross-competition assays. Nodes with a “+” represent eleven nanobodies used as a representative panel for clustering analysis in (8A). Blue nodes represent the other nanobodies in the dataset. The average shortest distance between any nanobody pair in the dataset is 1.64. An average clustering coefficient of 0.831 suggests that the measurements are well-distributed across the dataset. The small world coefficient of 1.031 indicates that the network is more connected than to be expected from random, but the average path length is what you would expect from a random network, together indicating that the relationship between nanobody pairs not actually measured can be inferred from the similar/neighboring nanobodies. (8E,8F) As in (8D) but for S1 non-RBD and S2 nanobodies, respectively. These are complete networks with every nanobody measured against the others in the dataset. (8G) Mass photometry (MP) analysis of spike S1 monomer incubated with different anti-spike S1 nanobodies. Two examples of an increase in mass as spike S1 monomers (black line) are incubated with one to three nanobodies. The accumulation in mass upon addition of each different nanobody on spike S1 monomer is due to each nanobody binding to non-overlapping space on spike S1, an observation consistent with Octet binning data. As a control, using MP, each individual nanobody was shown to bind spike S1 monomers on its own (data not shown).

FIG. 9. Nanobody binding activity against spike S1 variants; related to FIGS. 7A-7G. Binding kinetics against wild-type spike S1 or two variants of concern were determined by SPR, with on rates, off rates, and K_{D}s determined by Langmuir fits to binding sensorgrams.

FIGS. 10A-10K. Diverse and potent nanobody-based neutralization of SARS-CoV-2. Nanobodies targeting the S1-RBD, S1 non-RBD, and S2 portions of spike effectively neutralize lentivirus pseudotyped with various SARS-CoV spikes and their variants from infecting ACE2 expressing HEK293T cells. (10A) Of the 116 nanobodies, monomers that neutralize SARS-CoV-2 pseudovirus with IC50 values 20 nM and lower are displayed. (10B) Representative nanobodies targeting the non-RBD portions of S1 and (10C) the S2 domain of SARS-CoV-2 neutralize SARS-CoV-2 pseudovirus. (10D-10F) Oligomerization of RBD, S1 non-RBD and S2 nanobodies significantly increases neutralization potency. (10G) Summary scatter plot of all nanobody IC50s across the major domains of SARS-CoV-2 spike and where tested, across SARS-CoV-2 variant 20H/501Y.V2 and SARS-CoV-1. Representative published nanobodies were also tested in the neutralization assays and show similar potency towards SARS-CoV-2 pseudovirus. The citations include Xiang et al., 2020, Science 370, 1479-1484 and Wrapp et al., 2020, Science 367, 1260-1263. (10H-10K) Representative SARS-CoV-2 RBD targeting nanobodies cross-neutralize the 201/501Y.V1/alpha variant with H69-, V70-, Y144-amino acid deletions and N501Y, A570D, D614G, P681H, T716I, S982A, and D1118H amino acid substitutions in spike (10H); the 20H/501Y.V2/beta variant with L18F, D80A, K417N, E484K, and N501Y amino acid substitutions in spike; 20J/501Y.V3/gamma variant with L18F, T20N, P26S, D138Y, R190S, K417T, E484K, N501Y, D614G, H655Y, T1027I, and V1176F amino acid substitutions in spike (10J); and SARS-CoV-1 spike pseudotyped lentivirus (10K). In all cases, ngreater than or equal to 2 biological replicates of each nanobody monomer/oligomer with a representative biological replicate with n=4 technical replicates per dilution displayed. See also FIGS. 5, 6, 11 and 12.

FIG. 11. Characterization of oligomerized spike nanobodies; related to FIGS. 10A-10K. Nanobody oligomers (1-4 nanobody repeats) were assayed for neutralization activity against a SARS-CoV-2 spike pseudotyped HIV-1 virus (PSV), with IC50s calculated from neutralization curves. Standard error of the mean (s.e.m.) is reported where replicates were available. Epitopes were determined by relative affinity for recombinant S1 or S1 RBD protein.

FIG. 12. Nanobody neutralization activity against spike variants; related to FIGS. 10A-10K. Nanobodies were assayed for neutralization activity against a pseudotyped HIV-1 virus (PSV) expressing SARS-CoV-1 or SARS-CoV-2 wild-type or variant spike, with IC50s calculated from neutralization curves. Standard error of the mean (s.e.m.) is reported where replicates were available.

FIG. 13. Authentic SARS-CoV-2 neutralization by anti-spike nanobodies. Nanobodies neutralize the authentic SARS-CoV-2 virus with similar kinetics as the SARS-CoV-2 pseudovirus. Neutralization curves are plotted from the results of a focus-forming reduction neutralization assay with the indicated nanobodies. Serial dilutions of each nanobody were incubated with SARS-CoV-2 (MOI 0.5) for 60 min and then overlaid on a monolayer of Vero E6 cells and incubated for 24 h. LaM2, an anti-mCherry nanobody (Fridy et al., 2014, Nat Methods 11, 1253-1260) was used as a non-neutralizing control. After 24 h, cells were collected and stained with anti-spike antibodies and the ratio of infected to uninfected cells was quantified by flow cytometry.

FIG. 14. Crosslinked residues used to map binding domains. The indicated nanobodies were bound to RBD, NTD, or the spike ectodomain and crosslinked with DSS (disuccinimidyl suberate). Crosslinked complexes were excised from SDS-PAGE gels, reduced, alkylated, and digested with either trypsin or chymotrypsin. Peptides were extracted and analyzed by mass spectrometry. Crosslinked residues (Iisted) were identified using pLink, and spectra were manually validated to eliminate false positives.

FIG. 15. Mass photometry of non-RBD S1 nanobodies; related to FIGS. 8A-8C. Mass photometry (MP) analysis of spike S1 monomer incubated with different non-RBD binding anti-spike S1 nanobodies. There is either an accumulation in mass upon addition of each different nanobody on spike S1 monomer which is due to each nanobody binding to non-overlapping space on spike S1 or not, suggesting overlapping epitopes. These results coupled with SPR binning data, assisted the binning analysis for non-RBD anti-S1 nanobodies.

FIG. 16. Nanobody synergy of neutralization activity; related to FIG. 18. Parameters from modeling the synergy observed for the indicated nanobody pairs. MuSyC, equivalent dose, and BRAID models were used to determine if statistically significant synergy was evident from the neutralization response in a 2D grid of nanobody concentrations (FIGS. 17A-17J).

FIGS. 17A-17J. Heatmaps of nanobody synergy; related to FIG. 18. Neutralization of pseudovirus harboring the SARS-CoV-2 spike by pairs of nanobodies. The normalized % neutralization is visualized on a 2D grid of nanobody concentrations where each nanobody has been titrated in the background of the other tested nanobody. Nanobody concentrations are indicated on their respective axes for: (17A) S1-23 and S1-27, (17B) S1-23 and S1-1, (17C) S1-RBD-15 and S1-23, (17D) S1-RBD-15 and S1-RBD-23, (17E) S1-23 and S1-46, (17F) S1-RBD-15 and S1-46 (17G) S1-49 and S1-1, (17H) S1-49 and S1-RBD-15, (17I) S1-23 and S2-10-dimer, and (17J) S1-RBD-15 and S2-10-dimer.

FIGS. 18A-18J. Synergistic neutralization of spike with nanobody cocktails. (18A) An example of additive effects between two anti-SARS-CoV2 spike nanobodies. S1-23 and S1-27 were prepared in a two-dimensional serial dilution matrix and then incubated with SARS-CoV-2 pseudovirus for 1 h before adding the mixture to cells. After 56 h, the expression of luciferase in each well was measured by addition of Steady-Glo reagent and read out on a spectrophotometer.

The left panel shows a heat map of pseudovirus neutralization by a two-dimensional serial dilution of combinations of S1-23 and S1-27. Lines and red numbers demarcate the % inhibition, that is, inhibitory concentration where X % of the virus is neutralized, e.g. IC50. Dark blue regions are concentrations that potently neutralize the pseudovirus, as per the heat map legend. The right panel shows neutralization curves (with 90% confidence interval bands) and the calculated IC50 of each nanobody alone, or in a 1:1 combination was determined along with a calculated IC50 based on the theoretical additive mixture model of the pair (curve with dotted gray line). The inset shows a difference (synergy) map calculated as the difference between the parameterized 2D neutralization response and that expected in a null model of only additive effects. Here, no difference is observed. (18B) S1-1 synergizes with S1-23 in neutralizing SARS-CoV-2 pseudovirus. The left panel shows the heatmap of pseudovirus neutralization observed by a two-dimensional serial dilution of combinations of S1-1 and S1-23. The middle panel shows a heat map mapping the synergy of neutralization observed for this pair. The lines bounding the darker purple areas demarcate regions in the heat map where the observed neutralization is greater than additive by the indicated percentages (yellow numbers), as per the heat map legend. (18C-18J) Examples of synergy between nanobodies binding the S1-RBD, or between the S1-RBD and S1-NTD or S2 domains of spike. The layout is as found in 18B, but comparing (18C) S1-RBD-15 with S1-23, (18D) S1-RBD-15 with S1-RBD-23, (18E) S1-23 with S1-46, (18F) S1-RBD-15 with S1-46, (18G) S1-49 with S1-1, (18H) S1-49 with S1-RBD-15, (18I) S1-23 with S2-10-dimer, and (18J) S1-RBD-15 with S2-10-dimer.

FIG. 19. Key Resources Table. Table of reagent or resource, the source, and identifier for the reagent or resource.

FIG. 20. DSS-Crosslinked Single-domain antibody-RBD Peptides Used for Modeling. S1-1, S1-23, and S1-RBD-15 single-domain antibodies were bound to RBD and crosslinked with DSS (disuccinimidyl suberate). Crosslinked complexes were excised from SDS-PAGE gels, reduced, alkylated, and digested with either trypsin or chymotrypsin. Peptides were extracted and analyzed by mass spectrometry. Crosslinked peptides (Iisted) and residues (indicated by asterisk) were identified using pLink, and spectra were manually validated to eliminate false positives.

FIG. 21. Additional single domain antibodies.

### DETAILED DESCRIPTION

Major immunotherapeutic strategies against SARS-CoV-2 to date have focused on immune sera and human monoclonal antibodies; however, these therapies now face the emergence of variants, including RBD point mutants, that have evolved to bypass the most potent neutralizing human antibodies, which are the very basis of immunotherapies (Garcia-Beltran et al., 2021, Cell 184(9):2372-2383; Liu et al., 2021, Cell Host Microbe 29, 477-488 e474; Starr et al., 2021, Science 371, 850-854; Wang et al., 2021, Nature 592(7855):616-622; Weisblum et al., 2020, Elife 9). A specific alternative class of single chain monoclonal antibodies, commonly called nanobodies, can be attractive alternatives to traditional monoclonal antibodies (Muyldermans, 2013, Annual review of biochemistry 82, 775-797). Nanobodies are the smallest single domain antigen binding proteins identified to date, possessing several potential advantages over conventional monoclonal antibodies. Nanobodies are naturally derived from the variable domain (VHH) of variant heavy chain-only IgGs (HCAb) found in camelids (e.g. llamas, alpacas, and camels), can bind in modes different from typical antibodies, covering more chemical space and binding with very high affinities (comparable to the very best antibodies) (Jovcevska and Muyldermans, 2020, BioDrugs 34, 11-26; Muyldermans, 2013, Annual review of biochemistry 82, 775-797). Their small size (often 15 kDa) allows them to bind tightly to otherwise inaccessible epitopes that may be (De Genst et al., 2006, Proc Natl Acad Sci USA 103, 4586-4591; Nam et al., 2016, Proc Natl Acad Sci USA 113, 14970-14975) obscured by the glycoprotein coat and so be unavailable to larger antibodies (Laursen et al., 2018, Science 362, 598-602; Zare et al., 2021, Mol Cell Probes 55, 101692). In certain embodiments, and as in conventional or classic antibodies, described below, VHH have three complementarity determining regions (CDRs) and four framework regions (FR). For additional information regarding VHH, see, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

Nanobodies are generally highly soluble, stable, lack glycans and are readily cloned and expressed in bacteria (Muyldermans, 2013, Annual review of biochemistry 82, 775-797). They have low immunogenicity (Bannas et al., 2017, Frontiers in immunology 8, 1603; Jovcevska and Muyldermans, 2020, BioDrugs 34, 11-26; Revets et al., 2005, Expert Opin Biol Ther 5, 111-124.) and can be readily modified to be “humanized” (including Fc addition), to change clearance rates, to add cargos such as drugs or fluorophores or to combine and improve characteristics by multimerization (Chanier and Chames, 2019, Antibodies (Basel) 8; Duggan, 2018, Drugs 78, 1639-1642; Vincke et al., 2009, J Biol Chem 284, 3273-3284). In the case of respiratory viruses like SARS-CoV-2, nanobodies' flexibility in drug delivery is a critical advantage. Beyond typical administration methods, a major advantage of nanobodies is their potential for direct delivery by nebulization deep into the lungs (W6lfel et al., 2020, Nature 581: 465-469). This route can provide a high local concentration in the airways and lungs to ensure rapid onset of therapeutic effects, while limiting the potential for unwanted systemic effects (Erreni et al., 2020, Biomolecules 10) as exemplified by clinical trials (Van Heeke et al., 2017, Pharmacol Ther 169, 47-56; Zare et al., 2021, Mol Cell Probes 55, 101692). Nonetheless, many potentially neutralizing nanobodies published to date suffer from the same problem as human monoclonals, in that they also recognize regions of RBD that are subject to escape variation, reducing their potential efficacy (Sun et al., 2021, bioRxiv; Wang et al., 2021, Nature 592(7855):616-622).

The current disclosure provides a large number of high affinity nanobodies to exploit the available epitope and vulnerability landscape of the SARS-CoV-2 Spike protein. The resulting library provides a repertoire of synergistically potent and escape resistant therapeutics.

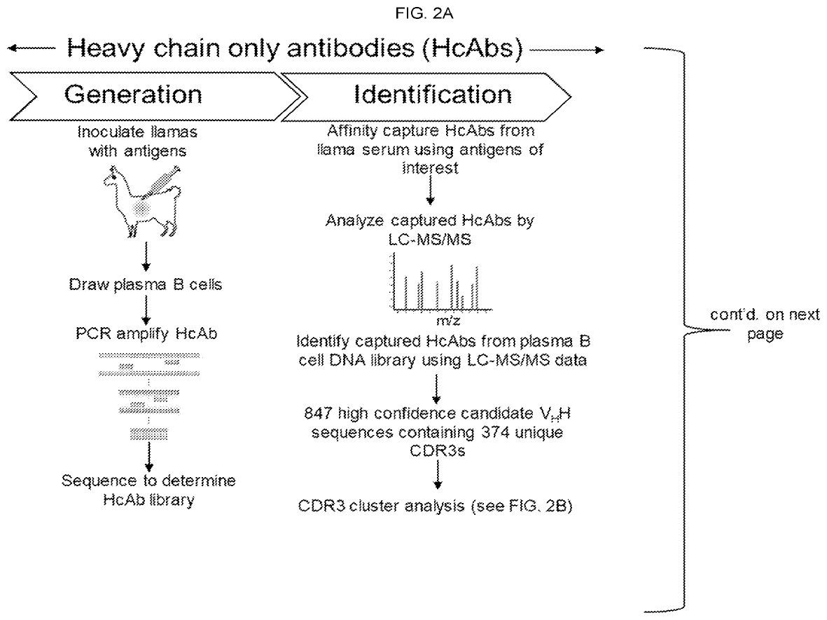

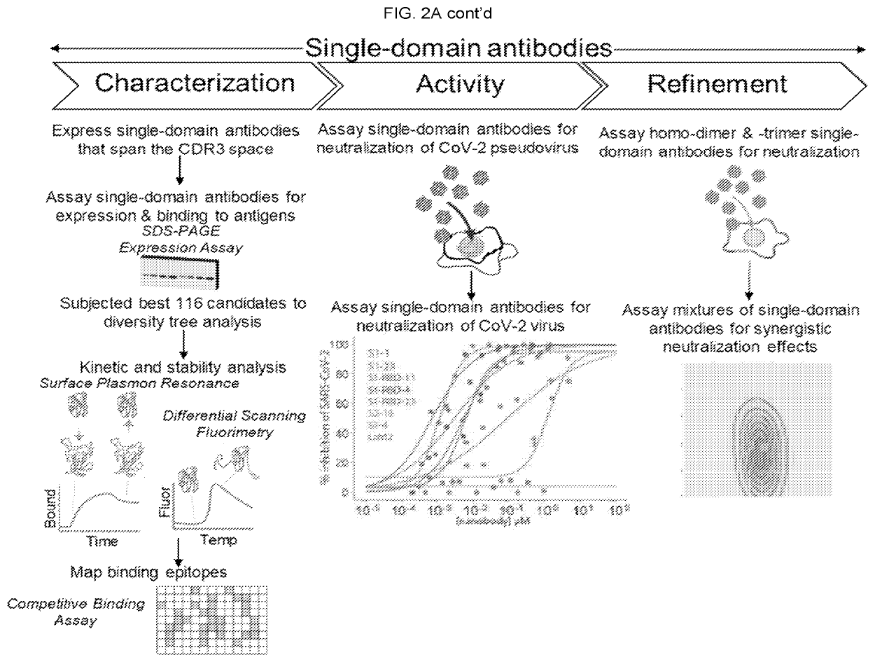

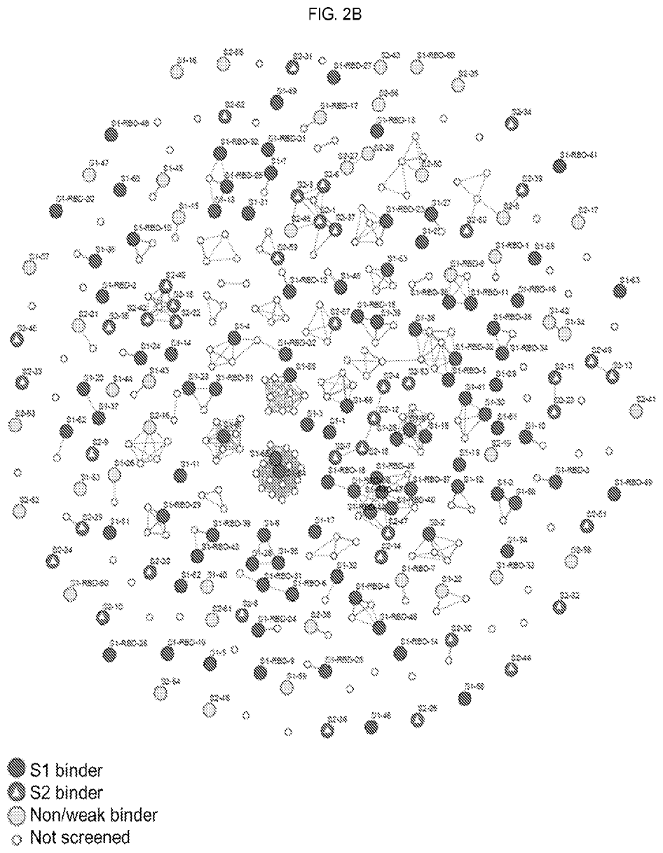

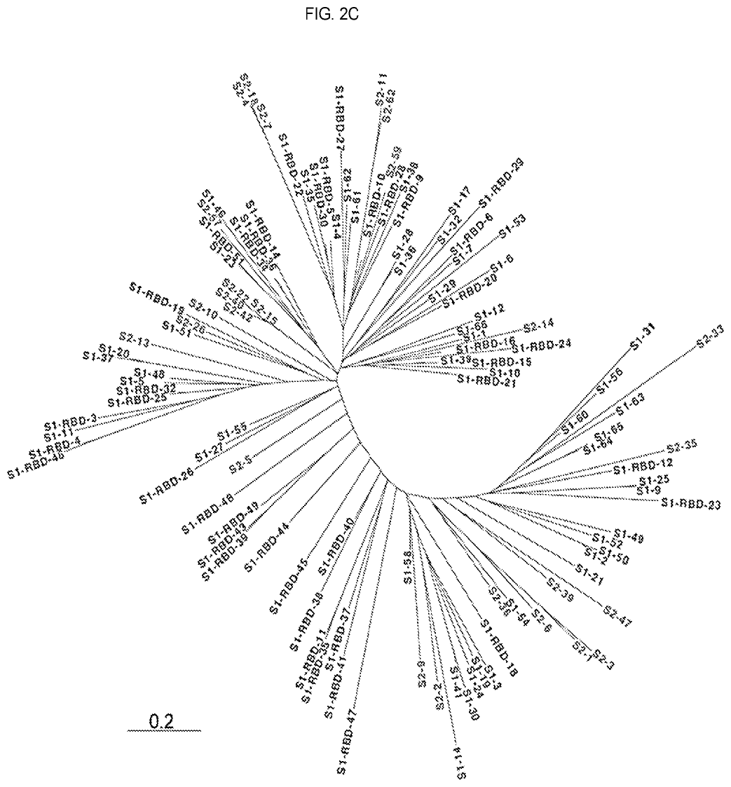

The protocol depicted in FIG. 2A was utilized to obtain an initial collection of single-domain antibodies. The depicted protocol allowed identification of 374 unique CDR3 sequences (from 847 unique VHH candidates). To maximize sequence diversity and thus the paratope space being explored, CDR sequences were clustered, revealing that many of the candidates form clusters likely to have similar antigen binding behavior. Here, partitioning of the clusters was performed by requiring that CDR3s in distinct clusters differ by a distance of more than three Damerau-Levenshtein edit operations (Bard, 2007, In Proceedings of the Fifth Australasian Symposium on ACSW Frontiers: Ballarat, Australia, Jan. 30-Feb. 2, 2007 Conferences in Research and Practice in Information Technology (Darlinghurst, Australia: Australian Computer Society, Inc.), pp. 117-124)—i.e., each operation being defined by insertion, deletion, or substitution of an amino acid, or transposition of two adjacent amino acids (FIG. 2B). This partitioning was found to be effective in that virtually no overlap was observed between those directed against S1 versus S2. The lengths of these CDR3 candidates also varied considerably, ranging from 3-22 amino acids in length. The use of two animals further expanded the paratope diversity in that only 4 out of 22 possible clusters from the second animal were observed to be shared with the first animal. In addition, relatively little overlap between disclosed CDR3 clusters and those observed by other groups were detected; for example, just 1 out of 109 S1 specific clusters (Damerau-Levenshtein s 3) were shared by (Xiang et al., 2020, Science 370, 1479-1484) and the present disclosure—indicating that the disclosed nanobody library is highly orthogonal.

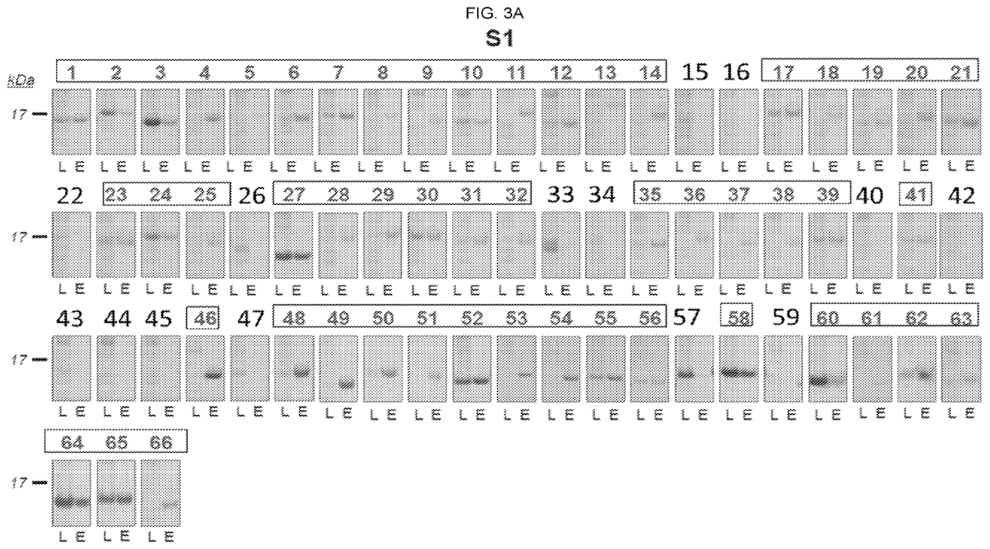

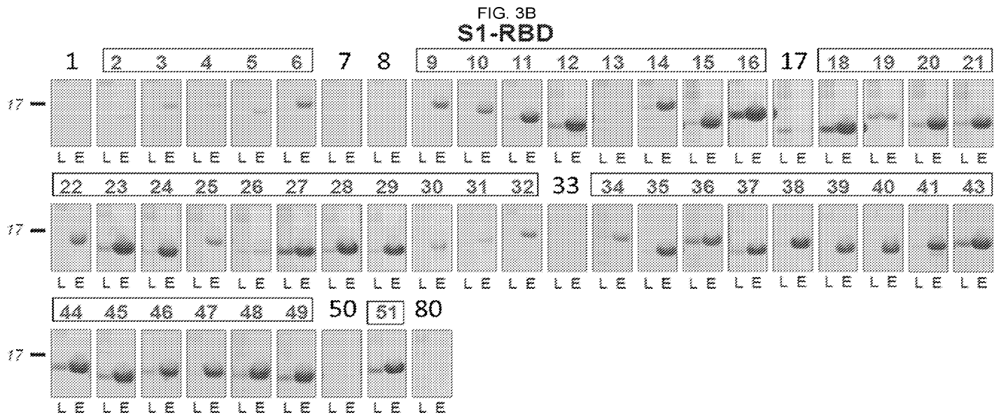

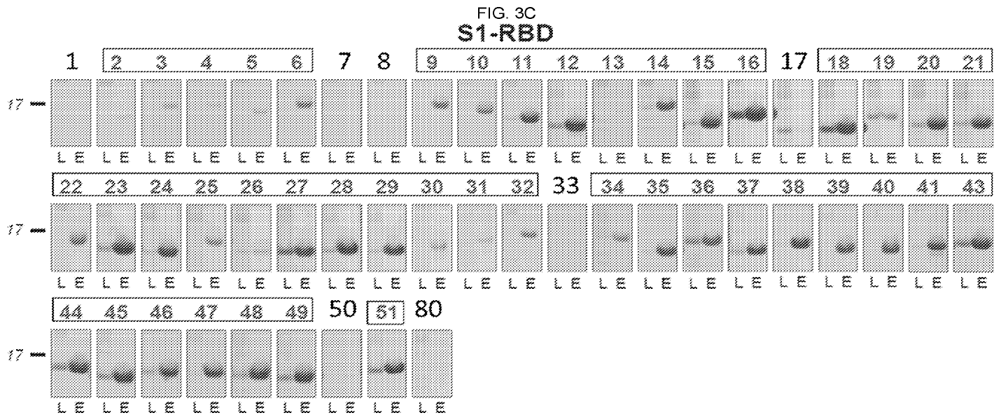

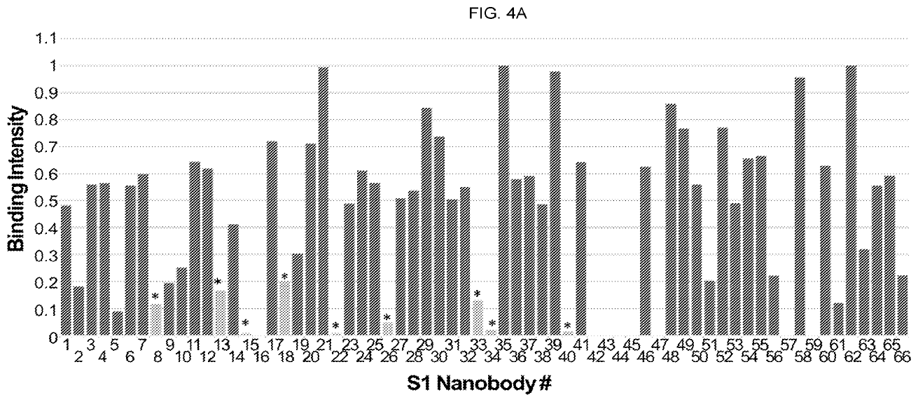

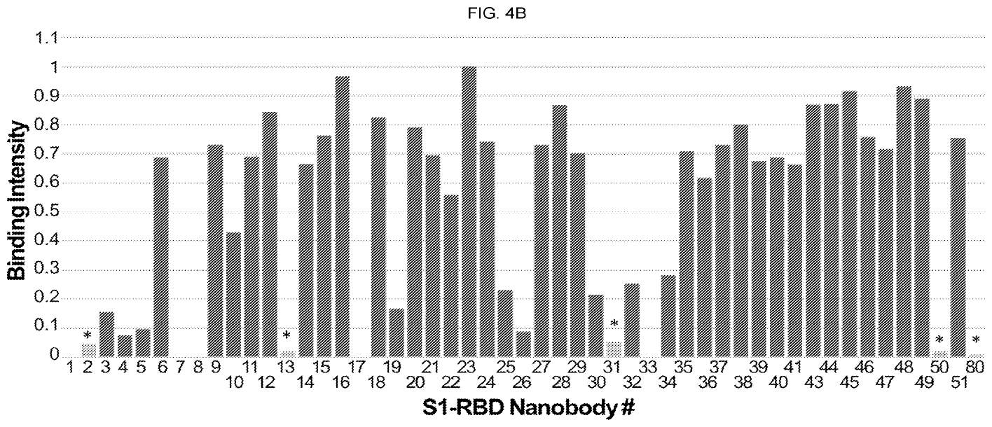

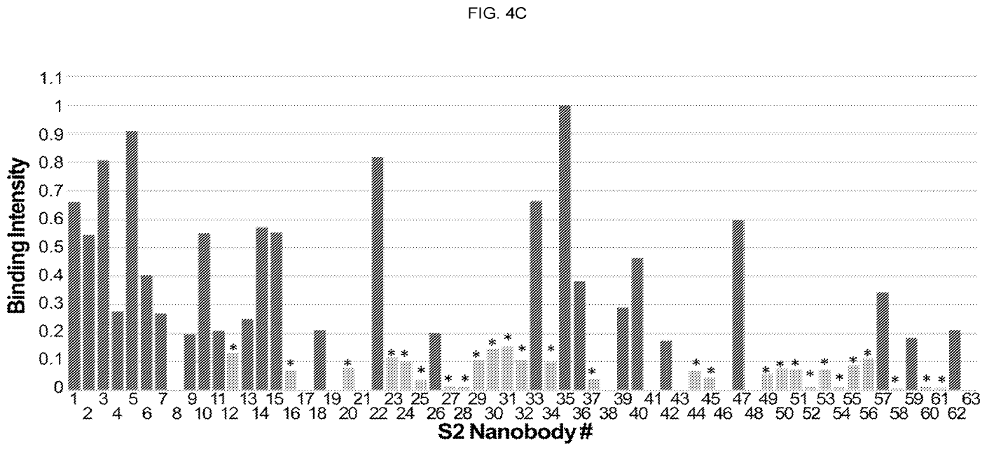

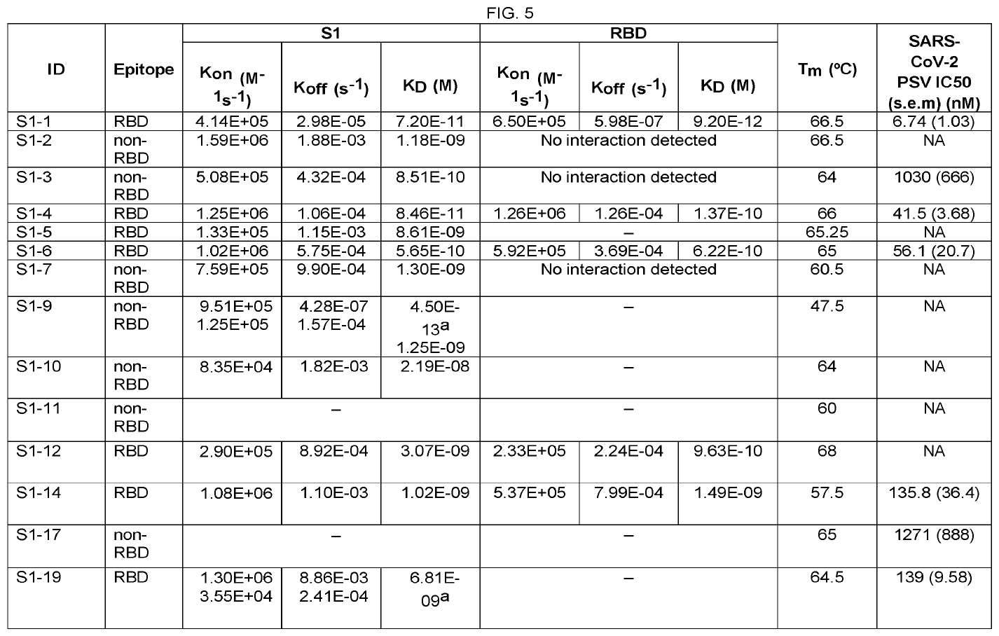

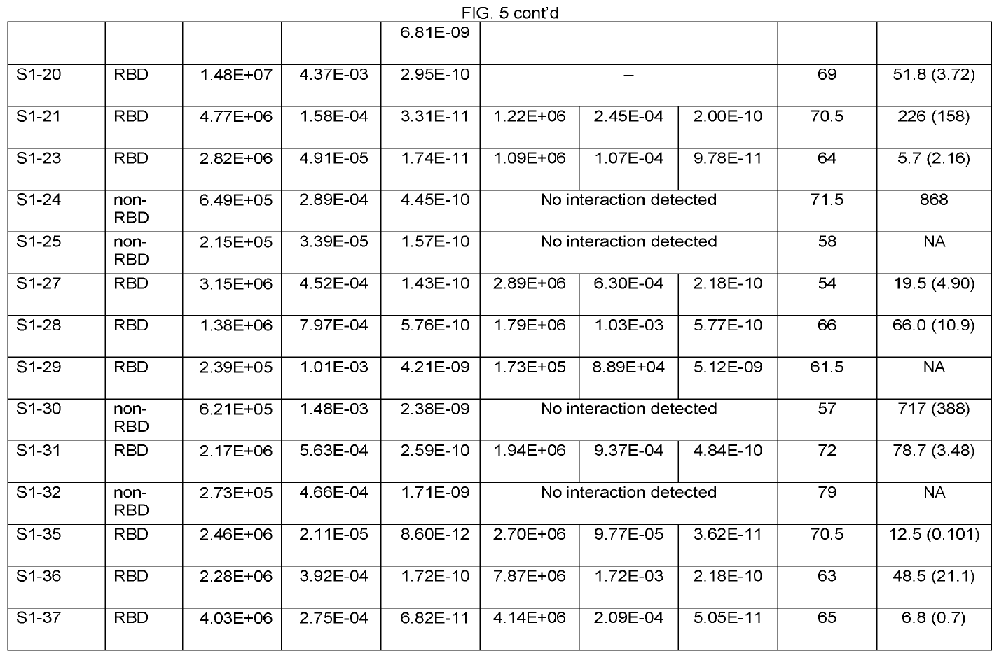

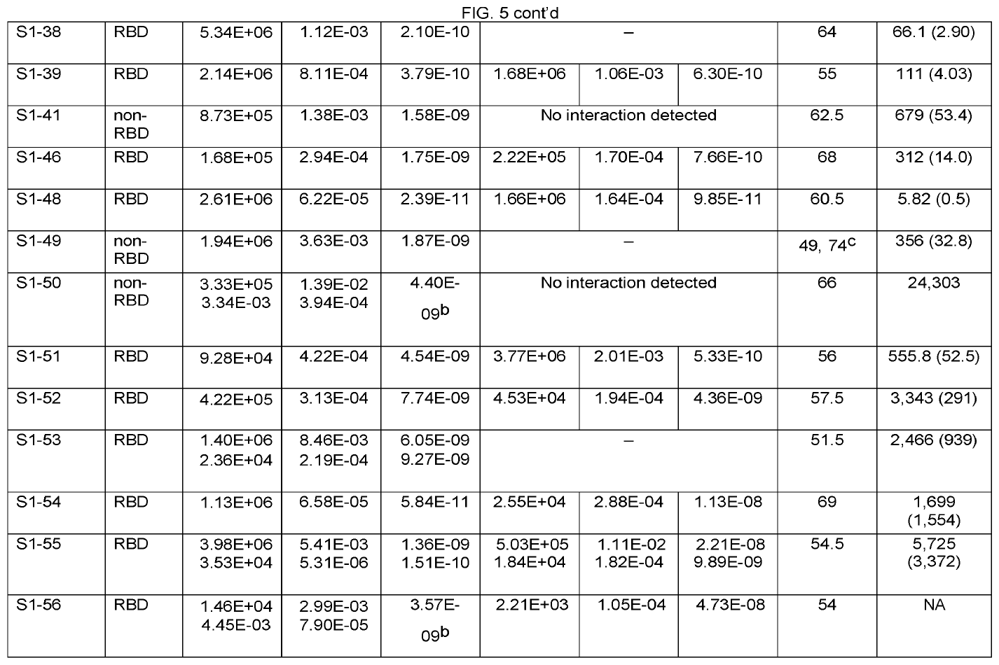

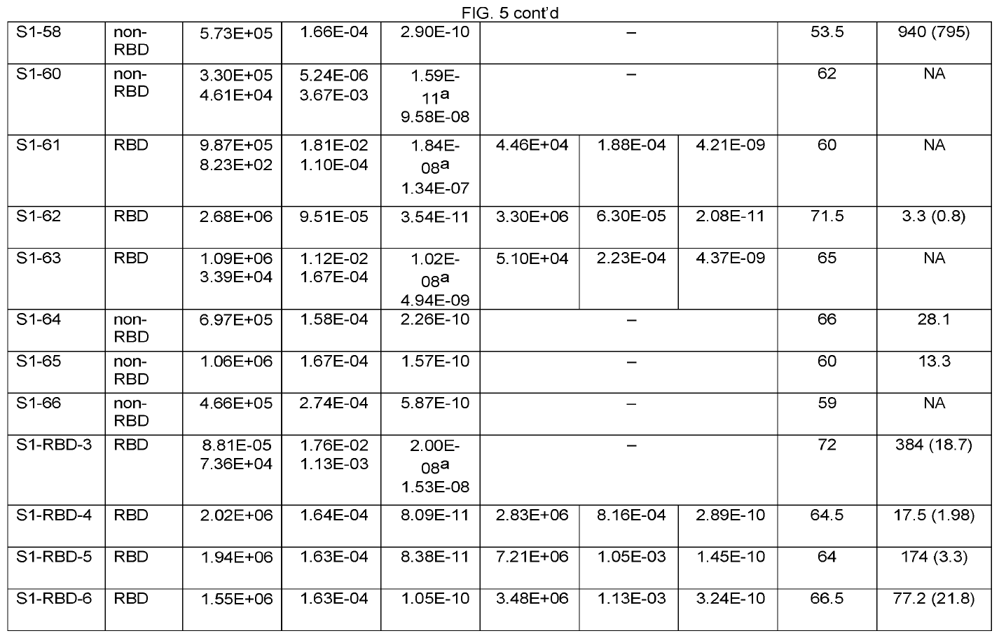

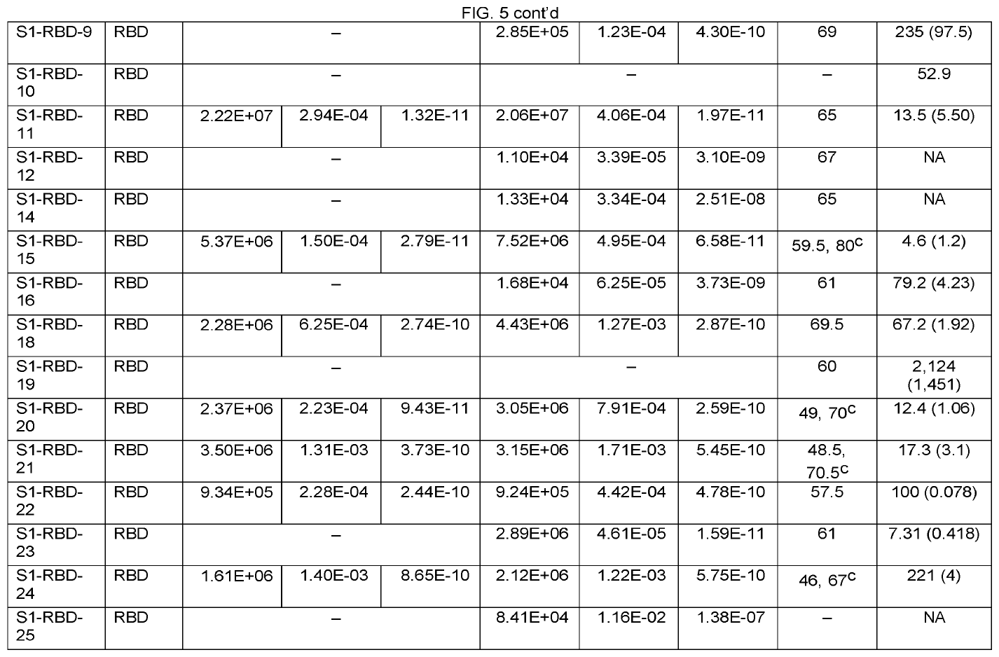

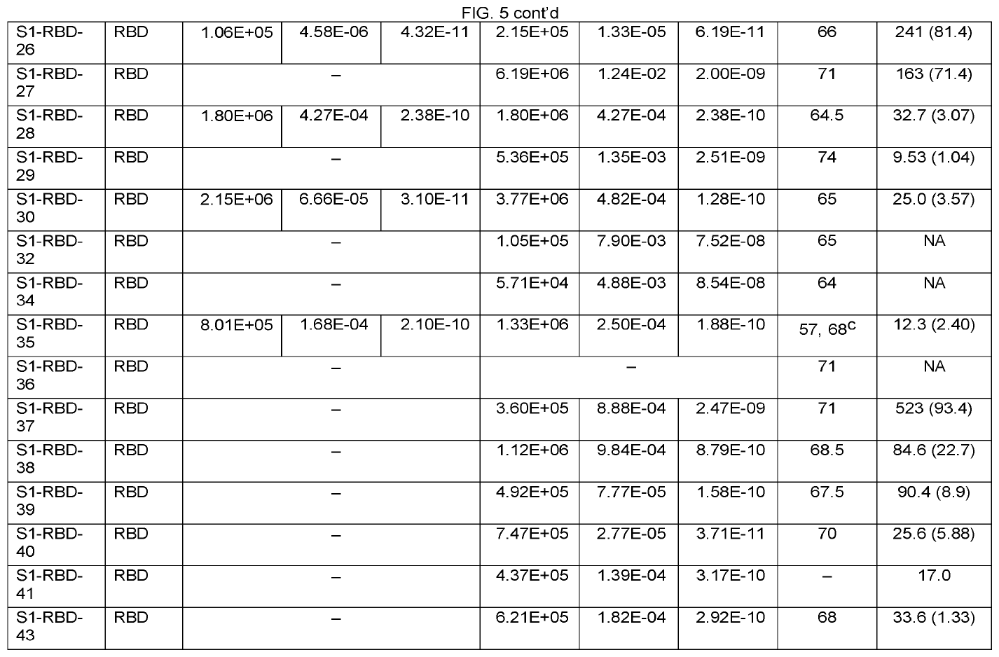

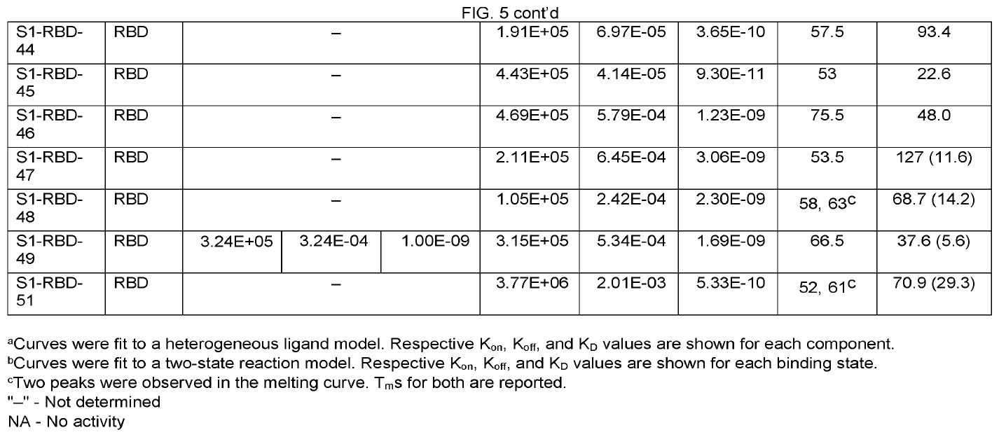

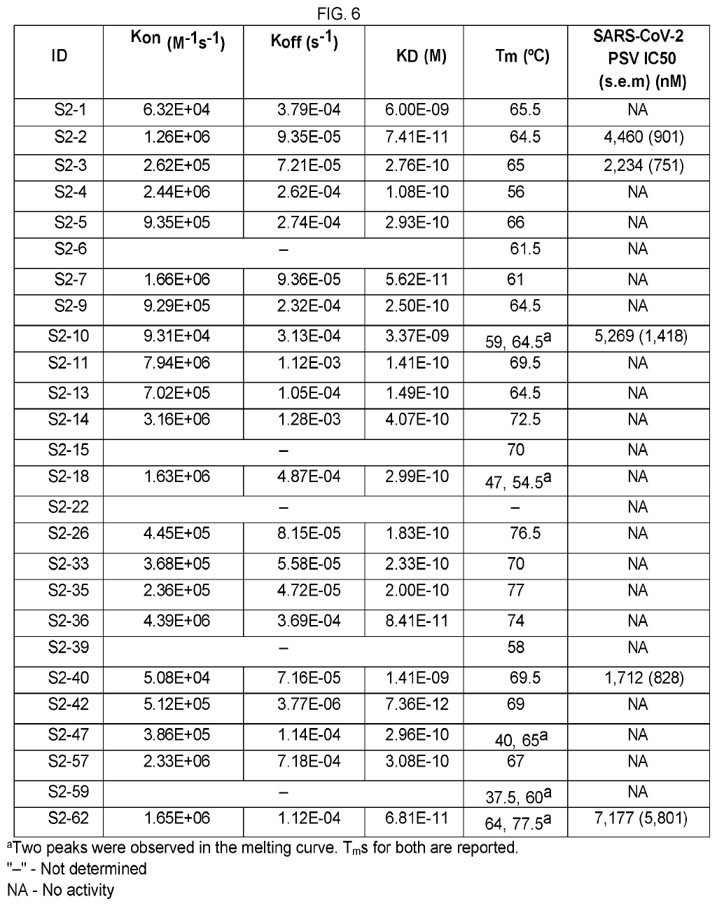

177 high-confidence candidates were selected for expression and screening. Of these, 63 were from S1 affinity purification, 63 from S2, and 51 from RBD, numbered S1-n, S2-n, and S1-RBD-n respectively. These were then expressed with periplasmic secretion in bacteria, and crude periplasmic fractions were bound to the corresponding immobilized spike antigen to assay recombinant expression, specific binding, and degree of binding (FIGS. 3A-3C, 4A-4C). 135 candidates were validated by this screen: 49 against S1, 42 against S2, and 44 against RBD (FIG. 2B). To eliminate candidates with the weakest expression and binding affinity, only nanobodies with binding intensity >20% of the observed maximum across all those screened were chosen for further development. This filtering identified the top 116 nanobodies that were purified for further characterization (FIGS. 5, 6). Note that these selections were designed to provide a strict cutoff in the interests of maximizing the quality of the library selected for thorough characterization but eliminated many additional bona fide nanobodies that bind to S1 and S2. While a few of these 116 nanobodies were chosen to share similar paratopes, overall, the group retained a high sequence and paratope diversity (FIG. 2C).

Particular single-domain antibodies disclosed herein include:

TABLE 1
Single-Domain Antibody & CDR* Sequences

| | Portion | | | | |
|---|---|---|---|---|---|
| | of SARS- | | | | |
| | CoV-2 | | | | |
| | spike | Variable Heavy | | | |
| Ab ID | protein | Domain | CDR1 | CDR2 | CDR3 |
| S1-1 | Bound | MAQVQLVESGGG | MPFSSYAM | AVNWNGIST | DSKGWSEW |
| | | LVQAGGSLRLSCA | G (SEQ ID | Y (SEQ ID | GMDY (SEQ |
| | | ASGMPFSSYAMG | NO: 117) | NO: 118) | ID NO: 119) |
| | | WFRQAPGKEREF | | | |
| | | VAAVNWNGISTYL | | | |
| | | ADSVKGRFTASR | | | |
| | | DNAKNTAYLQMN | | | |
| | | SLKPEDTAVYYCA | | | |
| | | ADSKGWSEWGM | | | |
| | | DYWGKGTLVTVS | | | |
| | | (SEQ ID NO: 1) | | | |
| S1-2 | non-RBD | MAQTQLEESGGG | SIIRPNVMA | SIHSSGTKN | RSTWATQP |
| | | LVQAGGSLRLSCA | (SEQ ID NO: | (SEQ ID NO: | DY (SEQ ID |
| | | ASGSIIRPNVMAW | 120) | 121) | NO: 122) |
| | | YRQGPGKQRELV | | | |
| | | ASIHSSGTKNYAD | | | |
| | | SVKGRFTISRDNA | | | |
| | | KNTVYLQMNSLKP | | | |
| | | EDTAVYYCATRST | | | |
| | | WATQPDYWGQG | | | |
| | | TQVTVS (SEQ ID | | | |
| | | NO: 2) | | | |
| S1-3 | non-RBD | MGQVQLVESGGG | STFSNTGLG | SISPSGSTS | LPRSGS |
| | | LVQAGGSLRLSCA | (SEQ ID NO: | (SEQ ID NO: | (SEQ ID NO: |
| | | ASGSTFSNTGLG | 123) | 124) | 125) |
| | | WYRQAPGKQRE | | | |
| | | WASISPSGSTSY | | | |
| | | LDSVRGRFIFSRD | | | |
| | | NAKNIAYLQMNSL | | | |
| | | KPEDTAVYYCNTL | | | |
| | | PRSGSWGQGTQV | | | |
| | | TVS (SEQ ID NO: | | | |
| | | 3) | | | |
| S1-4 | RBD | MAQVQLVESGGG | RTFSSYFMA | AVDWSGGD | DRGYSLYY |
| | | LVQAGGSLRLSCA | (SEQ ID NO: | TL (SEQ ID | YQAREYEY |
| | | AAGRTFSSYFMA | 126) | NO: 127) | (SEQ ID NO: |
| | | WFRQAPGKEREF | | | 128) |
| | | VAAVDWSGGDTL | | | |
| | | YTDSVKGRFTISR | | | |
| | | DNGANTVYLQMN | | | |
| | | NLKREDTAVYYCA | | | |
| | | ADRGYSLYYYQA | | | |
| | | REYEYWGQGTQV | | | |
| | | TVS (SEQ ID NO: | | | |
| | | 4) | | | |
| S1-5 | RBD | MAQVQLVESGGG | FTLGYYAIG | CIGSSDGST | KGPPQGVL |
| | | LVQPGGSLRLSCA | (SEQ ID NO: | Y (SEQ ID | RCSISGRYD |
| | | ASGFTLGYYAIGW | 129) | NO: 130) | F (SEQ ID |
| | | FRQAPGKEREGV | | | NO: 131) |
| | | SCIGSSDGSTYYA | | | |
| | | DSVKGRFTISRDN | | | |
| | | AKNTVYLQINSLK | | | |
| | | PEDTAVYYCATKG | | | |
| | | PPQGVLRCSISGR | | | |
| | | YDFWGQGTQVTV | | | |
| | | S (SEQ ID NO: 5) | | | |
| S1-6 | RBD | MAQVQLVESGGG | RTSSFFSMA | VIRGSGGVT | SDFGDHGP |
| | | LVQAGDSLRLSCV | (SEQ ID NO: | F (SEQ ID | LSGRTDFSS |
| | | VSGRTSSFFSMA | 132) | NO: 133) | (SEQ ID NO: |
| | | WFRQAPGKEREF | | | 134) |
| | | VAVIRGSGGVTFY | | | |
| | | SESVKGRFTISRD | | | |
| | | NAKNTVSLQMSSL | | | |
| | | KPEDTAVYYCAGS | | | |
| | | DFGDHGPLSGRT | | | |
| | | DFSSWGQGTQVT | | | |
| | | VS (SEQ ID NO: 6) | | | |
| S1-7 | non-RBD | MAQVQLAESGGG | RTYTSSMY | VIGWNTY | RKAAGLAS |
| | | LVQVGDSLRLSCA | GMA (SEQ | (SEQ ID NO: | GYPSPSYY |
| | | ASGRTYTSSMYG | ID NO: 135) | 136) | DY (SEQ ID |
| | | MAWFRQAPGKER | | | NO: 137) |
| | | EFVAVIGWNTYYA | | | |
| | | DPVKGRFTISRDN | | | |
| | | AKNIVYLQMNNLK | | | |
| | | PEDTAVYYCASRK | | | |
| | | AAGLASGYPSPSY | | | |
| | | YDYWGQGTQVTV | | | |
| | | S (SEQ ID NO: 7) | | | |
| S1-9 | non-RBD | MAQVRLVESGGG | VSGSAFRF | SISSGGSTN | RADFFSSTR |
| | | LVQPGGSLRLSCV | NRLG (SEQ | (SEQ ID NO: | TGD (SEQ |
| | | VSGVSGSAFRFN | ID NO: 138) | 139) | ID NO: 140) |
| | | RLGWYRQAPGKE | | | |
| | | RELVASISSGGST | | | |
| | | NYADSVKGRFTIS | | | |
| | | RDNAKSTVSLQM | | | |
| | | NSLKPEDTAVYYC | | | |
| | | NIRADFFSSTRTG | | | |
| | | DWGQGTQVTVS | | | |
| | | (SEQ ID NO: 8) | | | |
| S1-10 | non-RBD | MAQVQLVESGGG | RTFSSAGM | TSNWNGGD | HLQKYGGD |
| | | LVQAGGSLRLSCA | A (SEQ ID | TD (SEQ ID | YYARI (SEQ |
| | | ASGRTFSSAGMA | NO: 141) | NO: 142) | ID NO: 143) |
| | | WFRQAPGKEREF | | | |
| | | VATSNWNGGDTD | | | |
| | | YADSVKGRFTISR | | | |
| | | DNAKNTVYLQMN | | | |
| | | NLKPEDTAVYYCA | | | |
| | | AHLQKYGGDYYA | | | |
| | | RIWGQGTQVTVS | | | |
| | | (SEQ ID NO: 9) | | | |
| S1-11 | non-RBD | MAEVQLVESGGG | FTLSPYDIG | CISVTGGTT | HTASFCRG |
| | | LVQFGGSLRLSCA | (SEQ ID NO: | D (SEQ ID | DYSASVAR |
| | | ASGFTLSPYDIGW | 144) | NO: 145) | Y (SEQ ID |
| | | FRQAPGKEREGV | | | NO: 146) |
| | | SCISVTGGTTDYT | | | |
| | | DSVKGRFTISRDH | | | |
| | | ARNTVYLQMNNL | | | |
| | | KPEDTAVYYCAAH | | | |
| | | TASFCRGDYSASV | | | |
| | | ARYWGQGTQVTV | | | |
| | | S (SEQ ID NO: 10) | | | |
| S1-12 | RBD | MAQVQLVESGGG | RTFSNYAM | AISWKSGLI | DPNNRRGT |
| | | LVQAGGSLRLSCA | G (SEQ ID | T (SEQ ID | LTSYYDH |
| | | ASGRTFSNYAMG | NO: 147) | NO: 148) | (SEQ ID NO: |
| | | WFRQAPGKEREF | | | 149) |
| | | VVAISWKSGLITAA | | | |
| | | NFLEGRFTISRDN | | | |
| | | AKNTMYLQMNNL | | | |
| | | KPEDAAVYYCAV | | | |
| | | DPNNRRGTLTSYY | | | |
| | | DHWGQGTQVTVS | | | |
| | | (SEQ ID NO: 11) | | | |
| S1-14 | RBD | MAQVQLVESGGG | STFSRLSM | RILPLGGPY | APFGTAWD |
| | | LVQAGGSLRLSCA | G (SEQ ID | (SEQ ID NO: | GPDNYDY |
| | | ASGSTFSRLSMG | NO: 150) | 151) | (SEQ ID NO: |
| | | WYRQVPGKQREL | | | 152) |
| | | VARILPLGGPYYR | | | |
| | | DFVQGRFTISRDN | | | |
| | | VKNMLYLQMNSL | | | |
| | | KPEDTAVYYCNRA | | | |
| | | PFGTAWDGPDNY | | | |
| | | DYWGQGTQVTVS | | | |
| | | (SEQ ID NO: 12) | | | |
| S1-17 | non-RBD | MAQVQLVESGGG | LASTYSAKG | AINRSGGIT | KVTLSDDTY |
| | | LVQAGGSLRLSCP | (SEQ ID NO: | S (SEQ ID | NQPGNYPS |
| | | ASGLASTYSAKG | 153) | NO: 154) | (SEQ ID NO: |
| | | WFRQAPGKEREF | | | 155) |
| | | VAAINRSGGITSYA | | | |
| | | DSVKGRFTISRDD | | | |
| | | AKNTVYLQMNNLK | | | |
| | | PEDTAVYYCAAKV | | | |
| | | TLSDDTYNQPGN | | | |
| | | YPSWGQGTQVTV | | | |
| | | S (SEQ ID NO: 13) | | | |
| S1-19 | RBD | MAQVQLVESGGG | STFSSYAM | GISPDGSTN | FLPEIPGRG |
| | | LVRAGGSLRLSCA | G (SEQ ID | (SEQ ID NO: | S (SEQ ID |
| | | ASVSTFSSYAMG | NO: 156) | 157) | NO: 158) |
| | | WYRQAPGNQREL | | | |
| | | VAGISPDGSTNYA | | | |
| | | DSVKGRFTISRDN | | | |
| | | AKNTLVLQMNSLK | | | |
| | | SEDTAVYYCRIFL | | | |
| | | PEIPGRGSWGQG | | | |
| | | TQVTVS (SEQ ID | | | |
| | | NO: 14) | | | |
| S1-20 | RBD | MAQVQLVESGGG | FPLDFYAIG | CISRRDDYT | VRTSSGTV |
| | | LVQPGGSLRLSCA | (SEQ ID NO: | S (SEQ ID | CQAYPRWY |
| | | VSGFPLDFYAIGW | 159) | NO: 160) | DT (SEQ ID |
| | | FRQASGKEREWV | | | NO: 161) |
| | | SCISRRDDYTSYV | | | |
| | | DSVKGRFTISRDN | | | |
| | | AENTVYLQMNSLK | | | |
| | | LEDTAVYYCAGVR | | | |
| | | TSSGTVCQAYPR | | | |
| | | WYDTWGQGTQV | | | |
| | | TVS (SEQ ID NO: | | | |
| | | 15) | | | |
| S1-21 | RBD | MAQVQLVESGGG | IAVRASTMG | TINIGGSTN | QYYTGYDY |
| | | LVQAGGSLRLSCA | (SEQ ID NO: | (SEQ ID NO: | (SEQ ID NO: |
| | | VSGIAVRASTMG | 162) | 163) | 164) |
| | | WYRQAPGKQREL | | | |
| | | VATINIGGSTNYAD | | | |
| | | SVKGRFTIARDNA | | | |
| | | ENTAYLQMNSLKP | | | |
| | | EDTAVYYCKAQYY | | | |
| | | TGYDYWGRGTQV | | | |
| | | TVS (SEQ ID NO: | | | |
| | | 16) | | | |
| S1-23 | RBD | MAQVQLVESGGG | RTSRPYRM | AIFWSGSHT | RNQATGNY |
| | | LVQAGGSLRLSCV | G (SEQ ID | L (SEQ ID | DV (SEQ ID |
| | | ASGRTSRPYRMG | NO: 165) | NO: 166) | NO: 167) |
| | | WFRQAPGKEREF | | | |
| | | VAAIFWSGSHTLY | | | |
| | | EDSVKGRFTISTD | | | |
| | | NAQNTVYLQMNS | | | |
| | | LKPEDTAVYYCAA | | | |
| | | RNQATGNYDVWG | | | |
| | | QGTQVTVS (SEQ | | | |
| | | ID NO: 17) | | | |
| S1-24 | non-RBD | MAQVQPVESGGG | STTTNYHM | AINAGGITN | GGGWDYR |
| | | LVQAGGSLRLSCV | G (SEQ ID | (SEQ ID NO: | NSYYIPRVD |
| | | ASGSTTTNYHMG | NO: 168) | 169) | S (SEQ ID |
| | | WYRQTPGEQREL | | | NO: 170) |
| | | VAAINAGGITNYA | | | |
| | | DSVKGRFTISRDN | | | |
| | | AKNTMYLQMNNL | | | |
| | | RFEDTAVYYCNIG | | | |
| | | GGWDYRNSYYIP | | | |
| | | RVDSWGQGTQVT | | | |
| | | VS (SEQ ID NO: | | | |
| | | 18) | | | |
| S1-25 | non-RBD | MAQVRLVESGGG | VSGSGFRF | SISSGGSTN | RADFFTSTR |
| | | LVQPGGSLRLSCV | NRLG (SEQ | (SEQ ID NO: | TGD (SEQ |
| | | VSGVSGSGFRFN | ID NO: 171) | 139) | ID NO: 173) |
| | | RLGWYRQAPGKE | | | |
| | | RELVASISSGGST | | | |
| | | NYADSVKGRFTIS | | | |
| | | RDNAKNSVSLQM | | | |
| | | NSLKPEDTAVYYC | | | |
| | | NIRADFFTSTRTG | | | |
| | | DWGQGTQVTVS | | | |
| | | (SEQ ID NO: 19) | | | |
| S1-27 | RBD | MAQVQLVESGGG | GTFGSSRM | AIMPSGSHT | RDPSSYTY |
| | | LAQPGGSLRLSCA | G (SEQ ID | N (SEQ ID | DR (SEQ ID |
| | | ASAGTFGSSRMG | NO: 174) | NO: 175) | NO: 176) |
| | | WFRQAPGKEREF | | | |
| | | VAAIMPSGSHTNY | | | |
| | | ADSVKGRFTISRD | | | |
| | | NAKDTIYLQMNSL | | | |
| | | KPEDTAVYYCGA | | | |
| | | RDPSSYTYDRWG | | | |
| | | QGTQVTVS (SEQ | | | |
| | | ID NO: 20) | | | |
| S1-28 | RBD | MAQVQLVESGGG | RTLSSYVM | AIRWNGGS | SPRTMYYA |
| | | LVQAGGSLKLSCA | G (SEQ ID | TF (SEQ ID | SYYYTRTSY |
| | | VSGRTLSSYVMG | NO: 177) | NO: 178) | DY (SEQ ID |
| | | WFRQAPGKEREF | | | NO: 179) |
| | | VAAIRWNGGSTFY | | | |
| | | ADSVQGRLTISRD | | | |
| | | NAKNMVYLQMDS | | | |
| | | LKPEDTAAYYCAA | | | |
| | | SPRTMYYASYYYT | | | |
| | | RTSYDYWGQGTQ | | | |
| | | VTVS (SEQ ID NO: | | | |
| | | 21) | | | |
| S1-29 | RBD | MAQVQLVESGGG | DTFSSAPM | GISWSALNT | RTRSHFYSE |
| | | QAQAGDSLRLSC | G (SEQ ID | Y (SEQ ID | VYRRASDY |
| | | AASGDTFSSAPM | NO: 180) | NO: 181) | DF (SEQ ID |
| | | GWFRQAPGKERE | | | NO: 182) |
| | | LIAGISWSALNTYY | | | |
| | | ADSVKGRFTISRE | | | |
| | | DAKNTVYLQMNSL | | | |
| | | KQEDTAVYHCAA | | | |
| | | RTRSHFYSEVYRR | | | |
| | | ASDYDFWGQGTQ | | | |
| | | VTVS (SEQ ID NO: | | | |
| | | 22) | | | |
| S1-30 | non-RBD | MAQVPLVESGGG | STFSTSHM | SISTEGTTE | GGWRYYGD |
| | | LVQAGGSLRLSCT | G (SEQ ID | (SEQ ID NO: | GSHYIPDFR |
| | | TSGSTFSTSHMG | NO: 183) | 184) | F (SEQ ID |
| | | WYRQAPGKQREL | | | NO: 185) |
| | | VASISTEGTTEYA | | | |
| | | DFVKGRFTISRNN | | | |
| | | AQNTVYLEMASLK | | | |
| | | PEDTAVYHCNRG | | | |
| | | GWRYYGDGSHYI | | | |
| | | PDFRFWGQGTQV | | | |
| | | TVS (SEQ ID NO: | | | |
| | | 23) | | | |
| S1-31 | RBD | MAQVQLVESGGG | SISSIIYRG | SIDTAGSTT | VTWAPRVE |
| | | LVQPGGSLRLSCA | (SEQ ID NO: | (SEQ ID NO: | Y (SEQ ID |
| | | ASGSISSIIYRGWF | 186) | 187) | NO: 188) |
| | | RQVPGKEGEFVA | | | |
| | | SIDTAGSTTYADS | | | |
| | | VKGRFTISRDNAK | | | |
| | | NTLYLQMNSLKPE | | | |
| | | DTAVYYCKYVTW | | | |
| | | APRVEYRGQGTQ | | | |
| | | VTVS (SEQ ID NO: | | | |
| | | 24) | | | |
| S1-32 | non-RBD | MAQVQLVESGGG | VAFSLYHMA | AINWNGGIA | KLGAYYTTY |
| | | LVQAGGSLRLSCT | (SEQ ID NO: | D (SEQ ID | RKANEYQY |
| | | ASGVAFSLYHMA | 189) | NO: 190) | (SEQ ID NO: |
| | | WFRQAPGKEREF | | | 191) |
| | | VTAINWNGGIADY | | | |
| | | VDSVKGRFTISRD | | | |
| | | NAKNTVYLQMNSL | | | |
| | | KPEDTAVYYCAAK | | | |
| | | LGAYYTTYRKANE | | | |
| | | YQYWGQGTQVTV | | | |
| | | S (SEQ ID NO: 25) | | | |
| S1-35 | RBD | MAQVHLIESGGGL | RTFSSLVMA | FIEWSGAET | DKGYSLYYY |
| | | VQAGGSLRLSCV | (SEQ ID NO: | Y (SEQ ID | RESDYEY |
| | | ASGRTFSSLVMA | 192) | NO: 193) | (SEQ ID NO: |
| | | WFRQAPGKEREF | | | 194) |
| | | VAFIEWSGAETYY | | | |
| | | TDSVKGRFTISRD | | | |
| | | NAKNTVILQMNSV | | | |
| | | KPEDTGVYYCAA | | | |
| | | DKGYSLYYYRES | | | |
| | | DYEYWGQGTRVT | | | |
| | | VS (SEQ ID NO: | | | |
| | | 26) | | | |
| S1-36 | RBD | MAQVQLVESGGG | RILSSYVMG | AIRWNGGS | SPRTMYLAS |
| | | LVQAGGSLRVSC | (SEQ ID NO: | TF (SEQ ID | YYYHRTSY |
| | | AASERILSSYVMG | 195) | NO: 178) | DY (SEQ ID |
| | | WFRQAPGKEREF | | | NO: 197) |
| | | VAAIRWNGGSTFY | | | |
| | | ADSVKGRLTISRD | | | |
| | | NAKNTVYLQMNSL | | | |
| | | KPEDTAVYYCAAS | | | |
| | | PRTMYLASYYYHR | | | |
| | | TSYDYWGQGTQV | | | |
| | | TVS (SEQ ID NO: | | | |
| | | 27) | | | |
| S1-37 | RBD | MAQVQLVESGGG | FPLDFFAIG | CISRRDDYT | VRTSSDTVC |
| | | LVQPGGSLRLSCV | (SEQ ID NO: | S (SEQ ID | QNYPRWYL |
| | | VSGFPLDFFAIGW | 198) | NO: 160) | T (SEQ ID |
| | | FRQASGKEREWV | | | NO: 200) |
| | | SCISRRDDYTSYV | | | |
| | | DSVNGRFTISRDN | | | |
| | | AENTVYLQMNSLK | | | |
| | | LEDTAVYYCAGVR | | | |
| | | TSSDTVCQNYPR | | | |
| | | WYLTWGQGTQVT | | | |
| | | VS (SEQ ID NO: | | | |
| | | 28 | | | |
| S1-38 | RBD | MAQVQLVESGGG | RTFSYAIMG | WMAWSGP | SKEITTMTT |
| | | LVQAGGSLRLSCA | (SEQ ID NO: | AKY (SEQ ID | RYDY (SEQ |
| | | ASGRTFSYAIMG | 201) | NO: 202) | ID NO: 203) |
| | | WFRQAPGKDREF | | | |
| | | VGWMAWSGPAK | | | |
| | | YYADSVKGRFSIS | | | |
| | | RDNAKNTVYLQM | | | |
| | | NSLKPEDTAVYYC | | | |
| | | AASKEITTMTTRY | | | |
| | | DYWGQGTQVTVS | | | |
| | | (SEQ ID NO: 29) | | | |
| S1-39 | RBD | MAQVQLVESGGG | RTFGSYAM | AINWSGDH | RGPNEWGL |
| | | LVQAGGSLRLSCA | G (SEQ ID | TY (SEQ ID | PREDLFYDY |
| | | ASGRTFGSYAMG | NO: 204) | NO: 205) | (SEQ ID NO: |
| | | WFRQAPGKEREC | | | 206) |
| | | VSAINWSGDHTYY | | | |
| | | ADSVKGRFTISRD | | | |
| | | NAKNTVYLQMNSL | | | |
| | | KPEDTAVYYCAAR | | | |
| | | GPNEWGLPREDL | | | |
| | | FYDYWGQGTQVT | | | |
| | | VS (SEQ ID NO: | | | |
| | | 30) | | | |
| S1-41 | non-RBD | MAQVPLVESGGG | STFSTYHM | AINSEGSTY | GGWRYYGD |
| | | LVQAGGSLRLSCT | G (SEQ ID | (SEQ ID NO: | GSRYIPDFR |
| | | TSGSTFSTYHMG | NO: 207) | 208) | S (SEQ ID |
| | | WYRQAPGKQREL | | | NO: 209) |
| | | VAAINSEGSTYYP | | | |
| | | DSVKGRFTISRDN | | | |
| | | AQNTVYLEMASLK | | | |
| | | PEDTAVYHCNRG | | | |
| | | GWRYYGDGSRYI | | | |
| | | PDFRSWGQGTQV | | | |
| | | TVS (SEQ ID NO: | | | |
| | | 31) | | | |
| S1-46 | RBD | MAQVQLVESGGG | HQFSDYVIG | AIWWSDDIT | RFRPVSTY |
| | | LVQAGGSLRVSC | (SEQ ID NO: | Y (SEQ ID | QVRSTDDY |
| | | VISGHQFSDYVIG | 210) | NO: 211) | GY (SEQ ID |
| | | WFRQAPGKEREF | | | NO: 212) |
| | | VGAIWWSDDITYY | | | |
| | | EDSVKGRFTVSR | | | |
| | | DNAKNTVYLQMN | | | |
| | | SLKPEDTAVYHCA | | | |
| | | VRFRPVSTYQVRS | | | |
| | | TDDYGYWGQGTQ | | | |
| | | VTVS (SEQ ID NO: | | | |
| | | 32) | | | |
| S1-48 | RBD | MAQVQLVESGGG | ITLDYYAIG | LISSSDGST | GSLTRYGS |
| | | LVQPGGSLRLSCA | (SEQ ID NO: | Y (SEQ ID | SWHVPFGS |
| | | ASGITLDYYAIGW | 213) | NO: 214) | RSYEN |
| | | FRQAPGKEREGV | | | (SEQ ID NO: |
| | | SLISSSDGSTYYA | | | 215) |
| | | DSVKGRFTISRDN | | | |
| | | AKNTVYLQMNSLK | | | |
| | | PEDTAVYYCATGS | | | |
| | | LTRYGSSWHVPF | | | |
| | | GSRSYENWGQGT | | | |
| | | QVTVS (SEQ ID | | | |
| | | NO: 33) | | | |
| S1-49 | non-RBD | MAQIQMQLVESG | SIFSIGAMS | QISSGGITN | DETTPWGA |
| | | GGLVQAGGSLRL | (SEQ ID NO: | (SEQ ID NO: | TRG (SEQ |
| | | SCTASGSIFSIGA | 216) | 217) | ID NO: 218) |
| | | MSWYRQAPGKQ | | | |
| | | RELVAQISSGGITN | | | |
| | | YADSVKGRFTISR | | | |
| | | DNAKNTVYLQMN | | | |
| | | TLKPEDTAVYYCA | | | |
| | | ADETTPWGATRG | | | |
| | | WGQGTQVTVS | | | |
| | | (SEQ ID NO: 34) | | | |
| S1-50 | non-RBD | MAQTQLEESGGG | SIIRPNVMA | SMHSSGTK | RSTWAGQP |
| | | LVQAGGSLRLSCA | (SEQ ID NO: | N (SEQ ID | DY (SEQ ID |
| | | ASGSIIRPNVMAW | 120) | NO: 220) | NO: 221) |
| | | YRQGPGKQRELV | | | |
| | | ASMHSSGTKNYA | | | |
| | | ESVKGRFTISRDN | | | |
| | | AKNTVYLQMNSLK | | | |
| | | PEDTAVYYCATRS | | | |
| | | TWAGQPDYWGQ | | | |
| | | GTQVTVS (SEQ ID | | | |
| | | NO: 35) | | | |
| S1-51 | RBD | MAQVQLVESGGG | FSFSNYYM | AITTWLSNT | EQSGRDWR |
| | | LVQAGGSLRLSCA | V (SEQ ID | Y (SEQ ID | RTSPSPYAY |
| | | ASGFSFSNYYMV | NO: 222) | NO: 223) | (SEQ ID NO: |
| | | WFRQAPGKEREF | | | 224) |
| | | VAAITTWLSNTYY | | | |
| | | TDSVKDRFTISRD | | | |
| | | NAKKMLYLQMNS | | | |
| | | LKPEDTALYYCAA | | | |
| | | EQSGRDWRRTSP | | | |
| | | SPYAYWGQGTQV | | | |
| | | TVS (SEQ ID NO: | | | |
| | | 36) | | | |
| S1-52 | RBD | MAQVQLVESGGG | SIFSLNAKA | RITGSGTITN | APGGLWTG |
| | | LVQAGGSLRLSCA | (SEQ ID NO: | (SEQ ID NO: | PAY (SEQ ID |
| | | ASGSIFSLNAKAW | 225) | 226) | NO: 227) |
| | | YRQAPGSQRELV | | | |
| | | ARITGSGTITNYAD | | | |
| | | SVKGRFTISRDNV | | | |
| | | KNTVTLQMNSLRP | | | |
| | | EDTAIYICAAAPGG | | | |
| | | LWTGPAYWGQGT | | | |
| | | QVTVS (SEQ ID | | | |
| | | NO: 37) | | | |
| S1-53 | RBD | MAQVQLVESGGG | YTITNYWMA | TITYGAGMK | PQRPYSGF |
| | | LVQAGDSLRLSCA | (SEQ ID NO: | Y (SEQ ID | GSDDY |
| | | ASGYTITNYWMA | 228) | NO: 229) | (SEQ ID NO: |
| | | WFRQAPGKERES | | | 230) |
| | | VATITYGAGMKYY | | | |
| | | ADSVKGRFTISRD | | | |
| | | NTKNTVYLQMESL | | | |
| | | KPEDTAVYYCRSP | | | |
| | | QRPYSGFGSDDY | | | |
| | | WGQGTQVTVS | | | |
| | | (SEQ ID NO: 38) | | | |
| S1-54 | RBD | MAQVQLVESGGG | IIFSKYAVA | TINHSGDTK | GIWIASAHS |
| | | LVQAGGSLRLSCA | (SEQ ID NO: | (SEQ ID NO: | PDGRADH |
| | | ASGIIFSKYAVAW | 231) | 232) | (SEQ ID NO: |
| | | YRQAPGKQRELV | | | 233) |
| | | ATINHSGDTKYAD | | | |
| | | SVKGRFTISRDNA | | | |
| | | KSTLYLQMNSLKP | | | |
| | | EDTAVYYCNIGIWI | | | |
| | | ASAHSPDGRADH | | | |
| | | WGRGTQVTVS | | | |
| | | (SEQ ID NO: 39) | | | |
| S1-55 | RBD | MAQVQVVESGGG | RTFGMRAM | VISTSGGDT | PRIGAWSR |
| | | LVQAGGSLRLSCA | G (SEQ ID | S (SEQ ID | SPRDYDI |
| | | VSGRTFGMRAMG | NO: 234) | NO: 235) | (SEQ ID NO: |
| | | WFRQAPGKEREF | | | 236) |
| | | VAVISTSGGDTSY | | | |
| | | ADSVKGRFTISRD | | | |
| | | NAKNTVYLQMNTL | | | |
| | | KPEDTAVYYCAVP | | | |
| | | RIGAWSRSPRDY | | | |
| | | DIWGQGTQVTVS | | | |
| | | (SEQ ID NO: 40) | | | |
| S1-56 | RBD | MAQVQLVESGGG | SIFSIDAMG | HIRNTGTTA | LKDFLGGVI |
| | | LVQPGGSLRLSCA | (SEQ ID NO: | (SEQ ID NO: | PLNS (SEQ |
| | | ASGSIFSIDAMGW | 237) | 238) | ID NO: 239) |
| | | FRQAPGKEREPV | | | |
| | | AHIRNTGTTAYAD | | | |
| | | SVKGRFTISRDNT | | | |
| | | KNTVYLQMNSLKP | | | |
| | | EDTAVYYCNELKD | | | |
| | | FLGGVIPLNSWGQ | | | |
| | | GTQVTVS (SEQ ID | | | |
| | | NO: 41) | | | |
| S1-58 | non-RBD | MAQVQLVESGGG | STFSAYSM | AISSGGSTN | VGWDYRYD |
| | | LVQAGGSLRLSCA | G (SEQ ID | (SEQ ID NO: | YPITGRNDY |
| | | ASGSTFSAYSMG | NO: 240) | 241) | (SEQ ID NO: |
| | | WYRQAPGKQREL | | | 242) |
| | | VAAISSGGSTNYA | | | |
| | | DSVKGRFTISRDN | | | |
| | | AKNTVYLQMNSLK | | | |
| | | PEDTAVYYCNTVG | | | |
| | | WDYRYDYPITGR | | | |
| | | NDYWGQGTQVTV | | | |
| | | S (SEQ ID NO: 42) | | | |
| S1-60 | non-RBD | MAQVQLVESGGG | STFSTNAM | EITTDGTTR | VTRFLGGLL |
| | | LVQAGGSLRLSCA | G (SEQ ID | (SEQ ID NO: | RPDY (SEQ |
| | | ASGSTFSTNAMG | NO: 243) | 244) | ID NO: 245) |
| | | WYRQAPGKEREL | | | |
| | | VAEITTDGTTRYA | | | |
| | | DSVKGRFTISRDN | | | |
| | | AKNTVYLQMNSLK | | | |
| | | SEDTAVYYCNTVT | | | |
| | | RFLGGLLRPDYW | | | |
| | | GQGTQVTVS | | | |
| | | (SEQ ID NO: 43) | | | |
| S1-61 | RBD | MAQVQLIESGGGL | RTFSSYAM | AISRRGMDT | KMVAPIFVV |
| | | VQAGGSLRLSCA | G (SEQ ID | Y (SEQ ID | VTTRDEYDY |
| | | ASGRTFSSYAMG | NO: 246) | NO: 247) | (SEQ ID NO: |
| | | WLRQAPGEEREF | | | 248) |
| | | VAAISRRGMDTYY | | | |
| | | ADSVKGRFTISRD | | | |
| | | NAKNTVYLQMNSL | | | |
| | | KPEDTAVYYCARK | | | |
| | | MVAPIFVVVTTRD | | | |
| | | EYDYWGQGTQVT | | | |
| | | VS (SEQ ID NO: | | | |
| | | 44) | | | |
| S1-62 | RBD | MAQVQLVESGGG | RTSNTYPM | AITRSVPNT | SSNVYLLTR |
| | | LVQAGGSLRLSCA | A (SEQ ID | Y (SEQ ID | DEYDY |
| | | PSGRTSNTYPMA | NO: 249) | NO: 250) | (SEQ ID NO: |
| | | WFRQAPGKEREF | | | 251) |
| | | VAAITRSVPNTYY | | | |
| | | ADSVKGRFTISRDI | | | |
| | | AKNTMYLQMNSL | | | |
| | | KPEDTAVYYCAGS | | | |
| | | SNVYLLTRDEYDY | | | |
| | | WGQGTQVTVS | | | |
| | | (SEQ ID NO: 45) | | | |
| S1-63 | RBD | MAQVQLVESGGG | IIFSINAMG | LIMNNGNTY | VAYRDYDR |
| | | LVQFGGSLRLSCA | (SEQ ID NO: | (SEQ ID NO: | KRYDY |
| | | ASGIIFSINAMGWY | 252) | 253) | (SEQ ID NO: |
| | | RQAPGKERESVA | | | 254) |
| | | LIMNNGNTYYENS | | | |
| | | VKGRFSISRDNAK | | | |
| | | NTVYLQMDSLKPE | | | |
| | | DTAVYYCNAVAYR | | | |
| | | DYDRKRYDYWGQ | | | |
| | | GTQVTVS (SEQ ID | | | |
| | | NO: 46) | | | |
| S1-64 | non-RBD | MAQVQLVESGGG | SIFSVNSMG | TITSHGITN | EDTGLWNS |
| | | LVQPGGSLRLSCA | (SEQ ID NO: | (SEQ ID NO: | KDTY (SEQ |
| | | ASGSIFSVNSMG | 255) | 256) | ID NO: 257) |
| | | WYRQAPGKEREL | | | |
| | | VATITSHGITNYAD | | | |
| | | SVKGRFTISRDNA | | | |
| | | KDTVYLQMNSLKP | | | |
| | | EDTAVYFCHREDT | | | |
| | | GLWNSKDTYWGQ | | | |
| | | GTQVTVS (SEQ ID | | | |
| | | NO: 47) | | | |
| S1-65 | non-RBD | MAQVQLVESGGG | SIFNVNSMG | TITTRGITN | EDTGLWNS |
| | | LVQPGGSLRLSCA | (SEQ ID NO: | (SEQ ID NO: | KDVY (SEQ |
| | | ASGSIFNVNSMG | 258) | 259) | ID NO: 260) |
| | | WYRQAPGKEREL | | | |
| | | VATITTRGITNYAD | | | |
| | | SVKGRFTISRGNA | | | |
| | | KDTVHLQMNSLKP | | | |
| | | EDTAVYYCNREDT | | | |
| | | GLWNSKDVYWG | | | |
| | | QGTQVTVS (SEQ | | | |
| | | ID NO: 48) | | | |
| S1-66 | non-RBD | MAHVQLVESGGG | RTFMG | AISWNGVSI | ADVVAGTR |
| | | LVQAGGSLRLSCV | (SEQ ID NO: | T (SEQ ID | WTDYNY |
| | | ASTRTFMGWFRQ | 261) | NO: 262) | (SEQ ID NO: |
| | | APGKERAFVAAIS | | | 263) |
| | | WNGVSITYADSVA | | | |
| | | GRFTISRDNAKNT | | | |
| | | VLLQMNSLKPEDT | | | |
| | | AVYYCAAADVVA | | | |
| | | GTRWTDYNYWG | | | |
| | | QGTQVTVS (SEQ | | | |
| | | ID NO: 49) | | | |
| S1-RBD- | RBD | MAEVQLVESGGG | FTLDTTAIG | CISTSGERT | KYASFQYLP |
| 3 | | LVQAGGSLRLSCA | (SEQ ID NO: | Y (SEQ ID | VGLPWSMY |
| | | ASGFTLDTTAIGW | 264) | NO: 265) | DV (SEQ ID |
| | | FRQAPGKEREGV | | | NO: 266) |
| | | SCISTSGERTYYA | | | |
| | | DSVKGRFTISRDN | | | |
| | | AKNMVYLQMNNL | | | |
| | | KPEDTAVYSCAAK | | | |
| | | YASFQYLPVGLP | | | |
| | | WSMYDVRGQGT | | | |
| | | QVTVS (SEQ ID | | | |
| | | NO: 50) | | | |
| S1-RBD- | RBD | MAEVQLVESGGG | FTLGYYAIG | FISRHGSTT | SKTTAWDTI |
| 4 | | LVQAGGSLRLSCA | (SEQ ID NO: | Y (SEQ ID | RVSAIEYDY |
| | | ASEFTLGYYAIGW | 129) | NO: 268) | (SEQ ID NO: |
| | | FRQAPGKEREGV | | | 269) |
| | | SFISRHGSTTYYT | | | |
| | | KSVRGRFTISRDN | | | |
| | | SENTVYLQMDGL | | | |
| | | KPEDTAVYYCAAS | | | |
| | | KTTAWDTIRVSAIE | | | |
| | | YDYWGQGTQVTV | | | |
| | | S (SEQ ID NO: 51) | | | |
| S1-RBD- | RBD | MAQVQLVESGGG | RTFSSLVM | FIEWNGGD | DRAYSLYYY |
| 5 | | LVQAGGSLRLSCA | G (SEQ ID | TY (SEQ ID | RVSEYQY |
| | | ASGRTFSSLVMG | NO: 270) | NO: 271) | (SEQ ID NO: |
| | | WFRQAPGKEREF | | | 272) |
| | | VAFIEWNGGDTYY | | | |
| | | TDAVKGRFTISRD | | | |
| | | NAKNTVFLQMNSL | | | |
| | | KPEDTAVYYCAAD | | | |
| | | RAYSLYYYRVSEY | | | |
| | | QYWGQGTRVTVS | | | |
| | | (SEQ ID NO: 52) | | | |
| S1-RBD- | RBD | MGQVQVAESGGG | RTFTTNAM | AISWSTGTT | RSAFNAYA |
| 6 | | LVQAGDSLRLSCA | G (SEQ ID | Y (SEQ ID | WTTERAYD |
| | | ASGRTFTTNAMG | NO: 273) | NO: 274) | Y (SEQ ID |
| | | WFRQAPGKEREF | | | NO: 275) |
| | | VAAISWSTGTTYY | | | |
| | | SDPVKGRFTISRD | | | |
| | | NAKNTVYLQMNSL | | | |
| | | KPGDTAVYYCTLR | | | |
| | | SAFNAYAWTTER | | | |
| | | AYDYWGQGTQVT | | | |
| | | VS (SEQ ID NO: | | | |
| | | 53) | | | |
| S1-RBD- | RBD | MAQVQLVESGGG | RTFSNYVVA | LITWSGADR | DRGSSYHY |
| 9 | | SVQAGGSLRLSC | (SEQ ID NO: | Y (SEQ ID | AESKNYDY |
| | | AASARTFSNYVVA | 276) | NO: 277) | (SEQ ID NO: |
| | | WFRQAPGKEREF | | | 278) |
| | | VALITWSGADRYY | | | |
| | | GDSVKGRFTISRD | | | |
| | | DAKNTVYLQMNSL | | | |
| | | KPEDTADYYCAAD | | | |
| | | RGSSYHYAESKN | | | |
| | | YDYWGQGTQVTV | | | |
| | | S (SEQ ID NO: 54) | | | |
| S1-RBD- | RBD | MAQVQLVESGGG | RTFRNYAM | VITATDDITY | DYGDSYYY |
| 10 | | LVQAGGSLRLSCA | G (SEQ ID | (SEQ ID NO: | TRRTEYGY |
| | | VSGRTFRNYAMG | NO: 279) | 280) | (SEQ ID NO: |
| | | WFRQAPGKEREF | | | 281) |
| | | VAVITATDDITYYA | | | |
| | | DSVKGRFTISRDN | | | |
| | | AKNTVYLQMNSLK | | | |
| | | PEDTAVYYCAADY | | | |
| | | GDSYYYTRRTEY | | | |
| | | GYWGQGTQVTVS | | | |
| | | (SEQ ID NO: 55) | | | |
| S1-RBD- | RBD | MAQVQVVESGGG | STDSNYVM | SINSGGETR | ERLAWDPS |
| 11 | | LVQAGGSLRLSCV | G (SEQ ID | (SEQ ID NO: | TS (SEQ ID |
| | | ASGSTDSNYVMG | NO: 282) | 283) | NO: 284) |
| | | WYRQTAGKQRE | | | |
| | | WASINSGGETRS | | | |
| | | VDSVKGRFTISGD | | | |
| | | NAKNTVYLQMNSL | | | |
| | | KPEDTAVYYCFYE | | | |
| | | RLAWDPSTSWGQ | | | |
| | | GTQVTVS (SEQ ID | | | |
| | | NO: 56) | | | |
| S1-RBD- | RBD | MAQVQLVESGGG | SILSINDMG | FITSGGSTN | DIRESGFVY |
| 12 | | LVQPGGSLRLSCA | (SEQ ID NO: | (SEQ ID NO: | GMNAWGKY |
| | | ASGSILSINDMGW | 285) | 286) | (SEQ ID NO: |
| | | YRQAPGKERELV | | | 287) |
| | | AFITSGGSTNYAE | | | |
| | | SVKGRFTVSRDN | | | |
| | | AKHAVYLQMNSL | | | |
| | | KPEDTAVYYCNAD | | | |
| | | IRESGFVYGMNA | | | |
| | | WGKYWGKGTLVT | | | |
| | | VS (SEQ ID NO: | | | |
| | | 57) | | | |
| S1-RBD- | RBD | MAQVQLVESGGG | RSFSINPMG | AISWSGSKT | ASRGPVYG |
| 14 | | LVQAGGSVRLSC | (SEQ ID NO: | V (SEQ ID | ANYVPGPH |
| | | AASGRSFSINPMG | 288) | NO: 289) | EYEY (SEQ |
| | | WFRQAPGKEREF | | | ID NO: 290) |
| | | VAAISWSGSKTVY | | | |
| | | VDSVKGRFSISRD | | | |
| | | NAKNTVYLQMNSL | | | |
| | | EPEDTAVYHCAVA | | | |
| | | SRGPVYGANYVP | | | |
| | | GPHEYEYWGQGT | | | |
| | | QVTVS (SEQ ID | | | |
| | | NO: 58) | | | |
| S1-RBD- | RBD | MAQVQLVESGGG | RTFSSYAM | SINWNGGN | TGPNEYGL |
| 15 | | LVQAGGSLRLSCA | G (SEQ ID | TY (SEQ ID | PREDLFYDY |
| | | ASGRTFSSYAMG | NO: 246) | NO: 292) | (SEQ ID NO: |
| | | WFRQAPGKEREF | | | 293) |
| | | VASINWNGGNTY | | | |
| | | YADFVKGRFTISR | | | |
| | | DNAKNTVYLQMN | | | |
| | | SLKPEDTAVYYCA | | | |
| | | ATGPNEYGLPRE | | | |
| | | DLFYDYWGQGTQ | | | |
| | | VTVS (SEQ ID NO: | | | |
| | | 59) | | | |
| S1-RBD- | RBD | MAQVQLVESGGG | RGFDRYPM | GINWNGAN | DQHYMGSP |
| 16 | | LVQSGGSLRLSCV | G (SEQ ID | TY (SEQ ID | PPKEYEYDY |
| | | ASGRGFDRYPMG | NO: 294) | NO: 295) | (SEQ ID NO: |
| | | WFRQAPGKEREF | | | 296) |
| | | VGGINWNGANTY | | | |
| | | YADSVKGRFTISR | | | |
| | | DNTKNTVYLQMN | | | |
| | | SLKPEDTALYYCA | | | |
| | | ADQHYMGSPPPK | | | |
| | | EYEYDYWGQGTQ | | | |
| | | VTVS (SEQ ID NO: | | | |
| | | 60) | | | |
| S1-RBD- | RBD | MAQVQLVESGGG | ITFSGYVMA | WISHGGIKD | KDAQSS |
| 18 | | LVQAGGSLRLSCT | (SEQ ID NO: | (SEQ ID NO: | (SEQ ID NO: |
| | | ASGITFSGYVMAW | 297) | 298) | 299) |
| | | YRQAPGKQRELV | | | |
| | | GWISHGGIKDYAD | | | |
| | | SVKGRFTISRDNP | | | |
| | | KNTVYLQMNSLKP | | | |
| | | EDTAVYYCNRKD | | | |
| | | AQSSWGQGTQVT | | | |
| | | VS (SEQ ID NO: | | | |
| | | 61) | | | |
| S1-RBD- | RBD | MAEVQLVESGGG | FSFSSYGM | TISGTGGNT | PPSWYTRR |
| 19 | | LVQPGGSLRLSCV | G (SEQ ID | A (SEQ ID | YINFAPSEV |
| | | ASGFSFSSYGMG | NO: 300) | NO: 301) | RH (SEQ ID |
| | | WFRQAPGKEREF | | | NO: 302) |
| | | VATISGTGGNTAY | | | |
| | | SDSVKDRFTISRD | | | |
| | | NAKNTVYLEMNSL | | | |
| | | KPEDTAVYYCAAP | | | |
| | | PSWYTRRYINFAP | | | |
| | | SEVRHWGQGTQV | | | |
| | | TVS (SEQ ID NO: | | | |
| | | 62) | | | |
| S1-RBD- | RBD | MAQVQLVESGGG | RTFSSGAM | VIRWSGSTY | TDRTHYDIV |
| 20 | | LVQAGDSRRLSC | A (SEQ ID | (SEQ ID NO: | EYTRDHFYS |
| | | AVSGRTFSSGAM | NO: 303) | 304) | N (SEQ ID |
| | | AWFRQAPGEERD | | | NO: 305) |
| | | FVAVIRWSGSTYY | | | |
| | | ADSVKDRFTISTD | | | |
| | | NVKNTVFLQMNNL | | | |
| | | APEDTAVYYCAAT | | | |
| | | DRTHYDIVEYTRD | | | |
| | | HFYSNWGQGTQV | | | |
| | | TVS (SEQ ID NO: | | | |
| | | 63) | | | |
| S1-RBD- | RBD | MAQVQLVESGGG | RAFSDYGM | TIDSTGSNT | DRFGGGRM |
| 21 | | SVQAGGSLRLSC | G (SEQ ID | Y (SEQ ID | EYRYEY |
| | | AASGRAFSDYGM | NO: 306) | NO: 307) | (SEQ ID NO: |
| | | GWFRQAPGKERE | | | 308) |
| | | FVATIDSTGSNTY | | | |
| | | YADSVKGRFTISR | | | |
| | | VNAENTVYLQMN | | | |
| | | SLKPEDTAVYYCA | | | |
| | | ADRFGGGRMEYR | | | |
| | | YEYWGQGTQVTV | | | |
| | | S (SEQ ID NO: 64) | | | |
| S1-RBD- | RBD | MAQVQLAESGGG | RTFSAYTLA | HIDWSGTET | DRAYSLMY |
| 22 | | LVQAGDSLRLSCV | (SEQ ID NO: | N (SEQ ID | YDTREYEY |
| | | ASGRTFSAYTLAW | 309) | NO: 310) | (SEQ ID NO: |
| | | FRLAPGKEREFVA | | | 311) |
| | | HIDWSGTETNYAD | | | |
| | | SNKGRFTISRDNA | | | |
| | | LNTVYLQMTGLKP | | | |
| | | EDTAVYYCAADRA | | | |
| | | YSLMYYDTREYEY | | | |
| | | WGQGTQVTVS | | | |
| | | (SEQ ID NO: 65) | | | |
| S1-RBD- | RBD | MAQVHLVESGGD | SGFENNAIT | GITSGGSTN | VAWDSRRR |
| 23 | | LVQPGGSLRLSCV | (SEQ ID NO: | (SEQ ID NO: | SVVAF (SEQ |
| | | ASGSGFENNAITW | 312) | 313) | ID NO: 314) |
| | | YRQAPGKERELV | | | |
| | | SGITSGGSTNYAD | | | |
| | | SVKGRFTISRDNA | | | |
| | | KNTVYLEMNSLKP | | | |
| | | EDTAVYLCQAVA | | | |
| | | WDSRRRSVVAFW | | | |
| | | GQGTQVTVS | | | |
| | | (SEQ ID NO: 66) | | | |
| S1-RBD- | RBD | MAQVQLVESGGG | HTFGSLTMA | GTDWSGIN | DRAASLYPL |
| 24 | | LVQAGGSLRLSCV | (SEQ ID NO: | (SEQ ID NO: | RDQADYMY |
| | | VSGHTFGSLTMA | 315) | 316) | (SEQ ID NO: |
| | | WFRQAPGKEREF | | | 317) |
| | | VAGTDWSGINYA | | | |
| | | DSVKGRFTISRDS | | | |
| | | TTNTLFLQMNSLK | | | |
| | | PEDTAVYYCAFDR | | | |
| | | AASLYPLRDQADY | | | |
| | | MYWGQGTQVTVS | | | |
| | | (SEQ ID NO: 67) | | | |
| S1-RBD- | RBD | MAEVQLVESGGG | FTLDYYTIG | YISSSGSNT | RRVWQETIV |
| 25 | | LVQPGGSLKLSCA | (SEQ ID NO: | Y (SEQ ID | VRGPEHYE |
| | | ASGFTLDYYTIGW | 318) | NO: 319) | Y (SEQ ID |
| | | FRQAPGKEREGV | | | NO: 320) |
| | | SYISSSGSNTYYE | | | |
| | | DSVKGRFTISRDN | | | |
| | | AKSTVYLQMNSLK | | | |
| | | PEDTAVYYCAARR | | | |
| | | VWQETIVVRGPEH | | | |
| | | YEYWGQGTQVTV | | | |
| | | S (SEQ ID NO: 68) | | | |
| S1-RBD- | RBD | MAQVQLVESGGG | RTFGGYGV | GITSTSFGT | SVLSPPPRT |
| 26 | | LVQPGGSLRLSCA | G (SEQ ID | S (SEQ ID | NSAYAY |
| | | ISGRTFGGYGVG | NO: 321) | NO: 322) | (SEQ ID NO: |
| | | WFRQAPGKEREF | | | 323) |
| | | VAGITSTSFGTSY | | | |
| | | ADAVKGRFTISRD | | | |
| | | NAKNTVTLQMNSL | | | |
| | | KFEDTAVYYCAAS | | | |
| | | VLSPPPRTNSAYA | | | |
| | | YWGQGIQVTVS | | | |
| | | (SEQ ID NO: 69) | | | |
| S1-RBD- | RBD | MAQVHVVESGGG | RTFGIYNMG | AITGDASDT | TGAITRATM |
| 27 | | LVQAGGSLRLSCA | (SEQ ID NO: | Y (SEQ ID | WKPNF |
| | | ASGRTFGIYNMG | 324) | NO: 325) | (SEQ ID NO: |
| | | WFRQAPGKEREF | | | 326 |
| | | VAAITGDASDTYY | | | |
| | | ADSVKGRFTISRQ | | | |
| | | NAKNTVFLQMDNL | | | |
| | | KPEDTAVYYCAAT | | | |
| | | GAITRATMWKPNF | | | |
| | | WGQGTQVTVS | | | |
| | | (SEQ ID NO: 70) | | | |
| S1-RBD- | RBD | MAQVQLAESGGG | RTISTDAMG | ISWRSDDTY | DQGRSYYY |
| 28 | | LVQAGGSLRLSCA | (SEQ ID NO: | (SEQ ID NO: | TRESEYNY |
| | | ASGRTISTDAMG | 327) | 328) | (SEQ ID NO: |
| | | WFRQAPGKEREF | | | 329) |
| | | VAISWRSDDTYYA | | | |
| | | DNVKGRFTMSRD | | | |
| | | NAKNTVYLQMNN | | | |
| | | LKPEDTAVYYCVA | | | |
| | | DQGRSYYYTRES | | | |
| | | EYNYWGQGTQVT | | | |
| | | VS (SEQ ID NO: | | | |
| | | 71) | | | |
| S1-RBD- | RBD | MAQVQLVESGGG | LASIVFNMG | GILGSGQST | GGRLDLVN |
| 29 | | LVQDGGSLRLSC | (SEQ ID NO: | Y (SEQ ID | TTGGYEPTY |
| | | AASGLASIVFNMG | 330) | NO: 331) | (SEQ ID NO: |
| | | WFRQAPGKEREF | | | 332) |
| | | VAGILGSGQSTYY | | | |
| | | GNSVKGRFTISRD | | | |
| | | SAKNTVYLQMNSL | | | |
| | | KPEDTAVYYCAAG | | | |
| | | GRLDLVNTTGGYE | | | |
| | | PTYWGQGTQVTV | | | |
| | | S (SEQ ID NO: 72) | | | |
| S1-RBD- | RBD | MAQVQLVESGGG | RTFSSLVM | FIEWSGAET | DAAYSLYYY |
| 30 | | LVQAGGSLRLSCA | G (SEQ ID | Y (SEQ ID | RVSEYEY |
| | | ASGRTFSSLVMG | NO: 270) | NO: 193) | (SEQ ID NO: |
| | | WFRQAPGKEREF | | | 335) |
| | | VAFIEWSGAETYY | | | |
| | | TDSVKGRFTISRD | | | |
| | | NAKNTVFLQMNSL | | | |
| | | KPEDTAVYYCAAD | | | |
| | | AAYSLYYYRVSEY | | | |
| | | EYWGQGTRVTVS | | | |
| | | (SEQ ID NO: 73) | | | |
| S1-RBD- | RBD | MAEVQLVESGGG | FTLDYYTIG | YISSSGSNS | RRVWQPTIV |
| 32 | | LVQPGGSLKLSCA | (SEQ ID NO: | Y (SEQ ID | VSGPEHYD |
| | | ASGFTLDYYTIGW | 318) | NO: 337) | Y (SEQ ID |
| | | FRQAPGKEREGV | | | NO: 338) |
| | | SYISSSGSNSYYV | | | |
| | | DSVKGRFTISRDN | | | |
| | | AKSTVYLQMNSLK | | | |
| | | PEDTAVYYCAARR | | | |
| | | VWQPTIVVSGPEH | | | |
| | | YDYWGQGTQVTV | | | |
| | | S (SEQ ID NO: 74) | | | |
| S1-RBD- | RBD | MAQVQLVESGGG | RTFSTTFMG | AINWNGGS | DPSDGRVV |
| 34 | | LVQAGDSLRLSCV | (SEQ ID NO: | TR (SEQ ID | GYNYVPVS |
| | | ASGRTFSTTFMG | 339) | NO: 340) | THEYDY |
| | | WFRQAPGKERDF | | | (SEQ ID NO: |
| | | VAAINWNGGSTR | | | 341) |
| | | YADSVKGRFTISR | | | |
| | | DNAKNMVYLQLN | | | |
| | | SLKPEDTAVYYCA | | | |
| | | ADPSDGRVVGYN | | | |
| | | YVPVSTHEYDYW | | | |
| | | GQGTQVTVS | | | |
| | | (SEQ ID NO: 75) | | | |
| S1-RBD- | RBD | MTQVQVVESGGG | STDSNYVM | SINSGGDTK | EKLAWDPS |
| 35 | | LVQAGGSLRLSCV | G (SEQ ID | (SEQ ID NO: | TT (SEQ ID |
| | | ASGSTDSNYVMG | NO: 282) | 343) | NO: 344) |
| | | WYRQASGKQRE | | | |
| | | WASINSGGDTKS | | | |
| | | VDSVKGRFTISIDN | | | |
| | | AKNTMYLQMNSL | | | |
| | | KPEDTAVYYCFYE | | | |
| | | KLAWDPSTTWGQ | | | |
| | | GTQVTVS (SEQ ID | | | |
| | | NO: 76) | | | |
| S1-RBD- | RBD | MAQVQLVESGGG | RTFSTTFMG | AINWSGGS | DPSDGRVF |
| 36 | | LVQAGDSLRLSCV | (SEQ ID NO: | TR (SEQ ID | GVNYVPVS |
| | | ASGRTFSTTFMG | 339) | NO: 346) | THEYDY |
| | | WFRQAPGKEREF | | | (SEQ ID NO: |
| | | VAAINWSGGSTRY | | | 347) |
| | | ADSVKGRFTISRD | | | |
| | | NAKNTVYLQLNSL | | | |
| | | KPEDTAVYYCAAD | | | |
| | | PSDGRVFGVNYV | | | |
| | | PVSTHEYDYWGQ | | | |
| | | GTQVTVS (SEQ ID | | | |
| | | NO: 77) | | | |
| S1-RBD- | RBD | MAQVQLVESGGG | DSFSFRWY | GISSAGSTN | ATQERDY |
| 37 | | LVQAGGSLRLSC | GMN (SEQ | (SEQ ID NO: | (SEQ ID NO: |
| | | GASGDSFSFRWY | ID NO: 348) | 349) | 350) |
| | | GMNWYRQGPGK | | | |
| | | PREWVAGISSAGS | | | |
| | | TNYADSVKGRFTI | | | |
| | | SRDNAKNTVYLQ | | | |
| | | MNSLKPEDTAVYY | | | |
| | | CYSATQERDYWG | | | |
| | | QGTQVTVS (SEQ | | | |
| | | ID NO: 78) | | | |
| S1-RBD- | RBD | MAQVQLVESGGG | STFDNYPVT | SIRSGHITN | VDAQSDY |
| 38 | | LVQAGGSLRLSCA | (SEQ ID NO: | (SEQ ID NO: | (SEQ ID NO: |
| | | ASGSTFDNYPVT | 351) | 352) | 353) |
| | | WYRQAPGKQREF | | | |
| | | VASIRSGHITNYAN | | | |
| | | SVKGRFTMSKDN | | | |
| | | AKNTVYLEMNNLK | | | |
| | | PEDTAVYYCYTVD | | | |
| | | AQSDYWGQGTQV | | | |
| | | TVS (SEQ ID NO: | | | |
| | | 79) | | | |
| S1-RBD- | RBD | MAQVQLVESGGG | STFSSDVM | SFSSSGSG | AHVNGGDS |
| 39 | | RVQAGGSLRLSC | A (SEQ ID | N (SEQ ID | (SEQ ID NO: |
| | | AVSGSTFSSDVM | NO: 354) | NO: 355) | 356) |
| | | AWYRQPPGKQRE | | | |
| | | WASFSSSGSGN | | | |
| | | YAASVKGRFTISR | | | |
| | | ENAKNTVYLQMN | | | |
| | | SLKPEDTGVYYCH | | | |
| | | AAHVNGGDSWG | | | |
| | | QGTQVTVS (SEQ | | | |
| | | ID NO: 80) | | | |
| S1-RBD- | RBD | MAQVQLVESGGG | ITFSNYGMV | SISSGRDTN | VTDRSDY |
| 40 | | LVQAGGSLRLSCA | (SEQ ID NO: | (SEQ ID NO: | (SEQ ID NO: |
| | | ASGITFSNYGMV | 357) | 358) | 359) |
| | | WWRQAPGKQRE | | | |
| | | FVASISSGRDTNY | | | |
| | | ADFVKGRFTISRD | | | |
| | | NAKNTVHLQMNS | | | |
| | | LKPEDTAVYYCYT | | | |
| | | VTDRSDYWGQGT | | | |
| | | QVTVS (SEQ ID | | | |
| | | NO: 81) | | | |
| S1-RBD- | RBD | MAQVQLVESGGG | ITFSRSAVG | AISNSGTTS | ADGLLGGD |
| 41 | | LVQAGGSLRLSCA | (SEQ ID NO: | (SEQ ID NO: | Y (SEQ ID |
| | | ASGITFSRSAVGW | 360) | 361) | NO: 362) |
| | | HRQAPGKPREWV | | | |
| | | AAISNSGTTSYTD | | | |
| | | PMKGRVTISRDNA | | | |
| | | KNTVYLQMINLKP | | | |
| | | EDTAVYYCYIADG | | | |
| | | LLGGDYWGQGTQ | | | |
| | | VTVS (SEQ ID NO: | | | |
| | | 82) | | | |
| S1-RBD- | RBD | MAQVQLVESGGG | STFSSDVIT | SIQSSGSRN | GHVTGGDS |
| 43 | | LVQAGGSLRLSCA | (SEQ ID NO: | (SEQ ID NO: | (SEQ ID NO: |
| | | ASGSTFSSDVITW | 363) | 364) | 365) |
| | | YRQPPGKQREWV | | | |
| | | ASIQSSGSRNYAA | | | |
| | | SVKGRFTISRENA | | | |
| | | KNTVYLQMNSLKP | | | |
| | | EDTGVYYCHAGH | | | |
| | | VTGGDSWGQGT | | | |
| | | QVTVS (SEQ ID | | | |
| | | NO: 83) | | | |
| S1-RBD- | RBD | MAQVQLVESGGG | STVSNSPM | TVQSGGNA | ADDRSDY |
| 44 | | LVQAGGSLRLSCA | D (SEQ ID | S (SEQ ID | (SEQ ID NO: |
| | | ASSSTVSNSPMD | NO: 366) | NO: 367) | 368) |
| | | WFRQAPGKQREF | | | |
| | | VATVQSGGNASY | | | |
| | | SYSVKGRFTISRD | | | |
| | | NAKKMVYLQMNS | | | |
| | | LKPEDTAVYYCHA | | | |
| | | ADDRSDYWGQGT | | | |
| | | QVTVS (SEQ ID | | | |
| | | NO: 84) | | | |
| S1-RBD- | RBD | MAQVQLVESGGG | ITWMPYGM | TISSNGRTN | ATALSDY |
| 45 | | LVQAGGSLRLSCL | E (SEQ ID | (SEQ ID NO: | (SEQ ID NO: |
| | | ASGITWMPYGME | NO: 369) | 370) | 371) |
| | | WYRQAPGKQRVF | | | |
| | | VATISSNGRTNYA | | | |
| | | ASVMGRFTISRDN | | | |
| | | AKNTVYLQMNSLK | | | |
| | | PEDTAVYYCHGAT | | | |
| | | ALSDYWGQGTQV | | | |
| | | TVS (SEQ ID NO: | | | |
| | | 85) | | | |
| S1-RBD- | RBD | MAEVQLVESGGG | STLGSYAIG | FISRDGSTT | SKITAWDTII |
| 46 | | LVQAGGSLRLSCA | (SEQ ID NO: | F (SEQ ID | VSAIEYTY |
| | | ASESTLGSYAIGW | 372) | NO: 373) | (SEQ ID NO: |
| | | FRQAPGKEREGV | | | 374) |
| | | SFISRDGSTTFYT | | | |
| | | SSVRGRFTISRDN | | | |
| | | TKNMVYLQMDGL | | | |
| | | KPEDTAVYYCAAS | | | |
| | | KITAWDTIIVSAIEY | | | |
| | | TYWGQGTQVTVS | | | |
| | | (SEQ ID NO: 86) | | | |
| S1-RBD- | RBD | MAQVQLVESGGG | ISFSTYDMN | GIGTLNSPR | ASDRDY |
| 47 | | LAQTGGSLRLSCA | (SEQ ID NO: | (SEQ ID NO: | (SEQ ID NO: |
| | | ASGISFSTYDMNW | 375) | 376) | 377) |
| | | FRQTPGKQQEWV | | | |
| | | AGIGTLNSPRYAN | | | |
| | | AVKGRFTISRDDA | | | |
| | | KNTVYLQMNSLKP | | | |
| | | EDTAVYYCNTASD | | | |
| | | RDYWGQGTQVTV | | | |
| | | S (SEQ ID NO: 87) | | | |
| S1-RBD- | RBD | MAQVQLVESGGG | RTFDNYNIG | TISRSGDITN | SMTTGGDY |
| 48 | | WWQAGGSLRLSC | FIE (SEQ ID | (SEQ ID NO: | (SEQ ID NO: |
| | | SASGRTFDNYNIG | NO: 378) | 379) | 380) |
| | | FIEWFRQAPGKER | | | |
| | | TFVATISRSGDITN | | | |
| | | YAGSVAGRFTISR | | | |
| | | DNNKNTVYLQMN | | | |
| | | SLKPEDTAVYYCH | | | |
| | | MSMTTGGDYWG | | | |
| | | QGTQVTVS (SEQ | | | |
| | | ID NO: 88) | | | |
| S1-RBD- | RBD | MAQVQLVESGGG | STFSIGAMG | TITSSGTTS | NNLHGGDY |
| 49 | | LVQAGGSLRLSCT | (SEQ ID NO: | (SEQ ID NO: | (SEQ ID NO: |
| | | SSASTFSIGAMGW | 381) | 382) | 383) |
| | | FRQAPGKQREFV | | | |
| | | ATITSSGTTSYAG | | | |
| | | SVKGRFTISRENA | | | |
| | | KNSVYLQMNTLKP | | | |
| | | EDTAVYYCYANNL | | | |
| | | HGGDYWGQGTQ | | | |
| | | VTVS (SEQ ID NO: | | | |
| | | 89) | | | |
| S1-RBD- | RBD | MAQVQLVESGGG | RTLSTYRM | AIFWSGGHT | RNPATGNY |
| 51 | | LVQAGGSLRLSCV | G (SEQ ID | V (SEQ ID | DY (SEQ ID |
| | | ASGRTLSTYRMG | NO: 384) | NO: 385) | NO: 386) |
| | | WFRQAPGKERKF | | | |
| | | VAAIFWSGGHTVY | | | |
| | | EDSVKGRFTISTD | | | |
| | | NAQNTVYLQMNS | | | |
| | | LKPEDTAAYYCAA | | | |
| | | RNPATGNYDYWG | | | |
| | | QGTQITVS (SEQ | | | |
| | | ID NO: 90) | | | |
| S2-1 | S2 | MAQVQVVESGGG | LVLGGYDM | SISNGGSTD | RSTYYGIDY |
| | | LVQAGGSLRLSCA | R (SEQ ID | (SEQ ID NO: | (SEQ ID NO: |
| | | ASTLVLGGYDMR | NO: 387) | 388) | 389) |
| | | WYRQVPGNGREL | | | |
| | | VASISNGGSTDYA | | | |
| | | DFVKGRFTISSDV | | | |
| | | AKNTVYLQMDSLK | | | |
| | | SEDTAVYYCNMR | | | |
| | | STYYGIDYWGRG | | | |
| | | TQVTVS (SEQ ID | | | |
| | | NO: 91) | | | |
| S2-2 | S2 | MAQVRLVESGGG | LQFSRITMA | TLSTRGTTN | GPGYKTDY |
| | | LAQAGGSLRLSCA | (SEQ ID NO: | (SEQ ID NO: | (SEQ ID NO: |
| | | ASGLQFSRITMAW | 390) | 391) | 392) |
| | | YRQAPGKQRELV | | | |
| | | ATLSTRGTTNYPD | | | |
| | | SVKGRFTISRDNA | | | |
| | | KNMVYLQMNSLK | | | |
| | | PEDTAVYYCNTGP | | | |
| | | GYKTDYWGQGTQ | | | |
| | | VTVS (SEQ ID NO: | | | |
| | | 92) | | | |
| S2-3 | S2 | MAQILVTQSGGGL | LVLGGYDM | SITNGGDTR | HSTYYGRD |
| | | VQTGGSLRLSCAA | R (SEQ ID | (SEQ ID NO: | F (SEQ ID |
| | | STLVLGGYDMRW | NO: 387) | 394) | NO: 395) |
| | | YRQAPGKERELV | | | |
| | | ASITNGGDTRYVD | | | |
| | | SVKGRFTISSDNA | | | |
| | | KNAVYLQMDSLK | | | |
| | | SEDTAVYYCNMH | | | |
| | | STYYGRDFWGQG | | | |
| | | TQVTVS (SEQ ID | | | |
| | | NO: 93) | | | |
| S2-4 | S2 | MTQVQLVESGGG | PTFSSRTM | TIPWWRGA | SKTWSPST |
| | | LVQAGDSLRLSCV | G (SEQ ID | LPY (SEQ ID | GVATEYEF |
| | | SSEPTFSSRTMG | NO: 396) | NO: 397) | (SEQ ID NO: |
| | | WFRQAPGKERDN | | | 398) |
| | | VATIPWWRGALPY | | | |
| | | YKDSVKGRFTISR | | | |
| | | DSAKNTMYLQMN | | | |
| | | SLKPEDTAVYYCA | | | |
| | | ASKTWSPSTGVA | | | |
| | | TEYEFRGQGTQV | | | |
| | | TVS (SEQ ID NO: | | | |
| | | 94) | | | |
| S2-5 | S2 | MAQVQLVESGGG | LTFKNYAM | TINSIGGPA | ERTPSTLLA |
| | | LVQAGGSLRLSC | G (SEQ ID | N (SEQ ID | GSSNPSRV |
| | | GASGLTFKNYAM | NO: 399) | NO: 400) | DV (SEQ ID |
| | | GWFRQPPGKERE | | | NO: 401) |
| | | FVATINSIGGPANY | | | |
| | | ANSVKGRFTISRD | | | |
| | | NAKNTVYLQMNSL | | | |
| | | KPEDTAVYYCAAE | | | |
| | | RTPSTLLAGSSNP | | | |
| | | SRVDVWGQGTQV | | | |
| | | TVS (SEQ ID NO: | | | |
| | | 95) | | | |
| S2-6 | S2 | MTQVQLVESGGG | GIFGGSTM | TISNSGSAN | HSIYYGRDY |
| | | LVQAGGSLRLSCA | G (SEQ ID | (SEQ ID NO: | (SEQ ID NO: |
| | | ASAGIFGGSTMG | NO: 402) | 403) | 404) |
| | | WYRQAPGEQRE | | | |
| | | MVATISNSGSANY | | | |
| | | ADSVKGRFTISRD | | | |
| | | HAKRTVYLQMDSL | | | |
| | | KPEDTAVYYCNKH | | | |
| | | SIYYGRDYWGQG | | | |
| | | TQVTVS (SEQ ID | | | |
| | | NO: 96) | | | |
| S2-7 | S2 | MTQVQLVESGGG | PTFSSRTM | AIPWWKGA | SKTWSPYT |
| | | LVQAGDSLRLSCV | G (SEQ ID | LPY (SEQ ID | DAATELEF |
| | | ASEPTFSSRTMG | NO: 396) | NO: 406) | (SEQ ID NO: |
| | | WFRQAPGKERES | | | 407) |
| | | VAAIPWWKGALPY | | | |
| | | YKDSVKGRFTISR | | | |
| | | DSAKNTMYLQMN | | | |
| | | SLKPEDTAVYYCA | | | |
| | | ASKTWSPYTDAAT | | | |
| | | ELEFRGQGTQVT | | | |
| | | VS (SEQ ID NO: | | | |
| | | 97) | | | |
| S2-9 | S2 | MLQVQLVESGGG | STFSTYGVA | SINSWGWIN | QNSRGDNY |
| | | LVQAGGSLRLSCA | (SEQ ID NO: | (SEQ ID NO: | (SEQ ID NO: |
| | | ASGSTFSTYGVA | 408) | 409) | 410) |
| | | WYRQAPGKQREL | | | |
| | | VASINSWGWINYA | | | |
| | | DSVKGRFTISRDN | | | |
| | | AKNTVSLQMNSLK | | | |
| | | PEDTAVYTCNSQ | | | |
| | | NSRGDNYWGQG | | | |
| | | TQVTVS (SEQ ID | | | |
| | | NO: 98) | | | |
| S2-10 | S2 | MAQVQLVESGGG | RIVSSSAMS | VIASSNSHT | RSHLTIAAW |
| | | LVQTGGSLRLSCA | (SEQ ID NO: | Y (SEQ ID | ERLKTYDD |
| | | ASGRIVSSSAMS | 411) | NO: 412) | (SEQ ID NO: |
| | | WFRQAPGKEREF | | | 413) |
| | | VAVIASSNSHTYY | | | |
| | | ADAVKGRFTISRD | | | |
| | | NAKNTVYLQMNSL | | | |
| | | KPEDTAIYYCAAR | | | |
| | | SHLTIAAWERLKT | | | |
| | | YDDWGQGTQVTV | | | |
| | | S (SEQ ID NO: 99) | | | |
| S2-11 | S2 | MAQVQLVESGGG | RTFSGATM | SILWRYEYT | AIERSGIYY |
| | | LVQPGGSLRLSCA | G (SEQ ID | N (SEQ ID | HEDSNKYQ |
| | | ASGRTFSGATMG | NO: 414) | NO: 415) | Y (SEQ ID |
| | | WFRQAPGKEREF | | | NO: 416) |
| | | VASILWRYEYTNY | | | |
| | | ANSVKGRFTISAD | | | |
| | | KAKNTVYLQMNSL | | | |
| | | KPEDTAVYYCACA | | | |
| | | IERSGIYYHEDSN | | | |
| | | KYQYWGQGTQVS | | | |
| | | VS (SEQ ID NO: | | | |
| | | 100) | | | |
| S2-13 | S2 | MAQVQLVESGGG | FTMTYYTMT | SSRITGNTY | GRNINYYTN |
| | | LVQPGGSLRLSCA | (SEQ ID NO: | (SEQ ID NO: | SAQYDY |
| | | ASGFTMTYYTMT | 417) | 418) | (SEQ ID NO: |
| | | WFRQAPGKEREW | | | 419) |
| | | VSSSRITGNTYYT | | | |
| | | DSVKGRFTISRDN | | | |
| | | AKSTVYLQMNSLK | | | |
| | | PEDTATYYCASGR | | | |
| | | NINYYTNSAQYDY | | | |
| | | WGRGTQVTVS | | | |
| | | (SEQ ID NO: 101) | | | |
| S2-14 | S2 | MAQVQLVESGGG | RTFSPYAM | GIGWYSGT | SKDRYGNM |
| | | LVQTGGSLRLSCA | G (SEQ ID | TY (SEQ ID | DPNGVRYD |
| | | PSGRTFSPYAMG | NO: 420) | NO: 421) | Y (SEQ ID |
| | | WFRQAPGKEREF | | | NO: 422) |
| | | VSGIGWYSGTTYY | | | |
| | | ADSVKGRFTISSDI | | | |
| | | IKNTLTLQMNSLK | | | |
| | | PEDTAVYYCAASK | | | |
| | | DRYGNMDPNGVR | | | |
| | | YDYWGQGTQVTV | | | |
| | | S (SEQ ID NO: | | | |
| | | 102) | | | |
| S2-15 | S2 | MAQVQLVESGGG | RTFSWYNM | GISLTDISRS | DDRRMPAAI |
| | | LVQAGGSLRLSCA | A (SEQ ID | GGNTY | MTSVRDYV |
| | | ASGRTFSWYNMA | NO: 423) | (SEQ ID NO: | Y (SEQ ID |
| | | WFRQAPGKEREF | | 424) | NO: 425) |
| | | VAGISLTDISRSGG | | | |
| | | NTYYADSVKGRFT | | | |
| | | ISRDNAKNTVYMQ | | | |
| | | MDSLKPEDTAVYY | | | |
| | | CAADDRRMPAAI | | | |
| | | MTSVRDYVYRGQ | | | |
| | | GTQVTVS (SEQ ID | | | |
| | | NO: 103) | | | |
| S2-18 | S2 | MTQVQLVESGGG | STFSSRTM | AIPWWIGAL | SKTWSPRT |
| | | LVQAGDSLRLSCV | G (SEQ ID | PN (SEQ ID | DVTELEF |
| | | ASESTFSSRTMG | NO: 426) | NO: 427) | (SEQ ID NO: |
| | | WFRQAPGKERDF | | | 428) |
| | | VAAIPWWIGALPN | | | |
| | | YKDSVKGRFTISR | | | |
| | | DSAKNTMYLQMN | | | |
| | | SLKPEDTAVYYCA | | | |
| | | ASKTWSPRTDVT | | | |
| | | ELEFRGQGTQVT | | | |
| | | VS (SEQ ID NO: | | | |
| | | 104) | | | |
| S2-22 | S2 | MAQVHLAESGGG | RTFSWYNL | GIGRIGGNIY | DDRRMPAAI |
| | | LVQAGGSLRLSCA | A (SEQ ID | (SEQ ID NO: | MTSARDYV |
| | | ASGRTFSWYNLA | NO: 429) | 430) | Y (SEQ ID |
| | | WFRQTPGKEREF | | | NO: 431) |
| | | VAGIGRIGGNIYYA | | | |
| | | DSVKGRFTISEDN | | | |
| | | AKNTVYLKMDSLK | | | |
| | | PEDTAVYHCAAD | | | |
| | | DRRMPAAIMTSAR | | | |
| | | DYVYWGQGTQVT | | | |
| | | VS (SEQ ID NO: | | | |
| | | 105) | | | |
| S2-26 | S2 | MAQVQLVESGGG | GTFSRYSM | AISSNDKAT | DPVLKMTP |
| | | LVQAGGSLRLSCA | G (SEQ ID | Y (SEQ ID | DIMASRYVY |
| | | VSGGTFSRYSMG | NO: 432) | NO: 433) | (SEQ ID NO: |
| | | WFRQAPGKEREF | | | 434) |
| | | VAAISSNDKATYY | | | |
| | | KDSVKDRFTISRD | | | |
| | | NAKNTVYLHMNTL | | | |
| | | KPEDTAVYYCAAD | | | |
| | | PVLKMTPDIMASR | | | |
| | | YVYWGQGTQVTV | | | |
| | | S (SEQ ID NO: | | | |
| | | 106) | | | |
| S2-33 | S2 | MAQVQLLESGGG | SIANMRVMK | TMFNPGPT | VDPRNYEY |
| | | LVQPGGSLRLSCV | (SEQ ID NO: | M (SEQ ID | (SEQ ID NO: |
| | | VSGSIANMRVMK | 435) | NO: 436) | 437) |
| | | WYRQAPGKERE | | | |
| | | WVGTMFNPGPTM | | | |
| | | YRDSVKGRFTISR | | | |
| | | DNAKNTVNLQMN | | | |
| | | SLKVEDTAVYYCN | | | |
| | | VVDPRNYEYWGQ | | | |
| | | GTQVTVS (SEQ ID | | | |
| | | NO: 107) | | | |
| S2-35 | S2 | MAQVQLVESGGG | RISSVEVLG | TLTRAGTTN | RDLIGQNF |
| | | LVQPGGSLRLSCA | (SEQ ID NO: | (SEQ ID NO: | (SEQ ID NO: |
| | | ASFRISSVEVLGW | 438) | 439) | 440) |
| | | YRQTPGKERELVA | | | |
| | | TLTRAGTTNYIDA | | | |
| | | VKGRFTISRDNAK | | | |
| | | NMLYLQMNSLKP | | | |
| | | EDTAVYYCNVRDL | | | |
| | | IGQNFWGQGTHV | | | |
| | | TVS (SEQ ID NO: | | | |
| | | 108) | | | |
| S2-36 | S2 | MAQVQLVESGGG | RIATNYVMD | SISNDGSTN | KHWWFGFA |
| | | LVQAGGSLRLSCA | (SEQ ID NO: | (SEQ ID NO: | KYDY (SEQ |
| | | ASGRIATNYVMD | 441) | 442) | ID NO: 443) |
| | | WYRQAPGKQQEL | | | |
| | | VASISNDGSTNYA | | | |
| | | DSVKGRFTISRDN | | | |
| | | AKNTLYLQMNSLK | | | |
| | | PEDTAVYLCHLKH | | | |
| | | WWFGFAKYDYW | | | |
| | | GQGTQVTVS | | | |
| | | (SEQ ID NO: 109) | | | |
| S2-39 | S2 | MAQVQLVESGGD | IIISRYAMD | EISSGGTTN | SYTTY (SEQ |
| | | LVQAGESLRLSCA | (SEQ ID NO: | (SEQ ID NO: | ID NO: 446) |
| | | ASGIIISRYAMDWY | 444) | 445) | |
| | | RQAPGKQREFVA | | | |
| | | EISSGGTTNYADS | | | |
| | | VKGRFTIFRDNAK | | | |
| | | NTMYLQMNSLKP | | | |
| | | EDTAVYYCKVSYT | | | |
| | | TYSGQGTRVTVS | | | |
| | | (SEQ ID NO: 110) | | | |
| S2-40 | S2 | MAQVQLVESGGG | RTFSWYNL | GISRIAGNT | DDRWMPAA |
| | | LVQAGGSLRLSCA | A (SEQ ID | Y (SEQ ID | TMTSVRDY |
| | | ASGRTFSWYNLA | NO: 429) | NO: 448) | VY (SEQ ID |
| | | WFRQAPGKEREF | | | NO: 449) |
| | | VAGISRIAGNTYYA | | | |
| | | DSVKGRFTISEDN | | | |
| | | AKNTVYMQMDSL | | | |
| | | KPEDTAVYHCAAD | | | |
| | | DRWMPAATMTSV | | | |
| | | RDYVYRGQGTQV | | | |
| | | TVS (SEQ ID NO: | | | |
| | | 111) | | | |
| S2-42 | S2 | MAQVQLVESGGG | RTFSWYNL | GISRTAGNT | DDRRMAAA |
| | | LVQAGGSLRLSCA | A (SEQ ID | Y (SEQ ID | TMTSVRDY |
| | | ASGRTFSWYNLA | NO: 429) | NO: 451) | VY (SEQ ID |
| | | WFRQAPGKEFEF | | | NO: 452) |
| | | VGGISRTAGNTYY | | | |
| | | ADSVKGRFTISED | | | |
| | | NAKNTVYIQMDSL | | | |
| | | KPEDTAVYHCAAD | | | |
| | | DRRMAAATMTSV | | | |
| | | RDYVYRGQGTQV | | | |
| | | TVS (SEQ ID NO: | | | |
| | | 112) | | | |
| S2-47 | S2 | MAQVRVEESGGD | ILNTWPMG | QITSGGTTN | RADRDY |
| | | LVQAGGSLRLSCT | (SEQ ID NO: | (SEQ ID NO: | (SEQ ID NO: |
| | | TSAILNTWPMGW | 453) | 454) | 455) |
| | | YRQAPGKQRELV | | | |
| | | AQITSGGTTNYAD | | | |
| | | SVKGRFTISRDGR | | | |
| | | QNIVYLQMNRLKP | | | |
| | | EDTAVYYCNLRAD | | | |
| | | RDYWGQGTQVTV | | | |
| | | S (SEQ ID NO: | | | |
| | | 113) | | | |
| S2-57 | S2 | MAQVQLVESGGG | RIFSSYAMA | TITWSGKLT | HEKGWKQV |
| | | LVQAGGSLRLSCV | (SEQ ID NO: | M (SEQ ID | SPDDVKY |
| | | ASGRIFSSYAMAW | 456) | NO: 457) | (SEQ ID NO: |
| | | FRQAPGKEREFV | | | 458) |
| | | ATITWSGKLTMYQ | | | |
| | | DSVKGRFTISKDN | | | |
| | | PKNTVYLQMNTLK | | | |
| | | PEDTALYYCAAHE | | | |
| | | KGWKQVSPDDVK | | | |
| | | YWGQGTQVTVS | | | |
| | | (SEQ ID NO: 114) | | | |
| S2-59 | S2 | MAQVQLVESGGG | RTISNYNMG | AAKWSDINE | AGGYYSDNI |
| | | QAQAGGSLRLSC | (SEQ ID NO: | S (SEQ ID | RQTDYNY |
| | | AASGRTISNYNMG | 459) | NO: 460) | (SEQ ID NO: |
| | | WFRQAPGKEREF | | | 461) |
| | | VAAAKWSDINESY | | | |
| | | ADSVKGRFTISRD | | | |
| | | NAKNTVYLQMNSL | | | |
| | | KPEDTAVYYCAAA | | | |
| | | GGYYSDNIRQTDY | | | |
| | | NYWGQGTQVTVS | | | |
| | | (SEQ ID NO: 115) | | | |
| S2-62 | S2 | MAQVQLVESGGG | RTFSSAMM | SIDWRYQY | QIQRDGIYY |
| | | LVQPGGSLRLSCA | G (SEQ ID | RY (SEQ ID | YEDSSKYQ |
| | | ASGRTFSSAMMG | NO: 462) | NO: 463) | Y (SEQ ID |
| | | WFRQAPGKEREF | | | NO: 464) |
| | | VASIDWRYQYRYY | | | |
| | | ADSVKGRFTISRD | | | |
| | | NAKNTVYLQMNSL | | | |
| | | KPDDTAVYYCAC | | | |
| | | QIQRDGIYYYEDS | | | |
| | | SKYQYWGQGTQV | | | |
| | | SVS (SEQ ID NO: | | | |
| | | 116) | | | |

The CDR and FR are computed by LlamaMagic (Fridy et al. Nature Methods 2014; 11(12): 1253-1260). The CDRs follow the Martin (enhanced Chothia) numbering scheme (Mol Immunol. 2008 August; 45(14):3832-3839; World Wide Web at bioinf.org.uk/abs/info.html).

While the CDR sequences provided above are based on LlamaMagic, other CDRs based on the provided chains can be determined by other methods known in the art. For example, definitive delineation of a CDR and identification of residues including the binding site of a nanobody can be accomplished by solving the structure of the nanobody and/or solving the structure of the nanobody-epitope complex. In particular embodiments, this can be accomplished by methods such as X-ray crystallography. Alternatively, CDRs are determined by comparison to known nanobodies (Iinear sequence) and without resorting to solving a crystal structure.

In addition to LlamaMagic, CDR sets can be based on, for example, Kabat numbering (Kabat et al. (1991), “Sequences of Proteins of Immunological Interest,” 5th Ed. Public Health Service, National Institutes of Health, Bethesda, Md. (“Kabat” numbering scheme)); Chothia (Al-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), IMGT (Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme)), AHo (Honegger A and Plückthun A, “Yet another numbering scheme for immunoglobulin variable domains: an automatic modeling and analysis tool,” J Mol Biol, 2001 Jun. 8; 309(3):657-70, (AHo numbering scheme)), North (North et al., J Mol Biol. 406(2):228-256 (2011), “A new clustering of antibody CDR loop conformations”), or other numbering schemes.

Software programs and bioinformatical tools, such as ABodyBuilder and Paratome can also be used to determine CDR sequences.

The single-domain antibodies (used interchangeably with nanobodies herein) can be utilized as individual binding domains and/or can be engineered into various formats of binding molecules for the research, diagnosis, and/or treatment of SARS-CoV-2 infection and COVID-19.

In particular embodiments, a single-domain antibody can be derived from or based on a disclosed V_{H} domain and can include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the disclosed V_{H} disclosed herein. An insertion, deletion or substitution may be anywhere in the V_{H} domain, including at the amino- or carboxy-terminus or both ends of this domain, provided that each CDR includes zero changes or at most one, two, or three changes and provided a V_{H} domain including the modified V_{H} domain can still specifically bind its target spike protein epitope with an affinity similar to its reference domain.

In particular embodiments, a variant includes or is a sequence that has at least 90%, at least 91%, at least 92%, at least 93%, at least 94%, at least 95%, at least 96%, at least 97%, at least 98%, at least 99%, at least 99.5% sequence identity to a heavy chain variable domain (V_{H}), wherein each CDR includes zero changes or at most one, two, or three changes, from the reference antibody disclosed herein or fragment or derivative thereof that specifically binds anSARS-CoV-2 epitope as described herein. For more information regarding sequence identity, see the Closing Paragraphs section of this disclosure.

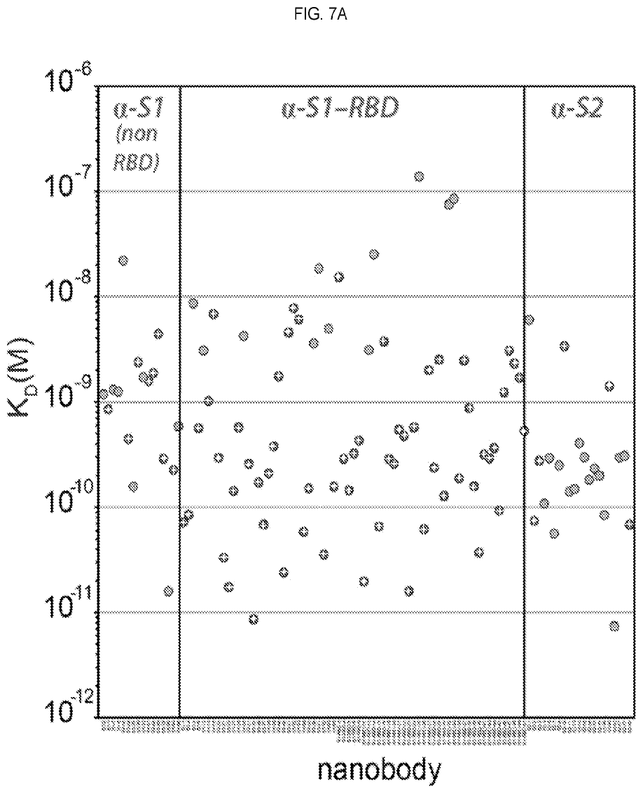

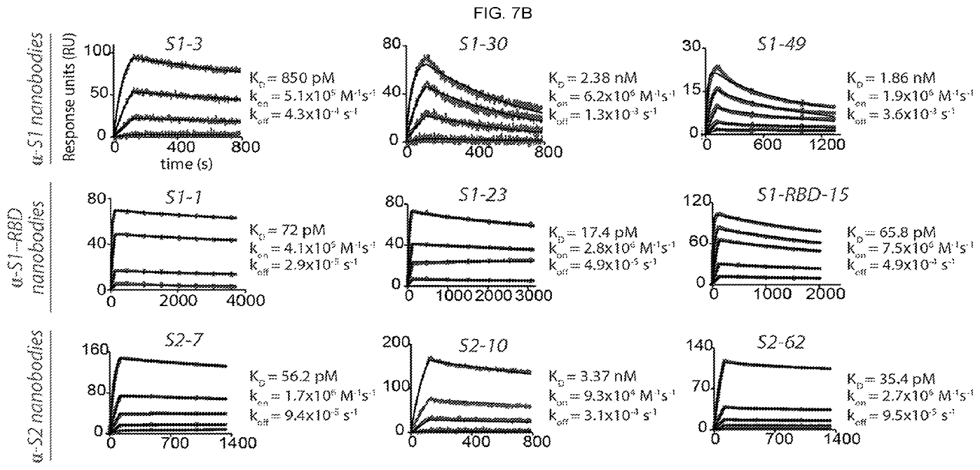

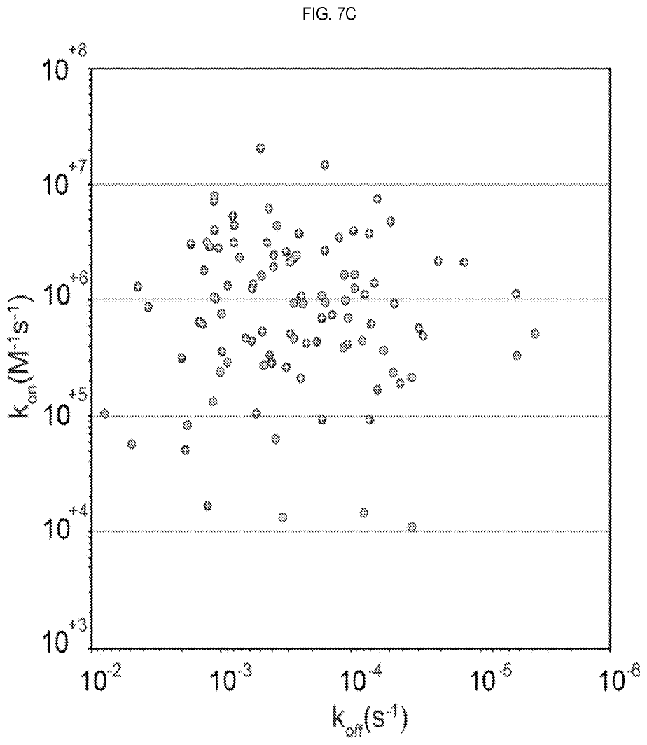

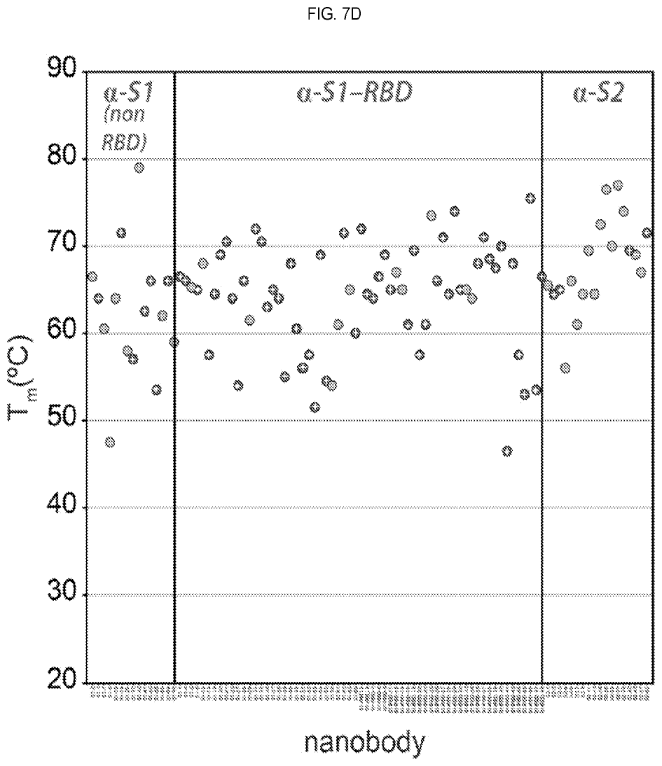

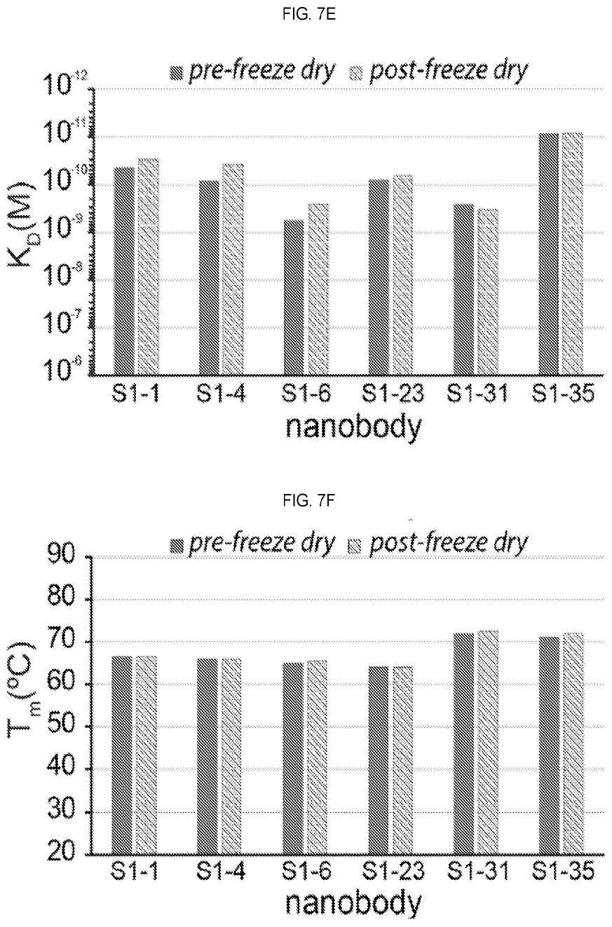

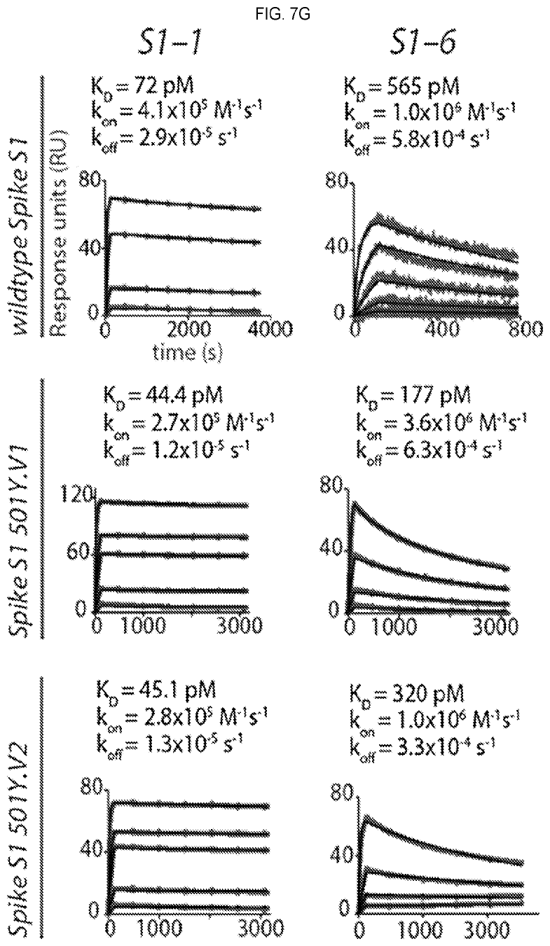

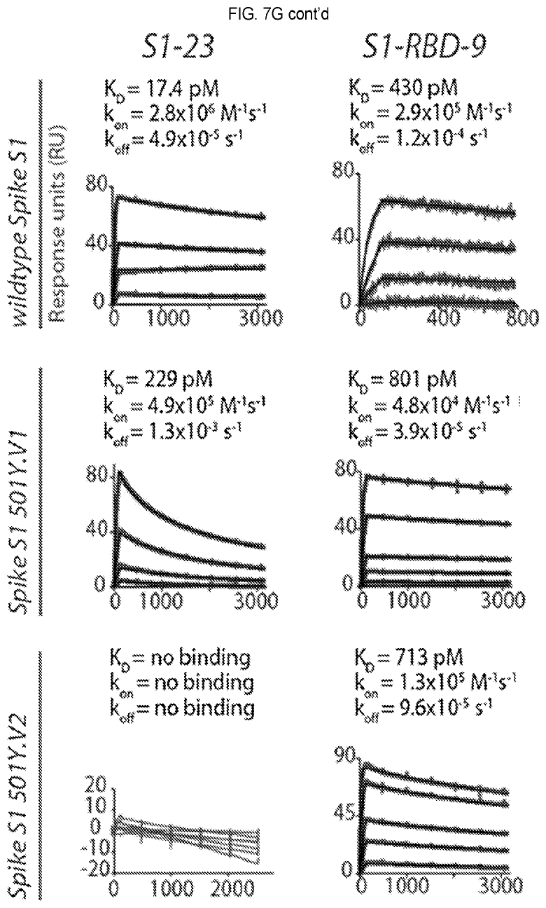

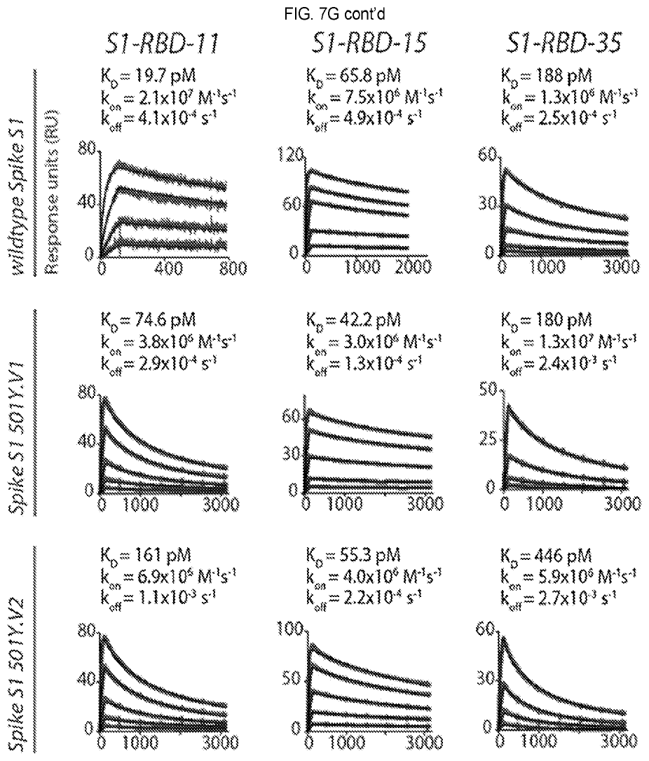

Within the current disclosure, surface plasmon resonance (SPR) was used to detail the kinetic properties and affinities of the selected nanobodies (FIGS. 5, 6). All bound with high affinity, with over 60% binding with K_{D}s less than 1 nM, and two with single digit picomolar affinities (FIGS. 7A-7G). While most S1 binding nanobodies bind RBD (71 nanobodies), 16 nanobodies targeted non-RBD regions of S1 and 26 bind S2 (FIGS. 7A-7G). The disproportionate number of non-RBD S1 and S2 nanobodies reveals the highly antigenic nature of the RBD and highlights likely occlusion of non-RBD S1 regions and S2 due to the glycan shield of SARS-CoV2 Spike (Grant et al., 2020, Sci Rep 10, 14991.; Watanabe et al., 2020, Science 369, 330-333). At the same time, no obvious bias in nanobody affinities for these different domains were observed. While both high on rates and low off rates contributed to these high affinities, kinetic analyses underscore the uniformly fast association rates (kₒₙ≥10⁺⁶) of these nanobodies (Iikely due to the nanobodies' small size and proportionally large paratope surface area), with many surpassing the kon rates of high-performing monoclonal antibodies (kₒₙ=10⁺⁵) (Tian et al., 2020, Emerg Microbes Infect 9, 382-385) (FIGS. 7A-7G), a property that would benefit translation of these nanobodies into rapid therapeutics and diagnostics (Carter, 2006, Nat Rev Immunol 6, 343-357). For those nanobodies with apparently homologous paratopes (FIG. 7C), no correlation in their kinetic properties were found (FIGS. 5, 6), demonstrating that even small paratope changes can strongly alter behaviors (Fridy et al., 2014, Nat Methods 11, 1253-1260).

A worrying recent development has been the emergence of viral variants, with mutations in RBD that minimize or nullify binding of many currently available monoclonal antibodies and nanobodies (Diamond et al., 2021, Res Sq. doi: 10.21203/rs.3.rs-228079/v1; Garcia-Beltran et al., 2021, medRxiv; Jangra et al., 2021, medRxiv; Liu et al., 2021, bioRxiv; Sun et al., 2021, bioRxiv; Wang et al., 2021, Nature 592(7855):616-622; Weisblum et al., 2020, Elife 9). Indeed in one study, the efficacy of 14 out of the 17 most potent monoclonal antibodies tested was compromised by such common RBD mutants (Wang et al., 2021, Nature 592(7855):616-622). Here, nanobodies of the disclosure alone and in combination show great potential to be particularly resistant to these variants (Sun et al., 2021, bioRxiv).

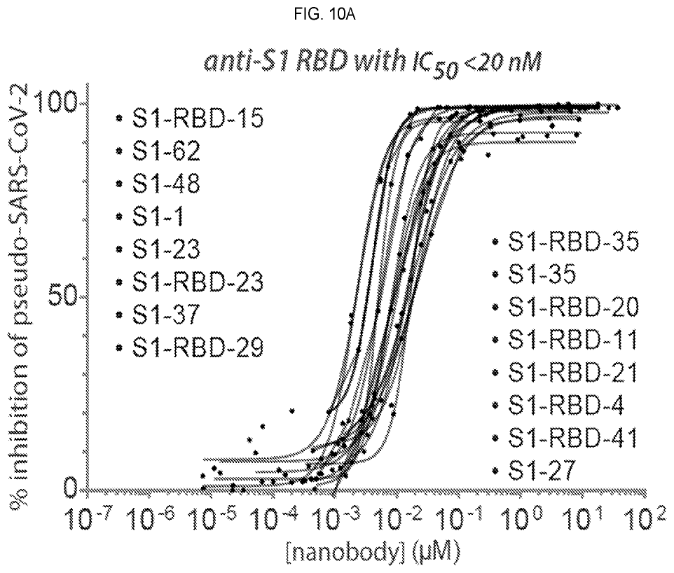

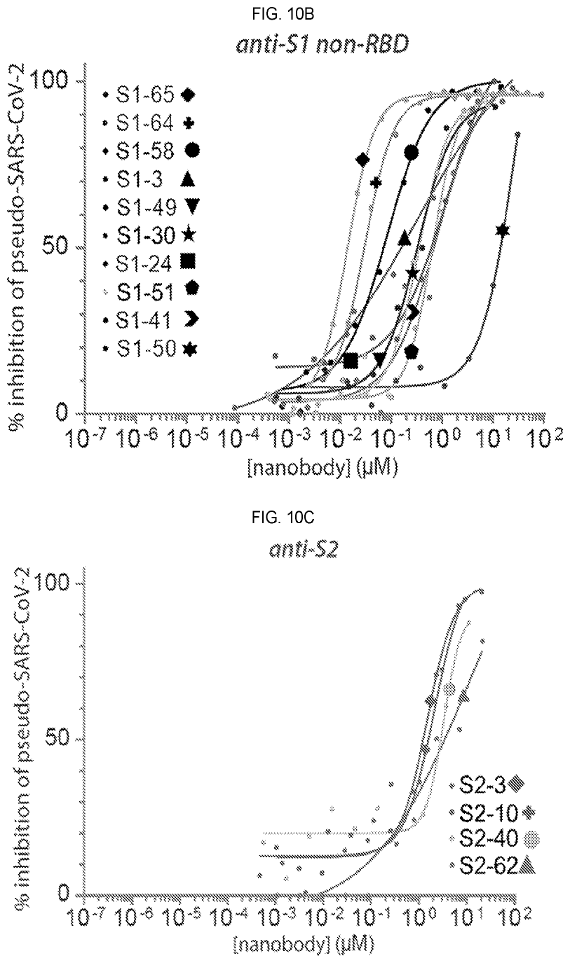

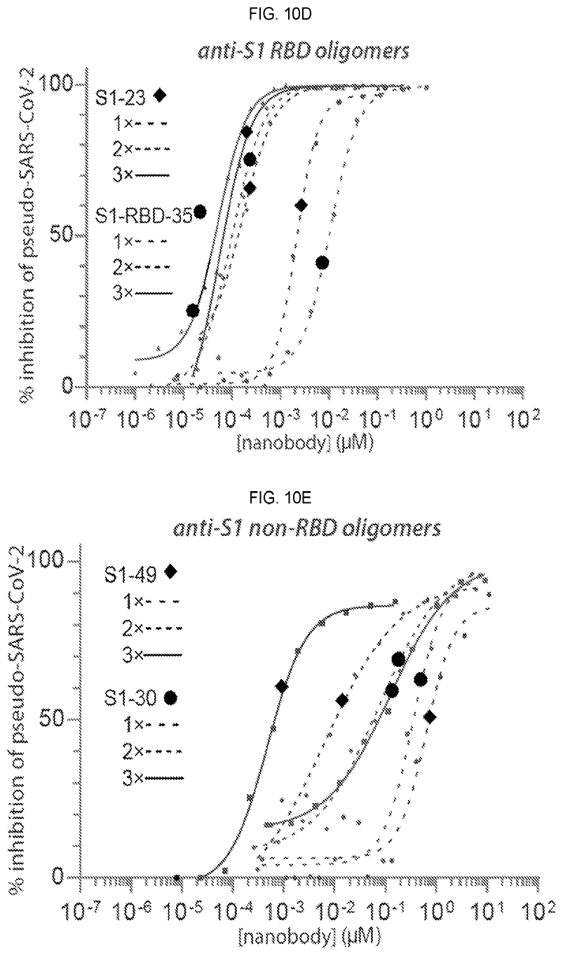

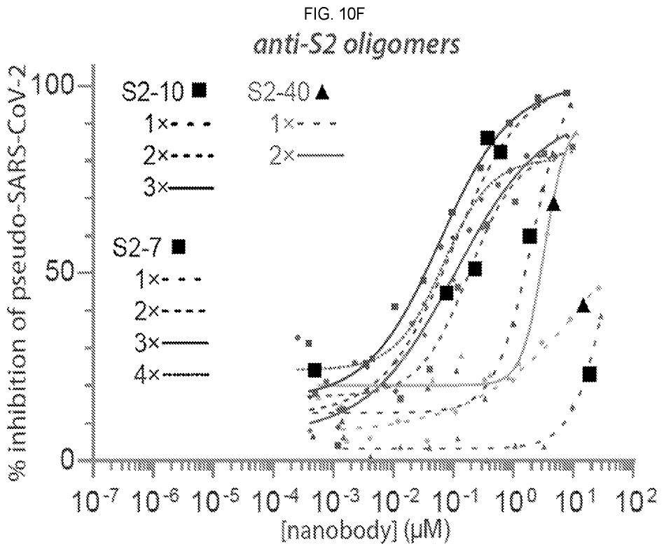

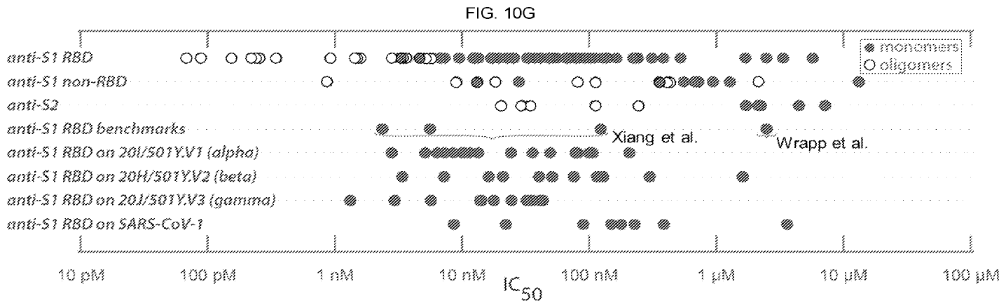

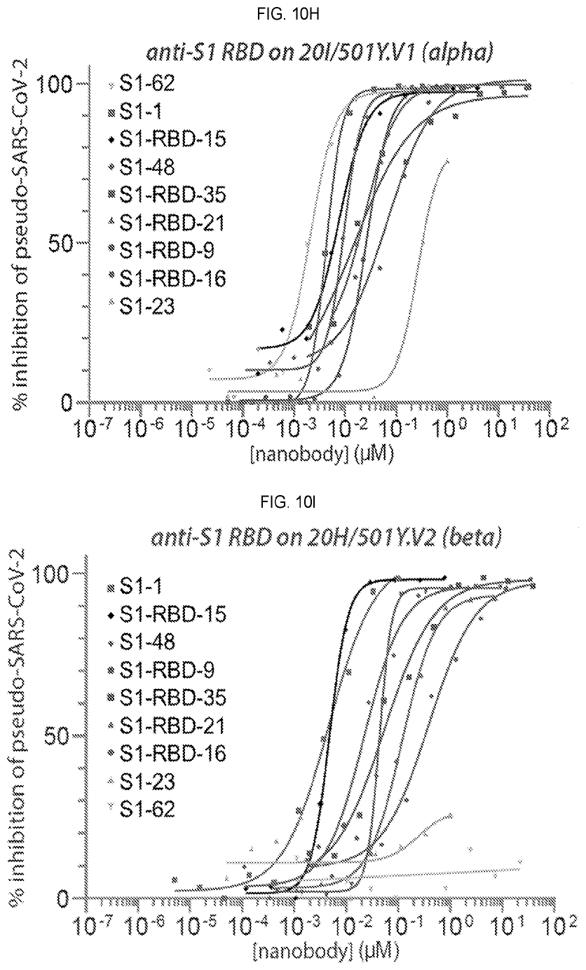

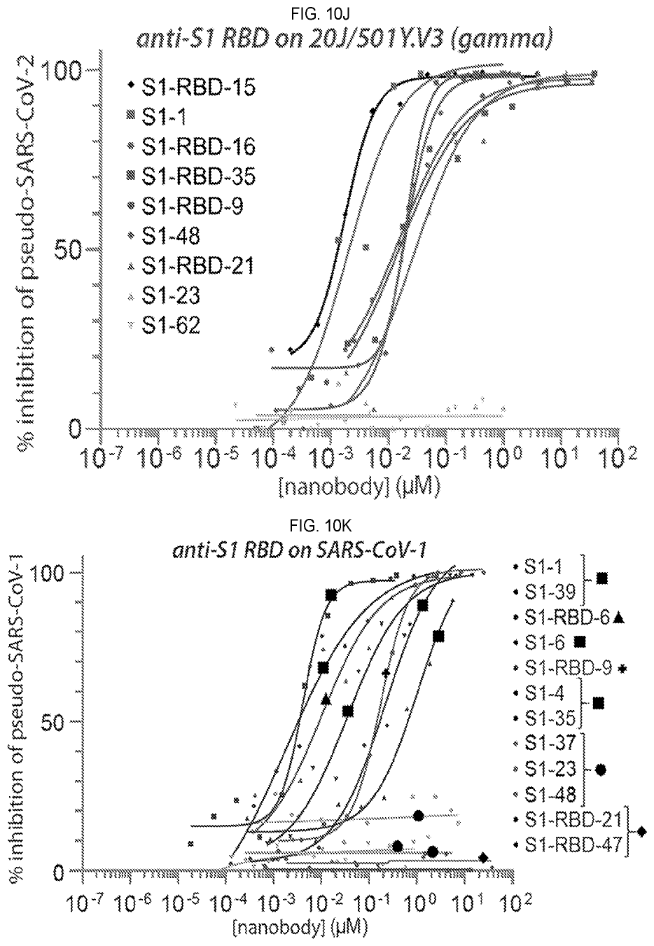

A SARS-CoV-2 pseudovirus neutralization assay was used to screen and characterize disclosed nanobodies for antiviral activities (FIGS. 10A-10J). The most potent neutralizing nanobodies mapped to the RBD. Notably, nanobodies mapping outside of the RBD on S1 (anti-S1, non RBD) and mapping to S2 also neutralized the pseudovirus (FIGS. 10B, 10C). This is the first evidence of nanobody neutralization activity mapping outside of the RBD. As nanobodies are monomeric, the mechanism of this neutralization does not involve viral aggregation and likely reflects disruption of the viral binding or spike driven fusion of viral and cellular membranes of fusion with target cell membranes.

Both SARS-CoV-1 and SARS-CoV-2 share the same host receptor, ACE2, and the RBDs of the viruses share 74% identity. As a result, some antibodies and nanobodies have been shown to be cross-neutralizing (Liu et al., 2020, Immunity 53, 1272-1280 e1275; Wrapp et al., 2020, Cell 181, 1004-1015 e1015). Of the tested nanobodies disclosed herein, many (8 of 23 tested) also displayed excellent neutralizing activities against the SARS-CoV-1 Spike pseudotyped virus (FIG. 10K).

Certain mutations appearing in ‘variants of concern’ (VOC) have been associated with rapidly increasing case numbers in certain locales, have been demonstrated to reduce the neutralization potency of some monoclonal antibodies and polyclonal plasma, increase the frequency of serious illness, and are spreading rapidly (19A; Wuhan-Hu-1) virus (Wang et al., 2021, Nature 592(7855):616-622; Wibmer et al., 2021, Nat Med 27:622-625). A subset of better neutralizing nanobodies was therefore tested against a pseudovirus carrying the Spike protein of the B.1.351/20H/501Y.V2 variant, that includes the three amino acid substitutions at positions K417N/T, E484K and N501Y (Stamatatos et al., 2021, Science 372(6549):1413-1418; Tegally et al., 2021, Nature 592:438-443). While nanobodies S1-23 and S1-37 failed to neutralize the pseudovirus variant, nanobody S1-1 was equally efficacious against the variant as against the pseudovirus carrying wild-type Spike (FIG. 10I). Both S1-RBD-21 and -35 also remained effective neutralizers of the 20H/501Y.V2 variant Spike pseudotypes, albeit with 10-fold reduction in their IC50s compared to the wild-type Spike (FIG. 10I). Remarkably though, two nanobodies, S1-RBD-9 and -15 showed increased neutralization activity against the 20H/502Y.V2 Spike pseudoviruses, with the activity of S1-RBD-15 increasing 10-fold (FIG. 10J). Overall, these data suggest that comprehensive mining of the disclosed library and multimerization can lead to nanobody-based therapies that remain fully effective against common and potentially yet-to-emerge variants of SARS-CoV-2 and with broad spectrum coronavirus inhibition activities.

Drugs are often combined to improve single drug efficacies and to reduce efficacious drug concentrations. Synergy occurs when the combination of drugs has a greater effect than the sum of the individual effects of each drug. A major advantage of a large library of nanobodies that bind to different epitopes on Spike is the potential for cooperative activity among nanobody pairs (or higher order combinations) leading to synergistic effects. Because the disclosed nanobody library provides for thousands of pairwise combinations and this scales exponentially when considering cocktails with three or more nanobodies, pairs of nanobodies were tested for synergistic activities. Combinations of S1-27 and S1-23 showed simple additive effects. These nanobodies belong to the same epitope bin (FIG. 7B); their additive effect is as expected for two nanobodies accessing the same site on S1-RBD, but effectively doubling the concentration of a single nanobody.

To move beyond additive effects, combinations that bind to different epitopes were considered. Indeed, synergistic effects were observed between S1-23 and S1-1, S1-RBD-15, and S2-40. The most dramatic synergistic effect was observed with S1-RBD-15 which improves the potency of S1-23 by 300-fold. Interestingly, nanobodies with synergistic neutralizing activity showed nearby, but non-overlapping epitopes on the RBD of Spike, and can provide a roadmap to the rational production of multimeric, even higher affinity reagents capable of neutralization at low doses while minimizing susceptibility to escape mutations.

With the emerging variants of concern, one goal is to develop nanobody multimers and cocktails that are maximally refractory to escape by such variants. Some of the most potently neutralizing nanobodies selected are resistant to VOCs (e.g. E484K) similarly to those selected by potent neutralizing antibodies that have been cloned from SARS-CoV-2 convalescents and vaccine recipients, confirming that the ACE2 binding site is a point of particular vulnerability for potent neutralization.

Beyond their enhanced combined activities, nanobody cocktails are expected to be resistant to escape (Baum et al., 2020, Science 369, 1014-1018; Gasparo et al., 2021, bioRxiv; Weisblum et al., 2020, Elife 9). Such mixtures or derived multimers may represent powerful escape resistant therapeutics, and even more escape resistance should be possible by the use of three or more carefully chosen nanobodies in cocktails or multimers.

As indicated, single-domain antibodies disclosed herein can be utilized as single binding domains or can be engineered into various binding molecule formats. As used herein, a binding molecule includes at least one single-domain antibody disclosed herein. Examples, described in more detail below, include (i) Multimerized Single-Domain Antibodies; (ii) Heavy Chain Only (HcAb) SARS-CoV-2 Antibodies; (iii) Multi-Specific Binding Molecules, (iv) Single-Domain Antibody Conjugates, and (v) Chimeric Antigen Receptors (CAR). The current disclosure also provides (vi) Compositions and Combination Cocktails, (vii) Methods of Use, (viii) Methods to Obtain and Use Single-Domain Antibodies; (ix) Exemplary Embodiments; (x) Experimental Examples, (xi) Closing Paragraphs, and (xii) References. While section headings are provided, these headings are for organizational purposes only and do not limit the scope or interpretation of the disclosure.
(i) Multimerized Single-Domain Antibodies. As indicated, single-domain antibodies disclosed herein can be multimerized. Multimerization strategies are described in this section (i) with additional strategies described in section (ii) below.

In certain aspects, multimerization is achieved by linking single-domain antibodies in a fusion protein with protein linkers. Fusion proteins include different protein domains (e.g., single-domain antibodies) linked to each other directly or through intervening linker segments such that the function of each included domain is retained.

Commonly used flexible linkers include linker sequences with the amino acids glycine and serine (Gly-Ser linkers). In particular embodiments, the linker sequence includes sets of glycine and serine repeats such as from one to ten repeats of (GlyₓSer_{y})ₙ (SEQ ID NO: 490), wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 489), (Gly₃Ser)ₙ(Gly₄Ser)ₙ (SEQ ID NO: 491), (Gly₃Ser)ₙ(Gly₂Ser)ₙ (SEQ ID NO: 492), and (Gly₃Ser)ₙ(Gly₄Ser), (SEQ ID NO: 493). In particular embodiments, the linker is (Gly₄Ser)₄ (SEQ ID NO: 494), (Gly₄Ser)₃ (SEQ ID NO: 495), (Gly₄Ser)₂ (SEQ ID NO: 496), (Gly₄Ser), (SEQ ID NO: 497), (Gly₃Ser)₂ (SEQ ID NO: 498), (Gly₃Ser), (SEQ ID NO: 499), (Gly₂Ser)₂ (SEQ ID NO: 500) or (Gly₂Ser)₁, GGSGGGSGGSG (SEQ ID NO: 501), GGSGGGSGSG (SEQ ID NO: 502), or GGSGGGSG (SEQ ID NO: 503).

Linkers can also include one or more antibody hinge regions and/or immunoglobulin heavy chain constant regions, such as CH3 alone or a CH2CH3 sequence. Additional examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target.

Certain examples include fusion protein with two or three copies of a single-domain antibody disclosed herein (e.g., S1-23), each linked with the Gly-Ser linker (Gly₄Ser)₄ (SEQ ID NO: 494).

Other examples of fusion proteins include 2, 3, 4, 5, 6, 7, 8, 9 or 10 copies of S1-1, S1-2, S1-3, S1-4, S1-5, S1-6, S1-7, S1-9, S1-10, S1-11, S1-12, S1-14, S1-17, S1-19, S1-20, S1-21, S1-23, S1-24, S1-25, S1-27, S1-28, S1-29, S1-30, S1-31, S1-32, S1-35, S1-36, S1-37, S1-38, S1-39, S1-41, S1-46, S1-48, S1-49, S1-50, S1-51, S1-52, S1-53, S1-54, S1-55, S1-56, S1-58, S1-60, S1-61, S1-62, S1-63, S1-64, S1-65, S1-66, S1-RBD-3, S1-RBD-4, S1-RBD-5, S1-RBD-6, S1-RBD-9, S1-RBD-10, S1-RBD-11, S1-RBD-12, S1-RBD-14, S1-RBD-15, S1-RBD-16, S1-RBD-18, S1-RBD-19, S1-RBD-20, S1-RBD-21, S1-RBD-22, S1-RBD-23, S1-RBD-24, S1-RBD-25, S1-RBD-26, S1-RBD-27, S1-RBD-28, S1-RBD-29, S1-RBD-30, S1-RBD-32, S1-RBD-34, S1-RBD-35, S1-RBD-36, S1-RBD-37, S1-RBD-38, S1-RBD-39, S1-RBD-40, S1-RBD-41, S1-RBD-43, S1-RBD-44, S1-RBD-45, S1-RBD-46, S1-RBD-47, S1-RBD-48, S1-RBD-49, S1-RBD-51, S2-1, S2-2, S2-3, S2-4, S2-5, S2-6, S2-7, S2-9, S2-10, S2-11, S2-13, S2-14, S2-15, S2-18, S2-22, S2-26, S2-33, S2-35, S2-36, S2-39, S2-40, S2-42, S2-47, S2-57, S2-59 or S2-62, each copy within the fusion protein linked to another copy through a protein linker (e.g., a Gly-Ser linker such as SEQ ID NO: 494).

A “multimerization domain” is a domain that causes two or more proteins (monomers) to interact with each other through covalent and/or non-covalent association(s). Multimerization domains are highly conserved protein sequences that can include different types of sequence motifs such as leucine zipper, helix loop-helix, ankyrin and PAS (Feuerstein et al, Proc. Natl. Acad. Sci. USA, 91:10655-10659, 1994). Multimerization domains present in proteins can bind to form dimers, trimers, tetramers, pentamers, hexamers, heptamers, etc., depending on the number of units/monomers incorporated into the multimer, and/or homomultimers or heteromultimers, depending on whether the binding monomers are the same type or a different type (U.S. patent Ser. No. 10/030,065).

Dimerization domains can include protein sequence motifs such as coiled coils, acid patches, zinc fingers, calcium hands, a CH1-CL pair, an “interface” with an engineered “knob” and/or “protruberance” (U.S. Pat. No. 5,821,333), leucine zippers (U.S. Pat. No. 5,932,448), SH2 and SH3 (Vidal et al., Biochemistry, 43:7336-44, 2004), PTB (Zhou et al., Nature, 378:584-592, 1995), WW (Sudol Prog Biochys MoL Bio, 65:113-132, 1996), PDZ (Kim et al., Nature, 378: 85-88, 1995; Komau et al., Science, 269:1737-1740, 1995) and WD40 (Hu et al., J Biol Chem., 273:33489-33494, 1998). Additional examples of molecules that contain dimerization domains/motifs are receptor dimer pairs such as the interleukin-8 receptor (IL-8R), integrin heterodimers such as LFA-I and GPIIIb/IIIa, dimeric ligand polypeptides such as nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa et al., J Biol. Chem., 269:27833-27839, 1994; Radziejewski et al., Biochem, 32: 1350, 1993) and variants of some of these domains with modified affinities (PCT Publication No. WO 2012/001647).

In particular embodiments, the sequence corresponding to a dimerization motif/domain includes the leucine zipper domain of Jun (U.S. Pat. No. 5,932,448; RIARLEEKVKTLKAQNSELASTANMLREQVAQLKQKVMN (SEQ ID NO: 544)), the dimerization domain of Fos (U.S. Pat. No. 5,932,448; LTDTLQAETDQLEDKKSALQTEIANLLKEKEKLEFILAA (SEQ ID NO: 545)), a consensus sequence for a WW motif (PCT Publication No. WO 1997/037223), the dimerization domain of the SH2B adapter protein from GenBank Accession no. AAF73912.1 (Nishi et al., Mol Cell Biol, 25: 2607-2621, 2005; WREFCESHARAAALDFARRFRLYLASHPQYAGPGAEAAFSRRFAELFLQHFEAEVARAS (SEQ ID NO: 546)), the SH3 domain of I1l from GenBank Accession no. AAD22543.1 (Kristensen el al., EMBO J., 25: 785-797, 2006; THRAIFRFVPRHEDELELEVDDPLLVELQAEDYWYEAYNMRTGARGVFPAYYAIE (SEQ ID. NO: 547)), the PTB domain of human DOK-7 from GenBank Accession no. NP_005535.1 (Wagner et al., Cold Spring Harb Perspect Biol. 5: a008987, 2013; LGEVHRFHVTVAPGTKLESGPATLHLCNDVLVLARDIPPAVTGQWKLSDLRRYGAVPSGFIFEG GTRCGYWAGVFFLSSAEGEQISFLFDCIVRGISPTKG (SEQ ID NO: 548)), the PDZ-like domain of SATB1 from UniProt Accession No. Q01826 (Galande et al., Mol Cell Biol. Aug; 21: 5591-5604, 2001; DCKEEHAEFVLVRKDMLFNQLIEMALLSLGYSHSSAAQAKGLIQVGKWNPVPLSYVTDAPDAT VADMLQDVYHVVTLKIQLHSCPKLEDLPPEQWSHTTVRNALKDLLKDMNQSS (SEQ ID NO: 549)), the WD40 repeats of APAF from UniProt Accession No. 014727 (Jorgensen et al., 2009. PLOS One. 4(12):e8463; CAPWPMVEKLIKQCLKENPQERPTSAQVFDILNSAELVCLTRRILLPKNVIVECMVATHHNSRN ASIWLGCGHTDRGQLSFLDLNTEGYTSEEVADSRILCLALVHLPVEKESWIVSGTQSGTLLVINT EDGKKRHTLEKMTDSVTCLYCNSFSKQSKQKNFLLVGTADGKLAIFEDKTVKLKGAAPLKILNIG NVSTPLMCLSESTNSTERNVMWGGCGSQLFSYAAFSDSNIITVVVDTALYIAKQNSPVVEVWD KKTEKLCGLIDCVHFLREVMVKETKIFSFSNDFTIQKLIETRTNKESKHKMSYSGRVKTLCLQKN TALWIGTGGGHILLLDLSTRRLIRVIYNFCNSVRVMMTAQLGSLKNVMLVLGYNRKNTEGTQKQ KEIQSCLTVWDINLPHEVQNLEKHIEVRKELAEKMRRTSVE (SEQ ID NO: 550)), the PAS motif of the dioxin receptor from UniProt Accession No. I6L9E7 (Pongratz et al., Mol Cell Biol, 18:4079-4088, 1998; DQELKHLILEAADGFLFIVSCETGRVVYVSDSVTPVLNQQQSEWFGSTLYDQVHPDDVDKLRE QLSTSENALTGR (SEQ ID NO: 551)) and the EF hand motif of parvalbumin from UniProt Accession No. P20472 (Jamalian et al., Int J Proteomics, 2014: 153712, 2014;

(SEQ ID NO: 552))
LSAKETKMLMAAGDKDGDGKIGVDEFSTLVAES.

In particular embodiments, the dimerization domain can be a dimerization and docking domain (DDD) on one nanobody and an anchoring domain (AD) on another nanobody to facilitate a stably tethered structure. In particular embodiments, the DDD (DDD1 and DDD2) are derived from the regulatory subunits of a cAMP-dependent protein kinase (PKA), and the AD (AD1 and AD2) are derived from a specific region found in various A-kinase anchoring proteins (AKAPs) that mediates association with the R subunits of PKA. In particular embodiments, DDD1 includes the amino acid sequence: SHIQIPPGLTELLQGYTVEVLRQQPPDLVEFAVEYFTRLREARA (SEQ ID NO: 553). In particular embodiments, DDD2 includes the amino acid sequence: CGHIQIPPGLTELLQGYTVEVLRQQPPDLVEFAVEYFTRLREARA (SEQ ID NO: 554). In particular embodiments, AD1 includes the amino acid sequence: QIEYLAKQIVDNAIQQA (SEQ ID NO: 555). In particular embodiments, AD2 includes the amino acid sequence: CGQIEYLAKQIVDNAIQQAGC (SEQ ID NO: 556). However, one skilled in the art will realize that other DDDs and ADs are known and can be used such as: the 4-helix bundle type DDD domains may be obtained from p53, DCoH (pterin 4 alpha carbinolamine dehydratase/dimerization cofactor of hepatocyte nuclear factor 1 alpha (TCF1)) and HNF-1 (hepatocyte nuclear factor 1). Other AD sequences of potential use may be found in Patent Publication No. US2003/0232420A1.

The X-type four-helix bundle dimerization motif that is a structural characteristic of the DDD (Newlon, et al. EMBO J. 2001; 20: 1651-1662; Newlon, et al. Nature Struct Biol. 1999; 3: 222-227) is found in other classes of proteins, such as the S100 proteins (for example, S100B and calcyclin), and the hepatocyte nuclear factor (HNF) family of transcriptional factors (for example, HNF-1a and HNF-1β). Over 300 proteins that are involved in either signal transduction or transcriptional activation also contain a module of 65-70 amino acids termed the sterile a motif (SAM) domain, which has a variation of the X-type four-helix bundle present on its dimerization interface. For S100B, this X-type four-helix bundle enables the binding of each dimer to two p53 peptides derived from the c-terminal regulatory domain (residues 367-388) with micromolar affinity (Rustandi, et al. Biochemistry. 1998; 37: 1951-1960). Similarly, the N-terminal dimerization domain of HNF-1α (HNF-p1) was shown to associate with a dimer of DCoH (dimerization cofactor for HNF-1) via a dimer of HNF-p1 (Rose, et al. Nature Struct Biol. 2000; 7: 744-748). In alternative embodiments, these naturally occurring systems can also be used to provide stable multimeric structures with multiple functions or binding specificities. Other binding events such as those between an enzyme and its substrate/inhibitor, for example, cutinase and phosphonates (Hodneland, et al. Proc Natl Acd Sci USA. 2002; 99: 5048-5052), may also be utilized to generate the two associating components (the “docking” step), which are subsequently stabilized covalently (the “lock” step).

In particular embodiments, dimerization of nanobodies can be induced by a chemical inducer. This method of dimerization requires one nanobody to contain a chemical inducer of dimerization binding domain 1 (CBD1) and the second nanobody to contain the second chemical inducer of dimerization binding domain (CBD2), wherein CBD1 and CBD2 are capable of simultaneously binding to a chemical inducer of dimerization (CID). If the CID is rapamycin, CBD1 and CBD2 can be the rapamycin binding domain of FK-binding protein 12 (FKBP12) and the FKBP12-Rapamycin Binding (FRB) domain of mTOR. In particular embodiments, FKBP12 includes the sequence:

(SEQ ID NO: 557)
MGVQVETISPGDGRTFPKRGQTCWHYTGMLEDGKKFDSSRDRNPFKFML
GKQEVIRGWEEGVAQMSVGQRAKLTISPDYAYGATGHPGIIPPHATLVF
DVELLKLE.

In particular embodiments, FRB includes the sequence: MASRILWHEMWHEGLEEASRLYFGERNVKGMFEVLEPLHAMMERGPQTLKETSFNQAYGRD LMEAQEWCRKYMKSGNVKDLTQAWDLYYHVFRRISKLES (SEQ ID NO: 558). If the CID is FK506/cyclosporin fusion protein or a derivative thereof, CBD1 and CBD2 can be the FK506 (Tacrolimus) binding domain of FK-binding protein 12 (FKBP12) and the cyclosporin binding domain of cylcophilin A. If the CID is estrone/biotin fusion protein or a derivative thereof, CBD1 and CBD2 can be an oestrogen-binding domain (EBD) and a streptavidin binding domain. If the CID is dexamethasone/methotrexate fusion molecule or a derivative thereof, CBD1 and CBD2 can be a glucocorticoid-binding domain (GBD) and a dihydrofolate reductase (DHFR) binding domain. If the CID is O⁶-benzylguanine derivative/methotrexate fusion molecule or a derivative thereof, CBD1 and CBD2 can be an O⁶-alkylguanine-DNA alkyltransferase (AGT) binding domain and a dihydrofolate reductase (DHFR) binding domain. If the CID is RSL1 or a derivative thereof, CBD1 and CBD2 can be a retinoic acid receptor domain and an ecodysone receptor domain. If the CID is AP1903 or a derivative thereof, CBD1 and CBD2 can be the FK506 binding protein (FKBP12) binding domains including a F36V mutation. Use of the CID binding domains can also be used to alter the affinity to the CID. For instance, altering amino acids at positions 2095, 2098, and 2101 of FRB can alter binding to Rapamycin: KTW has high, KHF intermediate and PLW is low (Bayle et al, Chemistry & Biology 13, 99-107, January 2006).

In particular embodiments, nanobodies can multimerize using a transmembrane polypeptide derived from a FcERI chain. In particular embodiments, a nanobody can include a part of a FcERI alpha chain and another nanobody can include a part of an FcERI beta chain or variant thereof such that said FcERI chains spontaneously dimerize together to form a dimeric nanobody. In particular embodiments, nanobodies can include a part of a FcERI alpha chain and a part of a FcERI gamma chain or variant thereof such that said FcERI chains spontaneously trimerize together to form a trimeric nanobody, and in another embodiment the multi-chain nanobody can include a part of FcERI alpha chain, a part of FcERI beta chain and a part of FcERI gamma chain or variants thereof such that said FcERI chains spontaneously tetramerize together to form a tetrameric nanobody.

In particular embodiments, additional methods of causing dimerization can be utilized. Additional modifications to generate a dimerization domain in nanobody could include: replacing the C-terminus domain with murine counterparts; generating a second interchain disulfide bond in the C-terminus domain by introducing a second cysteine residue into both nanobodies; swapping interacting residues in each of the nanobodies in the C-terminus domains (“knob-in-hole”); and fusing the variable domains of the nanobodies directly to CD3ζ (CD3ζ fusion) (Schmitt et al., Hum. Gene Ther. 2009. 20:1240-1248).

Particular embodiments can utilize multimerization domains, such as C4b multimerization domains or ferritin multimerization domains. Full-length native C4b includes seven α-chains linked together by a multimerization (i.e., heptamerization) domain at the C-terminus of the α-chains. Blom et al., (2004) Mol Immunol 40: 1333-1346. Ferritin is an iron storage protein found in almost all living organisms, and has been extensively studied and engineered for a number of biochemical/biomedical purposes (US 20090233377; Meldrum, et al. Science 257, 522-523 (1992); U.S. 20110038025; Yamashita, Biochim Biophys Acta 1800, 846-857 (2010), including as a multimerizing vaccine platform for displaying peptide epitopes (US 20060251679 (2006); Li, et al. Industrial Biotechnol 2, 143-147 (2006)).

Mutlimerization with encapsulin and lumazine synthase can also be performed. Both can be linked to nanobodies to create self-assembling 60mer particles (Jardine et al., 2013, Science 340, 711-716 and Kanekiyo et al., 2015, Cell 162, 1090-1100).
(ii) Heavy Chain Only (HcAb) SARS-CoV-2 Antibodies. HcAb include a single-domain antibody described herein linked to an Fc region of an antibody. For completeness, the following discussion of “classic” antibodies is provided. One of ordinary skill in the art will recognize portions of the discussion related to “classic” antibodies that equally apply to single-domain antibodies, and those portions that only apply to “classic” antibodies (e.g., discussion of light chains, which has relevance to examples of various multi-specific binding molecules described herein).

Naturally occurring human antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md., 1987 and 1991), Chothia (Chothia & Lesk, J. Mol. Biol., 196:901-917, 1987; Chothia et al., Nature, 342:878-883, 1989), or Martin (enhanced Chothia; Mol Immunol. 2008 August; 45(14):3832-3839). In particular embodiments, the CDR numbering is according to Martin.

Definitive delineation of a CDR and identification of residues including the binding site of an antibody can be accomplished by solving the structure of the antibody and/or solving the structure of the antibody-epitope complex. In particular embodiments, this can be accomplished by methods such as X-ray crystallography and cryoelectron microscopy. Alternatively, CDRs are determined by comparison to known antibodies (Iinear sequence) and without resorting to solving a crystal structure. To determine residues involved in binding, a co-crystal structure of the Fab (antibody fragment) bound to the target can optionally be determined. Software programs, such as ABodyBuilder can also be used.

The carboxy-terminal portion of each chain defines a constant region (the Fc region), which is responsible for effector function of the antibody. Examples of effector functions include: C1q binding and complement dependent cytotoxicity (CDC); antibody-dependent cell-mediated cytotoxicity (ADCC); phagocytosis; down regulation of cell surface receptors (e.g. B-cell receptors); and B-cell activation. A portion of an Fc region is a fragment of an Fc region. The fragment can include 10% of an Fc region, 20% of an Fc region, 30% of an Fc region, 40% of an Fc region, 50% of an Fc region, 60% of an Fc region, 70% of an Fc region, 80% of an Fc region, 90% of an Fc region, or 95% of an Fc region. A portion of an Fc region can also include a characterized segment of an Fc region, such as a CH2 region or a CH3 region.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2. IgG causes opsonization and cellular cytotoxicity and crosses the placenta, IgA functions on the mucosal surface, IgM is most effective in complement fixation, and IgE mediates degranulation of mast cells and basophils. The function of IgD is still not well understood. Resting B cells, which are immunocompetent but not yet activated, express IgM and IgD. Once activated and committed to secrete antibodies these B cells can express any of the five isotypes. The heavy chain isotypes of IgG, IgA, IgM, IgD and IgE are respectively designated the γ, α, μ, δ, and ε chains.

Antibodies and antibody-like molecules that can multimerize, such as IgA and IgM antibodies, have emerged as promising drug candidates in the fields of, e.g., immuno-oncology and infectious diseases allowing for improved specificity, improved avidity, and the ability to bind to multiple binding targets. See, e.g., U.S. Pat. Nos. 9,951,134, 10,400,038, and 9,938,347, U.S. Patent Application Publication Nos. US20190100597A1, US20180118814A1, US20180118816A1, US20190185570A1, and US20180265596A1, and PCT Publication Nos. WO 2018/017888, WO 2018/017763, WO 2018/017889, WO 2018/017761, and WO 2019/165340.

As indicated, antibodies bind epitopes on antigens. The term antigen refers to a molecule or a portion of a molecule capable of being bound by an antibody. An epitope is a region of an antigen that is bound by the variable region of an antibody. Epitope determinants can include chemically active surface groupings of molecules such as amino acids, sugar side chains, phosphoryl or sulfonyl groups, and can have specific three-dimensional structural characteristics, and/or specific charge characteristics. When the antigen is a protein or peptide, the epitope includes specific amino acids within that protein or peptide that contact the variable region of an antibody.

In particular embodiments, an epitope denotes the binding site on the SARS-CoV-2 spike protein bound by a corresponding variable region of an antibody. The variable region either binds to a linear epitope (e.g., an epitope including a stretch of 5 to 12 consecutive amino acids), or the variable region binds to a three-dimensional structure formed by the spatial arrangement of several short stretches of the protein target. Three-dimensional epitopes recognized by a variable region, e.g. by the epitope recognition site or paratope of an antibody or antibody fragment, can be thought of as three-dimensional surface features of an epitope molecule. These features fit precisely (in) to the corresponding binding site of the variable region and thereby binding between the variable region and its target protein (more generally, antigen) is facilitated. In particular embodiments, an epitope can be considered to have two levels: (i) the “covered patch” which can be thought of as the shadow an antibody variable region would cast on the antigen to which it binds; and (ii) the individual participating side chains and backbone residues that facilitate binding. Binding is then due to the aggregate of ionic interactions, hydrogen bonds, and hydrophobic interactions. For information regarding binding values and methods to measure the same, see the Closing Paragraphs section of this disclosure.

A monoclonal antibody refers to an antibody obtained from a population of substantially homogeneous antibodies, i.e., the individual antibodies including the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, monoclonal antibodies can be made by a variety of techniques, including the hybridoma method, recombinant DNA methods, phage-display methods, and methods utilizing transgenic animals containing all or part of the human immunoglobulin loci.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al. (supra). In particular embodiments, for the V_{H}, the subgroup is subgroup Ill as in Kabat et al. (supra).

When linked to an Fc fragment, a single-domain antibody can be referred to as a heavy chain only antibody (HcAb). As one example, the Fc portion of human IgG1 includes the sequence

(SEQ ID NO: 465)
THTCPPCPAPEFFGGPSVFFFPPKPKDTFMISRTPEVTCVVVDVSHEDP
EVKFNWYVDGVEVHNAKTKPREEQYNSTYRVVSVETVFHQDWENGKEYK
CKVSNKAFPVPIEKTISKAKGQPREPQVYTLPPSREEMTKNQVSLTCLV
KGFYPSDIAVEWESNGQPENNYKTTPPVLDSDGPFFLYSKLTVDKSRWQ
QGNVFSCSVMHEALHNHYTQKSLSLSPGK.

The human IgG2 Fc region includes the amino acid sequence:

(SEQ ID NO: 587)
PAPPVAGPSVFLFPPKPKDTLMISRTPEVTCVVVDVSHEDPEVQFNWYV
DGVEVHNAKTKPREEQFNSTFRVVSVLTVVHQDWLNGKEYKCKVSNKGL
PAPIEKTISKTKGQPREPQVYTLPPSREEMTKNQVSLTCLVKGFYPSDI
AVEWESNGQPENNYKTTPPMLDSDGSFFLYSKLTVDKSRWQQGNVFSCS
VMHEALHNHYTQKSLSLSPGK Referring to SEQ ID NO: 587,

the human CH2 region extends from amino acid 10 to amino acid 107 and the human CH3 region extends from amino acid 116 to amino acid 212.

The human IgG3 Fc region includes the amino acid sequence: PAPELLGGPSVFLFPPKPKDTLMISRTPEVTCVVVDVSHEDPEVQFKWYVDGVEVHNAKTKPR EEQFNSTFRVVSVLTVLHQDWLNGKEYKCKVSNKALPAPIEKTISKTKGQPREPQVYTLPPSRE EMTKNQVSLTCLVKGFYPSDIAVEWESSGQPENNYNTTPPMLDSDGSFFLYSKLTVDKSRWQ QGNIFSCSVMHEALHNRFTQKSLSLSPGK (SEQ ID NO: 588). Referring to SEQ ID NO: 588, the human CH2 region extends from amino acid 11 to amino acid 108 and the human CH3 region extends from amino acid 117 to amino acid 212.

The human IgG4 Fc region includes the amino acid sequence: PAPEFLGGPSVFLFPPKPKDTLMISRTPEVTCVVVDVSQEDPEVQFNWYVDGVEVHNAKTKPR EEQFNSTYRVVSVLTVLHQDWLNGKEYKCKVSNKGLPSSIEKTISKAKGQPREPQVYTLPPSQ EEMTKNQVSLTCLVKGFYPSDIAVEWESNGQPENNYKTTPPVLDSDGSFFLYSRLTVDKSRW QEGNVFSCSVMHEALHNHYTQKSLSLSLGK (SEQ ID NO: 589). Referring to SEQ ID NO: 589, the human CH2 region extends from amino acid 1 to amino acid 111 and the human CH3 region extends from amino acid 112 to amino acid 218.

Certain examples of HcAb include single-domain antibodies linked to a human Fc region selected from IgG, IgA, IgM, IgD and IgE.

Particular embodiments include immunoglobulin constant region domains that allow the binding portion of molecules provided herein to readily multimerize into dimers, pentamers or hexamers. Basic immunoglobulin structures in vertebrate systems are described above and well understood. (See, e.g., Harlow et al., Antibodies: A Laboratory Manual, Cold Spring Harbor Laboratory Press, 2nd ed. 1988).

Immunoglobulin A (IgA), as the major class of antibody present in the mucosal secretions of most mammals, represents a key first line of defense against invasion by inhaled and ingested pathogens. IgA is also found at significant concentrations in the serum of many species, where it functions as a second line of defense mediating elimination of pathogens that have breached the mucosal surface. Receptors specific for the Fc region of IgA, FcaR, are key mediators of IgA effector function. Native IgA is a tetrameric protein including two identical light chains (K or A) and two identical heavy chains. IgA, similarly to IgG, contains three constant domains (CA1-CA3), with a hinge region between the CA1 and CA2 domains. The main difference between IgA1 and IgA2 resides in the hinge region that lies between the two Fab arms and the Fc region. IgA1 has an extended hinge region due to the insertion of a duplicated stretch of amino acids, which is absent in IgA2. Both forms of IgA have the capacity to form dimers, in which two monomer units, are arranged in an end-to-end configuration stabilized by disulfide bridges and incorporation of a J-chain. J-chains are also part of IgM pentamers and are discussed in more detail below.

Both IgA and IgM (discussed further below in relation to pentamers and hexamers) possess an 18-amino acid extension in the C terminus called the “tail-piece” (tp). The IgA and IgM tp is highly conserved among various animal species. The conserved penultimate cysteine residue in the IgA and IgM tp has been demonstrated to be involved in multimerization by forming a disulfide bond between heavy chains to permit formation of a multimer. Both tp contain an N-linked carbohydrate addition site, the presence of which is required for dimer formation in IgA and J-chain incorporation and pentamer formation in IgM. However, the structure and composition of the N-linked carbohydrates in the tp differ, suggesting differences in the accessibility of the glycans to processing by glycosyltransferases. Particularly, the IgA (atp) and IgM (ptp) tp differ at seven amino acid positions.

The human IgA1 constant region typically includes the amino acid sequence: ASPTSPKVFPLSLCSTQPDGNVVIACLVQGFFPQEPLSVTWSESGQGVTARNFPPSQDASGDL YTTSSQLTLPATQCLAGKSVTCHVKHYTNPSQDVTVPCPVPSTPPTPSPSTPPTPSPSCCHPR LSLHRPALEDLLLGSEANLTCTLTGLRDASGVTFTWTPSSGKSAVQGPPERDLCGCYSVSSVL PGCAEPWNHGKTFTCTAAYPESKTPLTATLSKSGNTFRPEVHLLPPPSEELALNELVTLTCLAR GFSPKDVLVRWLQGSQELPREKYLTWASRQEPSQGTTTFAVTSILRVAAEDWKKGDTFSCMV GHEALPLAFTQKTIDRLAGKPTHVNVSVVMAEVDGTCY (SEQ ID NO: 466). Referring to this SEQ ID NO: 466, the human CA1 domain extends from amino acid 6 to amino acid 98; the human IgA1 hinge region extends from amino acid 102 to amino acid 124, the human CA2 domain extends from amino acid 125 to amino acid 219, the human CA3 domain extends from amino acid 228 to amino acid 330, and the tp extends from amino acid 331 to amino acid 352.

The human IgA2 constant region typically includes the amino acid sequence ASPTSPKVFPLSLDSTPQDGNVVVACLVQGFFPQEPLSVTWSESGQNVTARNFPPSQDASGD LYTTSSQLTLPATQCPDGKSVTCHVKHYTNPSQDVTVPCPVPPPPPCCHPRLSLHRPALEDLL LGSEANLTCTLTGLRDASGATFTWTPSSGKSAVQGPPERDLCGCYSVSSVLPGCAQPWNHG ETFTCTAAHPELKTPLTANITKSGNTFRPEVHLLPPPSEELALNELVTLTCLARGFSPKDVLVRW LQGSQELPREKYLTWASRQEPSQGTTTFAVTSILRVAAEDWKKGDTFSCMVGHEALPLAFTQK TIDRLAGKPTHVNVSVVMAEVDGTCY (SEQ ID NO: 467). Referring to this SEQ ID NO: 467, the human CA1 domain extends from amino acid 6 to amino acid 98, the human IgA2 hinge region extends from amino acid 102 to amino acid 111, the human CA2 domain extends from amino acid 113 to amino acid 206, the human CA3 domain extends from amino acid 215 to amino acid 317, and the tp extends from amino acid 318 to amino acid 340.

As indicated, two IgA binding units can form a complex with two additional polypeptide chains, the J chain (e.g., SEQ ID NO: 482, the mature human J chain) and the secretory component to form a bivalent secretory IgA (slgA)-derived binding molecule. An exemplary precursor secretory component includes the sequence MLLFVLTCLLAVFPAISTKSPIFGPEEVNSVEGNSVSITCYYPPTSVNRHTRKYWCRQGARGGC ITLISSEGYVSSKYAGRANLTNFPENGTFVVNIAQLSQDDSGRYKCGLGINSRGLSFDVSLEVS QGPGLLNDTKVYTVDLGRTVTINCPFKTENAQKRKSLYKQIGLYPVLVIDSSGYVNPNYTGRIRL DIQGTGQLLFSVVINQLRLSDAGQYLCQAGDDSNSNKKNADLQVLKPEPELVYEDLRGSVTFH CALGPEVANVAKFLCRQSSGENCDVVVNTLGKRAPAFEGRILLNPQDKDGSFSVVITGLRKED AGRYLCGAHSDGQLQEGSPIQAWQLFVNEESTIPRSPTVVKGVAGGSVAVLCPYNRKESKSIK YWCLWEGAQNGRCPLLVDSEGWVKAQYEGRLSLLEEPGNGTFTVILNQLTSRDAGFYWCLTN GDTLWRTTVEIKIIEGEPNLKVPGNVTAVLGETLKVPCHFPCKFSSYEKYWCKWNNTGCQALP SQDEGPSKAFVNCDENSRLVSLTLNLVTRADEGWYWCGVKQGHFYGETAAVYVAVEERKAA GSRDVSLAKADAAPDEKVLDSGFREIENKAIQDPRLFAEEKAVADTRDQADGSRASVDSGSSE EQGGSSRALVSTLVPLGLVLAVGAVAVGVARARHRKNVDRVSIRSYRTDISMSDFENSREFGA NDNMGASSITQETSLGGKEEFVATTESTTETKEPKKAKRSSKEEAEMAYKDFLLQSSTVAAEA QDGPQEA (SEQ ID NO: 468). An exemplary mature secretory component includes KSPIFGPEEVNSVEGNSVSITCYYPPTSVNRHTRKYWCRQGARGGCITLISSEGYVSSKYAGR ANLTNFPENGTFVVNIAQLSQDDSGRYKCGLGINSRGLSFDVSLEVSQGPGLLNDTKVYTVDL GRTVTINCPFKTENAQKRKSLYKQIGLYPVLVIDSSGYVNPNYTGRIRLDIQGTGQLLFSVVINQL RLSDAGQYLCQAGDDSNSNKKNADLQVLKPEPELVYEDLRGSVTFHCALGPEVANVAKFLCR QSSGENCDVVVNTLGKRAPAFEGRILLNPQDKDGSFSVVITGLRKEDAGRYLCGAHSDGQLQE GSPIQAWQLFVNEESTIPRSPTVVKGVAGGSVAVLCPYNRKESKSIKYWCLWEGAQNGRCPLL VDSEGWVKAQYEGRLSLLEEPGNGTFTVILNQLTSRDAGFYWCLTNGDTLWRTTVEIKIIEGEP NLKVPGNVTAVLGETLKVPCHFPCKFSSYEKYWCKWNNTGCQALPSQDEGPSKAFVNCDEN SRLVSLTLNLVTRADEGWYWCGVKQGHFYGETAAVYVAVEERKAAGSRDVSLAKADAAPDEK VLDSGFREIENKAIQDPR (SEQ ID NO: 469). While not wishing to be bound by theory, and as indicated above, the assembly of two IgA binding units into a dimeric IgA-derived binding molecule is thought to involve the CA3 and tp domains. See, e.g., Braathen, R., el al., J. Biol. Chem. 277:42755-42762 (2002). Accordingly, a multimerizing dimeric IgA-derived binding molecule provided in this disclosure typically includes IgA constant regions that include at least the CA3 and tp domains.

An engineered IgA heavy chain constant region can additionally include a CA2 domain or a fragment thereof, an IgA hinge region or fragment thereof, a CA1 domain or a fragment thereof, and/or other IgA (or other immunoglobulin, e.g., IgG) heavy chain domains, including, e.g., an IgG hinge region. In certain embodiments, a binding molecule as provided herein can include a complete IgA heavy chain constant domain (e.g., SEQ ID NO: 466 or SEQ ID NO: 467), or a variant, derivative, or analog thereof.

In particular embodiments, the IgA heavy chain constant regions can include amino acids 125 to 353 of SEQ ID NO: 466 or amino acids 113 to 340 of SEQ ID NO: 467. In particular embodiments, the IgA heavy chain constant regions can each further include an IgA or IgG hinge region situated N-terminal to the IgA CA2 domains. For example, the IgA heavy chain constant regions can include amino acids 102 to 353 of SEQ ID NO: 466 or amino acids 102 to 340 of SEQ ID NO: 467. In particular embodiments, the IgA heavy chain constant regions can each further include an IgA CA1 domain situated N-terminal to the IgA hinge region.

Each of the strategies discussed above can be used to create IgA antibody-based dimers.

Particular embodiments include IgM immunoglobulin constant region domains that allow the binding portion of molecules provided herein to readily multimerize into pentamers or hexamers.

Particular embodiments include IgM constant regions (or variants thereof). These embodiments have the ability to form hexamers, or in association with a J-chain, form pentamers. Embodiments with an IgM constant region typically include at least the Cμ4-tp domains of the IgM constant region but can include heavy chain constant region domains from other antibody isotypes, e.g., IgG, from the same species or from a different species. In particular embodiments, one or more constant region domains can be deleted so long as the IgM antibody is capable of forming hexamers and/or pentamers. Thus, an IgM antibody can be, e.g., a hybrid IgM/lgG antibody or can be a “multimerizing fragment” of an IgM-derived binding molecule.

The assembly of five or six IgM binding units into a pentameric or hexameric IgM antibody is thought to involve the Cμ4 and tp domains. See, e.g., Braathen, R., et al., J Biol. Chem. 277:42755-42762 (2002). Accordingly, a pentameric or hexameric IgM antibody described in this disclosure typically includes at least the Cμ4 and/or tp domains (also referred to herein collectively as Cμ4-tp). A “multimerizing fragment” of an IgM heavy chain constant region thus includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains.

Five IgM monomers form a complex with a J-chain to form a native IgM molecule. The J-chain is considered to facilitate polymerization of μ chains before IgM is secreted from antibody-producing cells. Sequences for the human IGJ gene are known in the art, for example, (IGMT Accession: J00256, X86355, M25625, AJ879487). The J chain establishes the disulfide bridges between IgM antibodies to form multimeric structures such as pentamers. See, for example, Sorensen et al. International Immunology, (2000), pages 19-27. While crystallization of IgM has proved to be notoriously challenging, Czajkowsky and Shao (PNAS 106(35): 14960-14965, 2009) published a homology-based structural model of IgM, based on the structure of the IgE Fc domain and the known disulfide pairings. The authors report that the human IgM pentamer is a mushroom-shaped molecule with a flexural bias. The IgM heavy (μ) chain contains five N-linked glycosylation sites: Asn-171, Asn-332, Asn-395, Asn-402 and Asn-563. In an IgM antibody where each binding unit is bivalent, the binding molecule itself can have 10 or 12 valencies.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, b-2 Microglobulins, Major Histocompatibility Antigens, Thy-I, Complement, C-Reactive Protein, Thymopoietin, Integrins, Post-gamma Globulin, a-2 Macroglobulins, and Other Related Proteins,” U.S. Dept of Health and Human Services (1991). IgM constant regions can be numbered sequentially (i.e., amino acid #1 starting with the first amino acid of the constant region) or by using the Kabat numbering scheme.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tp, as indicated above.

In particular embodiments, each binding unit of a multimeric binding molecule as provided herein includes two IgM heavy chain constant regions or multimerizing fragments or variants thereof, each including at least an IgM Cμ4 domain and an IgM tp domain. In certain embodiments the IgM heavy chain constant regions can each further include an IgM Cμ3 domain situated N-terminal to the IgM Cμ4 and IgM tp domains.

In particular embodiments, the IgM heavy chain constant regions can each further include an IgM Cμ2 domain situated N-terminal to the IgM Cμ3 domain. Exemplary multimeric binding molecules provided herein include human IgM constant regions that include the wild-type human Cμ2, Cμ3, and Cμ4-tp domains as follows:

(SEQ ID NO: 470)
VIAELPPKVSVFVPPRDGFFGNPRKSKLICQATGFSPRQIQVSWLREGK
QVGSGVTTDQVQAEAKESGPTTYKVTSTLTIKESDWLSQSMFTCRVDHR
GLTFQQNASSMCVPDQDTAIRVFAIPPSFASIFLTKSTKLTCLVTDLTT
YDSVTISWTRQNGEAVKTHTNISESHPNATFSAVGEASICEDDWNSGER
FTCTVTHTDLPSPLKQTISRPKGVALHRPDVYLLPPAREQLNLRESATI
TCLVTGFSPADVFVQWMQRGQPLSPEKYVTSAPMPEPQAPGRYFAHSIL
TVSEEEWNTGETYTCVVAHEALPNRVTERTVDKSTGKPTLYNVSLVMSD
TAGTCY.

In certain IgM-derived multimeric binding molecules as provided herein each IgM constant region can include, instead of, or in addition to an IgM Cμ2 domain, an IgG hinge region or functional variant thereof situated N-terminal to the IgM Cμ3 domain. An exemplary variant human IgG1 hinge region amino acid sequence in which the cysteine at position 6 is substituted with serine is VEPKSSDKTHTCPPCPAP (SEQ ID NO: 471). An exemplary IgM constant region of this type includes the variant human IgG1 hinge region fused to a multimerizing fragment of the human IgM constant region including the Cμ3, Cμ4, and tp domains, and includes the amino acid sequence:

(SEQ ID NO: 472)
VEPKSSDKTHTCPPCPAPDQDTAIRVFAIPPSFASIFLTKSTKLTCLVT
DLTTYDSVTISWTRQNGEAVKTHTNISESHPNATFSAVGEASICEDDWN
SGERFTCTVTHTDLPSPLKQTISRPKGVALHRPDVYLLPPAREQLNLRE
SATITCLVTGFSPADVFVQWMQRGQPLSPEKYVTSAPMPEPQAPGRYFA
HSILTVSEEEWNTGETYTCVVAHEALPNRVTERTVDKSTGKPTLYNVSL
VMSDTAGTCY.

Human IgM constant regions, and also certain non-human primate IgM constant regions, as provided herein typically include five (5) naturally-occurring asparagine (N)-linked glycosylation motifs or sites. As used herein “an N-linked glycosylation motif” includes the amino acid sequence N-X1-S/T, wherein N is asparagine, X1 is any amino acid except proline (P), and S/T is serine (S) or threonine (T). The glycan is attached to the nitrogen atom of the asparagine residue. See, e.g., Drickamer K, Taylor ME (2006), Introduction to Glycobiology (2nd ed.). Oxford University Press, USA. N-linked glycosylation motifs occur in the human IgM heavy chain constant regions of SEQ ID NO: 473 or SEQ ID NO: 474 starting at positions 46 (“Ni”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Each of these sites in the human IgM heavy chain constant region, except for N4, can be mutated to prevent glycosylation at that site, while still allowing IgM expression and assembly into a hexamer or pentamer.

The human IgM constant region typically includes the amino acid sequence GSASAPTLFPLVSCENSPSDTSSVAVGCLAQDFLPDSITFSWKYKNNSDISSTRGFPSVLRGGK YAATSQVLLPSKDVMQGTDEHVVCKVQHPNGNKEKNVPLPVIAELPPKVSVFVPPRDGFFGNP RKSKLICQATGFSPRQIQVSWLREGKQVGSGVTTDQVQAEAKESGPTTYKVTSTLTIKESDWL SQSMFTCRVDHRGLTFQQNASSMCVPDQDTAIRVFAIPPSFASIFLTKSTKLTCLVTDLTTYDSV TISWTRQNGEAVKTHTNISESHPNATFSAVGEASICEDDWNSGERFTCTVTHTDLPSPLKQTIS RPKGVALHRPDVYLLPPAREQLNLRESATITCLVTGFSPADVFVQWMQRGQPLSPEKYVTSAP MPEPQAPGRYFAHSILTVSEEEWNTGETYTCVVAHEALPNRVTERTVDKSTGKPTLYNVSLVM SDTAGTCY (SEQ ID NO: 473; identical to, e.g., GenBank Accession Nos. pirIIS37768, CAA47708.1, and CAA47714.1). Referring to this SEQ ID NO: 473, the human Cμ1 region ranges from amino acid 5 to amino acid 102; the human Cμ2 region ranges from amino acid 114 to amino acid 205, the human Cμ3 region ranges from amino acid 224 to amino acid 319, the Cμ4 region ranges from amino acid 329 to amino acid 430, and the tp ranges from amino acid 431 to amino acid 453.

In particular embodiments, an IgM heavy chain constant region includes the sequence: GSASAPTLFPLVSCENSPSDTSSVAVGCLAQDFLPDSITFSWKYKNNSDISSTRGFPSVLRGGK YAATSQVLLPSKDVMQGTDEHVVCKVQHPNGNKEKNVPLPVIAELPPKVSVFVPPRDGFFGNP RKSKLICQATGFSPRQIQVSWLREGKQVGSGVTTDQVQAEAKESGPTTYKVTSTLTIKESDWL GQSMFTCRVDHRGLTFQQNASSMCVPDQDTAIRVFAIPPSFASIFLTKSTKLTCLVTDLTTYDS VTISWTRQNGEAVKTHTNISESHPNATFSAVGEASICEDDWNSGERFTCTVTHTDLPSPLKQTI SRPKGVALHRPDVYLLPPAREQLNLRESATITCLVTGFSPADVFVQWMQRGQPLSPEKYVTSA PMPEPQAPGRYFAHSILTVSEEEWNTGETYTCVVAHEALPNRVTERTVDKSTGKPTLYNVSLV MSDTAGTCY (SEQ ID NO: 474; (UniProt ID P01871)-allele IGHM*04). This sequence differs from SEQ ID NO: 473 by one amino acid at position 191.

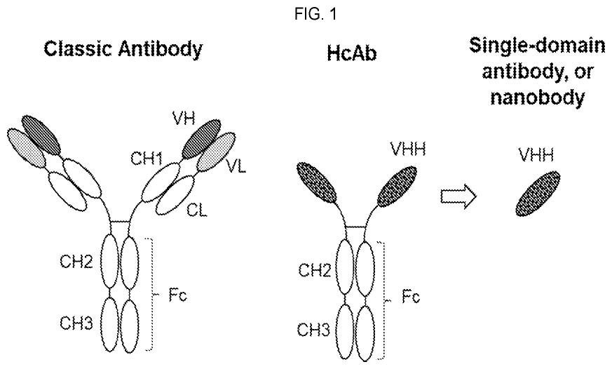

Other forms of the human IgM constant region with minor sequence variations exist, including GenBank Accession Nos. P01871.4, CAB37838.1, and pir|IMHHU. The amino acid substitutions, insertions, and/or deletions at positions corresponding to SEQ ID NO: 473 described herein can likewise be incorporated into alternate human IgM sequences, as well as into IgM constant region amino acid sequences of other species, e.g., those shown in FIG. 1 of PCT/US2019/020374.

In certain aspects, a variant human IgM constant region includes an amino acid substitution corresponding to the wild-type human IgM constant region at position P311, P313, R344, E345, S401, E402, and/or E403 of SEQ ID NO: 473. These positions correspond to the Kabat numbering system as follows: S401 of SEQ ID NO: 473 corresponds to S524 of Kabat; E402 of SEQ ID NO: 473 corresponds to E525 of Kabat; E403 of SEQ ID NO: 473 corresponds to E526 of Kabat; R344 of SEQ ID NO: 473 corresponds to R467 of Kabat; and E345 of SEQ ID NO: 473 corresponds to E468 of Kabat.

In particular embodiments, “corresponds to” means the designated position of SEQ ID NO: 473 and the amino acid in the sequence of the IgM constant region of any species which is homologous to the specified position. See FIG. 1 of PCT/US2019/020374.

In particular embodiments, P311 of SEQ ID NO: 473 can be substituted, e.g., with alanine (P311A), serine (P311S), or glycine (P311G) and/or P313 of SEQ ID NO: 473 can be substituted, e.g., with alanine (P313A), serine (P313S), or glycine (P313G). P311 and P313 of SEQ ID NO: 473 can be substituted with alanine (P311A) and serine (P313S), respectively as shown in the following sequence: (mutations in bold underline)

(SEQ ID NO: 475)
GSASAPTLFPLVSCENSPSDTSSVAVGCLAQDFLPDSITFSWKYKNNSD
ISSTRGFPSVLRGGKYAATSQVLLPSKDVMQGTDEHVVCKVQHPNGNKE
KNVPLPVIAELPPKVSVFVPPRDGFFGNPRKSKLICQATGFSPRQIQVS
WLREGKQVGSGVTTDQVQAEAKESGPTTYKVTSTLTIKESDWLSQSMFT
CRVDHRGLTFQQNASSMCVPDQDTAIRVFAIPPSFASIFLTKSTKLTCL
VTDLTTYDSVTISWTRQNGEAVKTHTNISESHPNATFSAVGEASICEDD
WNSGERFTCTVTHTDLASSLKQTISRPKGVALHRPDVYLLPPAREQLNL
RESATITCLVTGFSPADVFVQWMQRGQPLSPEKYVTSAPMPEPQAPGRY
FAHSILTVSEEEWNTGETYTCVVAHEALPNRVTERTVDKSTGKPTLYNV
SLVMSDTAGTCY.

In certain aspects, S401 of SEQ ID NO: 473 can be substituted with any amino acid. In certain aspects, S401 of SEQ ID NO: 473 can be substituted with alanine (A) as follows (alanine substitution indicated by bold underline):

(SEQ ID NO: 476)
GSASAPTLFPLVSCENSPSDTSSVAVGCLAQDFLPDSITFSWKYKNNSD
ISSTRGFPSVLRGGKYAATSQVLLPSKDVMQGTDEHVVCKVQHPNGNKE
KNVPLPVIAELPPKVSVFVPPRDGFFGNPRKSKLICQATGFSPRQIQVS
WLREGKQVGSGVTTDQVQAEAKESGPTTYKVTSTLTIKESDWLSQSMFT
CRVDHRGLTFQQNASSMCVPDQDTAIRVFAIPPSFASIFLTKSTKLTCL
VTDLTTYDSVTISWTRQNGEAVKTHTNISESHPNATFSAVGEASICEDD
WNSGERFTCTVTHTDLPSPLKQTISRPKGVALHRPDVYLLPPAREQLNL
RESATITCLVTGFSPADVFVQWMQRGQPLSPEKYVTSAPMPEPQAPGRY
FAHSILTVAEEEWNTGETYTCVVAHEALPNRVTERTVDKSTGKPTLYNV
SLVMSDTAGTCY.

In certain aspects, E402 of SEQ ID NO: 473 can be substituted with any amino acid. In certain aspects, E402 of SEQ ID NO: 473 can be substituted with alanine (A) as follows (alanine substitution indicated by bold underline):

(SEQ ID NO: 477)
GSASAPTLFPLVSCENSPSDTSSVAVGCLAQDFLPDSITFSWKYKNNSD
ISSTRGFPSVLRGGKYAATSQVLLPSKDVMQGTDEHVVCKVQHPNGNKE
KNVPLPVIAELPPKVSVFVPPRDGFFGNPRKSKLICQATGFSPRQIQVS
WLREGKQVGSGVTTDQVQAEAKESGPTTYKVTSTLTIKESDWLSQSMFT
CRVDHRGLTFQQNASSMCVPDQDTAIRVFAIPPSFASIFLTKSTKLTCL
VTDLTTYDSVTISWTRQNGEAVKTHTNISESHPNATFSAVGEASICEDD
WNSGERFTCTVTHTDLPSPLKQTISRPKGVALHRPDVYLLPPAREQLNL
RESATITCLVTGFSPADVFVQWMQRGQPLSPEKYVTSAPMPEPQAPGRY
FAHSILTVSAEEWNTGETYTCVVAHEALPNRVTERTVDKSTGKPTLYNV
SLVMSDTAGTCY.

In certain aspects, E403 of SEQ ID NO: 473 can be substituted with any amino acid. In certain aspects, E403 of SEQ ID NO: 473 can be substituted with alanine (A) as follows (alanine substitution indicated by bold underline):

(SEQ ID NO: 478)
GSASAPTLFPLVSCENSPSDTSSVAVGCLAQDFLPDSITFSWKYKNNSD
ISSTRGFPSVLRGGKYAATSQVLLPSKDVMQGTDEHVVCKVQHPNGNKE
KNVPLPVIAELPPKVSVFVPPRDGFFGNPRKSKLICQATGFSPRQIQVS
WLREGKQVGSGVTTDQVQAEAKESGPTTYKVTSTLTIKESDWLSQSMFT
CRVDHRGLTFQQNASSMCVPDQDTAIRVFAIPPSFASIFLTKSTKLTCL
VTDLTTYDSVTISWTRQNGEAVKTHTNISESHPNATFSAVGEASICEDD
WNSGERFTCTVTHTDLPSPLKQTISRPKGVALHRPDVYLLPPAREQLNL
RESATITCLVTGFSPADVFVQWMQRGQPLSPEKYVTSAPMPEPQAPGRY
FAHSILTVSEAEWNTGETYTCVVAHEALPNRVTERTVDKSTGKPTLYNV
SLVMSDTAGTCY.

In certain aspects, R344 of SEQ ID NO: 473 can be substituted with any amino acid. In certain aspects, R344 of SEQ ID NO: 473 can be substituted with alanine (A) as follows (alanine substitution indicated by bold underline):

(SEQ ID NO: 479)
GSASAPTLFPLVSCENSPSDTSSVAVGCLAQDFLPDSITFSWKYKNNSD
ISSTRGFPSVLRGGKYAATSQVLLPSKDVMQGTDEHVVCKVQHPNGNKE
KNVPLPVIAELPPKVSVFVPPRDGFFGNPRKSKLICQATGFSPRQIQVS
WLREGKQVGSGVTTDQVQAEAKESGPTTYKVTSTLTIKESDWLSQSMFT
CRVDHRGLTFQQNASSMCVPDQDTAIRVFAIPPSFASIFLTKSTKLTCL
VTDLTTYDSVTISWTRQNGEAVKTHTNISESHPNATFSAVGEASICEDD
WNSGERFTCTVTHTDLPSPLKQTISRPKGVALHRPDVYLLPPAREQLNL
AESATITCLVTGFSPADVFVQWMQRGQPLSPEKYVTSAPMPEPQAPGRY
FAHSILTVSEEEWNTGETYTCVVAHEALPNRVTERTVDKSTGKPTLYNV
SLVMSDTAGTCY.

In certain aspects, E345 of SEQ ID NO: 473 can be substituted with any amino acid. In certain aspects, E345 of SEQ ID NO: 473 can be substituted with alanine (A) as follows (alanine substitution indicated by bold underline):

(SEQ ID NO: 480)
GSASAPTLFPLVSCENSPSDTSSVAVGCLAQDFLPDSITFSWKYKNNSD
ISSTRGFPSVLRGGKYAATSQVLLPSKDVMQGTDEHVVCKVQHPNGNKE
KNVPLPVIAELPPKVSVFVPPRDGFFGNPRKSKLICQATGFSPRQIQVS
WLREGKQVGSGVTTDQVQAEAKESGPTTYKVTSTLTIKESDWLSQSMFT
CRVDHRGLTFQQNASSMCVPDQDTAIRVFAIPPSFASIFLTKSTKLTCL
VTDLTTYDSVTISWTRQNGEAVKTHTNISESHPNATFSAVGEASICEDD
WNSGERFTCTVTHTDLPSPLKQTISRPKGVALHRPDVYLLPPAREQLNL
RASATITCLVTGFSPADVFVQWMQRGQPLSPEKYVTSAPMPEPQAPGRY
FAHSILTVSEEEWNTGETYTCVVAHEALPNRVTERTVDKSTGKPTLYNV
SLVMSDTAGTCY.

As indicated, five IgM binding units can form a complex with a J-chain to form a pentameric IgM antibody. The precursor form of the human J-chain includes: MKNHLLFWGVLAVFIKAVHVKAQEDERIVLVDNKCKCARITSRIIRSSEDPNEDIVERNIRIIVPLN NRENISDPTSPLRTRFVYHLSDLCKKCDPTEVELDNQIVTATQSNICDEDSATETCYTYDRNKC YTAVVPLVYGGETKMVETALTPDACYPD (SEQ ID NO: 481). The signal peptide extends from amino acid 1 to amino acid 22 of SEQ ID NO: 481 and the mature human J-chain extends from amino acid 23 to amino acid 159 of SEQ ID NO: 481.

The mature human J-chain includes the amino acid sequence

(SEQ ID NO: 482)
QEDERIVLVDNKCKCARITSRIIRSSEDPNEDIVERNIRIIVPLNNREN
ISDPTSPLRTRFVYHLSDLCKKCDPTEVELDNQIVTATQSNICDEDSAT
ETCYTYDRINKCYTAVVPLVYGGETKMVETALTPDACYPD.

The term “J-chain” as used herein refers to the J-chain of native sequence IgM or IgA antibodies of any animal species. When specified, it can also refer to any functional fragment thereof, derivative thereof, and/or variant thereof, including a mature human J-chain amino acid sequence provided herein as SEQ ID NO: 482. A functional fragment, derivative, and/or variant of a J-chain has at least 90% sequence identity to the reference J-chain and retains the multimerizing function of the reference J-chain.

In certain aspects, the J-chain of the IgM antibody as provided herein includes an amino acid substitution at the amino acid position corresponding to amino acid Y102, T103, N49 or S51 of SEQ ID NO: 482.

By “an amino acid corresponding to” a position of SEQ ID NO: 482 is meant the amino acid in the sequence of the J-chain of any species which is homologous to the referenced residue in the human J-chain. For example, the position corresponding to Y102 in SEQ ID NO: 482 is conserved in the J-chain amino acid sequences of at least 43 other species. The position corresponding to T103 in SEQ ID NO: 482 is conserved in the J-chain amino acid sequences of at least 37 other species. The positions corresponding to N49 and S51 in SEQ ID NO: 482 are conserved in the J-chain amino acid sequences of at least 43 other species. See FIG. 4 of U.S. Pat. No. 9,951,134 and FIG. 2 of PCT/US2019/020374.

In certain aspects, the amino acid corresponding to Y102 of SEQ ID NO: 482 can be substituted with any amino acid. In certain aspects, the amino acid corresponding to Y102 of SEQ ID NO: 482 can be substituted with alanine (alanine substitution indicated by bold underline):

(SEQ ID NO: 483)
QEDERIVLVDNKCKCARITSRIIRSSEDPNEDIVERNIRIIVPLNNREN
ISDPTSPLRTRFVYHLSDLCKKCDPTEVELDNQIVTATQSNICDEDSAT
ETCATYDRNKCYTAVVPLVYGGETKMVETALTPDACYPD,

With serine (serine substitution indicated by bold underline):

(SEQ ID NO: 484)
QEDERIVLVDNKCKCARITSRIIRSSEDPNEDIVERNIRIIVPLNNREN
ISDPTSPLRTRFVYHLSDLCKKCDPTEVELDNQIVTATQSNICDEDSAT
ETCSTYDRINKCYTAVVPLVYGGETKMVETALTPDACYPD,

Or with arginine (arginine substitution indicated by bold underline):

(SEQ ID NO: 485)
QEDERIVLVDNKCKCARITSRIIRSSEDPNEDIVERNIRIIVPLNNREN
ISDPTSPLRTRFVYHLSDLCKKCDPTEVELDNQIVTATQSNICDEDSAT
ETCRTYDRINKCYTAVVPLVYGGETKMVETALTPDACYPD.

In certain aspects, the amino acid corresponding to T103 of SEQ ID NO: 482 can be substituted with any amino acid. In a particular aspect, the amino acid corresponding to T103 of SEQ ID NO: 482 can be substituted with alanine as follows (alanine substitution indicated by bold underline):

(SEQ ID NO: 486)
QEDERIVLVDNKCKCARITSRIIRSSEDPNEDIVERNIRIIVPLNNREN
ISDPTSPLRTRFVYHLSDLCKKCDPTEVELDNQIVTATQSNICDEDSAT
ETCYAYDRNKCYTAVVPLVYGGETKMVETALTPDACYPD.

In certain aspects, the variant J-chain or functional fragment thereof of the IgM antibody as provided herein includes an amino acid substitution at the amino acid position corresponding to amino acid N49 or amino acid S51 of SEQ ID NO: 482, provided that S51 is not substituted with threonine (T), or wherein the J-chain includes amino acid substitutions at the amino acid positions corresponding to both amino acids N49 and S51 of SEQ ID NO: 482.

The amino acids corresponding to N49 and S51 of SEQ ID NO: 482 along with the amino acid corresponding to 150 of SEQ ID NO: 482 include an N-linked glycosylation motif in the J-chain. Accordingly, mutations at N49 and/or S51 (with the exception of a single threonine substitution at S51) can prevent glycosylation at this motif. In certain aspects, the asparagine at the position corresponding to N49 of SEQ ID NO: 482 can be substituted with any amino acid. In certain aspects, the asparagine at the position corresponding to N49 of SEQ ID NO: 482 can be substituted with alanine (A), glycine (G), threonine (T), serine (S) or aspartic acid (D). In a particular aspect the position corresponding to N49 of SEQ ID NO: 482 can be substituted with alanine (A). In a particular aspect the J-chain is a variant human J-chain and includes the amino acid sequence:

(SEQ ID NO: 487)
QEDERIVLVDNKCKCARITSRIIRSSEDPNEDIVERNIRIIVPLNNREA
ISDPTSPLRTRFVYHLSDLCKKCDPTEVELDNQIVTATQSNICDEDSAT
ETCYTYDRINKCYTAVVPLVYGGETKMVETALTPDACYPD.

In certain aspects, the serine at the position corresponding to S51 of SEQ ID NO: 482 can be substituted with any amino acid except threonine. In certain aspects, the serine at the position corresponding to S51 of SEQ ID NO: 482 can be substituted with alanine (A) or glycine (G). In a particular aspect the position corresponding to S51 of SEQ ID NO: 482 can be substituted with alanine (A). In a particular aspect the variant J-chain or functional fragment thereof is a variant human J-chain and includes the amino acid sequence:

(SEQ ID NO: 488)
EDERIVLVDNKCKCARITSRIIRSSEDPNEDIVERNIRIIVPLNNRENI
ADPTSPLRTRFVYHLSDLCKKCDPTEVELDNQIVTATQSNICDEDSATE
TCYTYDRNKCYTAVVPLVYGGETKMVETALTPDACYPD.

Particular embodiments include a heterologous polypeptide (e.g., a single-domain antibody binding domain) fused to the J-chain or functional fragment thereof via a peptide linker, e.g., a peptide linker including at least 5 amino acids, but no more than 25 amino acids. In certain aspects, the peptide linker includes (GGGGS)n (SEQ ID NO: 489) wherein n is 1-5.

A single-domain antibody binding domain can be introduced into the J-chain at any location that allows the binding of the binding domain to its binding target without interfering with J-chain function or the function of an associated IgA, IgM, or hybrid IgG antibody. Insertion locations include at or near the C-terminus, at or near the N-terminus or at an internal location that, based on the three-dimensional structure of the J-chain, is accessible. In certain aspects, the antigen-binding domain can be introduced into the mature human J-chain of SEQ ID NO: 482 between cysteine residues 92 and 101 of SEQ ID NO: 482. In a further aspect, the antigen-binding domain can be introduced into the human J-chain of SEQ ID NO: 482 at or near a glycosylation site. In a further aspect, the antigen-binding domain can be introduced into the human J-chain of SEQ ID NO: 482 within 10 amino acid residues from the C-terminus, or within 10 amino acids from the N-terminus.

In particular embodiments, the single-domain antibody is introduced into the native human J-chain sequence of SEQ ID NO: 482 by chemical or chemo-enzymatic derivatization. In particular embodiments, the single-domain antibody is introduced into the native human J-chain sequence of SEQ ID NO: 482 by a chemical linker. In some embodiments, the chemical linker is a cleavable or non-cleavable linker. In particular embodiments, the cleavable linker is a chemically labile linker or an enzyme-labile linker. In some embodiments, the linker is selected from the group including N-succinimidyl-3-(2-pyridyldithio) propionate (SPDP), succinimidyl-4-(N-maleimidomethyl) cyclohexane-I-carboxylate (SMCC), N-succinimidyl-4-(2-pyridylthio) pentanoate (SPP), iminothiolane (IT), afunctional derivatives of imidoesters, active esters, aldehydes, bis-azido compounds, bis-diazonium derivatives, diisocyanates, and bis-active fluorine compounds. In particular embodiments, the modified J-chain is modified by insertion of an enzyme recognition site, and by post-translationally attaching a binding moiety at the enzyme recognition site through a peptide or non-peptide linker.

In certain aspects the modified J-chain can include the formula X[Lₙ]J or J[Lₙ]X, where J includes a mature native J-chain or functional fragment thereof, X includes a heterologous binding domain, and [Lₙ] is a linker sequence including n amino acids, where n is a positive integer from 1 to 100, 1 to 50, or 1 to 25. In certain aspects N is 5, 10, 15, or 20.

J-chains from the following species can also be used in certain embodiments: Pan troglodytes, Pongo abelii, Callithrix jacchus, Macaca mulatta, Papio Anubis, Saimiri boliviensis, Tupaia chinensis, Tursiops truncatus, Orcinus orca, Loxodonta Africana, Leptonychotes weddellii, Ceratotherium sinun, Felis catus, Canis familiaris, Ailuropoda melanoleuca, Mustela furo, Equus caballus, Cavia porcellus, Camelus ferus, Capra hircus, Chinchilla lanigera, Mesocricetus auratus, Ovis aries, Myotis lucifugus, Pantholops hodgsonii, Bos taurus, Mus musculus, Rattus norvegicus, Echinops telfairi, Oryctolagus cuniculus, Monodelphis domestica, Alligator mississippiensis, Chrysemys picta, Sarcophilus harrisii, Ornithorhynchus anatinus, Melopsittacus undulatus, Anas platyrhynchos, Gallus gallus, Meleagris gallopavo, Falco peregrinus, Zonotrichia albicollis, and Pteropus alecto.

When HcAbs are utilized additional modifications to Fc regions can be incorporated. In particular embodiments, one or more amino acid modifications may be introduced into the Fc region of an HcAb, thereby generating an Fc region variant. The Fc region variant may include a human Fc region sequence (e.g., a human IgG1, IgG2, IgG3 or IgG4 Fc region) including an amino acid modification (e.g., a substitution) at one or more amino acid positions. Numerous Fc variants are described below that provide administration benefits.

In particular embodiments, variants have been modified from a reference sequence to produce an administration benefit. Exemplary administration benefits can include (1) reduced susceptibility to proteolysis, (2) reduced susceptibility to oxidation, (3) altered binding affinity for forming protein complexes, (4) altered binding affinities, (5) reduced immunogenicity; and/or (6) extended half-live.

In particular embodiments, the HcAb can be mutated to increase their affinity for Fc receptors. Exemplary mutations that increase the affinity for Fc receptors include: G236A/S239D/A330L/1332E (GASDALIE). Smith et al., Proceedings of the National Academy of Sciences of the United States of America, 109(16), 6181-6186, 2012. In particular embodiments, an HcAb variant includes an Fc region with one or more amino acid substitutions which improve ADCC, e.g., substitutions at positions 298, 333, and/or 334 of the Fc region (EU numbering of residues). In particular embodiments, alterations are made in the Fc region that result in altered C1q binding and/or Complement Dependent Cytotoxicity (CDC), e.g., as described in U.S. Pat. No. 6,194,551, WO 99/51642, and Idusogie et al., J. Immunol. 164: 4178-4184, 2000. In particular embodiments, it may be desirable to create cysteine engineered antibodies, e.g., “thioMAbs,” in which one or more residues of an HcAb are substituted with cysteine residues. In particular embodiments, the substituted residues occur at accessible sites of the HcAb. By substituting those residues with cysteine, reactive thiol groups are thereby positioned at accessible sites of the HcAb and may be used to conjugate the HcAb to other moieties, such as drug moieties or linker-drug moieties, to create an immunoconjugate, as described further below. In particular embodiments, residue 5400 (EU numbering) of the heavy chain Fc region is selected. Cysteine engineered antibodies may be generated as described, e.g., in U.S. Pat. No. 7,521,541.

HcAb variants are provided having a carbohydrate structure that lacks fucose attached (directly or indirectly) to an Fc region. For example, the amount of fucose in such HcAb may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at position 297 in the Fc region (Eu numbering of Fc region residues); however, Asn297 may also be located ±3 amino acids upstream or downstream of position 297, i.e., between positions 294 and 300, due to minor sequence variations in antibodies. Such fucosylation variants may have improved ADCC function. See, e.g., WO2000/61739; WO 2001/29246; WO2002/031140; US2002/0164328; WO2003/085119; WO2003/084570; US2003/0115614; US2003/0157108; US2004/0093621; US2004/0110704; US2004/0132140; US2004/0110282; US2004/0109865; WO2005/035586; WO2005/035778; WO2005/053742; Okazaki et al. J. Mol. Biol. 336:1239-1249 (2004); and Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545, 1986, and knockout cell lines, such as alpha-1,6-fucosyltransferase gene, FUT8, knockout CHO cells (see, e.g., Yamane-Ohnuki et al., Biotech. Bioeng. 87: 614, 2004; Kanda et al., Biotechnol. Bioeng., 94(4):680-688, 2006; and WO2003/085107).

In particular embodiments, modified HcAb include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N—X-S/T motifs during expression in mammalian cells) or modified by synthetic means. The modified amino acid can be within the sequence or at the terminal end of a sequence. Modifications also include nitrited constructs.

In particular embodiments, variants include glycosylation variants wherein the number and/or type of glycosylation site has been altered compared to the amino acid sequences of a reference sequence. In particular embodiments, glycosylation variants include a greater or a lesser number of N-linked glycosylation sites than the reference sequence. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (e.g., those that are naturally occurring) are eliminated and one or more new N-linked sites are created. Additional HcAb variants include cysteine variants wherein one or more cysteine residues are deleted from or substituted for another amino acid (e.g., serine) as compared to the reference sequence. These cysteine variants can be useful when HcAb must be refolded into a biologically active conformation such as after the isolation of insoluble inclusion bodies. These cysteine variants generally have fewer cysteine residues than the reference sequence, and typically have an even number to minimize interactions resulting from unpaired cysteines.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, A. et al., Cancer Biochem. Biophys., 1984, 7:175-186; Hershfield, M. S. et al., N. Engl. J. Medicine, 1987, 316:589-596; and Meyers, F. J. et al., Clin. Pharmacol. Ther., 49:307-313, 1991). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta 788:248, 1984), alter protein immunogenicity (Abuchowski et al., J. Biol. Chem. 252: 3582, 1977), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

In particular embodiments, the HcAb can be fused or coupled to an Fc polypeptide that includes amino acid alterations that extend the in vivo half-life of an antibody that contains the altered Fc polypeptide as compared to the half-life of a similar antibody containing the same Fc polypeptide without the amino acid alterations. In particular embodiments, Fc polypeptide amino acid alterations can include M252Y, T252L, T253S, S254T, T254F, T256E, T256N, E294delta, T307P, A379V, S383N, M428L, N434S, N434A, N434Y, and/or R435H. The R435H mutation is described in more detail in Stapleton et al., Nat. Comm. (2011) 2:599. The N434A mutation is described in more detail in Shields et al., J. Biol. Chem. (2001) 276: 6591-604. The E294delta mutation is described in more detail in Monnet et al., Mabs (2014) 6:422-36 and Bas et al., J. Immunol. (2019) 202:1582094. M428L/N434S is a pair of mutations that increase the half-life of antibodies in serum, as described in Zalevsky et al., Nature Biotechnology 28, 157-159, 2010. M252Y/S254T/T256E are a trio of mutations described in Dall'acqua et al., J. Immunol. (2002) 169: 5171-80. T252L/T253S/T254F is a trio described in Ghetie et al., Nat. Biotechnol. (1997) 15:637-40. Additional combinations include E294delta/T307P/N434Y (Monnet et al., Mabs (2014) 6:422-36) and T256N/A379V/S383N/N434Y (Monnet et al., Mabs (2014) 6:422-36). Other alterations that can be helpful are described in U.S. Pat. Nos. 7,083,784, 7,670,600, US Publication No. 2010/0234575, PCT/US2012/070146, and Zwolak, Scientific Reports 7: 15521, 2017. In particular embodiments, any substitution at one of the following amino acid positions in an Fc polypeptide can be considered an Fc alteration that extends half-life: 250, 251, 252, 253, 254, 256, 294, 259, 307, 308, 332, 378, 379, 380, 383,428, 430, 434, 435, 436. Each of these alterations or combinations of these alterations can be used to extend the half-life of an HcAb described herein.

For additional information regarding Fc mutations that create administration benefits, see Saunders, Conceptual Approaches to Modulating Antibody Effector Functions and Circulation Half-Life, Frontiers in Immunology (2019) Vol. 10, Article 1296.
(iii) SARS-CoV-2 Multi-Specific Binding Molecules. Multi-specific binding molecules include at least two linked binding domains, at least one binding domain including a single-domain antibody disclosed herein. When a multi-specific binding molecule format is utilized, all available binding domains can bind an epitope found on SARS-CoV-2. All binding domains in a multi-specific binding molecule that binds SARS-CoV-2 can be disclosed herein or, in other embodiments, at least one is disclosed herein and others are derived from other SARS-CoV-2 binding domains (e.g., the 47D11 antibody or CR3022).

In certain examples, multi-specific binding molecules include a combination of single-domain antibodies disclosed herein as follows: S1-2 and S2-1; S1-3 and S2-2; S1-7 and S2-3; S1-9 and S2-4; S1-10 and S2-5; S1-11 and S2-6; S1-17 and S2-7; S1-24 and S2-9; S1-25 and S2-10; S1-30 and S2-11; S1-32 and S2-13; S1-41 and S2-14; S1-49 and S2-15; S1-50 and S2-18; S1-58 and S2-22; S1-60 and S2-26; S1-64 and S2-33; S1-65 and S2-35; S1-66 and S2-36; S1-24 and S2-39; S1-32 and S2-40; S1-60 and S2-42; S1-7 and S2-47; S1-11 and S2-57; S1-3 and S2-59; S1-66 and S2-62; S1-2 and S1-1; S1-3 and S1-4; S1-7 and S1-5; S1-9 and S1-6; S1- 10 and S1-12; S1-11 and S1-14; S1-17 and S1-19; S1-24 and S1-20; S1-25 and S1-21; S1-30 and S1-23; S1-32 and S1-27; S1-41 and S1-28; S1-49 and S1-29; S1-50 and S1-31; S1-58 and S1-35; S1-60 and S1-36; S1-64 and S1-37; S1-65 and S1-38; S1-66 and S1-39; S1-2 and S1-46; S1-3 and S1-48; S1-7 and S1-51; S1-9 and S1-52; S1-10 and S1-53; S1-11 and S1-54; S1-17 and S1-55; S1-24 and S1-56; S1-25 and S1-61; S1-30 and S1-62; S1-32 and S1-63; S1-41 and S1-RBD-3; S1-49 and S1-RBD-4; S1-50 and S1-RBD-5; S1-58 and S1-RBD-6; S1-60 and S1-RBD-9; S1-64 and S1-RBD-10; S1-65 and S1-RBD-11; S1-66 and S1-RBD-12; S1-2 and S1-RBD-14; S1-3 and S1-RBD-15; S1-7 and S1-RBD-16; S1-9 and S1-RBD-18; S1-10 and S1-RBD-19; S1-11 and S1-RBD-20; S1-17 and S1-RBD-21; S1-24 and S1-RBD-22; S1-25 and S1-RBD-23; S1-30 and S1-RBD-24; S1-32 and S1-RBD-25; S1-41 and S1-RBD-26; S1-49 and S1-RBD-27; S1-50 and S1-RBD-28; S1-58 and S1-RBD-29; S1-60 and S1-RBD-30; S1-64 and S1-RBD-32; S1-65 and S1-RBD-34; S1-66 and S1-RBD-35; S1-2 and S1-RBD-36; S1-3 and S1-RBD-37; S1-7 and S1-RBD-38; S1-9 and S1-RBD-39; S1-10 and S1-RBD-40; S1-11 and S1-RBD-41; S1-17 and S1-RBD-43; S1-24 and S1-RBD-44; S1-25 and S1-RBD-45; S1-30 and S1-RBD-46; S1-32 and S1-RBD-47; S1-41 and S1-RBD-48; S1-49 and S1-RBD-49; S1-50 and S1-RBD-51; S2-1 and S1-1; S2-2 and S1-4; S2-3 and S1-5; S2-4 and S1-6; S2-5 and S1-12; S2-6 and S1-14; S2-7 and S1-19; S2-9 and S1-20; S2-10 and S1-21; S2-11 and S1-23; S2-13 and S1-27; S2-14 and S1-28; S2-15 and S1-29; S2-18 and S1-31; S2-22 and S1-35; S2-26 and S1-36; S2-33 and S1-37; S2-35 and S1-38; S2-36 and S1-39; S2-39 and S1-46; S2-40 and S1-48; S2-42 and S1-51; S2-47 and S1-52; S2-57 and S1-53; S2-59 and S1-54; S2-62 and S1-55; S2-1 and S1-56; S2-2 and S1-61; S2-3 and S1-62; S2-4 and S1-63; S2-5 and S1-RBD-3; S2-6 and S1-RBD-4; S2-7 and S1-RBD-5; S2-9 and S1-RBD-6; S2-10 and S1-RBD-9; S2-11 and S1-RBD-10; S2-13 and S1-RBD-11; S2-14 and S1-RBD-12; S2-15 and S1-RBD-14; S2-18 and S1-RBD-15; S2-22 and S1-RBD-16; S2-26 and S1-RBD-18; S2-33 and S1-RBD-19; S2-35 and S1-RBD-20; S2-36 and S1-RBD-21; S2-39 and S1-RBD-22; S2-40 and S1-RBD-23; S2-42 and S1-RBD-24; S2-47 and S1-RBD-25; S2-57 and S1-RBD-26; S2-59 and S1-RBD-27; S2-62 and S1-RBD-28; S2-1 and S1-RBD-29; S2-2 and S1-RBD-30; S2-3 and S1-RBD-32; S2-4 and S1-RBD-34; S2-5 and S1-RBD-35; S2-6 and S1-RBD-36; S2-7 and S1-RBD-37; S2-9 and S1-RBD-38; S2-10 and S1-RBD-39; S2-11 and S1-RBD-40; S2-13 and S1-RBD-41; S2-14 and S1-RBD-43; S2-15 and S1-RBD-44; S2-18 and S1-RBD-45; S2-22 and S1-RBD-46; S2-26 and S1-RBD-47; S2-33 and S1-RBD-48; S2-35 and S1-RBD-49; S2-36 and S1-RBD-51.S1-2, S2-14 and S1-52; S1-3, S2-15 and S1-53; S1-7, S2-18 and S1-54; S1-9, S2-22 and S1-55; S1-10, S2-26 and S1-56; S1-11, S2-33 and S1-61; S1-17, S2-35 and S1-62; S1-24, S2-36 and S1-63; S1-25, S2-39 and S1-RBD-3; S1-30, S2-40 and S1-RBD-4; S1-32, S2-42 and S1-RBD-5; S1-41, S2-47 and S1-RBD-6; S1-49, S2-57 and S1-RBD-9; S1-50, S2-59 and S1-RBD-10; S1-58, S2-62 and S1-RBD-11; S1-60, S2-1 and S1-RBD-12; S1-64, S2-2 and S1-RBD-14; S1-65, S2-3 and S1-RBD-15; S1-66, S2-4 and S1-RBD-16; S1-2, S2-5 and S1-RBD-18; S1-3, S2-6 and S1-RBD-19; S1-7, S2-7 and S1-RBD-20; S1-9, S2-9 and S1-RBD-21; S1-10, S2-10 and S1-RBD-22; S1-11, S2-11 and S1-RBD-23; S1-17, S2-13 and S1-RBD-24; S1-24, S2-14 and S1-RBD-25; S1-25, S2-15 and S1-RBD-26; S1-30, S2-18 and S1-RBD-27; S1-32, S2-22 and S1-RBD-28; S1-41, S2-26 and S1-RBD-29; S1-49, S2-33 and S1-RBD-30; S1-50, S2-35 and S1-RBD-32; S1-58, S2-36 and S1-RBD-34; S1-60, S2-39 and S1-RBD-35; S1-64, S2-40 and S1-RBD-36; S1-65, S2-42 and S1-RBD-37; S1-66, S2-47 and S1-RBD-38; S1-2, S2-57 and S1-RBD-39; S1-3, S2-59 and S1-RBD-40; S1-7, S2-62 and S1-RBD-41; S1-9, S2-1 and S1-RBD-43; S1-10, S2-2 and S1-RBD-44; S1-11, S2-3 and S1-RBD-45; S1-17, S2-4 and S1-RBD-46; S1-24, S2-5 and S1-RBD-47; S1-25, S2-6 and S1-RBD-48; S1-30, S2-7 and S1-RBD-49; S1-32, S2-9 and S1-RBD-51; S1-41, S2-10 and S1-1; S1-49, S2-11 and S1-4; S1-50, S2-13 and S1-20; S1-58, S2-14 and S1-21; S1-60, S2-15 and S1-23; S1-64, S2-18 and S1-27; S1-65, S2-1 and S1-28; S1-66, S2-2 and S1-29; S1-2, S2-3 and S1-31; S1-3, S2-4 and S1-35; S1-7, S2-5 and S1-36; S1-9, S2-6 and S1-37; S1-10, S2-7 and S1-38; S1-11, S2-9 and S1-39; S1-17, S2-10 and S1-46; S1-24, S2-11 and S1-48; or S1-25, S2-13 and S1-51.

In certain examples, multi-specific binding molecules include a combination of single-domain antibodies including S1-23 and S1-27, S1-23 and S1-1, S1-23 and S1-RBD-15, or S1-23 in combination with S1-1, S1-RBD-15, and/or S2-40.

In other embodiments, and as indicated below in the discussion of bi-specific binding molecules and immune-activating multi-specifics (1-AMS), multiple different binding domains can be included. Particular embodiments include at least one single-domain antibody binding domain disclosed herein that binds an epitope on SARS-CoV-2 and a binding domain that binds an epitope on a different viral protein, such as a different respiratory virus. Other respiratory viruses that can be targeted with multi-specific formats described herein include, for example, human adenovirus, human boca virus (HBoV), other human coronavirus (HCoV, including SARS-CoV, MERS-CoV, coronavirus 229E, coronavirus OC43, coronavirus NL63, coronavirus HKU1, coronavirus NL, coronavirus NH), influenza (groups A and B), human parainfluenza virus (HPIV2 or 4), and/or human rhinovirus (HRV A-HRVC).

Exemplary binding fragments that can be used in an engineered format that binds a secondary virus include: 8C4, 5Hx-1, 5Hx-2, 5Hx-3, 5Hx-4, 5Hx-5, 5.100K-1, 5PB-1, 5Fb-1, and 1E11 to bind to adenovirus; EPR23305-44 to bind to coxsackie adenovirus; CDC2-A2, G2, 5F9, FIB-H1, and JC57-13 to bind to MERS-CoV; 32D6 to bind to H1N1 influenza virus; CH65 to bind to H1 influenza virus; CR9114, MAb 22/1, MAb70/l, MAb 110/1, MAb 264/2, MAb W18/1, MAb 14/3, MAb 24/4, MAb 47/8, MAb 198/2, MAb 215/2, H2/6A5, H3/4C4, H2/6C4, H2/4B3, H9/B20, H2/4B1, CA6261, 6F12, CR9114, and PEG-1 to bind to influenza A virus; CR8033, CR8071, 113/2, 124/4, 128/2, 134/1, 146/1, 152/2, 160/1, 162/1, 195/3, 206/2, 238/4, and 280/2 to bind to influenza B virus; PAR2 (boca231/9F) to bind to HPIV2; or TCN-711 to bind to rhinovirus.

SARS-CoV-2 bispecific binding molecules bind at least two epitopes wherein at least one of the epitopes is located on SARS-CoV-2. SARS-CoV-2 trispecific binding molecules bind at least 3 epitopes, wherein at least one of the epitopes is located on SARS-CoV-2, and so on. Tri-specific binding molecules are described in, for example, WO2016/105450, WO 2010/028796; WO 2009/007124; WO 2002/083738; US 2002/0051780; and WO 2000/018806. The SARS-CoV-2 epitope can be on the S1 non-RBD segment of the spike protein, the S2 segment of the spike protein, or the RBD segment of the spike protein.

Bispecific binding molecules can be prepared utilizing antibody binding domain fragments. For example, WO 1996/016673 describes a bispecific ErbB2/Fc gamma RIII antibody; U.S. Pat. No. 5,837,234 describes a bispecific ErbB2/Fc gamma RI antibody; WO 1998/002463 describes a bispecific ErbB2/Fc alpha antibody; and U.S. Pat. No. 5,821,337 describes a bispecific ErbB2/CD3 antibody.

Some additional exemplary bispecific binding molecules have two heavy chains (each having three heavy chain CDRs, followed by (N-terminal to C-terminal) a CH1 domain, a hinge, a CH2 domain, and a CH3 domain). However, additional architectures are envisioned, including bi-specific binding molecules in which light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes.

As indicated previously in relation to oligomerization, two different single-domain antibodies can be linked through a linker. Commonly used flexible linkers include the Gly-Ser linkers as described above. Linkers can also include one or more antibody hinge regions and/or immunoglobulin heavy chain constant regions, such as CH3 alone or a CH2CH3 sequence. Additional examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target.

Other forms of bispecific binding molecules include the single chain “Janusins” described in Traunecker et al. (Embo Journal, 10, 3655-3659, 1991).

Bispecific binding molecules with extended half-lives are described in, for example, U.S. Pat. No. 8,921,528 and US Patent Publication No. 2014/0308285.

Because albumin has an extended serum half-life, it can be of use in improving the pharmacokinetics of administered single domain antibodies. In particular embodiments, single domain antibodies can be linked to albumin. In other particular embodiments, single domain antibodies can be linked to albumin-binding domains (ABDs). ABDs include, for example, albumin-binding peptides, antibodies, antibody fragments, single domain antibodies, and designed ankyrin repeat proteins (DARPins).

In particular embodiments, multi-specific binding molecules with extended half-lives include multi-specific binding molecules wherein at least one binding domain binds albumin. In particular embodiments, the multi-specific binding molecule that binds albumin includes a binding domain that binds SARS-CoV2 linked to a binding domain that binds albumin. In particular embodiments, the multi-specific binding molecule that binds albumin includes a single domain antibody that binds SARS-CoV2 linked to a single domain antibody that binds albumin.

In particular embodiments, an albumin-binding domain has the sequence: DITGAALLEAKEAAINELKQYGISDYYVTLINKAKTVEGVNALKAEILSALP (SEQ ID NO: 594). In particular embodiments, an albumin-binding domain includes a variant of the sequence as set forth in SEQ ID NO: 594, wherein the variant sequence is modified by at least one amino acid substitution selected from the group including: E12D, T29H-K35D, and A45D.

In particular embodiments, an albumin-binding domain includes the sequence: LKEAKEKAIEELKKAGITSDYYFDLINKAKTVEGVNALKDEILKA (SEQ ID NO: 595). In particular embodiments, an albumin-binding domain includes a variant of the sequence as set forth in SEQ ID NO: 595, wherein the variant sequence is modified by at least one amino acid substitution selected from the group including: Y21, Y22, L25, K30, T31, E33, G34, A37, L38, E41, 142 and A45.

Additional binding domains that bind albumin include CA645 as described in Adams et al., 2016 MAbs 8(7): 1336-1346 (see, e.g., Protein Data Bank accession codes 5FUZ and 5FUO); anti-HSA Nanobody™ (Ablynx, Ghent, Belgium), AlbudAb™ (GlaxoSmithKline, Brentford, United Kingdom), and other high-affinity albumin nanobody sequences as described in Shen et al., 2020 bioRxivdoi: http://doi.org/10.1101/2020.8.19.257725; Mester, et al., 2021 mAbs. 13:1; Tijink et al., 2008 Mol Cancer Ther (7) (8) 2288-2297; and Roovers et al., Cancer Immunol Immunother 2007; 56: 303-317.

In particular embodiments, binding domains disclosed herein can be used to create bi-tri, (or more) specific immune cell engaging molecules. Immune cell engaging molecules have at least one binding domain that binds a receptor on an immune cell and alters the activation state of the immune cell. Examples of multi-specific immune cell engaging molecules include those which bind both SARS-CoV-2 and an immune cell (e.g., T-cell or NK-cells) activating epitope, with the goal of bringing immune cells to SARS-CoV-2-infected cells to destroy them. See, for example, US 2008/0145362. Such molecules are referred to herein as immune-activating multi-specifics or I-AMS). BiTEs® (Amgen, Thousand Oaks, CA) are one form of I-AMS. Immune cells that can be targeted for localized activation by I-AMS within the current disclosure include, for example, B-cells, T-cells, natural killer (NK) cells, and macrophages which are discussed in more detail herein.

I-AMS disclosed herein can target any T-cell activating epitope that upon binding induces T-cell activation. Examples of such T-cell activating epitopes are on T-cell markers including CD2, CD3, CD7, CD27, CD28, CD30, CD40, CD83, 4-1BB (CD137), OX40, lymphocyte function-associated antigen-1 (LFA-1), LIGHT, NKG2C, and B7-H3.

In particular embodiments, the CD3 binding domain (e.g., scFv) is derived from the OKT3 antibody (the same as the one utilized in blinatumomab). The OKT3 antibody is described in detail in U.S. Pat. No. 5,929,212. Additional examples of CD3 antibodies, binding domains, and CDRs can be found in WO2016/116626. TR66 may also be used.

In particular embodiments, a binding domain is “derived from” a reference antibody when the binding domain includes the CDRs of the reference antibody, according to a known numbering scheme (e.g., Kabat, Chothia, Martin, or others).

CD28 binds to B7-1 (CD80) and B7-2 (CD86) and is the most potent of the known co-stimulatory molecules (June et al., Immunol. Today 15:321, 1994; Linsley et al., Ann. Rev. Immunol. 11:191, 1993). In particular embodiments, the CD28 binding domain (e.g., scFv) is derived from CD80, CD86 or the 9D7 antibody. Additional antibodies that bind CD28 include 9.3, KOLT-2, 15E8, 248.23.2, and EX5.3D10.

Activated T-cells express 4-1BB (CD137). In particular embodiments, the 4-1BB binding domain includes a variable light chain including a CDRL1 sequence including RASQSVS (SEQ ID NO: 504), a CDRL2 sequence including ASNRAT (SEQ ID NO: 505), and a CDRL3 sequence including QRSNWPPALT (SEQ ID NO: 506) and a variable heavy chain including a CDRH1 sequence including YYWS (SEQ ID NO: 507), a CDRH2 sequence including INH, and a CDRH3 sequence including YGPGNYDWYFDL (SEQ ID NO: 508).

Particular embodiments disclosed herein including binding domains that bind epitopes on CD8. In particular embodiments, the CD8 binding domain (e.g., scFv) is derived from the OKT8 antibody.

In particular embodiments natural killer cells (also known as NK-cells, K-cells, and killer cells) are targeted for localized activation by I-AMS. NK cells can induce apoptosis or cell lysis by releasing granules that disrupt cellular membranes and can secrete cytokines to recruit other immune cells.

Examples of commercially available antibodies that bind to an NK cell receptor and induce and/or enhance activation of NK cells include: 5C6 and 1D11, which bind and activate NKG2D (available from BioLegend® San Diego, CA); mAb 33, which binds and activates KIR2DL4 (available from BioLegend®); P44-8, which binds and activates NKp44 (available from BioLegend®); SK1, which binds and activates CD8; and 3G8 which binds and activates CD16.

Binding domains of I-AMS and other engineered formats described herein may be joined through a linker. As indicated previously, a linker is an amino acid sequence which can provide flexibility and room for conformational movement between the binding domains of a I-AM. Any appropriate linker may be used.
(iv) Single-Domain Antibody Conjugates. Single-domain antibody conjugates include a single-domain antibody or HcAb disclosed herein linked to another molecule. Examples of single-domain antibody conjugates include single-domain antibody immunotoxins, single-domain antibody-drug conjugates (ADCs), single-domain antibody radioisotope conjugates, and single-domain antibody detectable label conjugates.

SARS-CoV-2 Single-Domain Antibody Immunotoxins. In particular embodiments, the single-domain antibody can be formed as a single-domain antibody immunotoxin. SARS-CoV-2 single-domain antibody immunotoxins include a SARS-CoV-2 single-domain antibody or HcAb disclosed herein conjugated to one or more cytotoxins (e.g., protein toxins, enzymatically active toxins of bacterial, fungal, plant, or animal origin, or fragments thereof). A toxin can be any agent that is detrimental to cells, such as virally-infected cells displaying relevant viral peptides. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). The toxin may be obtained from essentially any source and can be a synthetic or a natural product.

Immunotoxins with multiple (e.g., four) cytotoxins per binding domain can be prepared by partial reduction of the binding domain with an excess of a reducing reagent such as dithiothreitol (DTT) or tris(2-carboxyethyl)phosphine (TCEP) at 37° C. for 30 min, then the buffer can be exchanged by elution through SEPHADEX G-25 resin with 1 mM DTPA (diethylene triamine penta-acetic acid) in Dulbecco's phosphate-buffered saline (DPBS). The eluent can be diluted with further DPBS, and the thiol concentration of the binding domain can be measured using 5,5′-dithiobis(2-nitrobenzoic acid) [Ellman's reagent]. An excess, for example 5-fold, of the linker-cytotoxin conjugate can be added at 4° C. for 1 hr, and the conjugation reaction can be quenched by addition of a substantial excess, for example 20-fold, of cysteine. The resulting immunotoxin mixture can be purified on SEPHADEX G-25 equilibrated in PBS to remove unreacted linker-cytotoxin conjugate, desalted if desired, and purified by size-exclusion chromatography. The resulting immunotoxin can then be sterile filtered, for example, through a 0.2 μm filter, and can be lyophilized if desired for storage.

Single-domain antibody-drug conjugates allow for the targeted delivery of a drug moiety to a cell expressing and displaying portions of SARS-CoV-2 proteins and, in particular embodiments intracellular accumulation therein, where systemic administration of unconjugated drugs may result in unacceptable levels of toxicity to normal cells (Polakis P. (2005) Current Opinion in Pharmacology 5:382-387).

In particular embodiments, single-domain antibody-drug conjugates refer to targeted molecules which combine properties of both antibodies and cytotoxic drugs by targeting potent cytotoxic drugs to antigen-expressing SARS-CoV-2 infected cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107). See also Kamath & lyer (Pharm Res. 32(11): 3470-3479, 2015), which describes considerations for the development of VHH/HcAb-drug conjugates.

The drug moiety (D) of a single-domain antibody-drug conjugate may include any compound, moiety or group that has a cytotoxic or cytostatic effect, for example against virally-infected cells. Drug moieties may impart their cytotoxic and cytostatic effects by mechanisms including tubulin binding, DNA binding or intercalation, and inhibition of RNA polymerase, protein synthesis, and/or topoisomerase. Exemplary drugs include actinomycin D, auristatin, camptothecin, colchicin, daunorubicin, dihydroxy dolastatin, doxorubicin, duocarmycin, emetine, etoposide, gramicidin D, glucocorticoids, maytansinoid mithramycin, mitomycin, nemorubicin, propranolol, puromycin, taxane, taxol, tetracaine, trichothecene, vinblastine, vinca alkaloid, vincristine, and stereoisomers, isosteres, analogs, and derivatives thereof that have cytotoxic activity.

In particular embodiments, the single-domain antibody-drug conjugates include a single-domain antibody or HcAb conjugated, i.e. covalently attached, to the drug moiety. In particular embodiments, the single-domain antibody or HcAb is covalently attached to the drug moiety through a linker. Linkers can be susceptible to cleavage (cleavable linker) or can be substantially resistant to cleavage (e.g., stable linker or noncleavable linker).

Methods described above to produce immunotoxins can similarly be used to prepare single-domain antibody-drug conjugates.

Single-domain antibody-radioisotope conjugates include a SARS-CoV-2 binding domain linked to a radioisotope for use in nuclear medicine. Nuclear medicine refers to the diagnosis and/or treatment of conditions by administering radioactive isotopes (radioisotopes or radionuclides) to a subject. Therapeutic nuclear medicine is often referred to as radiation therapy or radioimmunotherapy (RIT).

Examples of radionuclides that are useful for radiation therapy include ²²⁵Ac and ²²⁷Th. ²²⁵Ac is a radionuclide with the half-life of ten days. As ²²⁵Ac decays the daughter isotopes ²²¹Fr, ²¹³Bi, and ²⁰⁹Pb are formed. ²²⁷Th has a half-life of 19 days and forms the daughter isotope ²²³Ra.

Additional examples of useful radioisotopes include ²²⁸Ac, ¹²⁴Am, ²¹¹As, ¹⁹⁴Au, ⁷Be, ²⁴⁵Bk, ⁷⁶Br, ¹¹C, ²⁵⁴Cf, ²⁴²Cm, ⁵¹Cr, ⁶⁷Cu, ¹⁵³Dy, ¹⁷¹Er, ²⁵⁰Es, ¹⁴⁷Eu, ⁵²Fe, ²⁵¹Fm, ⁶⁶Ga, ¹⁴⁶Gd, ⁶⁸Ge, ¹⁷⁰Hf, ¹⁹³Hg, ¹³¹I, ¹⁸⁵Ir, ⁴²K, ⁷⁹Kr, ¹³²La, ²⁶²Lr ¹⁶⁹Lu, ²⁶⁰Md, ⁵²Mn, ⁹⁰Mo, ²⁴Na, ⁹⁵Nb, ¹³⁸Nd, ⁵⁷Ni, ¹⁵O, ¹⁸²Os ³²P ²⁰¹Pb, ¹⁰¹Pd, ¹⁴³Pr, ¹⁹¹Pt, ²⁴³Pu ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tₘ, ²³⁰U, ⁴⁸V, ¹⁷⁸W, ¹²⁵Xe, ⁸⁶Y, ¹⁶⁹Yb, ⁶⁵Zn, ⁹⁵Zr, and/or ⁹⁷Zr.

Single-domain antibody-detectable label conjugates include a single-domain antibody or HcAb linked to a detectable label. Detectable labels can include any suitable label or detectable group detectable by, for example, optical, spectroscopic, photochemical, biochemical, immunochemical, electrical, optical or chemical means. In particular embodiments, detectable labels can include chemiluminescent labels, spectral colorimetric labels, affinity tags, enzymatic labels, and fluorescent labels.

Chemiluminescent labels can include lucigenin, luminol, luciferin, isoluminol, theromatic acridinium ester, imidazole, acridinium salt, or oxalate ester.

Spectral colorimetric labels can include colloidal gold or colored glass or plastic (e.g., polystyrene, polypropylene, and latex) beads.

Affinity tags can include, for example, His tag (HHHHHH (SEQ ID NO: 526)), Flag tag (DYKDDDD (SEQ ID NO: 527), Xpress tag (DLYDDDDK (SEQ ID NO: 528)), Avi tag (GLNDIFEAQKIEWHE (SEQ ID NO: 529)), Calmodulin binding peptide (CBP) tag (KRRWKKNFIAVSAANRFKKISSSGAL (SEQ ID NO: 530)), Polyglutamate tag (EEEEEE (SEQ ID NO: 531)), HA tag (YPYDVPDYA (SEQ ID NO: 532)), Myc tag (EQKLISEEDL (SEQ ID NO: 533)), Strep tag (WRHPQFGG (SEQ ID NO: 534)), STREP® tag II (WSHPQFEK (SEQ ID NO: 535); IBA Institut fur Bioanalytik, Germany; see, e.g., U.S. Pat. No. 7,981,632), Softag 1 (SLAELLNAGLGGS (SEQ ID NO: 536)), Softag 3 (TQDPSRVG (SEQ ID NO: 537)), and V5 tag (GKPIPNPLLGLDST (SEQ ID NO: 538)).

Enzymatic labels can produce, for example, a chemiluminescent signal, a color signal, or a fluorescent signal. Enzymes can include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Fluorescent labels can be particularly useful in cell staining, identification, imaging, and isolation uses. Exemplary fluorescent labels include blue fluorescent proteins (e.g. eBFP, eBFP2, Azurite, mKalama1, GFPuv, Sapphire, T-sapphire); cyan fluorescent proteins (e.g. eCFP, Cerulean, CyPet, AmCyanl, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen)), CopGFP, AceGFP, avGFP, ZsGreenl, Oregon Green™(Thermo Fisher Scientific)); Luciferase; orange fluorescent proteins (mOrange, mKO, Kusabira-Orange, Monomeric Kusabira-Orange, mTangerine, tdTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowl); and tandem conjugates.

Single-domain antibodies or HcAb can also be attached to microparticles to form single-domain antibody-microparticle conjugates. Microparticles refer to small discrete particles including microbeads, nanobeads, nanoshells, or nanodots. Microparticles can include, for example, latex beads, polystyrene beads, fluorescent beads, and/or colored beads, and can be made from organic matter and/or inorganic matter. They can be made of any suitable materials that allow for the conjugation of capture proteins, such as VHH/HcAb to their surface. Examples of suitable materials include: ceramics, glass, polymers, and magnetic materials. Suitable polymers include polystyrene, poly-(methyl methacrylate), poly-(lactic acid), (poly-(lactic-co-glycolic acid)), polyesters, polyethers, polyolefins, polyalkylene oxides, polyamides, polyurethanes, polysaccharides, celluloses, polyisoprenes, methylstyrene, acrylic polymers, thoria sol, latex, nylon, Teflon cross-linked dextrans (e.g., Sepharose), chitosan, agarose, and cross-linked micelles. Additional examples include carbon graphited, titanium dioxide, and paramagnetic materials. See, e.g., “Microsphere Detection Guide” from Bangs Laboratories, Fishers Ind. In particular embodiments, microparticles can be made of one or more materials. In particular embodiments, microparticles are paramagnetic microparticles. Particular embodiments utilize carboxy-modified polystyrene latex (CML) flow cytometry beads and/or magnetic MagPlex® (Luminex, Austin, TX) flow cytometry beads.
(v) Chimeric Antigen Receptors. Single-domain antibodies disclosed herein can be utilized within chimeric antigen receptors (CAR). CAR include several distinct subcomponents that allow genetically modified cells (e.g., regulatory T cells) to recognize and kill virally-infected cells. The subcomponents include at least an extracellular component and an intracellular component. The extracellular component includes a binding domain that specifically binds a viral protein epitope that is preferentially present on the surface of virally-infected cells or in the area thereof. When the binding domain binds such viral protein epitopes, the intracellular component activates the cell to destroy the bound cell. CAR additionally include a transmembrane domain that directly or indirectly links the extracellular component to the intracellular component, and other subcomponents that can increase the CAR's function. For example, the inclusion of a spacer region and/or one or more linker sequences can allow the CAR to have additional conformational flexibility, often increasing the binding domain's ability to bind the targeted viral protein epitope.

Particular embodiments of binding domains include a VHH domain and/or the CDRs thereof as disclosed herein, such as those provided in Table 1 for S1-1, S1-2, S1-3, S1-4, S1-5, S1-6, S1-7, S1-9, S1-10, S1-11, S1-12, S1-14, S1-17, S1-19, S1-20, S1-21, S1-23, S1-24, S1-25, S1-27, S1-28, S1-29, S1-30, S1-31, S1-32, S1-35, S1-36, S1-37, S1-38, S1-39, S1-41, S1-46, S1-48, S1-49, S1-50, S1-51, S1-52, S1-53, S1-54, S1-55, S1-56, S1-58, S1-60, S1-61, S1-62, S1-63, S1-64, S1-65, S1-66, S1-RBD-3, S1-RBD-4, S1-RBD-5, S1-RBD-6, S1-RBD-9, S1-RBD-10, S1-RBD-11, S1-RBD-12, S1-RBD-14, S1-RBD-15, S1-RBD-16, S1-RBD-18, S1-RBD-19, S1-RBD-20, S1-RBD-21, S1-RBD-22, S1-RBD-23, S1-RBD-24, S1-RBD-25, S1-RBD-26, S1-RBD-27, S1-RBD-28, S1-RBD-29, S1-RBD-30, S1-RBD-32, S1-RBD-34, S1-RBD-35, S1-RBD-36, S1-RBD-37, S1-RBD-38, S1-RBD-39, S1-RBD-40, S1-RBD-41, S1-RBD-43, S1-RBD-44, S1-RBD-45, S1-RBD-46, S1-RBD-47, S1-RBD-48, S1-RBD-49, S1-RBD-51, S2-1, S2-2, S2-3, S2-4, S2-5, S2-6, S2-7, S2-9, S2-10, S2-11, S2-13, S2-14, S2-15, S2-18, S2-22, S2-26, S2-33, S2-35, S2-36, S2-39, S2-40, S2-42, S2-47, S2-57, S2-59 and S2-62.

Spacer regions are used to create appropriate distances and/or flexibility from other CAR sub-components. Spacer regions typically include 10 to 250 amino acids, 10 to 200 amino acids, 10 to 150 amino acids, 10 to 100 amino acids, 10 to 50 amino acids, or 10 to 25 amino acids. Exemplary spacer regions include all or a portion of an immunoglobulin hinge region.

Transmembrane domains typically have a three-dimensional structure that is thermodynamically stable in a cell membrane, and generally ranges in length from 15 to 30 amino acids. The structure of a transmembrane domain can include an a helix, a β barrel, a β sheet, a β helix, or any combination thereof. Transmembrane domains can include at least the transmembrane region(s) of the α, β or ζ chain of a T-cell receptor, CD28, CD27, CD3, CD45, CD4, CD5, CD8, CD9, CD16, CD22; CD33, CD37, CD64, CD80, CD86, CD134, CD137 and CD154.

A transmembrane domain can include one or more additional amino acids adjacent to the transmembrane region, e.g., one or more amino acid within the extracellular region of the CAR (e.g., up to 15 amino acids of the extracellular region) and/or one or more additional amino acids within the intracellular region of the CAR (e.g., up to 15 amino acids of the intracellular components).

The intracellular effector domains of a CAR are responsible for activation of the cell in which the CAR is expressed. The term “effector domain” is thus meant to include any portion of the intracellular domain sufficient to transduce an activation signal.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CD3γ, CD3δ, CD3ε, CD3ζ, CD27, CD28, DAP10, ICOS, LAG3, NKG2D, NOTCH1, OX40, ROR2, SLAMF1, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), LIGHT, NKG2C, B7-H3, a ligand that specifically binds with CD83, CDS, ICAM-1, GITR, BAFFR, SLAMF7, NKp80 (KLRF1), CD127, CD19, CD4, CD8α, CD8P, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRy (FCER1G), FcγRIIa, FcRβ (Fcε Rib), DAP10, and DAP12. In particular embodiments, variants of CD3ζ retain at least one, two, three, or all ITAM regions.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD137), OX40, PD-1, ICOS, lymphocyte function-associated antigen-1 (LFA-1), NKG2C, and a ligand that specifically binds with CD83.

Transduction markers may be selected from, for example, at least one of a truncated CD19 (tCD19; see Budde et al., Blood 122: 1660, 2013); a truncated human EGFR (tEGFR; see Wang et al., Blood 118: 1255, 2011); an extracellular domain of human CD34; and/or RQR8 which combines target epitopes from CD34 (see Fehse et al, Mol. Therapy 1 (5 Pt 1); 448-456, 2000) and CD20 antigens (see Philip et al, Blood 124: 1277-1278). Methods to genetically modify cells to express CAR are well-known in the art.
(vi) Compositions and Combination Cocktails. Any of the single-domain antibody or HcAb in any of the described formats or combinations can be formulated alone or in combination into compositions for administration to subjects. These are referred to as “active ingredients”. Salts and/or pro-drugs of the active ingredients can also be used.

Of note, the disclosed single-domain antibody library has favorable stability properties under potentially denaturing conditions. This is a key consideration for biological therapeutics and diagnostics for SARS-CoV-2. To address this, differential scanning fluorimetry (DSF) experiments were performed to determine the thermal stability (Tₘ) of the disclosed nanobodies. These studies revealed a thermal stability range between 50° C. and 80° C., similar to published results of other properly folded nanobodies and indicative of the high stability generally associated with nanobodies (Muyldermans, 2013, Annual review of biochemistry 82, 775-797). In contrast to many conventional antibodies, nanobodies are also reported to remain fully active upon reconstitution after lyophilization, particularly in buffers lacking cryoprotectants (Schoof et al., 2020, bioRxiv; Xiang et al., 2020, Science 370, 1479-1484). A representative sample from the disclosed nanobody library was thus freeze-dried without cryoprotectants, reconstituted, then analyzed via SPR and DSF to determine whether their properties were compromised due to lyophilization. The results revealed no significant effect on stability, kinetics and affinity (FIGS. 7A-7G). Taken together, these data suggest that the disclosed nanobodies, are highly robust and able to withstand various temperatures and storage conditions without affecting their stability and binding. These are essential requirements for downstream applications (e.g. use in a nebulizer) and ease of storage—important considerations when used for mass distribution to populations across the globe, including in resource-poor settings (Peeling and McNerney, 2014, Expert Rev Mol Diagn 14, 525-534).

Compositions disclosed herein can include combinations of different single-domain antibodies, HcAb, and/or cells genetically modified to express different single-domain antibodies, HcAb.

Certain embodiments include combinations of nanobodies. In particular embodiments, nanobody combinations include at least one type nanobody that binds the S1 epitope (RBD or non-RBD), at least one type of nanobody that binds the S2 epitope, and/or at least one type of nanobody that binds the receptor binding domain (RBD) of Spike. In particular embodiments, nanobody combinations include at least two types of nanobodies that bind the S1 epitope (RBD or non-RBD), at least two types of nanobodies that bind the S2 epitope, and/or at least two types of nanobodies that bind the receptor binding domain (RBD) of Spike. In particular embodiments, nanobody combinations include at least three types of nanobodies that bind the S1 epitope (RBD or non-RBD), at least three types of nanobodies that bind the S2 epitope, and/or at least three types of nanobodies that bind the receptor binding domain (RBD) of Spike. In particular embodiments, nanobody combinations include at least four types of nanobodies that bind the S1 epitope (RBD or non-RBD), at least four types of nanobodies that bind the S2 epitope, and/or at least four types of nanobodies that bind the receptor binding domain (RBD) of Spike.

Certain combinations described herein include combinations of nanobodies that bind distinct epitopes. Distinct epitopes are those that do not completely match. One type of distinct epitopes includes non-overlapping epitopes where there is no commonality between epitopes. Another type of distinct epitopes includes partially-overlapping epitopes, wherein there is some commonality between epitopes (e.g., a commonly bound residue).

In particular embodiments, the nanobody that binds the S1 non-RBD epitope includes S1-2, S1-3, S1-7, S1-9, S1-10, S1-11, S1-17, S1-24, S1-25, S1-30, S1-32, S1-41, S1-49, S1-50, S1-58, S1-60, S1-64, S1-65, and/or S1-66.

In particular embodiments, the nanobody that binds the S2 epitope includes S2-1, S2-2, S2-3, S2-4, S2-5, S2-6, S2-7, S2-9, S2-10, S2-11, S2-13, S2-14, S2-15, S2-18, S2-22, S2-26, S2-33, S2-35, S2-36, S2-39, S2-40, S2-42, S2-47, S2-57, S2-59, and/or S2-62.

In particular embodiments, the nanobody that binds the RBD of Spike includes S1-1, S1-4, S1-5, S1-6, S1-12, S1-14, S1-19, S1-20, S1-21, S1-23, S1-27, S1-28, S1-29, S1-31, S1-35, S1-36, S1-37, S1-38, S1-39, S1-46, S1-48, S1-51, S1-52, S1-53, S1-54, S1-55, S1-56, S1-61, S1-62, S1-63, S1-RBD-3, S1-RBD-4, S1-RBD-5, S1-RBD-6, S1-RBD-9, S1-RBD-10, S1-RBD-11, S1-RBD-12, S1-RBD-14, S1-RBD-15, S1-RBD-16, S1-RBD-18, S1-RBD-19, S1-RBD-20, S1-RBD-21, S1-RBD-22, S1-RBD-23, S1-RBD-24, S1-RBD-25, S1-RBD-26, S1-RBD-27, S1-RBD-28, S1-RBD-29, S1-RBD-30, S1-RBD-32, S1-RBD-34, S1-RBD-35, S1-RBD-36, S1-RBD-37, S1-RBD-38, S1-RBD-39, S1-RBD-40, S1-RBD-41, S1-RBD-43, S1-RBD-44, S1-RBD-45, S1-RBD-46, S1-RBD-47, S1-RBD-48, S1-RBD-49, and/or S1-RBD-51.

In particular embodiments, nanobody combinations include 2, 3, 4, 5, 6, 7, 8, 9, 10, or more types of nanobodies.

In particular embodiments, nanobody compositions include S1-23 and another nanobody. In particular embodiments, nanobody compositions include S1-27 and S1-23. In particular embodiments, nanobody compositions include S1-23 and S1-1. In particular embodiments, nanobody compositions include S1-23 and S1-RBD-15. In particular embodiments, nanobody compositions include S1-23 and S2-40. Particular combinations include S1-23 in combination with S1-1, S1-RBD-15, and/or S2-40.

In particular embodiments, nanobody compositions including nanobodies that bind S1 and S2 epitopes include S1-2 and S2-1; S1-3 and S2-2; S1-7 and S2-3; S1-9 and S2-4; S1-10 and S2-5; S1-11 and S2-6; S1-17 and S2-7; S1-24 and S2-9; S1-25 and S2-10; S1-30 and S2-11; S1- 32 and S2-13; S1-41 and S2-14; S1-49 and S2-15; S1-50 and S2-18; S1-58 and S2-22; S1-60 and S2-26; S1-64 and S2-33; S1-65 and S2-35; S1-66 and S2-36; S1-24 and S2-39; S1-32 and S2-40; S1-60 and S2-42; S1-7 and S2-47; S1-11 and S2-57; S1-3 and S2-59; and/or S1-66 and S2-62.

In particular embodiments, nanobody compositions including nanobodies that bind S1 and RBD epitopes include S1-2 and S1-1; S1-3 and S1-4; S1-7 and S1-5; S1-9 and S1-6; S1-10 and S1-12; S1-11 and S1-14; S1-17 and S1-19; S1-24 and S1-20; S1-25 and S1-21; S1-30 and S1-23; S1-32 and S1-27; S1-41 and S1-28; S1-49 and S1-29; S1-50 and S1-31; S1-58 and S1-35; S1-60 and S1-36; S1-64 and S1-37; S1-65 and S1-38; S1-66 and S1-39; S1-2 and S1-46; S1-3 and S1-48; S1-7 and S1-51; S1-9 and S1-52; S1-10 and S1-53; S1-11 and S1-54; S1-17 and S1-55; S1-24 and S1-56; S1-25 and S1-61; S1-30 and S1-62; S1-32 and S1-63; S1-41 and S1-RBD-3; S1-49 and S1-RBD-4; S1-50 and S1-RBD-5; S1-58 and S1-RBD-6; S1-60 and S1-RBD-9; S1-64 and S1-RBD-10; S1-65 and S1-RBD-11; S1-66 and S1-RBD-12; S1-2 and S1-RBD-14; S1-3 and S1-RBD-15; S1-7 and S1-RBD-16; S1-9 and S1-RBD-18; S1-10 and S1-RBD-19; S1-11 and S1-RBD-20; S1-17 and S1-RBD-21; S1-24 and S1-RBD-22; S1-25 and S1-RBD-23; S1-30 and S1-RBD-24; S1-32 and S1-RBD-25; S1-41 and S1-RBD-26; S1-49 and S1-RBD-27; S1-50 and S1-RBD-28; S1-58 and S1-RBD-29; S1-60 and S1-RBD-30; S1-64 and S1-RBD-32; S1-65 and S1-RBD-34; S1-66 and S1-RBD-35; S1-2 and S1-RBD-36; S1-3 and S1-RBD-37; S1-7 and S1-RBD-38; S1-9 and S1-RBD-39; S1-10 and S1-RBD-40; S1-11 and S1-RBD-41; S1-17 and S1-RBD-43; S1-24 and S1-RBD-44; S1-25 and S1-RBD-45; S1-30 and S1-RBD-46; S1-32 and S1-RBD-47; S1-41 and S1-RBD-48; S1-49 and S1-RBD-49; and/or S1-50 and S1-RBD-51.

In particular embodiments, nanobody compositions including nanobodies that bind S2 and RBD epitopes include S2-1 and S1-1; S2-2 and S1-4; S2-3 and S1-5; S2-4 and S1-6; S2-5 and S1-12; S2-6 and S1-14; S2-7 and S1-19; S2-9 and S1-20; S2-10 and S1-21; S2-11 and S1-23; S2-13 and S1-27; S2-14 and S1-28; S2-15 and S1-29; S2-18 and S1-31; S2-22 and S1-35; S2-26 and S1-36; S2-33 and S1-37; S2-35 and S1-38; S2-36 and S1-39; S2-39 and S1-46; S2-40 and S1-48; S2-42 and S1-51; S2-47 and S1-52; S2-57 and S1-53; S2-59 and S1-54; S2-62 and S1-55; S2-1 and S1-56; S2-2 and S1-61; S2-3 and S1-62; S2-4 and S1-63; S2-5 and S1-RBD-3; S2-6 and S1-RBD-4; S2-7 and S1-RBD-5; S2-9 and S1-RBD-6; S2-10 and S1-RBD-9; S2-11 and S1-RBD-10; S2-13 and S1-RBD-11; S2-14 and S1-RBD-12; S2-15 and S1-RBD-14; S2-18 and S1-RBD-15; S2-22 and S1-RBD-16; S2-26 and S1-RBD-18; S2-33 and S1-RBD-19; S2-35 and S1-RBD-20; S2-36 and S1-RBD-21; S2-39 and S1-RBD-22; S2-40 and S1-RBD-23; S2-42 and S1-RBD-24; S2-47 and S1-RBD-25; S2-57 and S1-RBD-26; S2-59 and S1-RBD-27; S2-62 and S1-RBD-28; S2-1 and S1-RBD-29; S2-2 and S1-RBD-30; S2-3 and S1-RBD-32; S2-4 and S1-RBD-34; S2-5 and S1-RBD-35; S2-6 and S1-RBD-36; S2-7 and S1-RBD-37; S2-9 and S1-RBD-38; S2-10 and S1-RBD-39; S2-11 and S1-RBD-40; S2-13 and S1-RBD-41; S2-14 and S1-RBD-43; S2-15 and S1-RBD-44; S2-18 and S1-RBD-45; S2-22 and S1-RBD-46; S2-26 and S1-RBD-47; S2-33 and S1-RBD-48; S2-35 and S1-RBD-49; and/or S2-36 and S1-RBD-51.

In particular embodiments, nanobody compositions including nanobodies that bind S1, S2, and RBD epitopes include S1-2, S2-14 and S1-52; S1-3, S2-15 and S1-53; S1-7, S2-18 and S1-54; S1-9, S2-22 and S1-55; S1-10, S2-26 and S1-56; S1-11, S2-33 and S1-61; S1-17, S2-35 and S1-62; S1-24, S2-36 and S1-63; S1-25, S2-39 and S1-RBD-3; S1-30, S2-40 and S1-RBD-4; S1-32, S2-42 and S1-RBD-5; S1-41, S2-47 and S1-RBD-6; S1-49, S2-57 and S1-RBD-9; S1-50, S2-59 and S1-RBD-10; S1-58, S2-62 and S1-RBD-11; S1-60, S2-1 and S1-RBD-12; S1-64, S2-2 and S1-RBD-14; S1-65, S2-3 and S1-RBD-15; S1-66, S2-4 and S1-RBD-16; S1-2, S2-5 and S1-RBD-18; S1-3, S2-6 and S1-RBD-19; S1-7, S2-7 and S1-RBD-20; S1-9, S2-9 and S1-RBD-21; S1-10, S2-10 and S1-RBD-22; S1-11, S2-11 and S1-RBD-23; S1-17, S2-13 and S1-RBD-24; S1-24, S2-14 and S1-RBD-25; S1-25, S2-15 and S1-RBD-26; S1-30, S2-18 and S1-RBD-27; S1-32, S2-22 and S1-RBD-28; S1-41, S2-26 and S1-RBD-29; S1-49, S2-33 and S1-RBD-30; S1-50, S2-35 and S1-RBD-32; S1-58, S2-36 and S1-RBD-34; S1-60, S2-39 and S1-RBD-35; S1-64, S2-40 and S1-RBD-36; S1-65, S2-42 and S1-RBD-37; S1-66, S2-47 and S1-RBD-38; S1-2, S2-57 and S1-RBD-39; S1-3, S2-59 and S1-RBD-40; S1-7, S2-62 and S1-RBD-41; S1-9, S2-1 and S1-RBD-43; S1-10, S2-2 and S1-RBD-44; S1-11, S2-3 and S1-RBD-45; S1-17, S2-4 and S1-RBD-46; S1-24, S2-5 and S1-RBD-47; S1-25, S2-6 and S1-RBD-48; S1-30, S2-7 and S1-RBD-49; S1-32, S2-9 and S1-RBD-51; S1-41, S2-10 and S1-1; S1-49, S2-11 and S1-4; S1-50, S2-13 and S1-20; S1-58, S2-14 and S1-21; S1-60, S2-15 and S1-23; S1-64, S2-18 and S1-27; S1-65, S2-1 and S1-28; S1-66, S2-2 and S1-29; S1-2, S2-3 and S1-31; S1-3, S2-4 and S1-35; S1-7, S2-5 and S1-36; S1-9, S2-6 and S1-37; S1-10, S2-7 and S1-38; S1-11, S2-9 and S1-39; S1-17, S2-10 and S1-46; S1-24, S2-11 and S1-48; and/or S1-25, S2-13 and S1-51.

A pharmaceutically acceptable salt includes any salt that retains the activity of the active ingredient and is acceptable for pharmaceutical use. A pharmaceutically acceptable salt also refers to any salt which may form in vivo as a result of administration of an acid, another salt, or a prodrug which is converted into an acid or salt. A prodrug includes an active ingredient which is converted to a therapeutically active compound after administration, such as by cleavage or by hydrolysis of a biologically labile group.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Certain compositions are formulated for pulmonary delivery. “Pulmonary delivery” includes drug delivery via nasal spray, nasal inhaling, an oral inhaler, or any other drug delivery techniques capable of causing a drug to be delivered to the respiratory track and/or lung.

In some examples, the composition may be formulated as a dispersion for nebulization that is prepared by admixing active ingredients with an aqueous carrier that is nebulized by a nebulizer, such as an air-jet nebulizer, an ultrasonic nebulizer or a micro-pump nebulizer. The respirable fraction of nebulized droplets can be generally greater than 40, 50, 60, 70, or 80% as measured by a non-viable 8-stage cascade impactor at an air flow rate of 28.3 L/min. The composition may be suitably adapted for delivery using a metered dose delivery device a dry powder inhalation device or a pressurized metered dose inhalation device.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas.

Particle size for inhalation affects location of delivery to the respiratory system following inhalation. Suitable particle sizes for pulmonary administration include 0.5 and 10 microns. Microparticles having a diameter of between 0.5 and 10 microns can reach the lungs, successfully passing most of the natural barriers. Particles can also be less than 10 μm in diameter, and less than 5 μm. Exemplary particle sizes that can reach the pulmonary alveoli range from 0.5 μm to 5.8 μm in diameter. Such particles can reach the pulmonary capillaries and can avoid extensive contact with the peripheral tissue in the lung. In one embodiment, compositions for pulmonary inhalation include rnicroparticles wherein from 35% to 75% of the microparticles have an aerodynamic diameter of between 0.5 and 10 microns or less than 5.8 μm.

Dry powder compositions may be formulated for delivery with any suitable dry powder inhaler (DPI). Dry powder inhalers are known in the art and particularly suitable inhaler systems are described in U.S. Pat. Nos. 7,305,986 and 7,464,706, both entitled “Unit Dose Capsules and Dry Powder Inhaler”. Other dry powder dispersion devices for pulmonary administration of dry powders include those described, for example, in U.S. Pat. Nos. 5,522,385 and 5,388,572 and EP Pat. Nos. 129985, 472598, and 467172. Also suitable for delivering the dry powders are inhalation devices such as the Astra-Draco “TURBUHALER”. This type of device is described in detail in U.S. Pat. Nos. 4,668,281, 4,667,668 and 4,805,811. Other suitable devices include dry powder inhalers such as the Rotahaler® (Glaxo), Discus® (Glaxo), Spiros™ inhaler (Dura Pharmaceuticals), and the Spinhaler® (Fisons).

For additional information regarding compositions, formulation, and devices for pulmonary delivery, see Pulmonary Drug Delivery”, Bechtold-Peters and Luessen, eds., supra, pages 125 and 126: Prime et al., Review of Dry Powder Inhalers, 26 Adv. Drug Delivery Rev., pp. 51-58 (1997); Hickey et al., A new millennium for inhaler technology, 21 Pharm. Tech., n. 6, pp. 116-125 (1997); and WO 97/41031.

In additional examples of compositions, generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, and benzalkonium halides.

Stabilizers refer to a broad category of excipients which can range in function from a bulking agent to an additive which solubilizes the antibodies or helps to prevent denaturation or adherence to the container wall. Typical stabilizers can include polyhydric sugar alcohols, amino acids, organic sugars or sugar alcohols, PEG, amino acid polymers, sulfur-containing reducing agents, low molecular weight polypeptides (i.e., <10 residues), proteins such as human serum albumin, bovine serum albumin, gelatin or immunoglobulins, hydrophilic polymers, monosaccharides, disaccharides, trisaccharides, and polysaccharides.

Cell-based compositions (e.g., cells genetically modified to express an active ingredient disclosed herein, for example, a VHH as part of a CAR) can be concentrated into a carrier in a therapeutically-effective amount. Exemplary carriers include saline, buffered saline, physiological saline, water, Hanks' solution, Ringer's solution, Nonnosol-R (Abbott Labs), PLASMA-LYTE A® (Baxter Laboratories, Inc., Morton Grove, IL), glycerol, ethanol, and combinations thereof.

In particular embodiments, carriers can be supplemented with human serum albumin (HSA) or other human serum components or fetal bovine serum. In particular embodiments, a carrier for infusion includes buffered saline with 5% HAS or dextrose. Additional isotonic agents include polyhydric sugar alcohols including trihydric or higher sugar alcohols, such as glycerin, erythritol, arabitol, xylitol, sorbitol, or mannitol.

Therapeutically effective amounts of cells within compositions or formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In compositions and formulations disclosed herein, cells are generally in a volume of a liter or less, 500 mls or less, 250 mls or less or 100 mls or less. Hence the density of administered cells is typically greater than 10⁴ cells/ml, 10⁷ cells/ml or 10⁸ cells/ml.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral, sublingual, and/or subcutaneous administration.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of active ingredient. Various sustained-release materials have been established and are well known by those of ordinary skill in the art.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.
(vii) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of an infection's development, progression, and/or resolution.

A composition with an active ingredient that exhibits neutralizing function can reduce or block a virus from infecting cells by, for example, reducing or preventing the virus' ability to interact with a host cell. In particular embodiments, neutralizing active ingredients reduce or prevent interaction of the SARS-CoV-2 receptor binding domain (RBD) with an ACE2 receptor of a host cell. In particular embodiments, neutralizing active ingredients reduce or prevent interaction of the SARS-CoV-2 spike protein with an ACE2 receptor of a host cell. Active ingredients can be tested for neutralization capabilities in assays such as plaque reduction neutralization assays (PRNT) and receptor-binding inhibition assays (RBD inhibition, sVNT); Tan et al., Nature Biotechnology, doi:10.1038/s41587-020-0631-z (2020); and as described herein).

In particular embodiments, a plaque reduction neutralization (PRNT) assay can include the following: heat inactivated plasma can be diluted 1:5 followed by four 4-fold serial dilutions and mixed 1:1 with SARS-CoV-2 WA-1 (a SARS-CoV-2 isolate available from, e.g., BEI Resources, Manassas, VA) in PBS+0.3% cold water fish skin gelatin (e.g., from Sigma Aldrich, St. Louis, MO). After 30 minutes of incubation, the plasma/virus mixtures can be added to 12 well plates of Vero cells and incubated for 1 hour at 37° C. All dilutions can be done in duplicate, along with virus only and no virus controls. Plates can then be washed with PBS and overlaid with a 1:1 mixture of 2.4% Avicel RC-591 (blend of microcrystalline cellulose and carboxymethylcellulose sodium available from, e.g., FMC, Philadelphia, PA) and 2×Mimimum Essential Medium (MEM) (available from, e.g., ThermoFisher Scientific, Waltham, MA) supplemented with 4% heat-inactivated fetal bovine serum (FBS) and Penicillin/Streptomycin. After a 48-hour incubation, the overlay can be removed, plates can be washed with PBS, fixed with 10% formaldehyde in PBS for 30 minutes at room temperature, and stained with 1% crystal violet in 20% ethanol. % Neutralization can be calculated as (1−#sample plaques/#positive control plaques)×100. In particular embodiments, a neutralizing antibody can show 30%, 35%, 40%, 45%, 50%, 55%, 60%, 65%, or greater neutralization in a PRNT assay at a given dilution of heat-inactivated plasma.

A “prophylactic treatment” includes a treatment administered to a subject who does not display signs or symptoms of an infection or displays only early signs or symptoms of an infection such that treatment is administered for the purpose of diminishing or decreasing the risk of developing the infection further. Thus, a prophylactic treatment functions as a preventative treatment against an infection. In particular embodiments, prophylactic treatments reduce, delay, or prevent the worsening of an infection.

A “therapeutic treatment” includes a treatment administered to a subject who displays symptoms or signs of an infection and is administered to the subject for the purpose of diminishing or eliminating those signs or symptoms of the infection. The therapeutic treatment can reduce, control, or eliminate the presence or activity of the infection and/or reduce control or eliminate side effects of the infection.

Function as an effective amount, prophylactic treatment or therapeutic treatment are not mutually exclusive, and in particular embodiments, administered dosages may accomplish more than one treatment type.

In particular embodiments, therapeutically effective amounts provide anti-infection effects. Anti-infection effects include a reducing or preventing a virus from infecting a cell, decreasing the number of infected cells, decreasing the volume of infected tissue, increasing lifespan, increasing life expectancy, reducing or eliminating infection-associated symptoms (e.g., reduced lung capacity, blood clotting, gastrointestinal distress, headaches, “COVID-toe”, reduced ability to taste or smell).

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of infection, stage of infection, previous or concurrent therapeutic interventions, idiopathy of the subject and route of administration.

Useful doses can range from 0.1 to 5 μg/kg or from 0.5 to 1 μg/kg. In other non-limiting examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other non-limiting examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

The pharmaceutical compositions described herein can be administered by, for example, injection, inhalation, infusion, perfusion, lavage, or ingestion. Routes of administration can include intravenous, intradermal, intraarterial, intraparenteral, intranasal, intralesional, intramuscular, oral, subcutaneous, and/or sublingual administration.
(viii) Methods to Obtain and Use Single-Domain Antibodies. The following discussion describes methods that can be used to obtain single-domain antibodies and uses and fusion proteins thereof. Examples of fusion proteins based on single-domain antibodies disclosed herein include HcAb, bi-specific binding molecules, tri-specific binding molecules, multi-specific binding molecules, immune cell engaging molecules, CAR etc. As indicated previously, fusion proteins include different protein domains (e.g., single-domain antibody and Fc region; CAR components; binding domains) linked to each other directly or through intervening linker segments such that the function of each included domain is retained.

In certain approaches, animals are immunized with a suitable amount of antigen (Ag) (e.g., SARS-CoV-2 spike protein or a fragment thereof), such as 5 mg, prepared with Complete Freund's Adjuvant. This is followed by three booster immunizations of 5 mg of antigen, prepared with Incomplete Freund's Adjuvant. Booster immunizations can be performed if desired using any suitable intervals, such as 21, 42, and 62 days after the first immunization. Immunizations could alternatively be performed with smaller amounts of antigen, from 0.1 mg to 5 mg. Test serum bleeds can be obtained after any suitable period after the first immunization, such as 52 days after the first immunization, wherein the animal's immune response is assessed by determining the specific activity of this serum against the antigen. A production serum bleed and bone marrow aspirate can be obtained after initial immunization.

Once a camelid has produced Coronavirus Ag-specific HCAbs, samples are obtained for analysis. For use in determining sequences encoding the non-specific and Ag-specific HCAbs the sample can include any lymphocytes that include DNA sequences encoding the HCAbs. In embodiments, the sample includes plasma cells, (i.e., plasma B cells). In embodiments, the sample includes bone marrow. In embodiments, the sample includes a bone marrow aspirate. In embodiments, the sample includes mononuclear cells that are separated from a bone marrow sample to provide a cell composition that is enriched for plasma cells.

In embodiments, the lymphocytes are processed to separate mRNA or total RNA for use in generating a cDNA library. As is well known in the art, generating a cDNA library includes reverse transcription of the RNA to obtain DNA templates from which the VHH variable regions encoding the plurality of Coronavirus Ag-specific HCAbs as well as the non-specific HCAbs are amplified. The cDNA library can be produced using any suitable techniques. The DNA sequences of the PCR amplicons from the cDNA library are then determined using any suitable technique. In general, high-throughput DNA sequencing methods are used, such as so-called deep sequencing, massively parallel sequencing and next generation sequencing, which are well known techniques and are offered commercially by a number of vendors. In embodiments, the amplified cDNAs are sequenced by high-throughput 454 sequencing or MiSeq sequencing or NovaSeq sequencing. In embodiments, the high-throughput sequencing results in at least 80,000 to 150,000,000 unique reads. Determining the sequences in this manner provides a catalog of sequences encoding the VHH variable regions of the Coronavirus Ag-specific and non-specific HCAbs. From this catalog, the amino acid sequences of these VHH variable regions may be deduced (translated in silico), thus providing a catalog of VHH variable regions, some of which are specific for one or more epitopes present on the Coronavirus antigen administered to the camelid, and many of which are not specific for the antigen. In embodiments, the translated reads can be subjected to computational analysis, which can include in silico protease digestion, the results of which can be stored in a text file or indexed in a searchable peptide database stored on a computer or other digitized media. The text file or searchable database can be configured to account for a variety of parameters, such as the distinct sequences of in silico digested peptides, the number of cDNA sequencing reads that relate to each of those peptides sequences, and the sequences of the complementarity determining regions (CDR1, CDR2 and CDR3), and the framework regions if desired.

In order to determine distinct VHH variable region sequences that are responsible for specificity for the Coronavirus antigen, a sample from the camelid is processed to separate Ag-specific HCAbs from non-specific HCAbs. In general any suitable sample including HCAbs can be used for this purpose. In embodiments, one or a series of serum samples is obtained and processed, for example, to obtain an antibody fraction, such as a fraction that is enriched for HCAbs. In embodiments, sequential purification of serum over immobilized Protein G and Protein A, results in separation of antibodies that includes Coronavirus Ag-specific and non-specific components. The HCAbs are then processed using an affinity purification approach, which involves use of the immunizing antigen as a capture agent.

Any suitable affinity capture approach can be used and will generally include fixing the antigen to a solid substrate, such as a bead or other material, and mixing the Ag-specific HCAbs and non-specific HCAbs with the substrate-fixed antigen such that only the Ag-specific HCAbs are retained on the substrate-fixed antigen. This process yields a composition including Ag-specific HCAbs reversibly and non-covalently bound to the substrate-fixed antigen. In embodiments, the antigen-specific HCAbs can be treated to remove the Fc portion, such as by exposure to papain or the enzyme IdeS, which consequently provides a composition that is enriched with antigen-specific VHH domains bound to the capture agent. The VHH domains can be eluted from the capture agent and purified if desired using any suitable approach. Thus, a composition including isolated and/or purified Coronavirus antigen specific VHH domains is provided for amino acid sequence analysis.

The amino acid sequence analysis of the VHH domains can be performed using any appropriate technique. In an embodiment, mass spectrometric (MS) analysis of the Coronavirus Ag-specific VHH regions is performed and is interpreted such that the CDR sequences or portions thereof are determined. In an embodiment, a computer/microprocessor implemented comparison of the amino acid sequences determined from the Coronavirus Ag-specific VHH domains and the amino acid sequences deduced from the cDNA analysis is performed so that matching sequences can be identified, thus identifying Ag-specific VHH amino acid sequences. In another embodiment, the fragment masses of the amino acid sequences deduced from the cDNA analysis are calculated and compared to tandem mass spectra of the Coronavirus Ag-specific VHH domains. In embodiments, this comparison can include data and rankings related to the MS coverage of complementarity determining regions, which in embodiments includes all of CDR1, CDR2 and CDR3 sequences, mass spectral counts, and expectation values of peptide sequences that match the cDNA and MS sequence data. Based on this analysis, the sequence of Ag-specific VHH domains can be identified.

Once the amino acid sequence of Ag-specific VHH domains are in hand, DNA sequences encoding them can be introduced into expression vectors so that Coronavirus Ag-specific VHH domains can be made recombinantly for further testing, or for use in a wide variety of other methods. In this regard, any suitable expression vector and protein expression system can be used. The expression vector is not particularly limiting other than by a requirement for the Coronavirus Ag-specific VHH domains to be driven from a suitable promoter, and many suitable expression vectors and systems are commercially available. In embodiments the expression systems can be eukaryotic or prokaryotic expression systems, such as bacterial, yeast, mammalian, plant and insect expression systems. In general the expression vector will include at least one promoter driving expression of the VHH domains mRNA from its gene, and may include other regulatory elements to effect and/or optimize expression of the inserted VHH domain coding region. The promoter can be a constitutive or inducible promoter. Suitable expression vectors can thus include prokaryotic and/or eukaryotic promoters, enhancer elements, origins of replication, selectable markers for use in maintaining the expression vectors in the desired cell type, polycloning sites, and may encode such features as visually detectable markers. More than one promoter can be included, and more than one VHH domain can be encoded by any particular expression vector, if desired. The expression vectors can also be adapted to express Coronavirus VHH domain-fusion proteins. The fusion proteins can include any other amino acid sequence that would be desirable for expressing in the same open reading frame as the VHH domains. The Coronavirus-specific VHH domain sequence can be configured N-terminal or C-terminal to the fused open reading frame, depending on the particular fusion protein to be produced. In one embodiment, the protein expression is a bacterial system. In embodiments, the Ag-specific VHH domains identified and produced recombinantly according to this disclosure can exhibit a KD in a sub-micromolar range, such as a nM range. All cDNA sequences encoding Coronavirus specific VHH domains identified by the methods of this disclosure are encompassed within its scope. The polynucleotide sequence encoding the Coronavirus Ag-specific VHH domains can be optimized, such as by optimizing the codon usage for the particular expression system to be used. Further, the Coronavirus Ag-specific VHH domain protein that is expressed can be configured such that it is a component of a fusion protein. Such fusion proteins can include components for use in facilitating purification of the Ag-specific VHH domains from the expression system, such as a HIS or FLAG tag, or can be designed to impart additional function to the VHH domains, such as by providing a detectable label or antiviral agent, or to improve solubility, secretion, or any other function. In an embodiment, the Coronavirus Ag-specific VHH domains and/or fragments thereof may be conjugated to a detectable label, such as a fluorescent moiety, enzyme amplification for use in, for example, enhanced chemiluminescence, or chromogenic substrates, for use detecting Coronavirus in a biological sample.

In embodiments, the Coronavirus Ag-specific VHH domains can be partially or fully humanized for use in prophylaxis and/or therapy of a condition that is positively associated with the presence of the Coronavirus antigen. In general, humanization involves replacing all or some of the camelid derived framework and constant regions of Coronavirus Ag-specific VHH domains with human counterpart sequence, with the aim being to reduce immunogenicity of the Ag-specific VHH domains in therapeutic applications. In some instances, the Framework Region residues of the human immunoglobulin are replaced by corresponding non-human residues. In general, a humanized Ag-specific VHH domains will include substantially all of at least one, and typically two, variable domains, in which all or substantially all of the hypervariable loops correspond to those of the non-human, camelid immunoglobulin and all or substantially all of the framework regions are those of a human immunoglobulin sequence.

(ix) Exemplary Embodiments.
1. A single-domain antibody including a set of complementarity determining regions (CDRs) including:
   (i) a CDR1 having the sequence as set forth in SEQ ID NO: 117, a CDR2 having the sequence as set forth in SEQ ID NO: 118, and a CDRH3 having the sequence as set forth in SEQ ID NO: 119;
   (ii) a CDR1 having the sequence as set forth in SEQ ID NO: 120, a CDR2 having the sequence as set forth in SEQ ID NO: 121, and a CDRH3 having the sequence as set forth in SEQ ID NO: 122;
   (iii) a CDR1 having the sequence as set forth in SEQ ID NO: 123, a CDR2 having the sequence as set forth in SEQ ID NO: 124, and a CDRH3 having the sequence as set forth in SEQ ID NO: 125;
   (iv) a CDR1 having the sequence as set forth in SEQ ID NO: 126, a CDR2 having the sequence as set forth in SEQ ID NO: 127, and a CDRH3 having the sequence as set forth in SEQ ID NO: 128;
   (v) a CDR1 having the sequence as set forth in SEQ ID NO: 129, a CDR2 having the sequence as set forth in SEQ ID NO: 130, and a CDRH3 having the sequence as set forth in SEQ ID NO: 131;
   (vi) a CDR1 having the sequence as set forth in SEQ ID NO: 132, a CDR2 having the sequence as set forth in SEQ ID NO: 133, and a CDRH3 having the sequence as set forth in SEQ ID NO: 134;
   (vii) a CDR1 having the sequence as set forth in SEQ ID NO: 135, a CDR2 having the sequence as set forth in SEQ ID NO: 136, and a CDRH3 having the sequence as set forth in SEQ ID NO: 137;
   (viii) a CDR1 having the sequence as set forth in SEQ ID NO: 138, a CDR2 having the sequence as set forth in SEQ ID NO: 139, and a CDRH3 having the sequence as set forth in SEQ ID NO: 140;
   (ix) a CDR1 having the sequence as set forth in SEQ ID NO: 141, a CDR2 having the sequence as set forth in SEQ ID NO: 142, and a CDRH3 having the sequence as set forth in SEQ ID NO: 143;
   (x) a CDR1 having the sequence as set forth in SEQ ID NO: 144, a CDR2 having the sequence as set forth in SEQ ID NO: 145, and a CDRH3 having the sequence as set forth in SEQ ID NO: 146;
   (xi) a CDR1 having the sequence as set forth in SEQ ID NO: 147, a CDR2 having the sequence as set forth in SEQ ID NO: 148, and a CDRH3 having the sequence as set forth
   (xii) a CDR1 having the sequence as set forth in SEQ ID NO: 150, a CDR2 having the sequence as set forth in SEQ ID NO: 151, and a CDRH3 having the sequence as set forth in SEQ ID NO: 152;
   (xiii) a CDR1 having the sequence as set forth in SEQ ID NO: 153, a CDR2 having the sequence as set forth in SEQ ID NO: 154, and a CDRH3 having the sequence as set forth in SEQ ID NO: 155;
   (xiv) a CDR1 having the sequence as set forth in SEQ ID NO: 156, a CDR2 having the sequence as set forth in SEQ ID NO: 157, and a CDRH3 having the sequence as set forth in SEQ ID NO: 158;
   (xv) a CDR1 having the sequence as set forth in SEQ ID NO: 159, a CDR2 having the sequence as set forth in SEQ ID NO: 160, and a CDRH3 having the sequence as set forth in SEQ ID NO: 161;
   (xvi) a CDR1 having the sequence as set forth in SEQ ID NO: 162, a CDR2 having the sequence as set forth in SEQ ID NO: 163, and a CDRH3 having the sequence as set forth in SEQ ID NO: 164;
   (xvii) a CDR1 having the sequence as set forth in SEQ ID NO: 165, a CDR2 having the sequence as set forth in SEQ ID NO: 166, and a CDRH3 having the sequence as set forth in SEQ ID NO: 167;
   (xviii) a CDR1 having the sequence as set forth in SEQ ID NO: 168, a CDR2 having the sequence as set forth in SEQ ID NO: 169, and a CDRH3 having the sequence as set forth in SEQ ID NO:170;
   (xix) a CDR1 having the sequence as set forth in SEQ ID NO: 171, a CDR2 having the sequence as set forth in SEQ ID NO: 139, and a CDRH3 having the sequence as set forth in SEQ ID NO: 173;
   (xx) a CDR1 having the sequence as set forth in SEQ ID NO: 174, a CDR2 having the sequence as set forth in SEQ ID NO: 175, and a CDRH3 having the sequence as set forth in SEQ ID NO: 176;
   (xxi) a CDR1 having the sequence as set forth in SEQ ID NO: 177, a CDR2 having the sequence as set forth in SEQ ID NO: 178, and a CDRH3 having the sequence as set forth in SEQ ID NO: 179;
   (xxii) a CDR1 having the sequence as set forth in SEQ ID NO: 180, a CDR2 having the sequence as set forth in SEQ ID NO: 181, and a CDRH3 having the sequence as set forth
   (xxiii) a CDR1 having the sequence as set forth in SEQ ID NO: 183, a CDR2 having the sequence as set forth in SEQ ID NO: 184, and a CDRH3 having the sequence as set forth in SEQ ID NO: 185;
   (xxiv) a CDR1 having the sequence as set forth in SEQ ID NO: 186, a CDR2 having the sequence as set forth in SEQ ID NO: 187, and a CDRH3 having the sequence as set forth in SEQ ID NO: 188;
   (xxv) a CDR1 having the sequence as set forth in SEQ ID NO: 189, a CDR2 having the sequence as set forth in SEQ ID NO: 190, and a CDRH3 having the sequence as set forth in SEQ ID NO: 191;
   (xxvi) a CDR1 having the sequence as set forth in SEQ ID NO: 192, a CDR2 having the sequence as set forth in SEQ ID NO: 193, and a CDRH3 having the sequence as set forth in SEQ ID NO: 194;
   (xxvii) a CDR1 having the sequence as set forth in SEQ ID NO: 195, a CDR2 having the sequence as set forth in SEQ ID NO: 178, and a CDRH3 having the sequence as set forth in SEQ ID NO: 197;
   (xxviii) a CDR1 having the sequence as set forth in SEQ ID NO: 198, a CDR2 having the sequence as set forth in SEQ ID NO: 160, and a CDRH3 having the sequence as set forth in SEQ ID NO: 200;
   (xxix) a CDR1 having the sequence as set forth in SEQ ID NO: 201, a CDR2 having the sequence as set forth in SEQ ID NO: 202, and a CDRH3 having the sequence as set forth in SEQ ID NO: 203;
   (xxx) a CDR1 having the sequence as set forth in SEQ ID NO: 204, a CDR2 having the sequence as set forth in SEQ ID NO: 205, and a CDRH3 having the sequence as set forth in SEQ ID NO: 206;
   (xxxi) a CDR1 having the sequence as set forth in SEQ ID NO: 207, a CDR2 having the sequence as set forth in SEQ ID NO: 208, and a CDRH3 having the sequence as set forth in SEQ ID NO: 209;
   (xxxii) a CDR1 having the sequence as set forth in SEQ ID NO: 210, a CDR2 having the sequence as set forth in SEQ ID NO: 211, and a CDRH3 having the sequence as set forth in SEQ ID NO: 212;
   (xxxiii) a CDR1 having the sequence as set forth in SEQ ID NO: 213, a CDR2 having the sequence as set forth in SEQ ID NO: 214, and a CDRH3 having the sequence as set forth
   (xxxiv) a CDR1 having the sequence as set forth in SEQ ID NO: 216, a CDR2 having the sequence as set forth in SEQ ID NO: 217, and a CDRH3 having the sequence as set forth in SEQ ID NO: 218;
   (xxxv) a CDR1 having the sequence as set forth in SEQ ID NO: 120, a CDR2 having the sequence as set forth in SEQ ID NO: 220, and a CDRH3 having the sequence as set forth in SEQ ID NO: 221;
   (xxxvi) a CDR1 having the sequence as set forth in SEQ ID NO: 222, a CDR2 having the sequence as set forth in SEQ ID NO: 223, and a CDRH3 having the sequence as set forth in SEQ ID NO: 224;
   (xxxvii) a CDR1 having the sequence as set forth in SEQ ID NO: 225, a CDR2 having the sequence as set forth in SEQ ID NO: 226, and a CDRH3 having the sequence as set forth in SEQ ID NO: 227;
   (xxxviii) a CDR1 having the sequence as set forth in SEQ ID NO: 228, a CDR2 having the sequence as set forth in SEQ ID NO: 229, and a CDRH3 having the sequence as set forth in SEQ ID NO: 230;
   (xxxix) a CDR1 having the sequence as set forth in SEQ ID NO: 231, a CDR2 having the sequence as set forth in SEQ ID NO: 232, and a CDRH3 having the sequence as set forth in SEQ ID NO: 233;
   (xI) a CDR1 having the sequence as set forth in SEQ ID NO: 234, a CDR2 having the sequence as set forth in SEQ ID NO: 235, and a CDRH3 having the sequence as set forth in SEQ ID NO: 236;
   (xIi) a CDR1 having the sequence as set forth in SEQ ID NO: 237, a CDR2 having the sequence as set forth in SEQ ID NO: 238, and a CDRH3 having the sequence as set forth in SEQ ID NO: 239;
   (xIii) a CDR1 having the sequence as set forth in SEQ ID NO: 240, a CDR2 having the sequence as set forth in SEQ ID NO: 241, and a CDRH3 having the sequence as set forth in SEQ ID NO: 242;
   (xIiii) a CDR1 having the sequence as set forth in SEQ ID NO: 243, a CDR2 having the sequence as set forth in SEQ ID NO: 244, and a CDRH3 having the sequence as set forth in SEQ ID NO: 245;
   (xIiv) a CDR1 having the sequence as set forth in SEQ ID NO: 246, a CDR2 having the sequence as set forth in SEQ ID NO: 247, and a CDRH3 having the sequence as set forth
   (xIv) a CDR1 having the sequence as set forth in SEQ ID NO: 249, a CDR2 having the sequence as set forth in SEQ ID NO: 250, and a CDRH3 having the sequence as set forth in SEQ ID NO: 251;
   (xIvi) a CDR1 having the sequence as set forth in SEQ ID NO: 252, a CDR2 having the sequence as set forth in SEQ ID NO: 253, and a CDRH3 having the sequence as set forth in SEQ ID NO: 254;
   (xIvii) a CDR1 having the sequence as set forth in SEQ ID NO: 255, a CDR2 having the sequence as set forth in SEQ ID NO: 256, and a CDRH3 having the sequence as set forth in SEQ ID NO: 257;
   (xIviii) a CDR1 having the sequence as set forth in SEQ ID NO: 258, a CDR2 having the sequence as set forth in SEQ ID NO: 259, and a CDRH3 having the sequence as set forth in SEQ ID NO: 260;
   (xIix) a CDR1 having the sequence as set forth in SEQ ID NO: 261, a CDR2 having the sequence as set forth in SEQ ID NO: 262, and a CDRH3 having the sequence as set forth in SEQ ID NO: 263;
   (I) a CDR1 having the sequence as set forth in SEQ ID NO: 264, a CDR2 having the sequence as set forth in SEQ ID NO: 265, and a CDRH3 having the sequence as set forth in SEQ ID NO: 266;
   (Ii) a CDR1 having the sequence as set forth in SEQ ID NO: 129, a CDR2 having the sequence as set forth in SEQ ID NO: 268, and a CDRH3 having the sequence as set forth in SEQ ID NO: 269;
   (Iii) a CDR1 having the sequence as set forth in SEQ ID NO: 270, a CDR2 having the sequence as set forth in SEQ ID NO: 271, and a CDRH3 having the sequence as set forth in SEQ ID NO: 272;
   (Iiii) a CDR1 having the sequence as set forth in SEQ ID NO: 273, a CDR2 having the sequence as set forth in SEQ ID NO: 274, and a CDRH3 having the sequence as set forth in SEQ ID NO: 275;
   (Iiv) a CDR1 having the sequence as set forth in SEQ ID NO: 276, a CDR2 having the sequence as set forth in SEQ ID NO: 277, and a CDRH3 having the sequence as set forth in SEQ ID NO: 278;
   (Iv) a CDR1 having the sequence as set forth in SEQ ID NO: 279, a CDR2 having the sequence as set forth in SEQ ID NO: 280, and a CDRH3 having the sequence as set forth
   (Ivi) a CDR1 having the sequence as set forth in SEQ ID NO: 282, a CDR2 having the sequence as set forth in SEQ ID NO: 283, and a CDRH3 having the sequence as set forth in SEQ ID NO: 284;
   (Ivii) a CDR1 having the sequence as set forth in SEQ ID NO: 285, a CDR2 having the sequence as set forth in SEQ ID NO: 286, and a CDRH3 having the sequence as set forth in SEQ ID NO: 287;
   (Iviii) a CDR1 having the sequence as set forth in SEQ ID NO: 288, a CDR2 having the sequence as set forth in SEQ ID NO: 289, and a CDRH3 having the sequence as set forth in SEQ ID NO: 290;
   (Iix) a CDR1 having the sequence as set forth in SEQ ID NO: 246, a CDR2 having the sequence as set forth in SEQ ID NO: 292, and a CDRH3 having the sequence as set forth in SEQ ID NO: 293;
   (Ix) a CDR1 having the sequence as set forth in SEQ ID NO: 294, a CDR2 having the sequence as set forth in SEQ ID NO: 295, and a CDRH3 having the sequence as set forth in SEQ ID NO: 296;
   (Ixi) a CDR1 having the sequence as set forth in SEQ ID NO: 297, a CDR2 having the sequence as set forth in SEQ ID NO: 298, and a CDRH3 having the sequence as set forth in SEQ ID NO: 299;
   (Ixii) a CDR1 having the sequence as set forth in SEQ ID NO: 300, a CDR2 having the sequence as set forth in SEQ ID NO: 301, and a CDRH3 having the sequence as set forth in SEQ ID NO: 302;
   (Ixiii) a CDR1 having the sequence as set forth in SEQ ID NO: 303, a CDR2 having the sequence as set forth in SEQ ID NO: 304, and a CDRH3 having the sequence as set forth in SEQ ID NO: 305;
   (Ixiv) a CDR1 having the sequence as set forth in SEQ ID NO: 306, a CDR2 having the sequence as set forth in SEQ ID NO: 307, and a CDRH3 having the sequence as set forth in SEQ ID NO: 308;
   (Ixv) a CDR1 having the sequence as set forth in SEQ ID NO: 309, a CDR2 having the sequence as set forth in SEQ ID NO: 310, and a CDRH3 having the sequence as set forth in SEQ ID NO: 311;
   (Ixvi) a CDR1 having the sequence as set forth in SEQ ID NO: 312, a CDR2 having the sequence as set forth in SEQ ID NO: 313, and a CDRH3 having the sequence as set forth in SEQ ID NO:314;
   (Ixvii) a CDR1 having the sequence as set forth in SEQ ID NO: 315, a CDR2 having the sequence as set forth in SEQ ID NO: 316, and a CDRH3 having the sequence as set forth in SEQ ID NO: 317;
   (Ixviii) a CDR1 having the sequence as set forth in SEQ ID NO: 318, a CDR2 having the sequence as set forth in SEQ ID NO: 319, and a CDRH3 having the sequence as set forth in SEQ ID NO: 320;
   (Ixix) a CDR1 having the sequence as set forth in SEQ ID NO: 321, a CDR2 having the sequence as set forth in SEQ ID NO: 322, and a CDRH3 having the sequence as set forth in SEQ ID NO: 323;
   (Ixx) a CDR1 having the sequence as set forth in SEQ ID NO: 324, a CDR2 having the sequence as set forth in SEQ ID NO: 325, and a CDRH3 having the sequence as set forth in SEQ ID NO: 326;
   (Ixxi) a CDR1 having the sequence as set forth in SEQ ID NO: 327, a CDR2 having the sequence as set forth in SEQ ID NO: 328, and a CDRH3 having the sequence as set forth in SEQ ID NO: 329;
   (Ixxii) a CDR1 having the sequence as set forth in SEQ ID NO: 330, a CDR2 having the sequence as set forth in SEQ ID NO: 331, and a CDRH3 having the sequence as set forth in SEQ ID NO: 332;
   (Ixxiii) a CDR1 having the sequence as set forth in SEQ ID NO: 270, a CDR2 having the sequence as set forth in SEQ ID NO: 193, and a CDRH3 having the sequence as set forth in SEQ ID NO: 335;
   (Ixxiv) a CDR1 having the sequence as set forth in SEQ ID NO: 318, a CDR2 having the sequence as set forth in SEQ ID NO: 337, and a CDRH3 having the sequence as set forth in SEQ ID NO: 338;
   (Ixxv) a CDR1 having the sequence as set forth in SEQ ID NO: 339, a CDR2 having the sequence as set forth in SEQ ID NO: 340, and a CDRH3 having the sequence as set forth in SEQ ID NO: 341;
   (Ixxvi) a CDR1 having the sequence as set forth in SEQ ID NO: 282, a CDR2 having the sequence as set forth in SEQ ID NO: 343, and a CDRH3 having the sequence as set forth in SEQ ID NO: 344;
   (Ixxvii) a CDR1 having the sequence as set forth in SEQ ID NO: 339, a CDR2 having the sequence as set forth in SEQ ID NO: 346, and a CDRH3 having the sequence as set forth
   (Ixxviii) a CDR1 having the sequence as set forth in SEQ ID NO: 348, a CDR2 having the sequence as set forth in SEQ ID NO: 349, and a CDRH3 having the sequence as set forth in SEQ ID NO: 350;
   (Ixxix) a CDR1 having the sequence as set forth in SEQ ID NO: 351, a CDR2 having the sequence as set forth in SEQ ID NO: 352, and a CDRH3 having the sequence as set forth in SEQ ID NO: 353;
   (Ixxx) a CDR1 having the sequence as set forth in SEQ ID NO: 354, a CDR2 having the sequence as set forth in SEQ ID NO: 355, and a CDRH3 having the sequence as set forth in SEQ ID NO: 356;
   (Ixxxi) a CDR1 having the sequence as set forth in SEQ ID NO: 357, a CDR2 having the sequence as set forth in SEQ ID NO: 358, and a CDRH3 having the sequence as set forth in SEQ ID NO: 359;
   (Ixxxii) a CDR1 having the sequence as set forth in SEQ ID NO: 360, a CDR2 having the sequence as set forth in SEQ ID NO: 361, and a CDRH3 having the sequence as set forth in SEQ ID NO: 362;
   (Ixxxiii) a CDR1 having the sequence as set forth in SEQ ID NO: 363, a CDR2 having the sequence as set forth in SEQ ID NO: 364, and a CDRH3 having the sequence as set forth in SEQ ID NO: 365;
   (Ixxxiv) a CDR1 having the sequence as set forth in SEQ ID NO: 366, a CDR2 having the sequence as set forth in SEQ ID NO: 367, and a CDRH3 having the sequence as set forth in SEQ ID NO: 368;
   (Ixxxv) a CDR1 having the sequence as set forth in SEQ ID NO: 369, a CDR2 having the sequence as set forth in SEQ ID NO: 370, and a CDRH3 having the sequence as set forth in SEQ ID NO: 371;
   (Ixxxvi) a CDR1 having the sequence as set forth in SEQ ID NO: 372, a CDR2 having the sequence as set forth in SEQ ID NO: 373, and a CDRH3 having the sequence as set forth in SEQ ID NO: 374;
   (Ixxxvii) a CDR1 having the sequence as set forth in SEQ ID NO: 375, a CDR2 having the sequence as set forth in SEQ ID NO: 376, and a CDRH3 having the sequence as set forth in SEQ ID NO: 377;
   (Ixxxviii) a CDR1 having the sequence as set forth in SEQ ID NO: 378, a CDR2 having the sequence as set forth in SEQ ID NO: 379, and a CDRH3 having the sequence as set forth
   (Ixxxix) a CDR1 having the sequence as set forth in SEQ ID NO: 381, a CDR2 having the sequence as set forth in SEQ ID NO: 382, and a CDRH3 having the sequence as set forth in SEQ ID NO: 383;
   (xc) a CDR1 having the sequence as set forth in SEQ ID NO: 384, a CDR2 having the sequence as set forth in SEQ ID NO: 385, and a CDRH3 having the sequence as set forth in SEQ ID NO: 386;
   (xci) a CDR1 having the sequence as set forth in SEQ ID NO: 387, a CDR2 having the sequence as set forth in SEQ ID NO: 388, and a CDRH3 having the sequence as set forth in SEQ ID NO: 389;
   (xcii) a CDR1 having the sequence as set forth in SEQ ID NO: 390, a CDR2 having the sequence as set forth in SEQ ID NO: 391, and a CDRH3 having the sequence as set forth in SEQ ID NO: 392;
   (xciii) a CDR1 having the sequence as set forth in SEQ ID NO: 387, a CDR2 having the sequence as set forth in SEQ ID NO: 394, and a CDRH3 having the sequence as set forth in SEQ ID NO: 395;
   (xciv) a CDR1 having the sequence as set forth in SEQ ID NO: 396, a CDR2 having the sequence as set forth in SEQ ID NO: 397, and a CDRH3 having the sequence as set forth in SEQ ID NO: 398;
   (xcv) a CDR1 having the sequence as set forth in SEQ ID NO: 399, a CDR2 having the sequence as set forth in SEQ ID NO: 400, and a CDRH3 having the sequence as set forth in SEQ ID NO: 401;
   (xcvi) a CDR1 having the sequence as set forth in SEQ ID NO: 402, a CDR2 having the sequence as set forth in SEQ ID NO: 403, and a CDRH3 having the sequence as set forth in SEQ ID NO: 404;
   (xcvii) a CDR1 having the sequence as set forth in SEQ ID NO: 396, a CDR2 having the sequence as set forth in SEQ ID NO: 406, and a CDRH3 having the sequence as set forth in SEQ ID NO: 407;
   (xcviii) a CDR1 having the sequence as set forth in SEQ ID NO: 408, a CDR2 having the sequence as set forth in SEQ ID NO: 409, and a CDRH3 having the sequence as set forth in SEQ ID NO: 410;
   (xcix) a CDR1 having the sequence as set forth in SEQ ID NO: 411, a CDR2 having the sequence as set forth in SEQ ID NO: 412, and a CDRH3 having the sequence as set forth
   (c) a CDR1 having the sequence as set forth in SEQ ID NO: 414, a CDR2 having the sequence as set forth in SEQ ID NO: 415, and a CDRH3 having the sequence as set forth in SEQ ID NO: 416;
   (ci) a CDR1 having the sequence as set forth in SEQ ID NO: 417, a CDR2 having the sequence as set forth in SEQ ID NO: 418, and a CDRH3 having the sequence as set forth in SEQ ID NO: 419;
   (cii) a CDR1 having the sequence as set forth in SEQ ID NO: 420, a CDR2 having the sequence as set forth in SEQ ID NO: 421, and a CDRH3 having the sequence as set forth in SEQ ID NO: 422;
   (ciii) a CDR1 having the sequence as set forth in SEQ ID NO: 423, a CDR2 having the sequence as set forth in SEQ ID NO: 424, and a CDRH3 having the sequence as set forth in SEQ ID NO: 425;
   (civ) a CDR1 having the sequence as set forth in SEQ ID NO: 426, a CDR2 having the sequence as set forth in SEQ ID NO: 427, and a CDRH3 having the sequence as set forth in SEQ ID NO: 428;
   (cv) a CDR1 having the sequence as set forth in SEQ ID NO: 429, a CDR2 having the sequence as set forth in SEQ ID NO: 430, and a CDRH3 having the sequence as set forth in SEQ ID NO: 431;
   (cvi) a CDR1 having the sequence as set forth in SEQ ID NO: 432, a CDR2 having the sequence as set forth in SEQ ID NO: 433, and a CDRH3 having the sequence as set forth in SEQ ID NO: 434;
   (cvii) a CDR1 having the sequence as set forth in SEQ ID NO: 435, a CDR2 having the sequence as set forth in SEQ ID NO: 436, and a CDRH3 having the sequence as set forth in SEQ ID NO: 437;
   (cviii) a CDR1 having the sequence as set forth in SEQ ID NO: 438, a CDR2 having the sequence as set forth in SEQ ID NO: 439, and a CDRH3 having the sequence as set forth in SEQ ID NO: 440;
   (cix) a CDR1 having the sequence as set forth in SEQ ID NO: 441, a CDR2 having the sequence as set forth in SEQ ID NO: 442, and a CDRH3 having the sequence as set forth in SEQ ID NO: 443;
   (cx) a CDR1 having the sequence as set forth in SEQ ID NO: 444, a CDR2 having the sequence as set forth in SEQ ID NO: 445, and a CDRH3 having the sequence as set forth
   (cxi) a CDR1 having the sequence as set forth in SEQ ID NO: 429, a CDR2 having the sequence as set forth in SEQ ID NO: 448, and a CDRH3 having the sequence as set forth in SEQ ID NO: 449;
   (cxii) a CDR1 having the sequence as set forth in SEQ ID NO: 429, a CDR2 having the sequence as set forth in SEQ ID NO: 451, and a CDRH3 having the sequence as set forth in SEQ ID NO: 452;
   (cxiii) a CDR1 having the sequence as set forth in SEQ ID NO: 453, a CDR2 having the sequence as set forth in SEQ ID NO: 454, and a CDRH3 having the sequence as set forth in SEQ ID NO: 455;
   (cxiv) a CDR1 having the sequence as set forth in SEQ ID NO: 456, a CDR2 having the sequence as set forth in SEQ ID NO: 457, and a CDRH3 having the sequence as set forth in SEQ ID NO: 458;
   (cxv) a CDR1 having the sequence as set forth in SEQ ID NO: 459, a CDR2 having the sequence as set forth in SEQ ID NO: 460, and a CDRH3 having the sequence as set forth in SEQ ID NO: 461; or
   (cxvi) a CDR1 having the sequence as set forth in SEQ ID NO: 462, a CDR2 having the sequence as set forth in SEQ ID NO: 463, and a CDRH3 having the sequence as set forth in SEQ ID NO: 464,
   each according to Martin numbering.
2. A single-domain antibody including
   a sequence as set forth in: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, or an amino acid sequence of FIG. 21; or
   a sequence having at least 90%, 91%, 92%, 93%, 94%, 95%, 96%, 97%, 98%, or 99%, sequence identity to a sequence as set forth in: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, or an amino acid sequence of FIG. 21.
3. The single-domain antibody of embodiment 2, with CDRs as predicted by Kabat, Chothia, Martin, Contact, IMGT, AHo, and/or North numbering schemes.
4. A fusion protein including at least two copies of the single-domain antibody of embodiments 1-3 wherein the at least two copies are joined by a protein linker.
5. The fusion protein of embodiment 4, wherein the protein linker is a Gly-Ser linker.
6. The fusion protein of embodiment 5, wherein the Gly-Ser linker is (GlyₓSer_{y})ₙ wherein x and y are independently an integer from 0 to 10 provided that x and y are not both 0 and wherein n is an integer of 1, 2, 3, 4, 5, 6, 7, 8, 9 or 10.
7. The fusion protein of embodiments 5 or 6, wherein the Gly-Ser linker is (Gly₄Ser)₄ (SEQ ID NO: 494).
8. The fusion protein of any of embodiments 4-7, including 2, 3, 4, 5, 6, 7, 8, 9, or 10 copies of the single-domain antibody of any of embodiments 1-3.
9. The fusion protein of embodiment 8, having two or three copies of S1-23, S1-49, S1-RBD-35, or S2-7.
10. A single-domain antibody of any of embodiments 1-3, wherein the single-domain antibody is multimerized.
11. The single-domain antibody of embodiment 10, wherein the multimerized single-domain antibody is a dimer, trimer, tetramer, pentamer, hexamer, or heptamer.
12. The single-domain antibody of embodiments 10 or 11, wherein the multimerized single-domain antibody is a dimer or trimer of S1-23, S1-49, S1-RBD-35, or S2-7.
13. A heavy chain only antibody (HcAb) including the single-domain antibody of any of embodiments 1-3 linked to an Fc region of an antibody.
14. The HcAb of embodiment 13, wherein the Fc region is an IgG Fc region, an IgA Fc region, an IgM Fc region, an IgD Fc region or an IgE Fc region.
15. The HcAb of embodiments 13 or 14, wherein the Fc region is an IgA Fc region, an IgM Fc region, or an IgG Fc region.
16. The HcAb of any of embodiments 13-15, wherein the Fc region is an IgM Fc region having the sequence as set forth in SEQ ID NOs: 470-480.
17. The HcAb of any of embodiments 13-15, wherein the Fc region is an IgA Fc region having the sequence as set forth in SEQ ID NOs: 466 and 467.
18. The HcAb of any of embodiments 13-15, wherein the Fc region is an IgG Fc region having the sequence as set forth in SEQ ID NOs: 465 and 587-589.
19. The HcAb of any of embodiments 13-17, wherein the Fc region includes a multimerizing fragment of the IgM Fc region or a multimerizing fragment of the IgA Fc region.
20. The HcAb of embodiment 19, wherein the multimerizing fragment of the IgM Fc region includes the IgM tailpiece.
21. The HcAb of embodiments 19 or 20, wherein the multimerizing fragment of the IgM Fc region includes the Cμ4 domain and the IgM tailpiece.
22. The HcAb of any of embodiments 19-21, wherein the multimerizing fragment of the IgM Fc region includes the Cμ3 domain, the Cμ4 domain, and the IgM tailpiece.
23. The HcAb of any of embodiments 19-22, wherein the multimerizing fragment of the IgM Fc region includes the Cμ2 domain, the Cμ3 domain, the Cμ4 domain, and the IgM tailpiece.
24. The HcAb of any of embodiments 19-23, wherein the multimerizing fragment of the IgM Fc region includes the Cμ1 domain, the Cμ2 domain, the Cμ3 domain, the Cμ4 domain, and the IgM tailpiece.
25. The HcAb of any of embodiments 19-24, wherein the multimerizing fragment of the IgM Fc region has the sequence as set forth in SEQ ID NO: 669.
26. The HcAb of any of embodiments 19-25, wherein the multimerizing fragment of the IgA Fc region includes the IgA tailpiece.
27. The HcAb of embodiment 26, wherein the IgA tailpiece has the sequence of residues 331-352 as set forth in SEQ ID NO: 644 or the sequence of residues 318-340 as set forth in SEQ ID NO: 645.
28. The HcAb of any of embodiments 19-27, wherein the multimerizing fragment of the IgA Fc region includes the IgA CA3 domain and the IgA tailpiece.
29. The HcAb of any of embodiments 19-28, wherein the multimerizing fragment of the IgA Fc region includes the IgA CA2 domain, the IgA CA3 domain, and the IgA tailpiece.
30. The HcAb of any of embodiments 19-29, wherein the multimerizing fragment of the IgA Fc region includes the IgA CA1 domain, the IgA CA2 domain, the IgA CA3 domain, and the IgA tailpiece.
31. The HcAb of any of embodiments 13-30, wherein the Fc region includes an IgG Fc region and a multimerizing fragment of the IgM Fc region or a multimerizing fragment of the IgA Fc region.
32. The HcAb of embodiment 31, wherein the multimerizing fragment of the IgM Fc region or the multimerizing fragment of the IgA Fc region is fused to the C-terminus of the IgG Fc region.
33. The HcAb of embodiments 31 or 32, wherein the IgG is IgG1, IgG2, IgG3 or IgG4.
34. The HcAb of any of embodiments 31-33, wherein the IgG Fc region and the IgM Fc region have the sequence as set forth in SEQ ID NO: 472.
35. The HcAb of any of embodiments 13-34, further including a J-chain.
36. The HcAb of embodiment 35, wherein the J-chain has the sequence as set forth in SEQ ID NOs: 482-488.
37. The HcAb of embodiments 35 or 36, wherein the binding domain is introduced into the J-chain.
38. The HcAb of embodiment 13, formatted into a multimer.
39. The HcAb of embodiment 38, wherein the multimer is an IgM multimer, an IgA multimer, or an IgG multimer.
40. The HcAb of any of embodiments 38 or 39, wherein the multimer is an IgA dimer or an IgM pentamer or hexamer.
41. The HcAb of embodiment 40, wherein the IgA dimer includes the IgA tail piece.
42. The HcAb of embodiment 40, wherein the IgM pentamer or hexamer includes the IgM tail piece.
43. The HcAb of embodiments 40 or 41, wherein the IgA dimer includes a J chain.
44. The HcAb of embodiments 40 or 42, wherein the IgM pentamer or hexamer includes a J chain.
45. The HcAb of any of embodiments 13-44, wherein the Fc region includes a modification to produce an administration benefit.
46. The HcAb of embodiment 45, wherein the administration benefit includes reduced susceptibility to proteolysis, reduced susceptibility to oxidation, altered binding affinity for forming protein complexes, altered binding affinities, reduced immunogenicity, and/or extended half-life.
47. The HcAb of embodiment 46, wherein the administration benefit is extended half-life and the modification includes M252Y, T252L, T253S, S254T, T254F, T256E, T256N, E294delta, T307P, A379V, S383N, M428L, N434S, N434A, N434Y, and/or R435H.
48. The HcAb of embodiments 46 or 47, wherein the administration benefit is extended half-life and the modification is R435H; N434A; E294delta; M428L/N434S; M252Y/S254T/T256E; T252L/T253S/T254F; E294delta/T307P/N434Y; or T256N/A379V/S383N/N434Y.
49. A multi-specific binding molecule including at least two binding domains that bind distinct epitopes wherein at least one binding domain includes a single-domain antibody of embodiments1-3 or 10-12, a fusion protein of any of embodiments 4-9, or an HcAb of any of embodiments 13-48.
50. The multi-specific binding molecule of embodiment 49, wherein all binding domains of the multi-specific binding molecule bind a SARS-CoV-2 spike protein epitope.
51. The multi-specific binding molecule of embodiments 49 or 50, wherein all binding domains of the multi-specific binding molecule bind distinct SARS-CoV-2 spike protein epitopes.
52. The multi-specific binding molecule of embodiment 51, wherein the distinct SARS-CoV-2 spike protein epitopes are within S1-non-RBD, RBD, and/or S2.
53. The multi-specific binding molecule of any of embodiments 49-52, wherein at least one binding domain binds an S1-non-RBD epitope and at least one binding domain binds an S1-non-RBD epitope, an RBD epitope, or an S2 epitope, at least one binding domain binds an RBD epitope and at least one binding domain binds an RBD epitope, an S1-non-RBD epitope, or an S2 epitope, or wherein at least one binding domain binds an S2 epitope and at least one binding domain binds an S2 epitope, an RBD epitope, or an S1-non-RBD epitope.
54. The multi-specific binding molecule of any of embodiments 49-53, wherein at least one binding domain includes S1-23.
55. The multi-specific binding molecule of any of embodiments 49-54, wherein at least one binding domain includes S1-1, S1-27, S1-RBD-15, or S2-40.
56. The multi-specific binding molecule of any of embodiments 49-55, wherein at least one binding domain includes S1-23 and at least one binding domain includes S1-1, S1-27, S1-RBD-15, or S2-40.
57. The multi-specific binding molecule of any of embodiments 49-56, wherein at least one binding domain includes S1-48 and at least one binding domain includes S1-RBD-15.
58. The multi-specific binding molecule of any of embodiments 49-56, wherein the binding domains include a combination including S1-2 and S2-1; S1-3 and S2-2; S1-7 and S2-3; S1-9 and S2-4; S1-10 and S2-5; S1-11 and S2-6; S1-17 and S2-7; S1-24 and S2-9; S1-25 and S2-10; S1-30 and S2-11; S1-32 and S2-13; S1-41 and S2-14; S1-49 and S2-15; S1-50 and S2-18; S1-58 and S2-22; S1-60 and S2-26; S1-64 and S2-33; S1-65 and S2-35; S1-66 and S2-36; S1-24 and S2-39; S1-32 and S2-40; S1-60 and S2-42; S1-7 and S2-47; S1-11 and S2-57; S1-3 and S2-59; S1-66 and S2-62; S1-2 and S1-1; S1-3 and S1-4; S1-7 and S1-5; S1-9 and S1-6; S1-10 and S1-12; S1-11 and S1-14; S1-17 and S1-19; S1-24 and S1-20; S1-25 and S1-21; S1-30 and S1-23; S1-32 and S1-27; S1-41 and S1-28; S1-49 and S1-29; S1-50 and S1-31; S1-58 and S1-35; S1-60 and S1-36; S1-64 and S1-37; S1-65 and S1-38; S1-66 and S1-39; S1-2 and S1-46; S1-3 and S1-48; S1-7 and S1-51; S1-9 and S1-52; S1-10 and S1-53; S1-11 and S1-54; S1-17 and S1-55; S1-24 and S1-56; S1-25 and S1-61; S1-30 and S1-62; S1-32 and S1-63; S1-41 and S1-RBD-3; S1-49 and S1-RBD-4; S1-50 and S1-RBD-5; S1-58 and S1-RBD-6; S1-60 and S1-RBD-9; S1-64 and S1-RBD-10; S1-65 and S1-RBD-11; S1-66 and S1-RBD-12; S1-2 and S1-RBD-14; S1-3 and S1-RBD-15; S1-7 and S1-RBD-16; S1-9 and S1-RBD-18; S1-10 and S1-RBD-19; S1-11 and S1-RBD-20; S1-17 and S1-RBD-21; S1-24 and S1-RBD-22; S1-25 and S1-RBD-23; S1-30 and S1-RBD-24; S1-32 and S1-RBD-25; S1-41 and S1-RBD-26; S1-49 and S1-RBD-27; S1-50 and S1-RBD-28; S1-58 and S1-RBD-29; S1-60 and S1-RBD-30; S1-64 and S1-RBD-32; S1-65 and S1-RBD-34; S1-66 and S1-RBD-35; S1-2 and S1-RBD-36; S1-3 and S1-RBD-37; S1-7 and S1-RBD-38; S1-9 and S1-RBD-39; S1-10 and S1-RBD-40; S1-11 and S1-RBD-41; S1-17 and S1-RBD-43; S1-24 and S1-RBD-44; S1-25 and S1-RBD-45; S1-30 and S1-RBD-46; S1-32 and S1-RBD-47; S1-41 and S1-RBD-48; S1-49 and S1-RBD-49; S1-50 and S1-RBD-51; S2-1 and S1-1; S2-2 and S1-4; S2-3 and S1-5; S2-4 and S1-6; S2-5 and S1-12; S2-6 and S1-14; S2-7 and S1-19; S2-9 and S1-20; S2-10 and S1-21; S2-11 and S1-23; S2-13 and S1-27; S2-14 and S1-28; S2-15 and S1-29; S2-18 and S1-31; S2-22 and S1-35; S2-26 and S1-36; S2-33 and S1-37; S2-35 and S1-38; S2-36 and S1-39; S2-39 and S1-46; S2-40 and S1-48; S2-42 and S1-51; S2-47 and S1-52; S2-57 and S1-53; S2-59 and S1-54; S2-62 and S1-55; S2-1 and S1-56; S2-2 and S1-61; S2-3 and S1-62; S2-4 and S1-63; S2-5 and S1-RBD-3; S2-6 and S1-RBD-4; S2-7 and S1-RBD-5; S2-9 and S1-RBD-6; S2-10 and S1-RBD-9; S2-11 and S1-RBD-10; S2-13 and S1-RBD-11; S2-14 and S1-RBD-12; S2-15 and S1-RBD-14; S2-18 and S1-RBD-15; S2-22 and S1-RBD-16; S2-26 and S1-RBD-18; S2-33 and S1-RBD-19; S2-35 and S1-RBD-20; S2-36 and S1-RBD-21; S2-39 and S1-RBD-22; S2-40 and S1-RBD-23; S2-42 and S1-RBD-24; S2-47 and S1-RBD-25; S2-57 and S1-RBD-26; S2-59 and S1-RBD-27; S2-62 and S1-RBD-28; S2-1 and S1-RBD-29; S2-2 and S1-RBD-30; S2-3 and S1-RBD-32; S2-4 and S1-RBD-34; S2-5 and S1-RBD-35; S2-6 and S1-RBD-36; S2-7 and S1-RBD-37; S2-9 and S1-RBD-38; S2-10 and S1-RBD-39; S2-11 and S1-RBD-40; S2-13 and S1-RBD-41; S2-14 and S1-RBD-43; S2-15 and S1-RBD-44; S2-18 and S1-RBD-45; S2-22 and S1-RBD-46; S2-26 and S1-RBD-47; S2-33 and S1-RBD-48; S2-35 and S1-RBD-49; S2-36 and S1-RBD-51.S1-2, S2-14 and S1-52; S1-3, S2-15 and S1-53; S1-7, S2-18 and S1-54; S1-9, S2-22 and S1-55; S1-10, S2-26 and S1-56; S1-11, S2-33 and S1-61; S1-17, S2-35 and S1-62; S1-24, S2-36 and S1-63; S1-25, S2-39 and S1-RBD-3; S1-30, S2-40 and S1-RBD-4; S1-32, S2-42 and S1-RBD-5; S1-41, S2-47 and S1-RBD-6; S1-49, S2-57 and S1-RBD-9; S1-50, S2-59 and S1-RBD-10; S1-58, S2-62 and S1-RBD-11; S1-60, S2-1 and S1-RBD-12; S1-64, S2-2 and S1-RBD-14; S1-65, S2-3 and S1-RBD-15; S1-66, S2-4 and S1-RBD-16; S1-2, S2-5 and S1-RBD-18; S1-3, S2-6 and S1-RBD-19; S1-7, S2-7 and S1-RBD-20; S1-9, S2-9 and S1-RBD-21; S1-10, S2-10 and S1-RBD-22; S1-11, S2-11 and S1-RBD-23; S1-17, S2-13 and S1-RBD-24; S1-24, S2-14 and S1-RBD-25; S1-25, S2-15 and S1-RBD-26; S1-30, S2-18 and S1-RBD-27; S1-32, S2-22 and S1-RBD-28; S1-41, S2-26 and S1-RBD-29; S1-49, S2-33 and S1-RBD-30; S1-50, S2-35 and S1-RBD-32; S1-58, S2-36 and S1-RBD-34; S1-60, S2-39 and S1-RBD-35; S1-64, S2-40 and S1-RBD-36; S1-65, S2-42 and S1-RBD-37; S1-66, S2-47 and S1-RBD-38; S1-2, S2-57 and S1-RBD-39; S1-3, S2-59 and S1-RBD-40; S1-7, S2-62 and S1-RBD-41; S1-9, S2-1 and S1-RBD-43; S1-10, S2-2 and S1-RBD-44; S1-11, S2-3 and S1-RBD-45; S1-17, S2-4 and S1-RBD-46; S1-24, S2-5 and S1-RBD-47; S1-25, S2-6 and S1-RBD-48; S1-30, S2-7 and S1-RBD-49; S1-32, S2-9 and S1-RBD-51; S1-41, S2-10 and S1-1; S1-49, S2-11 and S1-4; S1-50, S2-13 and S1-20; S1-58, S2-14 and S1-21; S1-60, S2-15 and S1-23; S1-64, S2-18 and S1-27; S1-65, S2-1 and S1-28; S1-66, S2-2 and S1-29; S1-2, S2-3 and S1-31; S1-3, S2-4 and S1-35; S1-7, S2-5 and S1-36; S1-9, S2-6 and S1-37; S1-10, S2-7 and S1-38; S1-11, S2-9 and S1-39; S1-17, S2-10 and S1-46; S1-24, S2-11 and S1-48; or S1-25, S2-13 and S1-51.
59. The multi-specific binding molecule of any of embodiments 49-58, wherein a binding domain of the multi-specific binding molecule binds a secondary respiratory virus.
60. The multi-specific binding molecule of embodiment 60, wherein the secondary respiratory virus includes a human adenovirus, human boca virus (HBoV), human coronavirus other than SARS-CoV-2, influenza virus, human parainfluenza virus, or human rhinovirus.
61. The multi-specific binding molecule of embodiments 32 or 33, wherein the binding domain that binds the secondary respiratory virus is derived from 8C4, 5Hx-I, 5Hx-2, 5Hx-3, 5Hx-4, 5Hx-5, 5.100K-1, 5PB-1, 5Fb-I, 1E11, EPR23305-44, 47D11, CR3022, CDC2-A2, G2, 5F9, FIB-H1, JC57-13, 32D6, CH65, CR9114, MAb 22/1, MAb70/I, MAb 110/1, MAb 264/2, MAb W18/1, MAb 14/3, MAb 24/4, MAb 47/8, MAb 198/2, MAb 215/2, H2/6A5, H3/4C4, H2/6C4, H2/4B3, H9/B20, H2/4B1, CA6261, 6F12, CR9114, PEG-1, CR8033, CR8071, 113/2, 124/4, 128/2, 134/1, 146/1, 152/2, 160/1, 162/1, 195/3, 206/2, 238/4, 280/2, PAR2, or TCN-711.
62. The multi-specific binding molecule of any of embodiments 49-61, wherein the multi-specific binding molecule includes an immune cell engaging molecule.
63. The multi-specific binding molecule of embodiment 62, wherein the immune cell engaging molecule activates a B cell, T cell, natural killer (NK) cell, or macrophage.
64. The multi-specific binding molecule of embodiments 62 or 63, wherein a binding domain of the immune cell engaging molecule binds CD2, CD3, CD7, CD8, CD27, CD28, CD30, CD40, CD83, 4-1BB (CD137), OX40, lymphocyte function-associated antigen-1 (LFA-1), LIGHT, NKG2C, and/or B7-H3 (e.g., CD3 and CD28; CD3 and 4-1BB, CD3 and CD8).
65. The multi-specific binding molecule of embodiment 64, wherein the binding domain binds CD3.
66. The multi-specific binding molecule of embodiment 64, wherein the binding domain binds CD28.
67. The multi-specific binding molecule of embodiment 64, wherein the binding domain binds 4-1BB.
68. The multi-specific binding molecule of embodiment 64, wherein the binding domain binds CD8.
69. The multi-specific binding molecule of embodiments any of embodiments 63-68, wherein a binding domain of the immune cell engaging molecule binds an NK cell receptor.
70. The multi-specific binding molecule of embodiment 69, wherein the binding domain binds:
   NKG2D and is derived from the 5C6 antibody or 1D11 antibody;
   KIR2DL4 and is derived from mAb 33;
   NKp44 and is derived from the P44-8 antibody;
   CD8 and is derived from the OKT8 antibody or the SK1 antibody; or
   CD16 and is derived from the 3G8 antibody.
71. The multi-specific binding molecule of any of embodiments 49-70, wherein a binding domain of the multi-specific binding molecule binds human albumin.
72. The multi-specific binding molecule of embodiment 71, wherein at least one binding domain includes a single domain antibody of any of embodiments 1-3 or 10-12 and at least one binding domain includes a single domain antibody that binds human albumin.
73. The multi-specific binding molecule of embodiments 71 or 72, wherein the binding domain that binds albumin includes CA645, anti-HSA Nanobody™ (Ablynx, Ghent, Belgium), or AlbudAb™ (GlaxoSmithKline, Brentford, United Kingdom).
74. The multi-specific binding molecule of embodiment 71 or 72, wherein the binding domain that binds albumin has the sequence as set forth in SEQ ID NO: 594 or SEQ ID NO: 595.
75. The multi-specific binding molecule of embodiment 74, wherein the binding domain that binds albumin includes a sequence with at least 95% sequence identity to SEQ ID NO: 594 or SEQ ID NO: 595.
76. The multi-specific binding molecule of embodiments 74 or 75, wherein the binding domain that binds albumin includes a sequence with 98% sequence identity to SEQ ID NO: 594 or SEQ ID NO: 595.
77. A conjugate including a single-domain antibody of embodiments 1-3 or 10-12, a fusion protein of embodiments 4-9, and/or an HcAb of any of embodiments 13-48 linked to an immunotoxin, a drug, a radioisotope, a detectable label, or a microparticle.
78. The conjugate of embodiment 77, including an immunotoxin.
79. The conjugate of embodiment 78, wherein the immunotoxin includes a plant toxin or bacterial toxin.
80. The conjugate of embodiment 79, wherein the plant toxin includes ricin, abrin, mistletoe lectin, modeccin, pokeweed antiviral protein, saporin, Bryodin 1, bouganin, or gelonin.
81. The conjugate of embodiment 79, wherein the bacterial toxin includes diphtheria toxin, or Pseudomonas exotoxin.
82. The conjugate of any of embodiments 77-81, including a drug.
83. The conjugate of embodiment 82, wherein the drug includes a cytotoxic drug.
84. The conjugate of embodiment 83, wherein the cytotoxic drug includes actinomycin D, auristatin, camptothecin, colchicin, daunorubicin, dihydroxy dolastatin, doxorubicin, duocarmycin, emetine, etoposide, gramicidin D, glucocorticoids, maytansinoid mithramycin, mitomycin, nemorubicin, propranolol, puromycin, taxane, taxol, tetracaine, trichothecene, vinblastine, vinca alkaloid, or vincristine.
85. The conjugate of any of embodiments 77-84, including a radioisotope.
86. The conjugate of embodiment 85, wherein the radioisotope includes ²²⁵Ac, ²²⁷Th, ²²⁸Ac, ¹²⁴Am, ²¹¹As, ¹⁹⁴Au, ⁷Be, ²⁴⁵Bk, ⁷⁶Br, ¹¹C, ²⁵⁴Cf, ²⁴²Cm, ⁵¹Cr, ⁶⁷Cu, ¹⁵³Dy, ¹⁷¹Er, ²⁵⁰Es, ¹⁴⁷Eu, ⁵²Fe, ²⁵¹Fm, ⁶⁶Ga, ¹⁴⁶Gd, ⁶⁸Ge, ¹⁷⁰Hf, ¹⁹³Hg, ¹³¹I, ¹⁸⁵Ir, ⁴²K, ⁷⁹Kr, ¹³²La, ²⁶²Lr, ¹⁶⁹Lu, ²⁶⁰Md, ⁵²Mn, ⁹⁰Mo, ²⁴Na, ⁹⁵Nb, ¹³⁸Nd, ⁵⁷Ni, ¹⁵O, ¹⁸²Os ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tₘ, ²³⁰U, ⁴⁸V, ¹⁷⁸W, ¹²⁵Xe, ⁸⁶Y, ¹⁶⁹Yb, ⁶⁵Zn, ⁹⁵Zr, or ⁹⁷Zr.
87. The conjugate of any of embodiments 77-86, including a detectable label.
88. The conjugate of embodiment 87, wherein the detectable label includes a chemiluminescent label, a spectral colorimetric label, an affinity tag, an enzymatic label, or a fluorescent label.
89. The conjugate of any of embodiments 77-88, including a microparticle.
90. The conjugate of embodiment 89, wherein the microparticle includes a latex bead, a polystyrene bead, a fluorescent bead, or a colored bead.
91. A chimeric antigen receptor (CAR) that, when expressed by a cell, includes an extracellular component linked to an intracellular component by a transmembrane domain, wherein the extracellular component includes a single-domain antibody of any of embodiments 1-3 or 10-12.
92. The CAR of embodiment 91, wherein the single-domain antibody of any of embodiments 1-3 or 10-12 includes S1-1, S1-6, S1-23, S1-27, S1-37, S1-39, S1-48, S1-49, S1-RBD-5, S1-RBD-6, S1-RBD-9, S1-RBD-11, S1-RBD-15, S1-RBD-21, S1-RBD-22, S1-RBD-35, S2-7, S2-10, or S2-40.
93. The CAR of embodiments 91 or 92, wherein the intracellular component includes an effector domain including: 4-1EE (CD137), CD3γ, CD3δ, CD3ε, CD3ζ, CD27, CD28, DAP10, ICOS, LAG3, NKG2D, NOTCH1, OX40, ROR2, SLAMF1, TCRα, TCRβ, TRIM, Wnt, Zap70, or a combination thereof.
94. The CAR of any of embodiments 91-93, wherein the transmembrane domain includes a transmembrane region of: the α, β or ζ chain of a T-cell receptor; CD28; CD27; CD3; CD45; CD4; CD5; CD8; CD9; CD16; CD22; CD33; CD37; CD64; CD80; CD86; CD134; CD137; CD154; or a combination thereof.
95. The CAR of any of embodiments 91-94, wherein the CAR further includes a spacer region.
96. A cell genetically modified to express a single-domain antibody of any of embodiments 1-3 or 10-12, a fusion protein of embodiments 4-9, an HcAb of any of embodiments 13-48, and/or a CAR of any of embodiments 91-95.
97. The cell of embodiment 96, wherein the cell is an immune cell.
98. The cell of embodiment 97, wherein the immune cell is a T cell, B cell, natural killer cell, or macrophage.
99. The cell of embodiment 96, wherein the cell is a bacterial cell, an insect cell, or a yeast cell.
100. A composition including a single-domain antibody of any of embodiments 1-3 or 10-12, a fusion protein of any of embodiments 4-9, an HcAb of any of embodiments—13-48, or a cell of any of embodiments 96-99 for administration to a subject.
101. The composition of embodiment 100, further including a pharmaceutically acceptable carrier.
102. The composition of embodiments 100 or 101, wherein the single-domain antibody of embodiments 1 or 2 includes S1-1, S1-6, S1-23, S1-27, S1-37, S1-39, S1-48, S1-49, S1-RBD-5, S1-RBD-6, S1-RBD-9, S1-RBD-11, S1-RBD-15, S1-RBD-21, S1-RBD-22, S1-RBD-35, S2-7, S2-10, or S2-40.
103. The composition of any of embodiments 100-102, including a fusion protein having two or three copies of S1-23, S1-49, S1-RBD-35, or S2-7.
104. The composition of any of embodiments 100-103, including a multimerized dimer or trimer of S1-23, S1-49, S1-RBD-35, or S2-7
105. The composition of any of embodiments 100-104, wherein the composition includes at least two single-domain antibodies of any of embodiments 1-3 or 10-12.
106. The composition of embodiment 105, wherein the at least two single-domain antibodies bind distinct epitopes.
107. The composition of embodiments 105 or 106, wherein
   one of the at least two single-domain antibodies binds an S1-non-RBD epitope and at least one of the at least two single-domain antibodies binds an S1-non-RBD epitope, an RBD epitope, or an S2 epitope,
   one of the at least two single-domain antibodies binds an RBD epitope and at least one of the at least two single-domain antibodies binds an RBD epitope, an S1-non-RBD epitope, or an S2 epitope, or wherein
   one of the at least two single-domain antibodies binds an S2 epitope and at least one of the at least two single-domain antibodies binds an S2 epitope, an RBD epitope, or an S1-non-RBD epitope.
108. The composition of any of embodiments 105-107, wherein the at least two single-domain antibodies include S1-23 and S1-1, S1-27, S1-RBD-15, or S2-40.
109. The composition of any of embodiments 105-108, wherein at least one single-domain antibody includes S1-23 and at least one single-domain antibody includes S1-1, S1-27, S1-RBD-15, or S2-40.
110. The composition of any of embodiments 105-107, wherein at least one single-domain antibody includes S1-48 and at least one single-domain antibody includes S1-RBD-15.
111. The composition of any of embodiments 105-107, including S1-2 and S2-1; S1-3 and S2-2; S1-7 and S2-3; S1-9 and S2-4; S1-10 and S2-5; S1-11 and S2-6; S1-17 and S2-7; S1-24 and S2-9; S1-25 and S2-10; S1-30 and S2-11; S1-32 and S2-13; S1-41 and S2-14; S1-49 and S2-15; S1-50 and S2-18; S1-58 and S2-22; S1-60 and S2-26; S1-64 and S2-33; S1-65 and S2-35; S1-66 and S2-36; S1-24 and S2-39; S1-32 and S2-40; S1-60 and S2-42; S1-7 and S2-47; S1-11 and S2-57; S1-3 and S2-59; S1-66 and S2-62; S1-2 and S1-1; S1-3 and S1-4; S1-7 and S1-5; S1-9 and S1-6; S1-10 and S1-12; S1-11 and S1-14; S1-17 and S1-19; S1-24 and S1-20; S1-25 and S1-21; S1-30 and S1-23; S1-32 and S1-27; S1-41 and S1-28; S1-49 and S1-29; S1-50 and S1-31; S1-58 and S1-35; S1-60 and S1-36; S1-64 and S1-37; S1-65 and S1-38; S1-66 and S1-39; S1-2 and S1-46; S1-3 and S1-48; S1-7 and S1-51; S1-9 and S1-52; S1-10 and S1-53; S1-11 and S1-54; S1-17 and S1-55; S1-24 and S1-56; S1-25 and S1-61; S1-30 and S1-62; S1-32 and S1-63; S1-41 and S1-RBD-3; S1-49 and S1-RBD-4; S1-50 and S1-RBD-5; S1-58 and S1-RBD-6; S1-60 and S1-RBD-9; S1-64 and S1-RBD-10; S1-65 and S1-RBD-11; S1-66 and S1-RBD-12; S1-2 and S1-RBD-14; S1-3 and S1-RBD-15; S1-7 and S1-RBD-16; S1-9 and S1-RBD-18; S1-10 and S1-RBD-19; S1-11 and S1-RBD-20; S1-17 and S1-RBD-21; S1-24 and S1-RBD-22; S1-25 and S1-RBD-23; S1-30 and S1-RBD-24; S1-32 and S1-RBD-25; S1-41 and S1-RBD-26; S1-49 and S1-RBD-27; S1-50 and S1-RBD-28; S1-58 and S1-RBD-29; S1-60 and S1-RBD-30; S1-64 and S1-RBD-32; S1-65 and S1-RBD-34; S1-66 and S1-RBD-35; S1-2 and S1-RBD-36; S1-3 and S1-RBD-37; S1-7 and S1-RBD-38; S1-9 and S1-RBD-39; S1-10 and S1-RBD-40; S1-11 and S1-RBD-41; S1-17 and S1-RBD-43; S1-24 and S1-RBD-44; S1-25 and S1-RBD-45; S1-30 and S1-RBD-46; S1-32 and S1-RBD-47; S1-41 and S1-RBD-48; S1-49 and S1-RBD-49; S1-50 and S1-RBD-51; S2-1 and S1-1; S2-2 and S1-4; S2-3 and S1-5; S2-4 and S1-6; S2-5 and S1-12; S2-6 and S1-14; S2-7 and S1-19; S2-9 and S1-20; S2-10 and S1-21; S2-11 and S1-23; S2-13 and S1-27; S2-14 and S1-28; S2-15 and S1-29; S2-18 and S1-31; S2-22 and S1-35; S2-26 and S1-36; S2-33 and S1-37; S2-35 and S1-38; S2-36 and S1-39; S2-39 and S1-46; S2-40 and S1-48; S2-42 and S1-51; S2-47 and S1-52; S2-57 and S1-53; S2-59 and S1-54; S2-62 and S1-55; S2-1 and S1-56; S2-2 and S1-61; S2-3 and S1-62; S2-4 and S1-63; S2-5 and S1-RBD-3; S2-6 and S1-RBD-4; S2-7 and S1-RBD-5; S2-9 and S1-RBD-6; S2-10 and S1-RBD-9; S2-11 and S1-RBD-10; S2-13 and S1-RBD-11; S2-14 and S1-RBD-12; S2-15 and S1-RBD-14; S2-18 and S1-RBD-15; S2-22 and S1-RBD-16; S2-26 and S1-RBD-18; S2-33 and S1-RBD-19; S2-35 and S1-RBD-20; S2-36 and S1-RBD-21; S2-39 and S1-RBD-22; S2-40 and S1-RBD-23; S2-42 and S1-RBD-24; S2-47 and S1-RBD-25; S2-57 and S1-RBD-26; S2-59 and S1-RBD-27; S2-62 and S1-RBD-28; S2-1 and S1-RBD-29; S2-2 and S1-RBD-30; S2-3 and S1-RBD-32; S2-4 and S1-RBD-34; S2-5 and S1-RBD-35; S2-6 and S1-RBD-36; S2-7 and S1-RBD-37; S2-9 and S1-RBD-38; S2-10 and S1-RBD-39; S2-11 and S1-RBD-40; S2-13 and S1-RBD-41; S2-14 and S1-RBD-43; S2-15 and S1-RBD-44; S2-18 and S1-RBD-45; S2-22 and S1-RBD-46; S2-26 and S1-RBD-47; S2-33 and S1-RBD-48; S2-35 and S1-RBD-49; S2-36 and S1-RBD-51.S1-2, S2-14 and S1-52; S1-3, S2-15 and S1-53; S1-7, S2-18 and S1-54; S1-9, S2-22 and S1-55; S1-10, S2-26 and S1-56; S1-11, S2- 33 and S1-61; S1-17, S2-35 and S1-62; S1-24, S2-36 and S1-63; S1-25, S2-39 and S1-RBD-3; S1-30, S2-40 and S1-RBD-4; S1-32, S2-42 and S1-RBD-5; S1-41, S2-47 and S1-RBD-6; S1-49, S2-57 and S1-RBD-9; S1-50, S2-59 and S1-RBD-10; S1-58, S2-62 and S1-RBD-11; S1-60, S2-1 and S1-RBD-12; S1-64, S2-2 and S1-RBD-14; S1-65, S2-3 and S1-RBD-15; S1-66, S2-4 and S1-RBD-16; S1-2, S2-5 and S1-RBD-18; S1-3, S2-6 and S1-RBD-19; S1-7, S2-7 and S1-RBD-20; S1-9, S2-9 and S1-RBD-21; S1-10, S2-10 and S1-RBD-22; S1-11, S2-11 and S1-RBD-23; S1-17, S2-13 and S1-RBD-24; S1-24, S2-14 and S1-RBD-25; S1-25, S2-15 and S1-RBD-26; S1-30, S2-18 and S1-RBD-27; S1-32, S2-22 and S1-RBD-28; S1-41, S2-26 and S1-RBD-29; S1-49, S2-33 and S1-RBD-30; S1-50, S2-35 and S1-RBD-32; S1-58, S2-36 and S1-RBD-34; S1-60, S2-39 and S1-RBD-35; S1-64, S2-40 and S1-RBD-36; S1-65, S2-42 and S1-RBD-37; S1-66, S2-47 and S1-RBD-38; S1-2, S2-57 and S1-RBD-39; S1-3, S2-59 and S1-RBD-40; S1-7, S2-62 and S1-RBD-41; S1-9, S2-1 and S1-RBD-43; S1-10, S2-2 and S1-RBD-44; S1-11, S2-3 and S1-RBD-45; S1-17, S2-4 and S1-RBD-46; S1-24, S2-5 and S1-RBD-47; S1-25, S2-6 and S1-RBD-48; S1-30, S2-7 and S1-RBD-49; S1-32, S2-9 and S1-RBD-51; S1-41, S2-10 and S1-1; S1-49, S2-11 and S1-4; S1-50, S2-13 and S1-20; S1-58, S2-14 and S1-21; S1-60, S2-15 and S1-23; S1-64, S2-18 and S1-27; S1-65, S2-1 and S1-28; S1-66, S2-2 and S1-29; S1-2, S2-3 and S1-31; S1-3, S2-4 and S1-35; S1-7, S2-5 and S1-36; S1-9, S2-6 and S1-37; S1-10, S2-7 and S1-38; S1-11, S2-9 and S1-39; S1-17, S2-10 and S1-46; S1-24, S2-11 and S1-48; or S1-25, S2-13 and S1-51.
112. The composition of any of embodiments 100-111, wherein the composition includes HcAb wherein HcAb within the composition includes at least two different single-domain antibodies of any of embodiments 1-3 or 10-12.
113. The composition of embodiments 100-112, wherein the composition includes cells of any of embodiments 96-99, wherein the cells within the composition express at least two different single-domain antibodies of embodiments 1-3 or 10-12, a fusion protein of embodiments 4-9, or HcAb wherein expressed HcAb include at least two different single-domain antibodies of any of embodiments 1-3 or 10-12.
114. The composition of any of embodiments 100-113, formulated for pulmonary administration.
115. The composition of any of embodiments 100-114, freeze-dried without a cryoprotectant.
116. A method of treating a subject in need thereof including administering a therapeutically effective amount of the composition of any of embodiments 100-115 thereby treating the subject in need thereof.
117. The method of embodiment 116, wherein the therapeutically effective amount provides a prophylactic or a therapeutic treatment against a viral infection.
118. The method of embodiment 117, wherein the therapeutically effective amount provides a prophylactic or a therapeutic treatment against a coronavirus infection.
119. The method of embodiment 117, wherein the therapeutically effective amount provides a prophylactic or a therapeutic treatment against a SARS coronavirus infection.
120. The method of embodiment 117, wherein the therapeutically effective amount provides a prophylactic or a therapeutic treatment against a SARS-CoV-2 or a SARS-CoV-1 coronavirus infection.
121. The method of embodiments 116-120, wherein the therapeutically effective amount provides an anti-infection effect against SARS-CoV-2.
122. The method of embodiment 121, wherein the anti-infection effect against SARS-CoV-2 increases lung capacity, increases ability to taste, increases ability to smell, decreases infection-induced blood clotting, decreases gastrointestinal distress, decreases headaches, and/or decreases “COVID-toe”.
123. The method of any of embodiments 116-122, wherein the administering is through inhalation.
   (x) Experimental Examples. Results and Discussion. Maximizing the Size and Diversity of Anti-SARS-CoV-2 spike Nanobody Repertoire._The was to isolate a large repertoire of highly diverse nanobodies against SARS-CoV-2 spike protein. Thus, existing nanobody generation pipeline was built on (Fridy et al., 2014, Nat Methods 11, 1253-1260), further optimizing each step, explicitly designing it to yield hundreds of high quality, highly diverse nanobody candidates (FIG. 2A). In this way, advantage was taken of both the straightforward procedure of llama immunization, and the powerful natural affinity maturation processes in vivo (Thompson et al., 2016, J Immunol Methods 430, 56-60).

To identify V_{H}H domains that bind spike, V_{H}H domains were affinity purified from the immunized animals' sera against spike S1, S2 or RBD domains, using independent domains in this purification step to maximize epitope accessibility. In parallel, lymphocyte RNA was taken from bone marrow aspirates, and used to amplify V_{H}H domain sequences by PCR, which were sequenced to generate an in silico library representative of all V_{H}H sequences expressed in the individual animal. The affinity-purified V_{H}H fragments were proteolyzed and the resulting peptides analyzed by LC-MS/MS. These data were searched against the V_{H}H sequence library to identify and rank candidate nanobody sequences using the Llama-Magic software package (Fridy et al., 2014, Nat Methods 11, 1253-1260) with a series of key improvements (see Methods).

To maximize sequence diversity and thus the paratope space being explored, CDR sequences were clustered, revealing that many of the candidates form clusters to have similar antigen binding behavior. Here, partitioning of the clusters was performed by requiring that CDR3s in distinct clusters differ by a distance of more than three Damerau-Levenshtein edit operations (Bard, 2007, In Proceedings of the Fifth Australasian Symposium on ACSW Frontiers: 2007, Ballarat, Australia, Jan. 30-Feb. 2, 2007. Conferences in Research and Practice in Information Technology, (Australian Computer Society, Inc.), pp. 117-124)—i.e., each operation being defined by insertion, deletion, or substitution of an amino acid residue, or transposition of two adjacent amino acid residues (FIG. 2B). This partitioning was found to be effective, in that virtually no overlap was observed between those directed against S1 versus S2. The lengths of these CDR3 candidates also varied considerably, ranging from 3-22 amino acids in length. The use of two animals further expanded the paratope diversity in that only 4 out of 22 possible clusters from the second animal were observed to be shared with the first animal. In addition, relatively little overlap was detected between the CDR3 clusters and those observed by other groups; for example, only 1 out of 109 S1 specific clusters (Damerau-Levenshtein s 3) were shared by (Xiang et al., 2020, Science 370, 1479-1484) and the present work—indicating that the repertoires sampled extended regions of the available paratope space (see also below).

Of the several hundred positives, 180 high-confidence candidates were selected for expression and screening. Of these, 66 were from S1 affinity purification, 63 from S2, and 51 from RBD, numbered S1-n, S2-n, and S1-RBD-n respectively. These were then expressed with periplasmic secretion in bacteria, and crude periplasmic fractions were bound to the corresponding immobilized spike antigen to assay recombinant expression, specific binding, and degree of binding (FIGS. 3A-3C, 4A-4C). 138 candidates were validated by this screen: 52 against S1, 42 against S2, and 44 against RBD (FIG. 2B). To eliminate candidates with the weakest expression and binding affinity, only nanobodies with binding intensity >20% of the observed maximum across all those screened were chosen for follow-up study. This filtering identified the top 116 nanobodies that were purified for further characterization (FIGS. 5 and 6). Note that these selections were designed to provide a strict cutoff in the interests of maximizing the quality of the repertoire selected for thorough characterization, but eliminated many additional nanobodies that nevertheless specifically bind to S1 and S2. While a few of these 116 nanobodies were chosen to share similar paratopes, overall, the group retained a high sequence and paratope diversity (FIG. 2C).

High Affinity Nanobodies Across the Entire spike Ectodomain that are Refractory to Common spike Escape Mutants. Surface plasmon resonance (SPR) was used to detail the kinetic properties and affinities of the selected nanobodies (FIGS. 5 and 6). All bound with high affinity, with >60% binding with K_{D}s<1 nM, and two with single digit picomolar affinities (FIGS. 7A-7G). While most S1 binding nanobodies bind RBD (71 nanobodies), 19 targeted non-RBD regions of S1 and 26 bind S2 (FIGS. 7A-7G). The lower number of non-RBD S1 and S2 nanobodies reflect the highly antigenic nature of the RBD and the occlusion of non-RBD S1 regions and S2 due to the glycan shield of SARS-CoV-2 spike (Grant et al., 2020, Sci Rep 10, 14991.; Watanabe et al., 2020, Science 369, 330-333). At the same time, no obvious bias was observed in nanobody affinities for these different domains. While both high on rates and low off rates contributed to these high affinities, kinetic analyses underscore the generally fast association rates (kₒₙ≥10⁺⁶) of these nanobodies (due to their small size and proportionally large paratope surface area), with many surpassing the kₒₙ of high-performing monoclonal antibodies (kₒₙ=10⁺⁵) (Tian et al., 2020, Emerg Microbes Infect 9, 382-385) (FIGS. 7A-7G), a property that would benefit translation of these nanobodies into rapid therapeutics and diagnostics (Carter, 2006, Nat Rev Immunol 6, 343-357). For those nanobodies with apparently homologous paratopes (FIG. 7C), no correlation was found in their kinetic properties (FIGS. 5 and 6), demonstrating that even small paratope changes can strongly alter behaviors (Fridy et al., 2014, Nat Methods 11, 1253-1260).

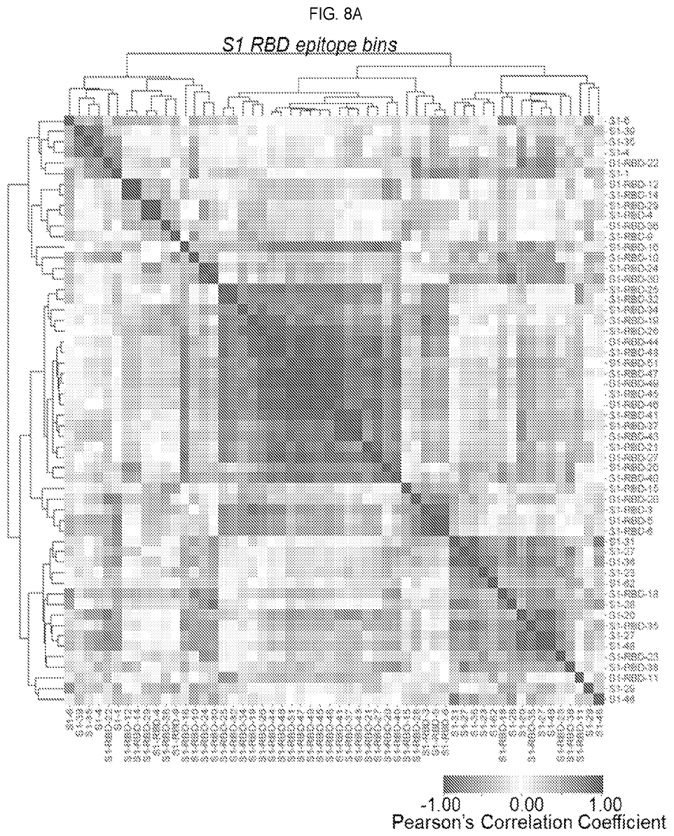

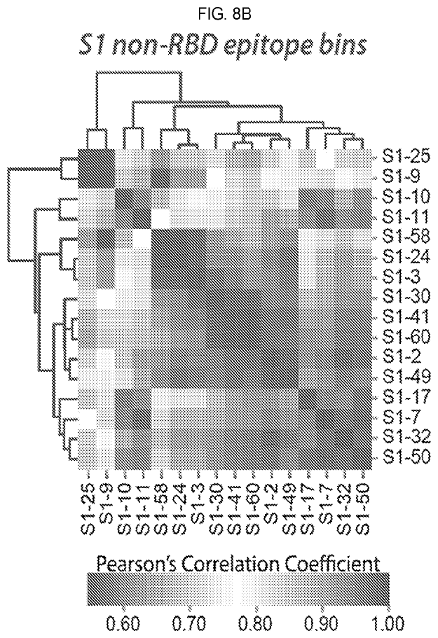

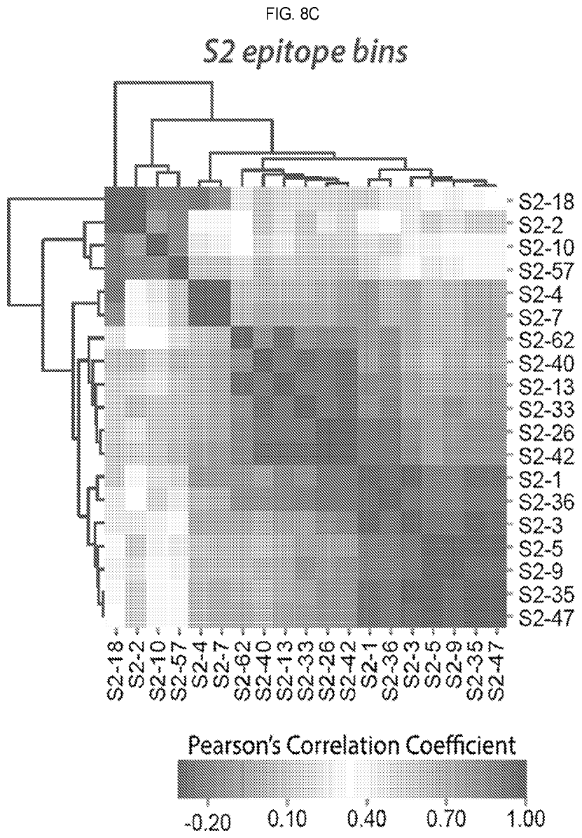

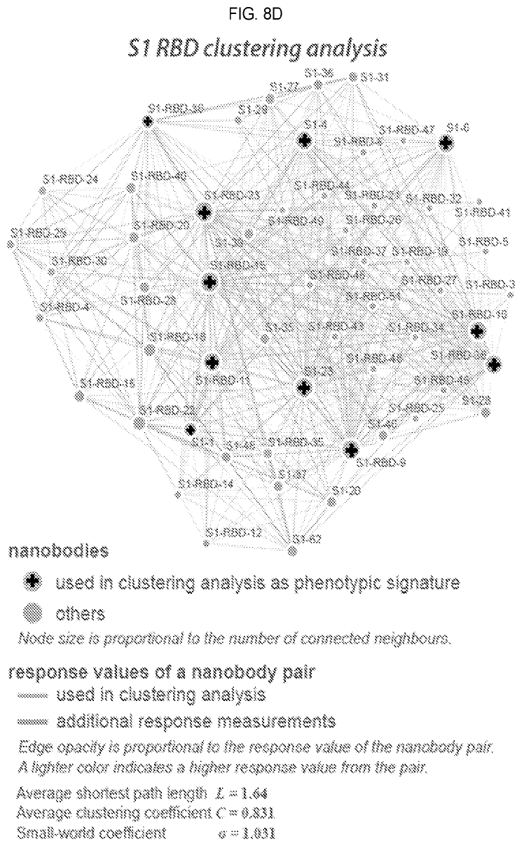

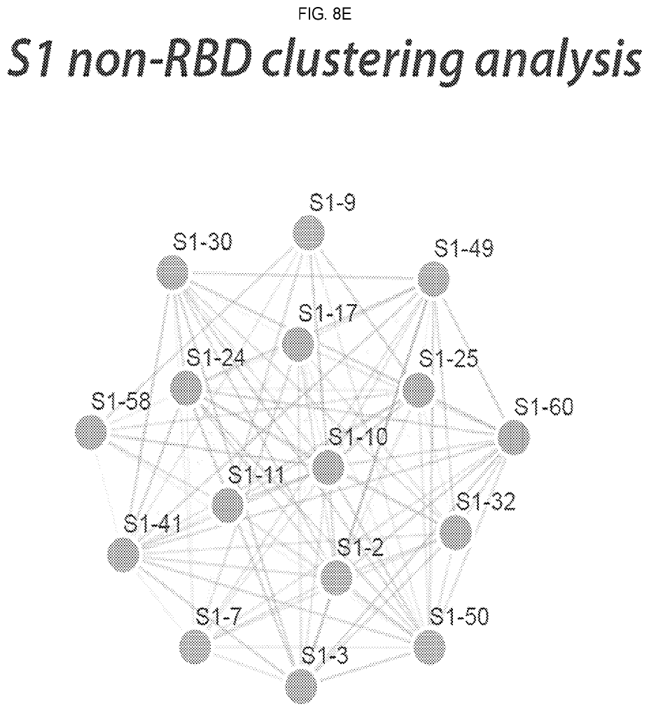

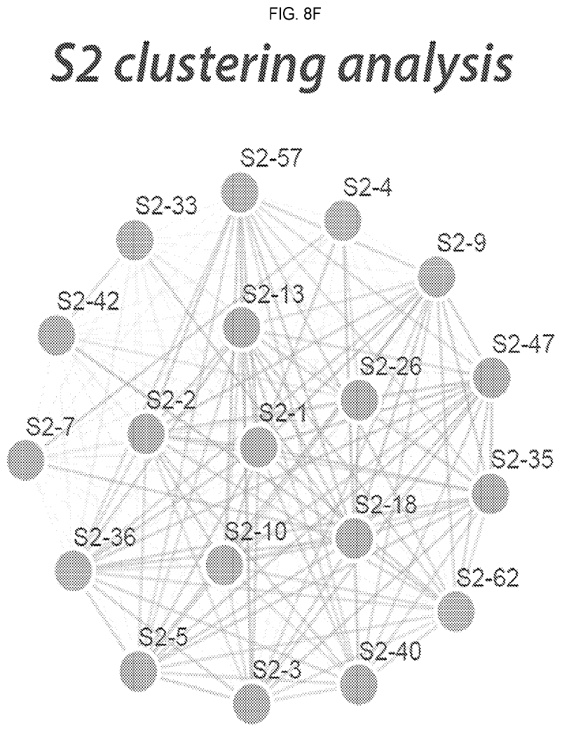

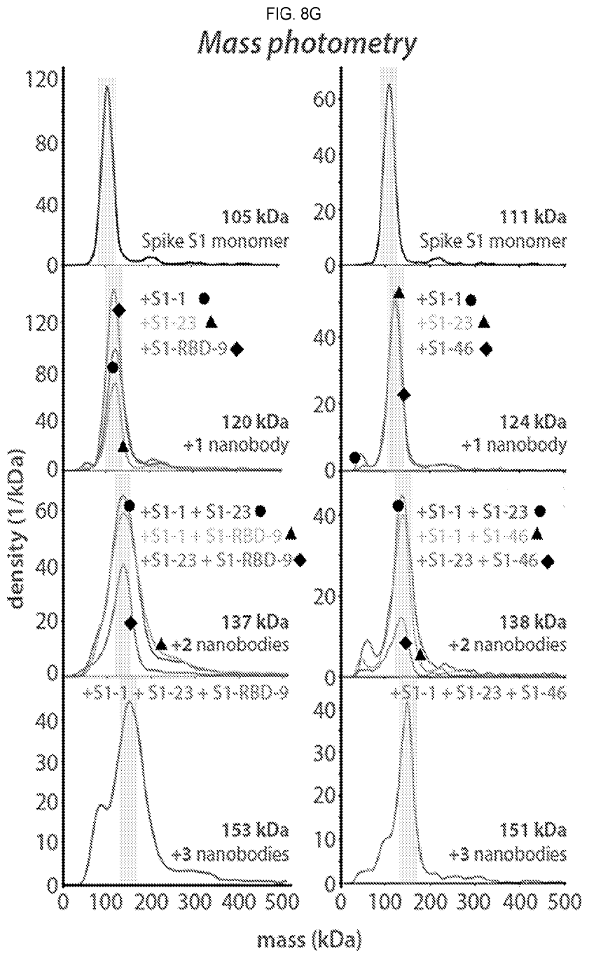

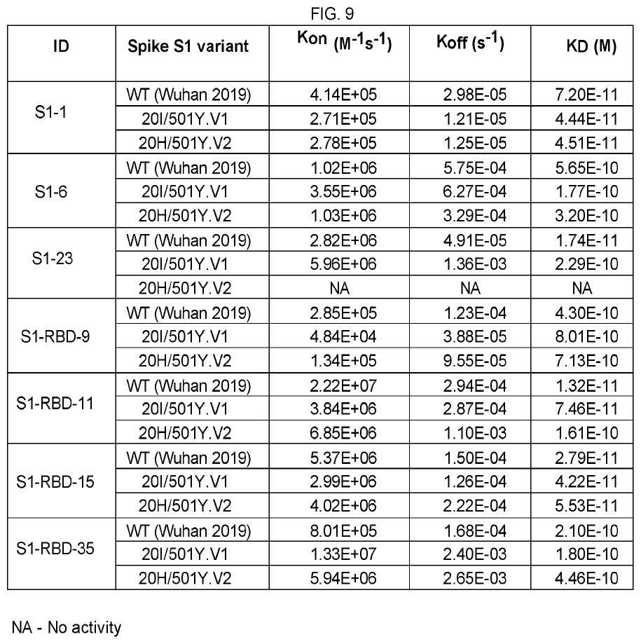

A worrying development is the continuing emergence of viral variants, including mutations in RBD that minimize or nullify binding of many currently available monoclonal antibodies and nanobodies, which solely target RBD (Diamond et al., 2021, Res Sq. doi: 10.21203/rs.3.rs-228079/v1; Garcia-Beltran et al., 2021, medRxiv. 10.1101/2021.02.14.21251704; Jangra et al., 2021, medRxiv. 10.1101/2021.01.26.21250543; Liu et al., 2021, bioRxiv. 10.1101/2021.02.16.431305; Sun et al., 2021, bioRxiv. 10.1101/2021.03.09.434592; Wang et al., 2021, Nature 592, 616-622; Weisblum et al., 2020, Elife 9). Indeed, in one study, the efficacy of 14 out of the 17 most potent monoclonal antibodies tested was compromised by such common RBD mutants (Wang et al., 2021, Nature 592(7855):616-622). Here, nanobodies show great potential to be particularly resistant to these variants (Sun et al., 2021, bioRxiv. 10.1101/2021.03.09.434592). RBD mutants represent a significant class of escape variants (Garcia-Beltran et al., 2021, Cell 184(9):2372-2383; Greaney, et al., 2021, Cell Host Microbe 29, 463-476 e466), leading us to employ two strategies to ensure the generation of numerous nanobodies whose binding (and virus neutralizing activities) are resistant to emerging variants. First, a large diversity of high quality anti-RBD nanobodies were isolated to maximize the probability of identifying ones that are refractory to escape. Second, to reveal additional nanobody neutralizing potential, non-RBD regions of spike were deliberately targeted (see below) (Elshabrawy et al., 2012, PLoS One 7, e50366; Greaney et al., 2021, Cell Host Microbe 29, 463-476 e466). To test the first strategy, RBD binding nanobodies covering non-overlapping epitopes on RBD were sampled (FIGS. 8A-8C) and their binding to SARS-CoV2 variants B.1.1.7/201/501Y.V1/alpha (United Kingdom) and B.1.351/20H/501Y.V2/beta (South Africa), currently among the most prevalent and concerning among those spreading in the population (Ho et al., 2021, Res Sq doi: 10.21203/rs.3.rs-155394/v1; Wang et al., 2021, doi: 10.1101/2021.01.25.428137) (FIGS. 7A-7G and 9) was examined. Of the seven nanobodies tested, six of these (S1-1, S1-6, S1-RBD-9, S1-RBD-11, S1-RBD-15, and S1-RBD-35) retained their very strong binding to both variants, with only a modest reduction in affinity for S1-RBD-11 binding to variant B.1.351/20H/501Y.V2/beta (20 pM to 161 pM). For the seventh nanobody, S1-23, binding to variant B.1.1.7/201/501Y.V1/alpha was only reduced from a K_{D} of 17 pM to a still-respectable 230 pM, although its binding to variant B.1.351/20H/501Y.V2/beta was abolished (FIGS. 7A-7G). As expected (Magnus, 2013, PLoS Comput Biol 9, e1002900; Steckbeck et al., 2005, J Virol 79, 12311-12320; VanCott et al., 1994, J Immunol 153, 449-459), it is the off rates that are most affected by these variants. Nevertheless, based on epitope mapping (below) and the identification of nanobodies that recognize epitopes not altered in the emerging variant strains, it is expected that a high percentage of the nanobodies will remain resistant to these escape mutants; this would now include the recently-emerged B.1.617.2/21AA/S:478K/delta variant (Campbell et al., 2021, Euro Surveill 26), making the collection a powerful resource for potential prophylactics and therapeutics.

The Nanobody Repertoire Has Favorable Stability Properties. A key consideration for possible biological therapeutics and diagnostics for SARS-CoV-2 is their stability under potentially denaturing conditions (McConnell et al., 2014, MAbs 6, 1274-1282). To address this, differential scanning fluorimetry (DSF) experiments were performed to determine the thermal stability (Tₘ) of each of the nanobodies. These studies revealed a thermal stability range between 50° C. and 80° C., similar to published results of other properly folded nanobodies and indicative of their generally high stability (Muyldermans, 2013, Annual review of biochemistry 82, 775-797). In contrast to many conventional antibodies, nanobodies are also reported to remain fully active upon reconstitution after lyophilization, particularly in buffers lacking cryoprotectants (Schoof et al., 2020, Science 370, 1473-1479; Xiang et al., 2020, Science 370, 1479-1484). A representative sample from the repertoire was thus freeze-dried without cryoprotectants, reconstituted, then analyzed via SPR and DSF to determine whether their properties were compromised due to lyophilization. The results revealed no significant effect on stability, kinetics, and affinity (FIGS. 7E, 7F). Taken together, these data suggest that the nanobodies, like those published in other contexts (Schoof et al., 2020, Science 370, 1473-1479; Xiang et al., 2020, Science 370, 1479-1484) are able to withstand various temperatures and storage conditions without affecting their stability and binding. These are essential requirements for downstream applications (e.g. use in a nebulizer) and ease of storage—important considerations if these are to be used for mass distribution, including in resource-poor settings (Peeling and McNerney, 2014, Expert Rev Mol Diagn 14, 525-534).

Nanobodies Explore the Major Domains of the spike Ectodomain. A multifaceted approach was applied to physically distinguish nanobodies that target common regions on the surface of the RBD. Using an eight-channel bio-layer interferometer pair-wise competitive binding of nanobodies that bind the RBD was tested for, as well as for those that bind outside of the RBD (i.e., within the S1 non-RBD and S2 domains) (FIGS. 8A-8G). Label-free binding of antibodies to antigens measured in a “dip-and-read” mode provides a real-time analysis of affinity and the kinetics of the competitive binding of nanobody pairs and can distinguish between those that bind to similar or overlapping epitopes versus distinct, non-overlapping epitopes (Estep et al., 2013, MAbs 5, 270-278). 56 anti-RBD nanobodies were screened in pairwise combinations. The response values were used to assist the discovery of nanobody groups that most bind non-overlapping epitopes, by ensuring that the least response of pairwise nanobodies within the group was maximized. Eleven representative nanobodies from this group were used as a foundation, selecting two or more representative nanobodies from each group to bin the remaining RBD nanobodies in the collection. Overlapping pairs from the foundation group and the remaining RBD binders were used to measure if a nanobody pair behaved similarly against other nanobodies measured in the dataset (FIG. 8A), to comprehensively map nanobody competition and epitope bins (FIG. 8E). Pearson correlation coefficients were derived based on their binding characteristics and the data were used to hierarchically cluster and group all RBD binders into bins. This approach revealed three large, mostly non-overlapping bins. However, each bin contained smaller, better-correlated clusters of nanobodies, reflected by the dendrogram, indicating the presence of numerous distinct sub-epitope bins present within each larger bin, i.e. discrete epitopes that partially overlap with other discrete epitopes in the same bin. The gap statistic (Tibshirani et al., 2001, Journal of the Royal Statistical Society: Series B (Statistical Methodology) 63, 411-423) was calculated to estimate the optimal cluster number, discerning at least 8 epitope bins (FIG. 8A).

Nanobodies binding to regions outside of the RBD of S1 were binned in a similar fashion. Using SPR, 16 non-RBD S1-binding nanobodies and 19 S2-binding nanobodies were binned in pairwise competition assays (FIGS. 8D-8G). Pearson correlation coefficients were used to hierarchically cluster these nanobodies, revealing as many as 4 S1-non-RBD bins and 5 S2 bins.

The binning data from pairwise combinations suggest numerous epitope bins, and thus it is reasonable to hypothesize that more than two nanobodies can bind a single domain at the same time. To test this hypothesis mass photometry (MP) was used (Soltermann et al., 2020, Angew Chem Int Ed Engl 59, 10774-10779; Wu and Piszczek, 2021, J Vis Exp. 10.3791/61784; Young et al., 2018, Science 360, 423-427), which can accurately measure multiple binding events to a single antigen. This allowed us to determine which nanobodies share epitope space on spike S1 monomer through detection of additive mass accumulation of a nanobody (or nanobodies) on spike S1 depending on whether or not nanobodies share epitope space on spike S1. Several representative nanobodies that sample across the epitope space of the nanobody repertoire that bind the RBD were chosen for MP studies based on the epitope binning data (FIG. 8C). These data confirmed the separation of the major epitope bins, and furthermore demonstrated that it can bind at least three different nanobodies simultaneously to the RBD, contrasting with the much larger conventional immunoglobulins, which may be too large to simultaneously bind either monomer or trimer S protein) (Corti et al., 2021, Cell 184, 3086-3108; Stewart et al., 1997, EMBO J 16, 1189-1198; Xu et al., 2021, Nature 595, 278-282). This is a critical consideration for the design of complementary nanobody cocktails and multimers with synergistic neutralizing activities (see below).

Anti-RBD Nanobodies are Highly Effective Neutralizing Agents. The SARS-CoV-2 pseudovirus neutralization assay was used to screen and characterize the nanobody repertoire for antiviral activities (FIGS. 10A-10K). The lentiviral-based, single round infection assay robustly measures the neutralization potential of a candidate nanobody and is a validated surrogate to replication competent SARS-CoV-2 (Riepler et al., 2020, Vaccines (Basel) 9; Schmidt et al., 2020, J Exp Med 217). Because measured IC50s are dependent on assay conditions and so cannot be readily compared across laboratories ((Cheng and Prusoff, 1973, Biochem Pharmacol 22, 3099-3108)), four other published nanobodies were included in this assay for comparison (Wrapp et al., 2020, Cell 181, 1004-1015 e1015; Xiang et al., 2020, Science 370, 1479-1484) (FIG. 10G). Overall, 36% of the monomeric nanobody repertoire neutralized with IC50s≤100 nM, while 23% showed neutralization with IC50s<50 nM and 17 potent neutralizers at 20 nM or lower (FIG. 10A). Similarly, the four published nanobodies span the range of neutralization observed within the repertoire from potent (<20 nM) to relatively weak (between 1-10 μM). The most potent neutralizing nanobodies mapped to the RBD; neutralizing activity mapped to each of the major epitope bins of the RBD and were of similar efficacy to the most potent of the comparison nanobodies; importantly, nanobodies binding outside of the RBD also possess neutralizing activity.

Nanobody-based Neutralization Beyond the RBD. Notably, nanobodies mapping outside of the RBD on S1 (anti-S1, non RBD) and to S2 also neutralized the pseudovirus in the assay, albeit with somewhat higher IC50s (FIGS. 10B, 10C). This is the first evidence of nanobody neutralization activity mapping outside of the RBD. As nanobodies are monomeric, the mechanism of this neutralization does not involve viral aggregation and reflects disruption of the virus binding or spike driven fusion of viral and cellular membranes. Such nanobodies, especially directed against relatively invariant regions of coronavirus spike proteins, may have broadly binding/neutralizing activities and are therefore important targets for optimization. Such optimization includes nanobody use in cocktails and as oligomers.

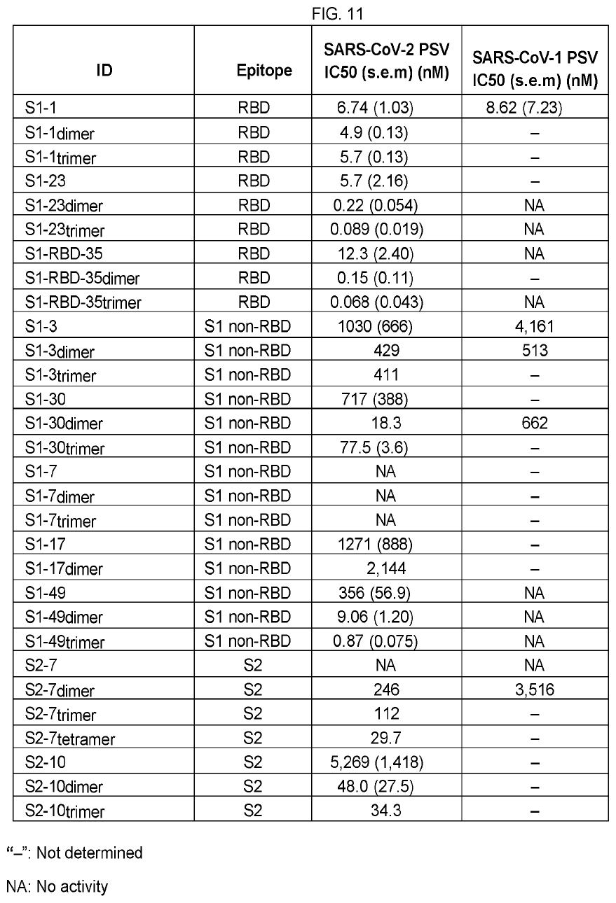

Oligomerization Strongly Enhances the Affinity and Neutralization Activity of Nanobodies. A distinct advantage of nanobodies is the facility by which oligomers can be produced to improve their affinities and avidities (Fridy et al., 2014, Nat Methods 11, 1253-1260). Oligomerization of most of the nanobodies significantly improved their IC50s and measured affinities (FIG. 11). For example, dimerization and trimerization of S1-RBD-35 improved neutralization activity from IC50s of 12 nM to 150 pM and 70 pM, respectively. Similar results were found with S1-23, improving neutralization from 6 nM to 220 pM and 90 pM, respectively (FIG. 10D). Dimerization of the anti-S1 non-RBD nanobody S1-49 improved IC50s from 350 nM to 9 nM, and trimerization improved its activity an additional 10-fold. Multimerization of some nanobodies directed against regions outside of the RBD on both S1 and S2 led to nanomolar range IC50s (FIGS. 10E, 10F). This includes S2-7, for which dimerization converted a nanobody that was considered to be a non-neutralizer to one having a respectable neutralizing activity (IC50 250 nM), with further potency achieved by trimerization and tetramerization, down to an IC50 of 30 nM (FIG. 10F). While these results show that multimerization can dramatically improve their activities, importantly this was not always the case (FIG. 11), indicating that enhancement by multimerization is not a given, but must be determined empirically.

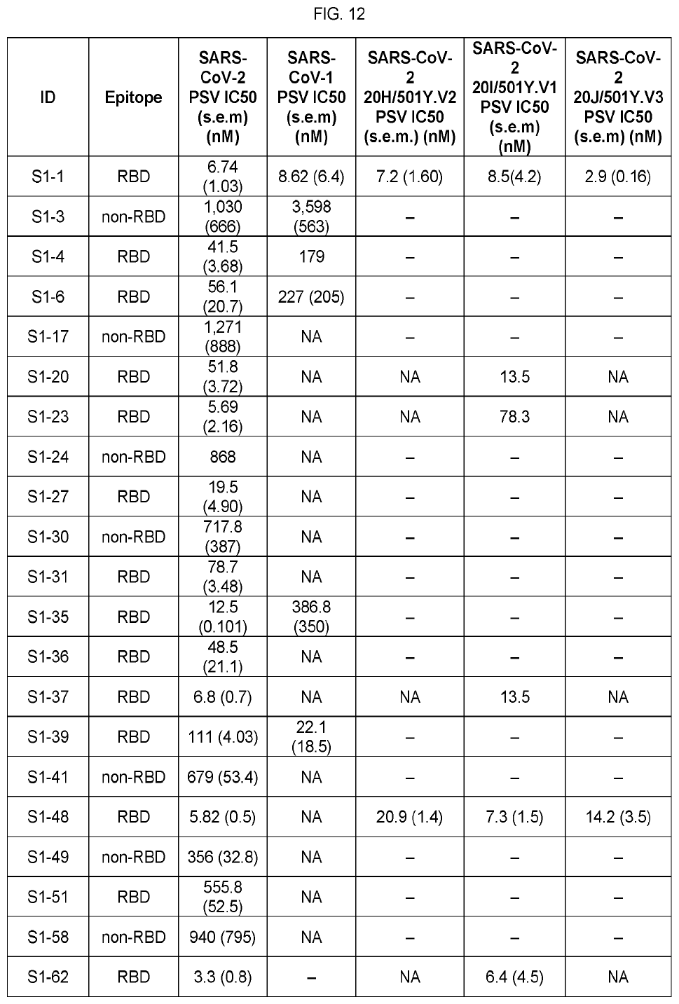

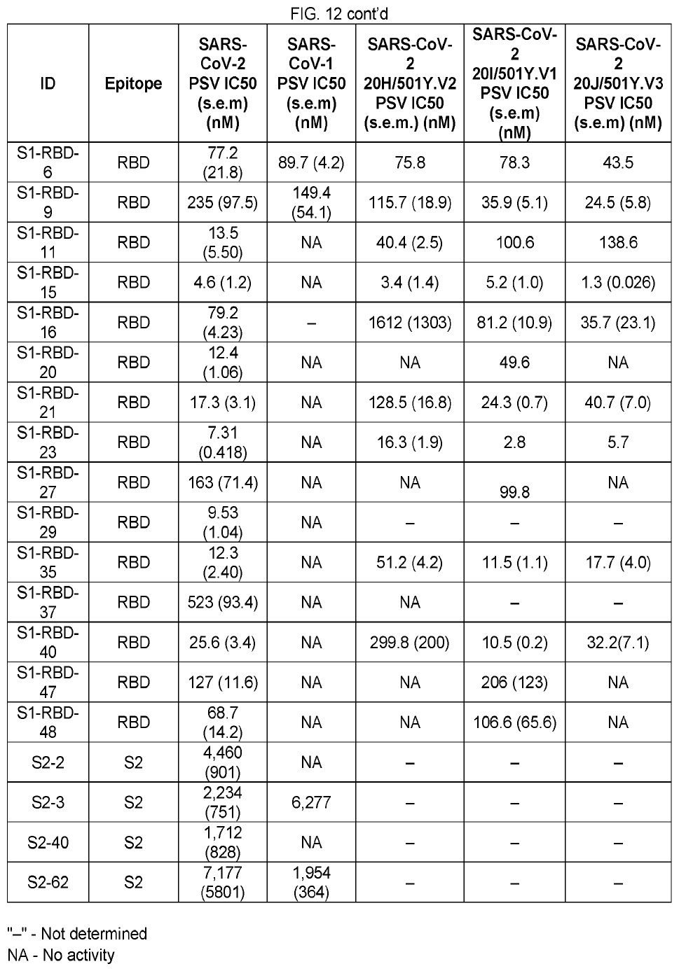

Nanobodies Neutralize SARS-CoV-2 variants and SARS-CoV-1. Certain mutations in spike, appearing in ‘variants of concern’ (VOC) associated with rapidly increasing case numbers in certain locales, have been demonstrated to reduce the neutralization potency of some monoclonal antibodies and polyclonal plasma, increase the frequency of serious illness, and are spreading rapidly (Wang et al., 2021, Nature 592(7855):616-622; Wibmer et al., 2021, Nat Med 27:622-625). Therefore a subset of the neutralizing nanobodies were tested against pseudovirus carrying the spike protein of the alpha variant (B.1.1.7/201/501Y.V1) (FIG. 10H); the beta variant (B.1.351/20H/501Y.V2) (Stamatatos et al., 2021, Science 372(6549):1413-1418; Tegally et al., 2021, Nature 592:438-443) (FIG. 10I); and the gamma variant (P.1/20J/501Y.V3) (FIGS. 10J and 12). These VOCs have mutations resulting in amino acid substitutions in spike, which impact the neutralization efficacy for some of the tested nanobodies. While all nanobodies neutralize the alpha variant, S1-23 showed an almost 14-fold drop in potency (FIGS. 10A and 10I). S1-23 and S1-62 failed to neutralize the beta and gamma variants, while S1-1 and S1-RBD-15 were as efficacious against all three VOCs as they were against wild-type spike (FIG. 10I). Both S1-RBD-21 and -35 also remained effective neutralizers of spike VOC pseudotypes, albeit with reduced IC50s compared to the wild-type spike (FIG. 10I). Remarkably, S1-RBD-9 showed increased neutralization activity against all three VOC, improving 2-fold against alpha, 6-fold against beta, and 10-fold against gamma (FIGS. 10H-10J). These results are in accord with the SPR studies which showed that S1-1, S1-RBD-9, -15, and -35 retained very strong binding to the alpha and beta VOC, whereas binding of S1-23 was reduced against alpha and completely abolished against beta (FIG. 7G). The B.1.617.2/21AA/S:478K/delta VOC has L452R and T478K as unique RBD amino acid substitutions (Campbell et al., 2021, Euro Surveill 26), which based on the epitope binning and escape mutants (below) would be predicted to impact neutralization of S1-RBD-11 and S1-RBD-35 (T478K) or S1-RBD-23 and S1-36 (L452F). However, the great majority of nanobodies in the repertoire would be predicted to show similar high neutralization efficacy against all these VOCs as compared to the wild-type virus. Overall, these data indicate that comprehensive mining of the repertoire and multimerization can lead to nanobody-based therapies that remain fully effective against common and even potentially yet-to-emerge variants of SARS-CoV-2 and with broad spectrum coronavirus inhibition activities.

Both SARS-CoV-1 and SARS-CoV-2 share the same host receptor, ACE2, and the RBDs of the viruses share 74% identity. As a result, some antibodies and nanobodies have been shown to be cross-neutralizing (Liu et al., 2020, Immunity 53, 1272-1280 e1275; Wrapp et al., 2020, Cell 181, 1004-1015 e1015). The ability of the nanobodies to neutralize SARS-CoV-1 in the pseudovirus assay was therefore tested. Of the nanobodies tested in this assay, several (7 of 27 tested) of the anti-RBD monomer nanobodies also displayed excellent neutralizing activities against SARS-CoV-1 spike pseudotyped virus (FIG. 10K). S1-1, -39 and S1-RBD-6 had similar IC50s against both pseudotypes, while S1-35 and S1-6 showed reduced activity against SARS-CoV-1 pseudotypes. Notably, S1-23, S1-37 and S1-48 showed no activity against SARS-CoV-1 spike pseudotypes. These three nanobodies are highly correlated with one another in the epitope binning analysis, indicating their binding to proximal epitopes on the RBD (FIG. 8A). Beyond nanobodies that bind to the RBD, 3 of 11 nanobodies that bind to non-RBD regions of S1 and S2 also neutralized SARS-CoV-1 spike pseudotypes (FIG. 12).

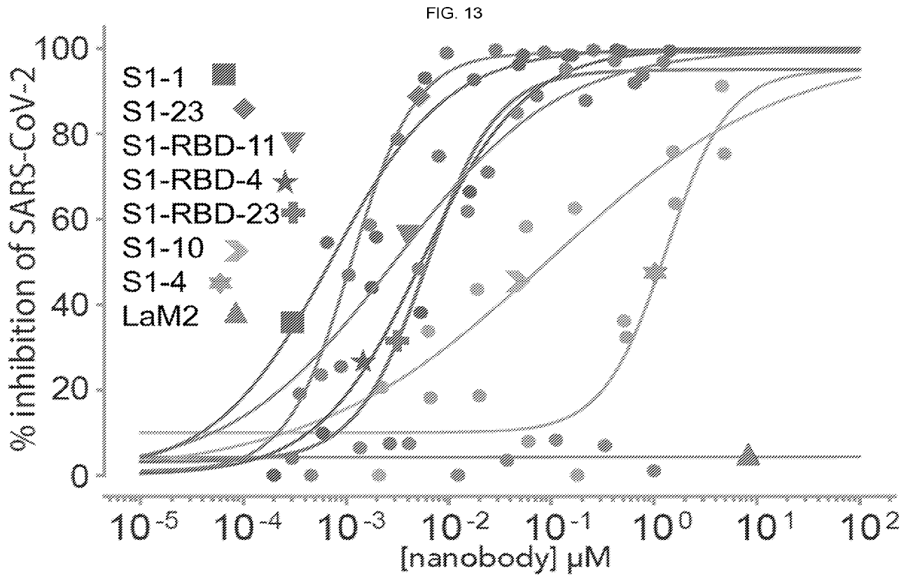

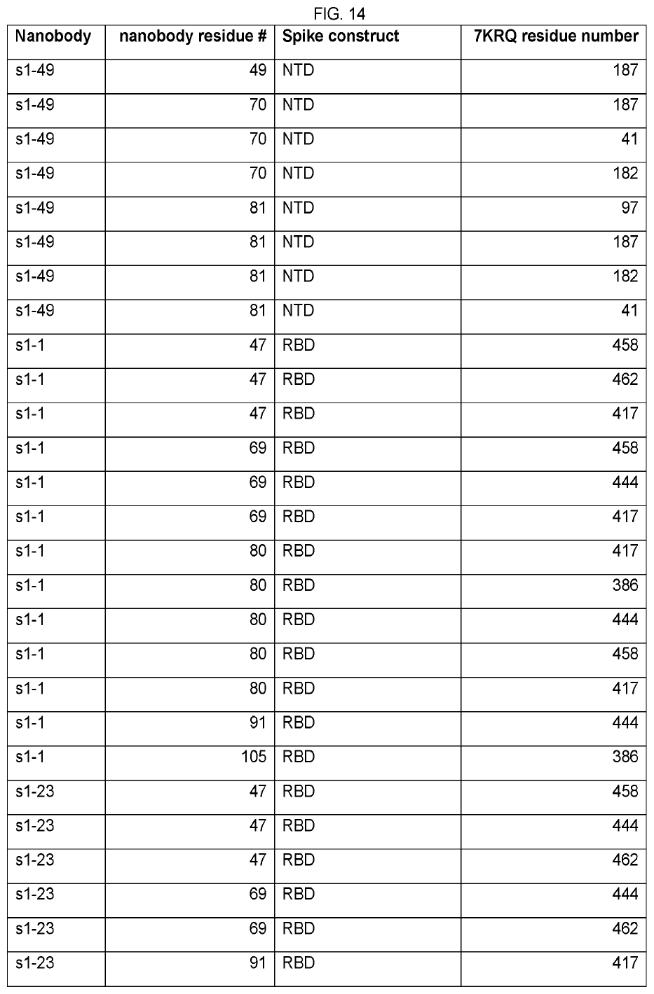

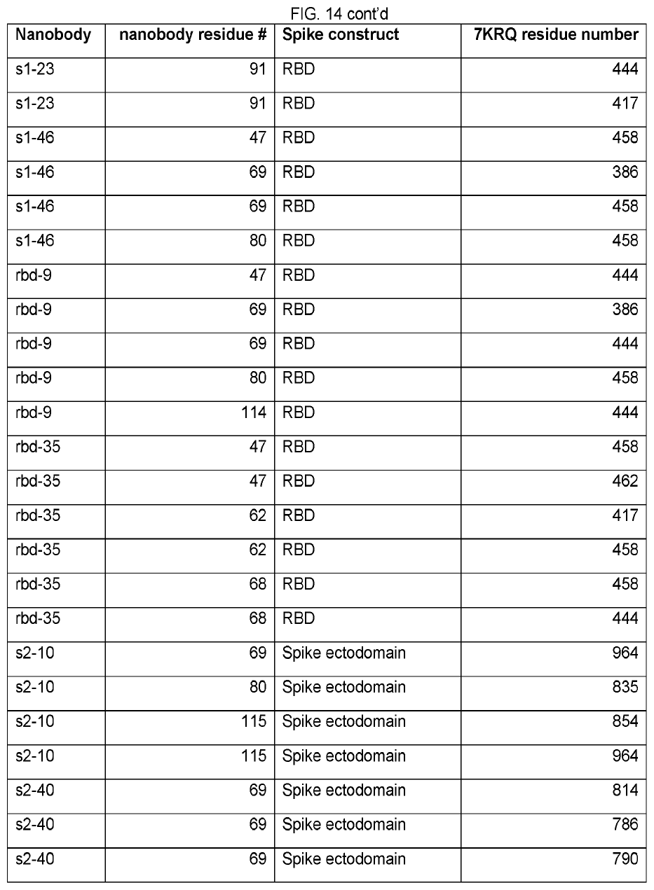

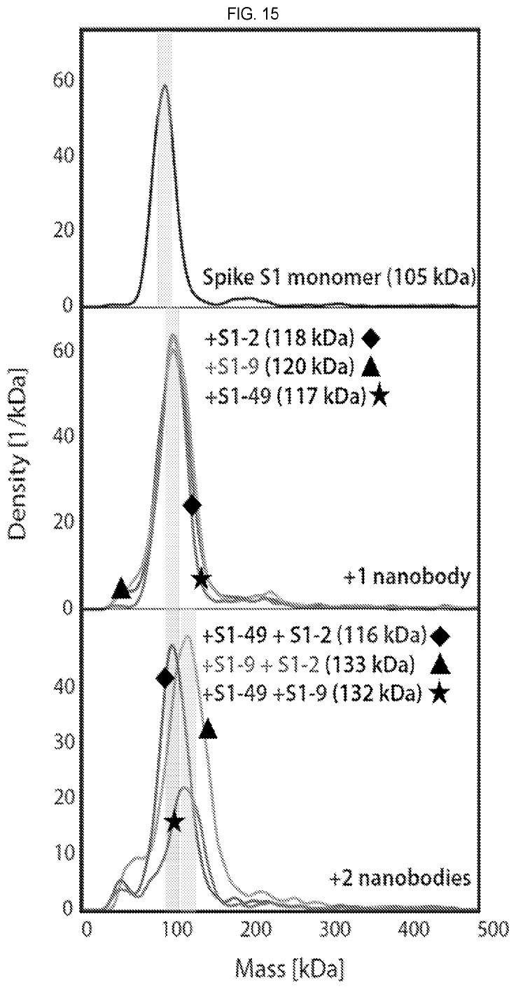

Nanobodies Effectively Neutralize SARS-CoV-2 Infection in Human Primary Airway Epithelium. Nanobody and antibody neutralizations have been reported to yield similar results when performed with pseudovirus versus authentic virus (Schmidt et al., 2020, J Exp Med 217; Schoof et al., 2020, Science 370, 1473-1479; Xiang et al., 2020, Science 370, 1479-1484). However, discrepancies have also been reported, particularly for antibodies targeting regions outside the RBD (Chi et al., 2020, Science 369, 650-655; Huo et al., 2020, Cell Host Microbe 28, 445-454 e446). Therefore a panel of exemplar (monomeric) nanobodies were selected, which target the RBD and domains outside of the RBD, to test for neutralization with authentic SARS-CoV-2. All nanobodies tested that neutralized pseudovirus also showed potent neutralization by plaque and focus reduction assays and correlated well with the pseudovirus assays (FIG. 13).

Nanobody cocktails are expected to be resistant to escape, as they recognize multiple epitopes (Baum et al., 2020, Science 369, 1014-1018; Gasparo et al., 2021, bioRxiv. 10.1101/2021.01.22.427567; Weisblum et al., 2020, Elife 9).

Among the entire repertoire, epitope binning shows that neutralization activity and mapped epitopes are heavily concentrated on the RBD (FIGS. 8A-8G and 10A-10K); 80% of the anti-RBD nanobodies are neutralizing, whereas 20% of the anti-S2 nanobodies and 60% of the non-RBD anti-S1 nanobodies are neutralizing. Based on the fact that glycosylation obscures a considerable fraction of the spike surface (Watanabe et al., 2020, Science 369, 330-333; Zhang et al., 2020, Mol Cell Proteomics, 100058), the repertoire explores much of the remaining available epitope space. The neutralization bias that was observed also likely reflects the most obvious mechanism of viral inhibition, namely, blocking the binding of spike's RBD domain to ACE2 on host membranes to preclude viral fusion, but the non-RBD based neutralization also underscores that other important mechanisms for viral inhibition exist.

RBD binding nanobodies fall into at least 7 groups. Many nanobodies bind epitopes that partially overlap with previously defined classes of IgG binding epitopes, but are more compact due to the smaller nanobody paratopes (Barnes et al., 2020, Nature 588, 682-687; Corti et al., 2021, Cell 184, 3086-3108; Xu et al., 2021, Nature 595, 278-282); however, many others define previously unreported binding sites.

Multiple Modes of RBD Binding and Neutralization. More than one mechanism of inhibition can exist. The RBD is tethered to spike through a hinge, allowing it to fluctuate between either a “down” conformation, hiding the vulnerable RBM from the host immune system, or an “up” conformation, exposing the RBM for potential ACE2 binding and so spike activation/disassembly (Cai et al., 2020, Science 369, 1586-1592; Corti et al., 2021, Cell 184, 3086-3108). Several nanobodies sharing similar epitope bins as S1-RBD-9, such as S1-RBD-34, S1-RBD-19, S1-RBD-25, S1-RBD-32, and S1-RBD-36 do not neutralize spike (FIG. 5) suggesting that neutralization may occur via an additional mechanism. The S2 domain is a prime, but largely unexplored, therapeutic target (Elshabrawy et al., 2012, PLoS One 7, e50366; Shah et al., 2021, Front Immunol 12, 637651). It is also not where the great majority of mutants in the current VOCs map, making it a particularly exciting target for potentially universal and VOC-resistant therapeutics. Here, the first neutralizing nanobodies that bind to S2 (FIGS. 7A-7G and 10A-10K) are presented. Some monoclonal antibodies that target S2 have been identified and shown to have neutralizing activity, but to the knowledge none have been structurally mapped (Andreano et al., 2021, Cell 184, 1821-1835 e1816; Li et al., 2020, Cell Mol Immunol 17, 1095-1097; Poh et al., 2020, Nat Commun 11, 2806; Song et al., 2020, bioRxiv; Wang et al., 2021, Nat Commun 12, 1715). Because S2's function is primarily membrane fusion rather than receptor binding, the nanobodies' neutralization mechanisms must differ from those discussed above.

Nanobodies, as monomeric proteins, can provide a unique opportunity to define possible mechanisms of activity that may otherwise be difficult to distinguish. For example, the dimeric nature of conventional antibodies can introduce ambiguities regarding the mechanisms of neutralization, because they can operate either as individual or pairwise binders. In the latter case, they may operate, for example, by aggregation (Thomas et al., 1986, J Virol 59, 479-485), increased avidity, enhanced steric hindrance via the larger binding entity, or by simultaneously binding and locking two separate moieties within a viral particle. In some cases, e.g., S1-7 and S1-25 (which are non-neutralizing as monomers), dimerization did not convert them into neutralizers. In other cases, dimerization and trimerization can engender several folds to orders of magnitude increase in neutralization potency (e.g., S1-RBD-35 and S1-23 respectively) (FIGS. 10A-10K and 11). There is also the case where a nanobody such as S2-7 that is essentially non-neutralizing as a monomer becomes strongly neutralizing upon dimerization (FIGS. 10A-10K). In this latter case, aggregation is a possible contributory mechanism, both between virions—which would lower effective virion concentration—or within a virion, with adjacent spike trimers being crosslinked to each other, inhibiting their function. Although a tremendous range in neutralization improvements by oligomerization is observed both by us and others, there is likely a limit to how much improvement can be induced by oligomerization as e.g. the trimers of S1-23 and S1-RBD-35 do not show a similar fold improvement as to what was observed for the monomer to dimer transition (Koenig et al., 2021, Science 371; Ma et al., 2021, J Virol; Schoof et al., 2020, Science 370, 1473-1479; Xiang et al., 2020, Science 370, 1479-1484; Xu et al., 2021a, bioRxiv. 10.1101/2021.03.04.433768).

Synergistic Activity with Nanobody Combinations. Drugs are often combined to improve single-agent therapies and dramatically enhance the therapeutic potential of either drug alone while reducing the drug concentrations to be administered. Synergy occurs when the combination of drugs has a greater effect than the sum of the individual effects of each drug. A major advantage of a large repertoire of nanobodies that bind to different epitopes on spike is the potential for cooperative activity among nanobody pairs (or higher order combinations) leading to synergistic viral neutralizing effects. The small size of nanobodies also provides a great advantage over much larger immunoglobulins in this context, as the binding of a nanobody has a lower chance of sterically occluding the binding of a second nanobody to a distinct epitope, and because they are monovalent. Moreover, nanobodies binding to the RBD may stabilize the otherwise “up”-“down” fluctuating RBD in its “up”, ACE2-engaging, position (Bracken et al., 2021, Nat Chem Biol 17, 113-121; Schoof et al., 2020, Science 370, 1473-147 albumin bi9; Xiang et al., 2020, Science 370, 1479-1484). This can have three effects, all of which can potentially promote nanobody synergy: first, it will increase the effective on-rate for spike trimer to that measured for monomer—and therefore, make it easier for complementary nanobodies to bind and inhibit; second, by stabilizing this “up” for any one of the three RBDs in each spike trimer, it destabilizes the “down” position for the remaining two RBDs, again making nanobody binding from the second class more likely; and third, the “up” position exposes additional nanobody epitopes that would otherwise be buried (Sun et al., 2021, bioRxiv. 10.1101/2021.03.09.434592; Xiang et al., 2020, Science 370, 1479-1484).

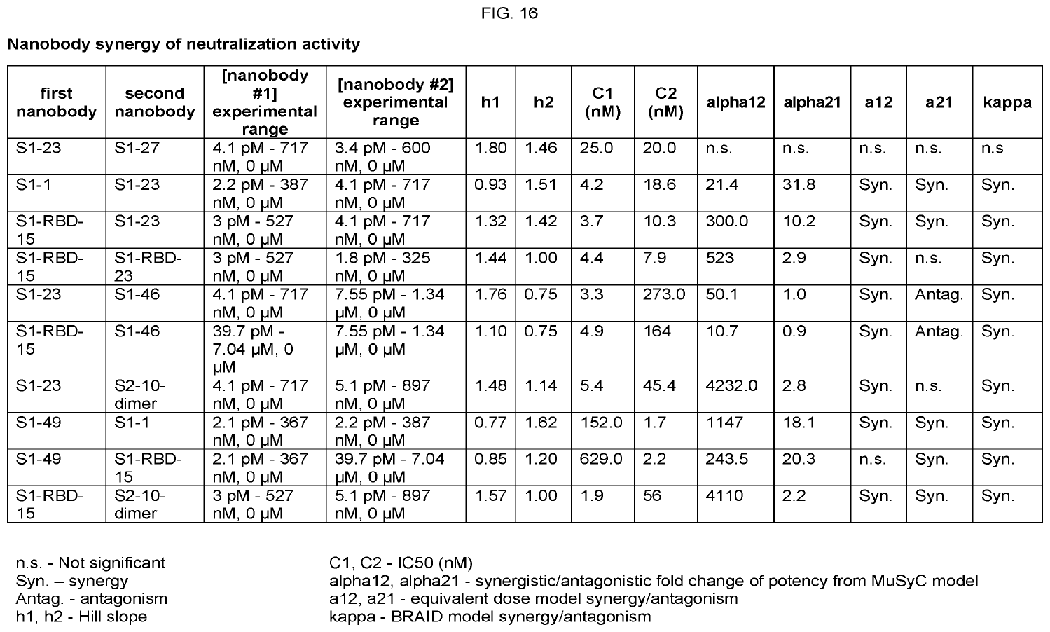

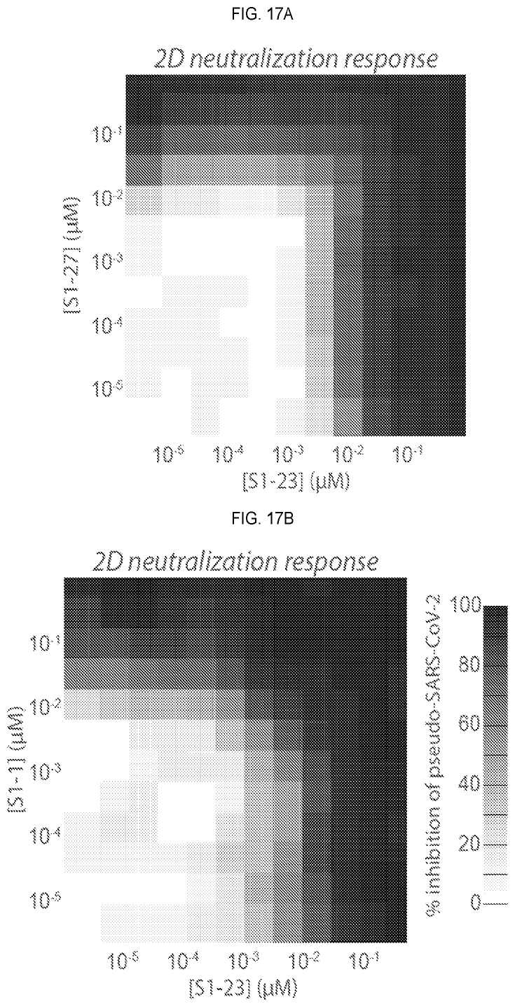

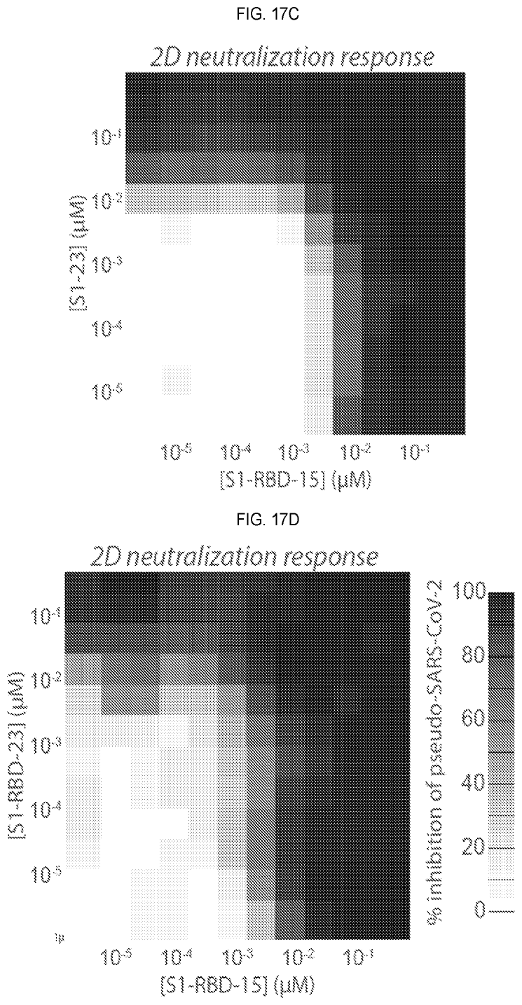

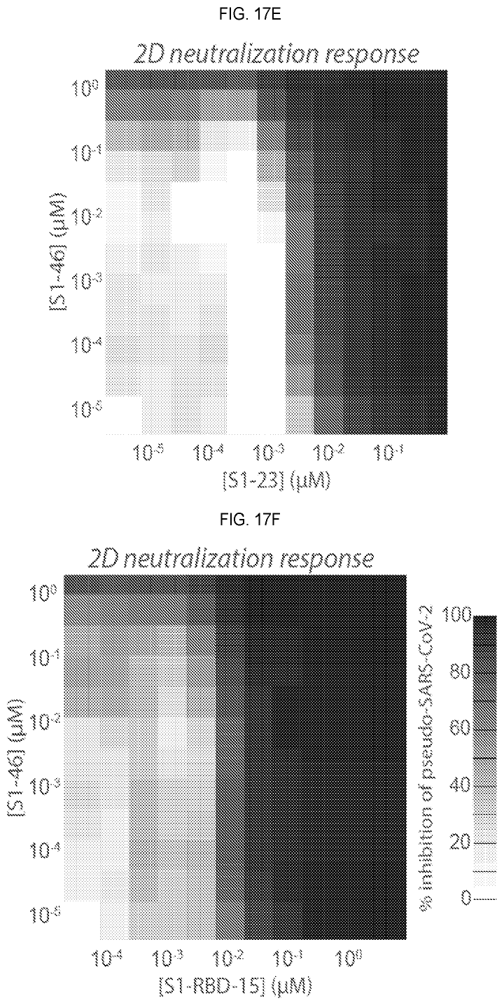

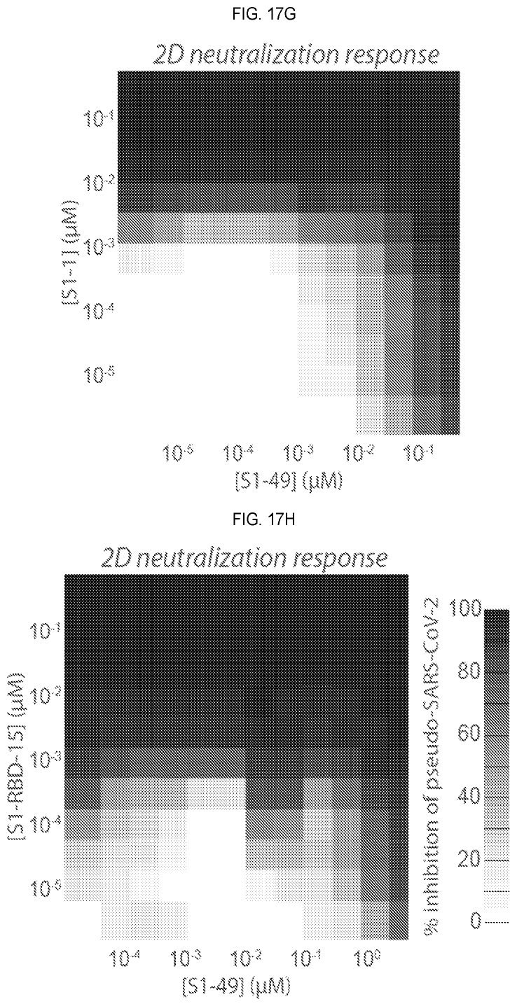

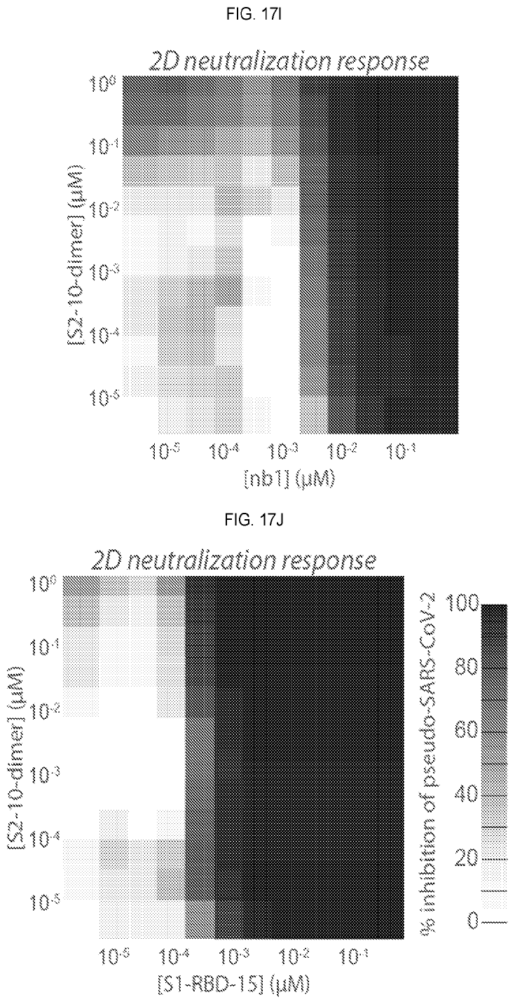

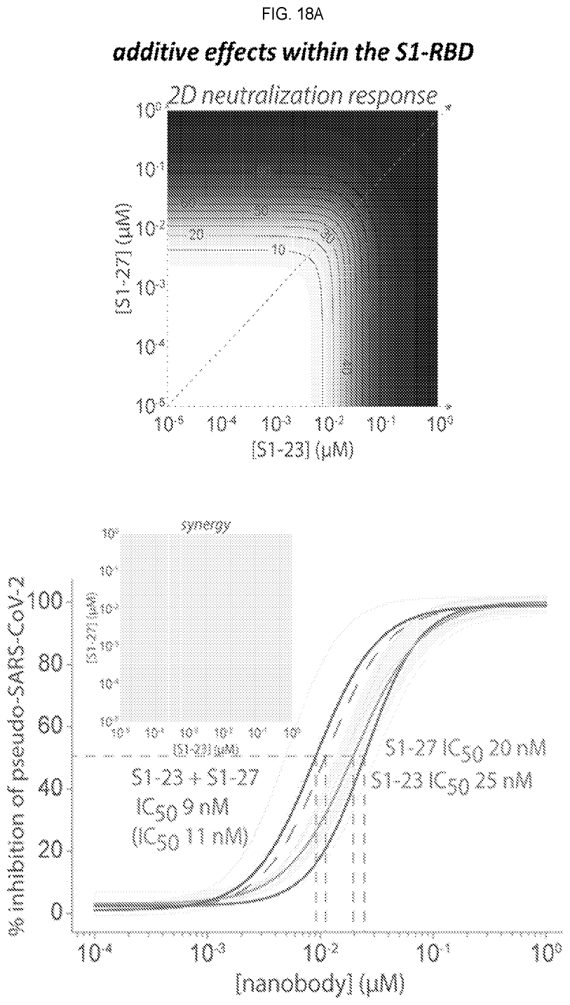

Using an automated platform, pairwise combinations of nanobodies were titrated in a 2D dilution format and measured their IC50s in the pseudovirus assay. IC50s were modeled using a multi-faceted synergy framework (Wooten and Albert, 2020, Bioinformatics 37, 1473-1474), including a parameterized version of the equivalent dose model (Zimmer et al., 2016, Proc Natl Acad Sci USA 113, 10442-10447), the Bivariate Response to Additive Interacting Doses (BRAID) model (Twarog et al., 2016, Sci Rep 6, 25523), and the multi-dimensional synergy of combinations (MuSyC) model, which models a two-dimensional (2D) Hill equation and extends it to a 2D surface plot (Meyer et al., 2019, Cell Syst 8, 97-108 e116). Synergy is evidenced by the parameters of the respective models (FIG. 16). To select nanobody pairs to test for synergy, epitope mapping, structural data, and biophysical characterization were used. Pairwise combinations of nanobodies that bind to similar epitopes, to different epitopes on RBD, and to regions outside and within the RBD were tested (FIGS. 16, 17, and 18A-18J).

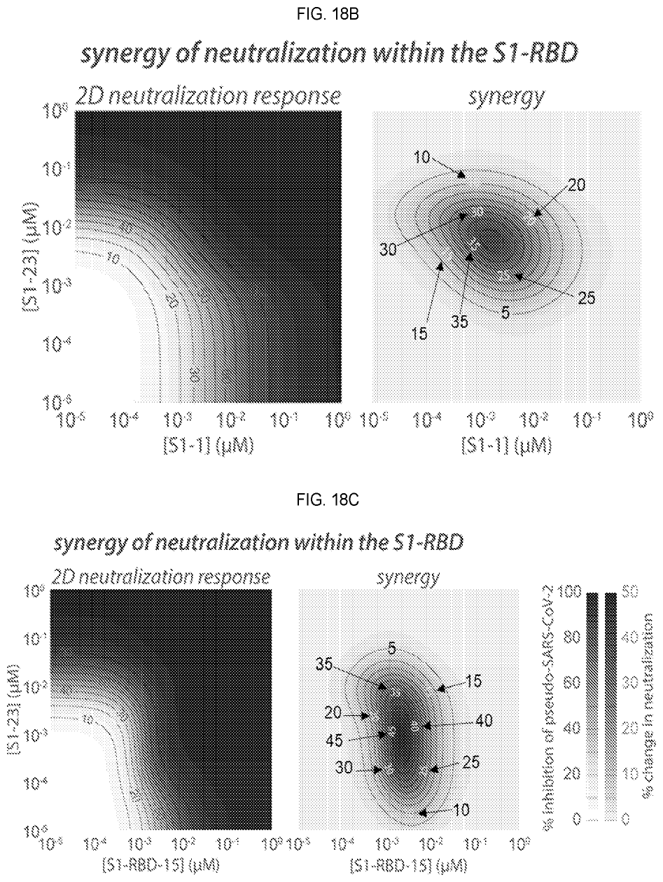

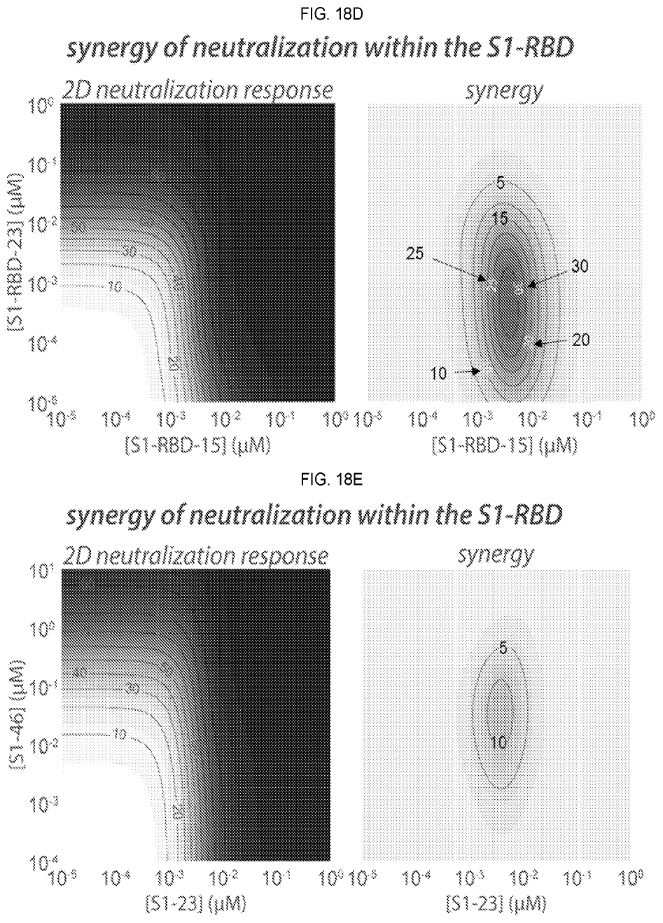

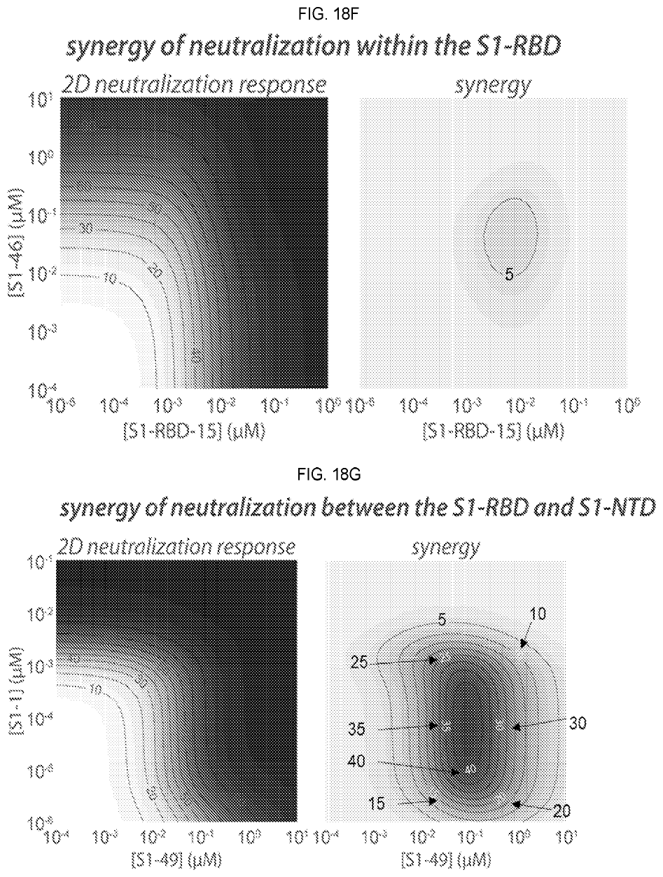

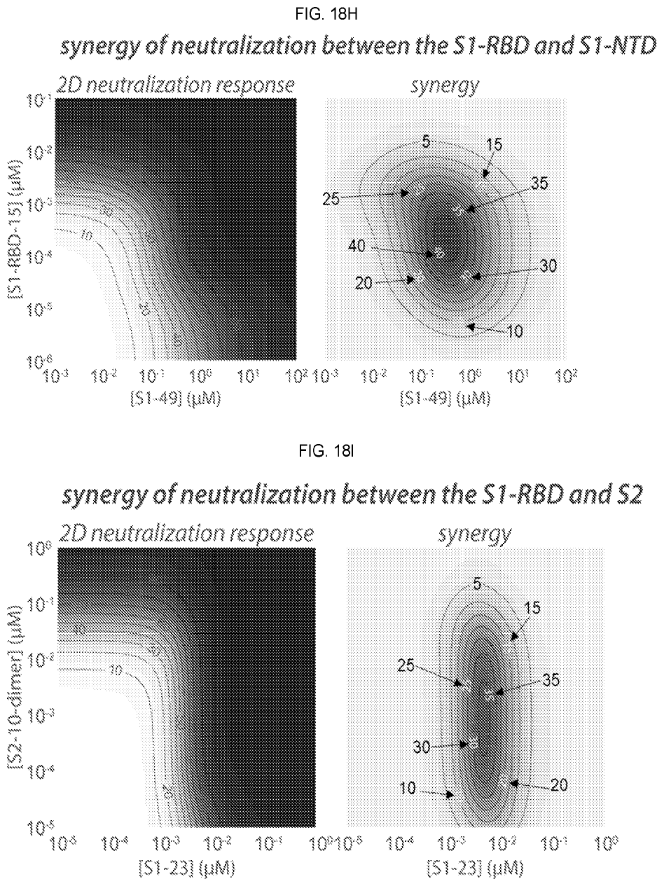

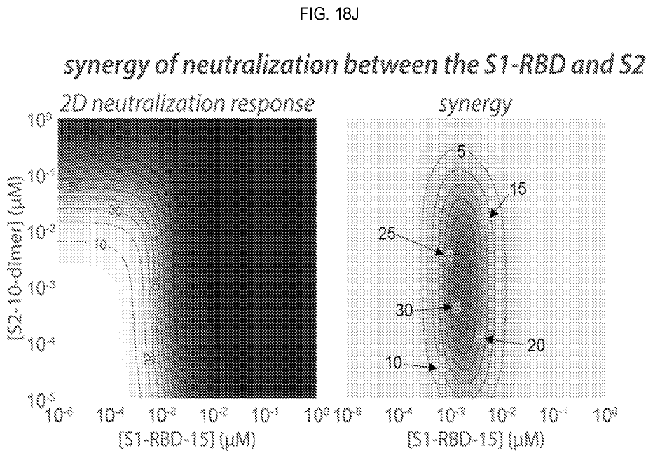

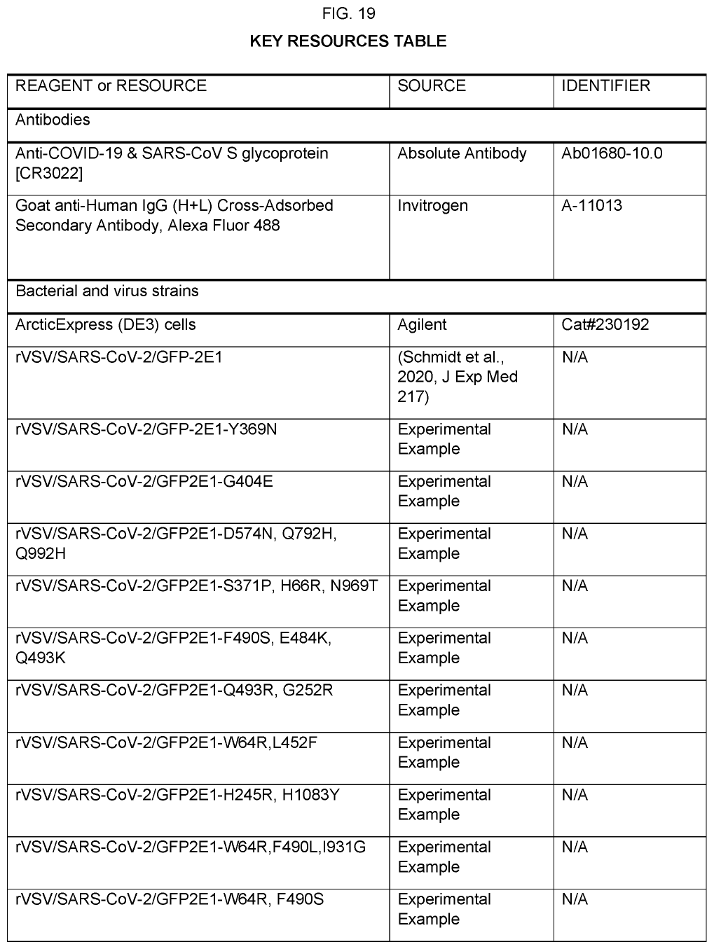

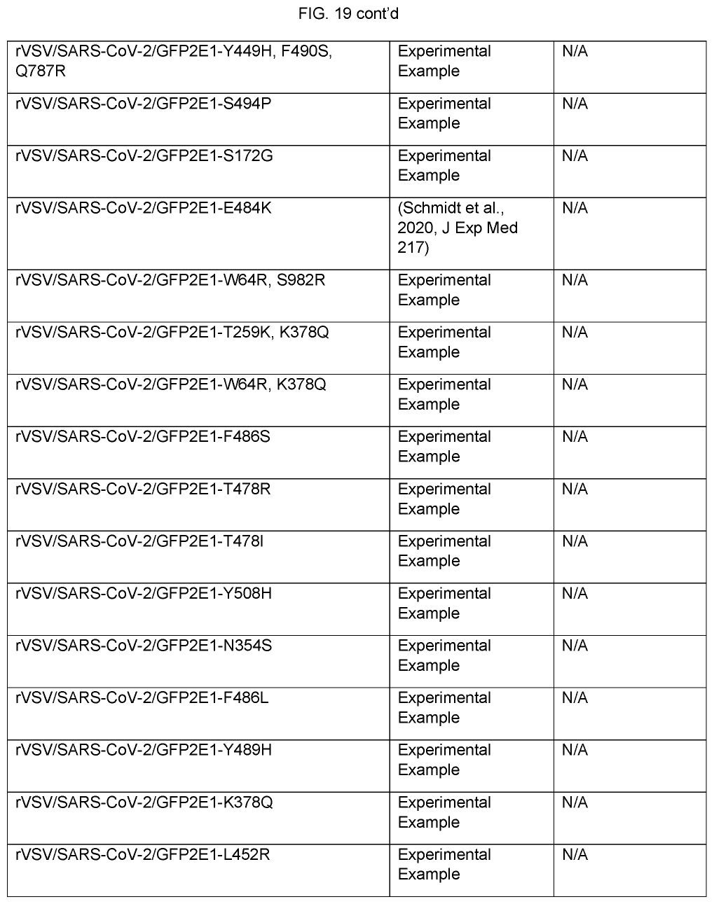

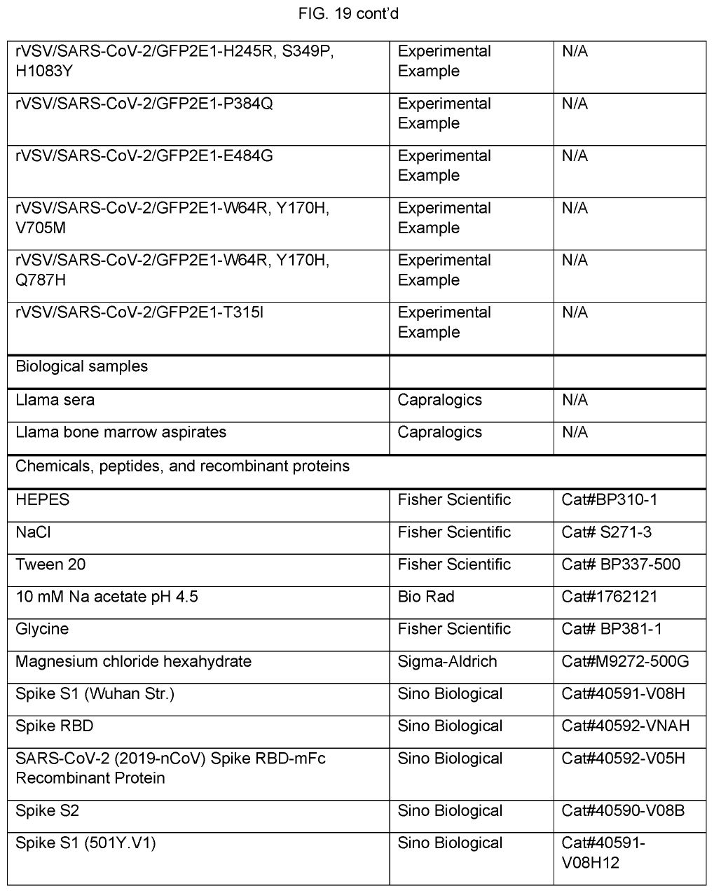

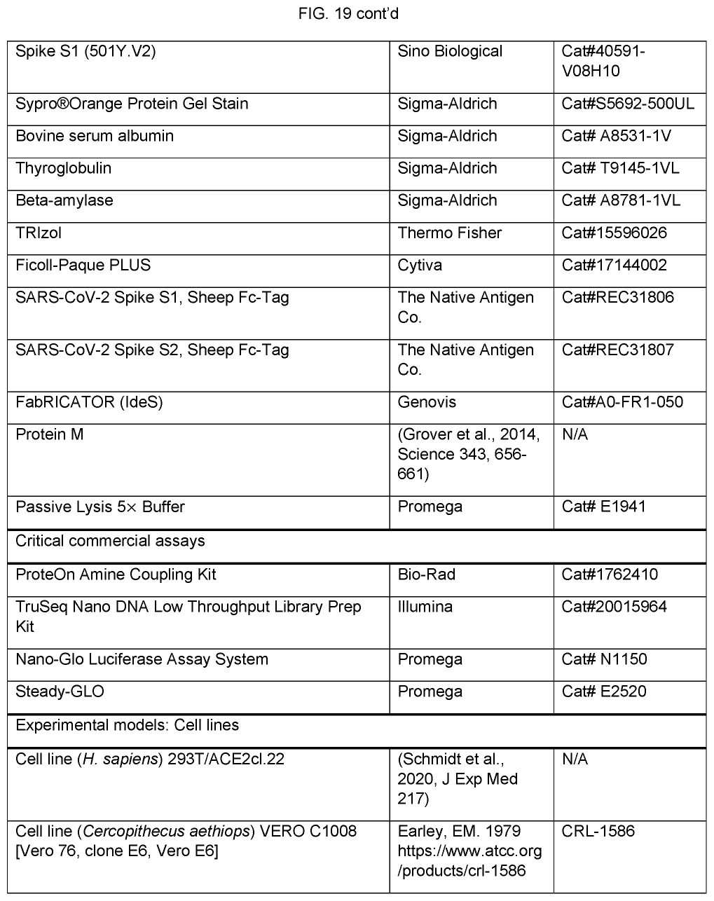

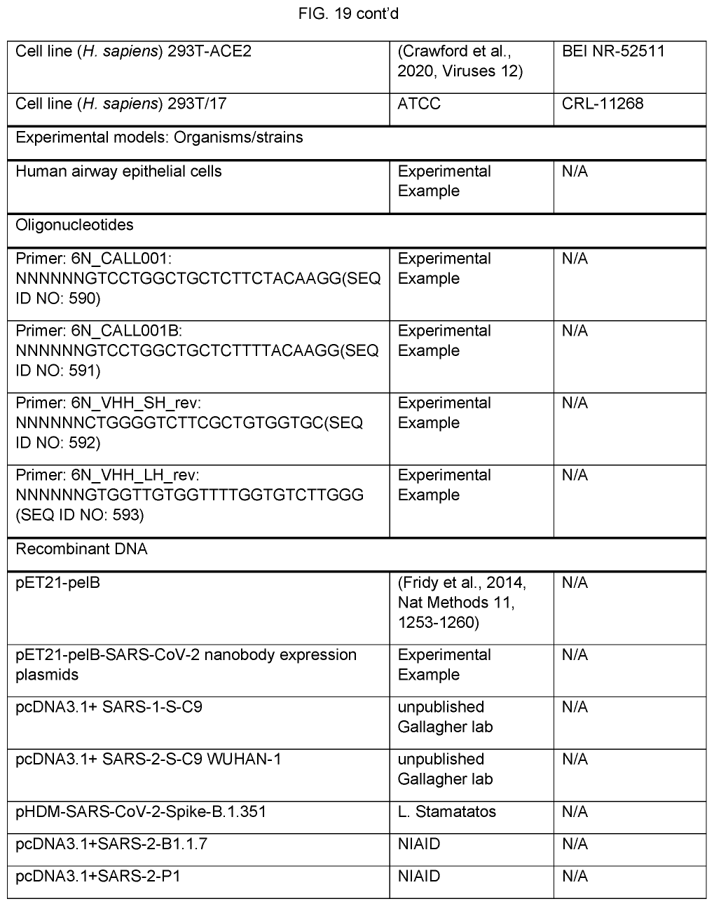

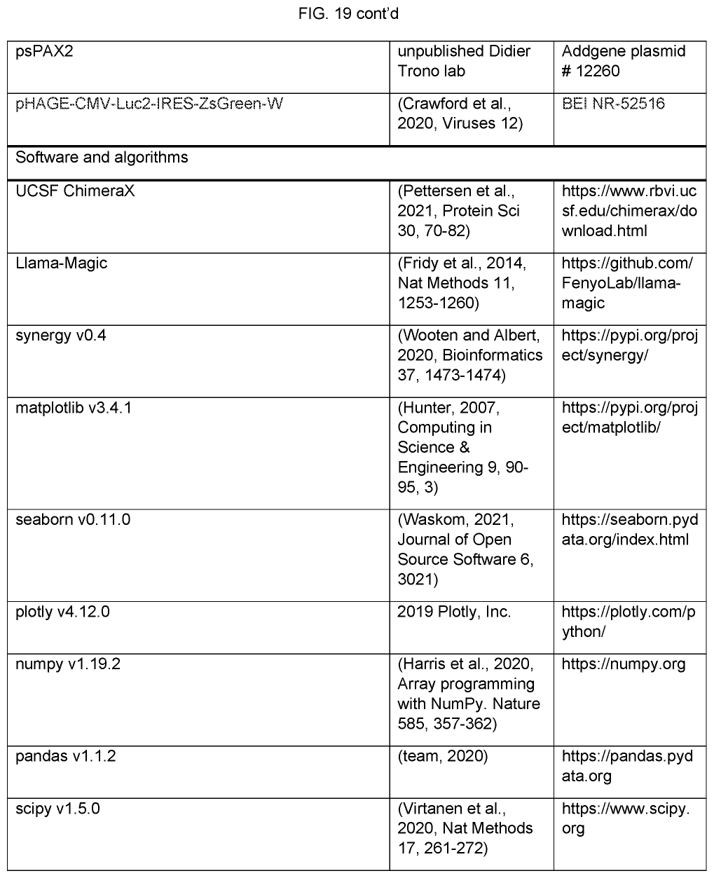

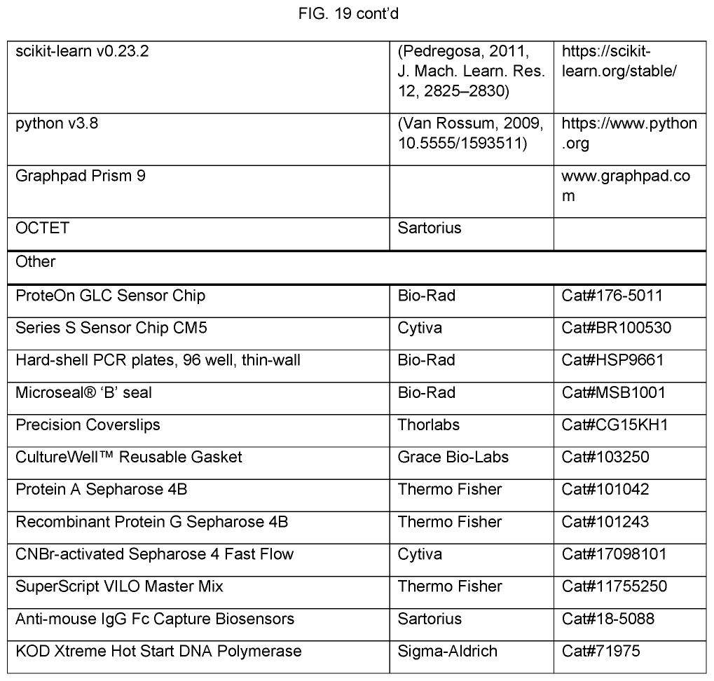

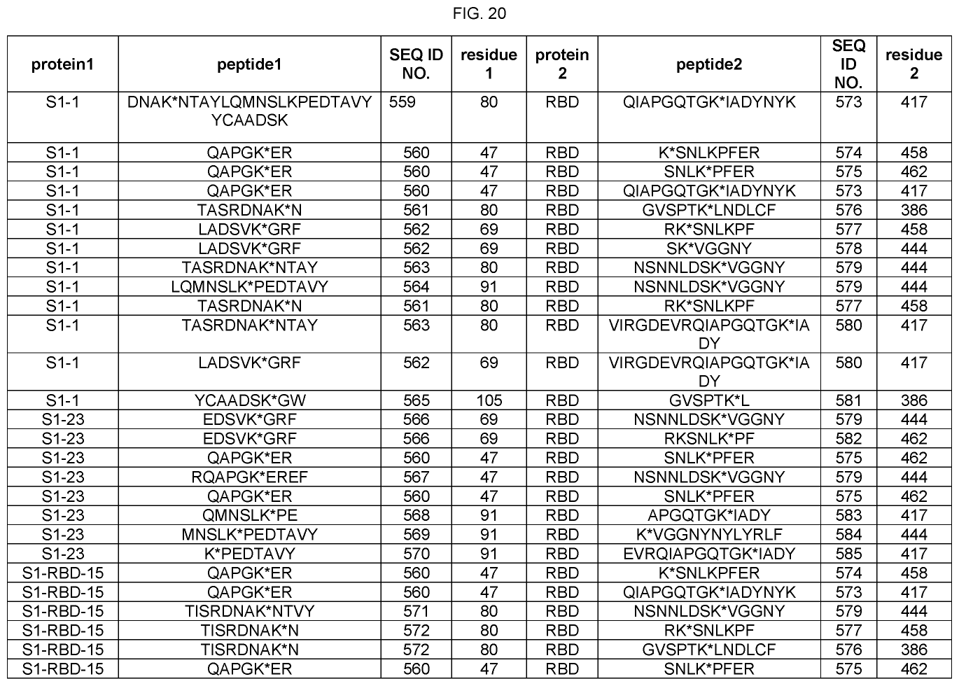

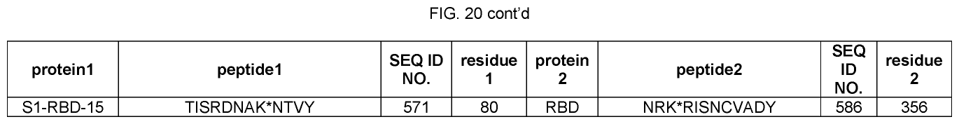

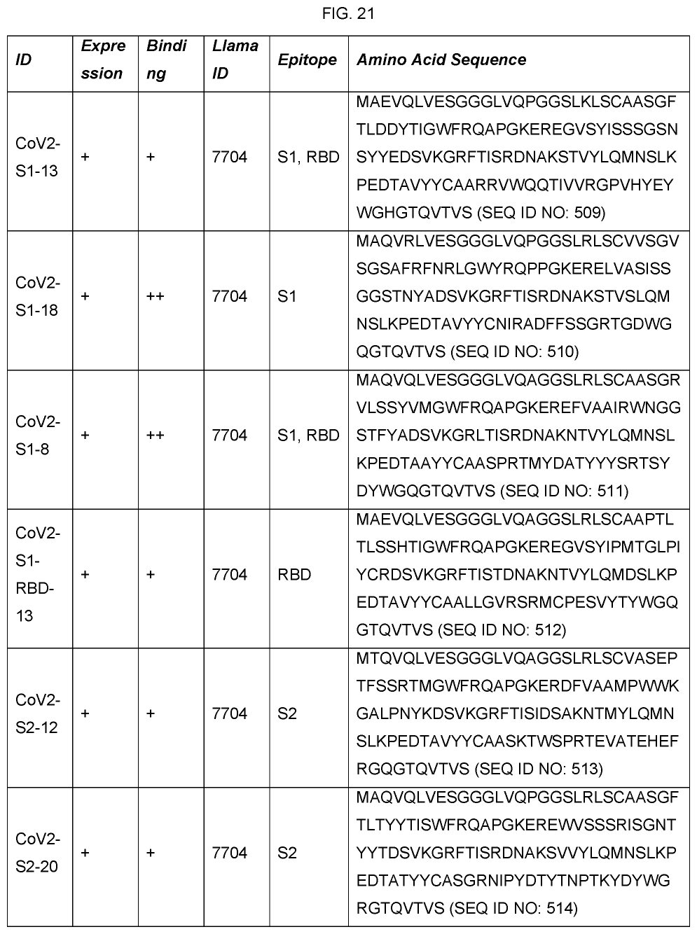

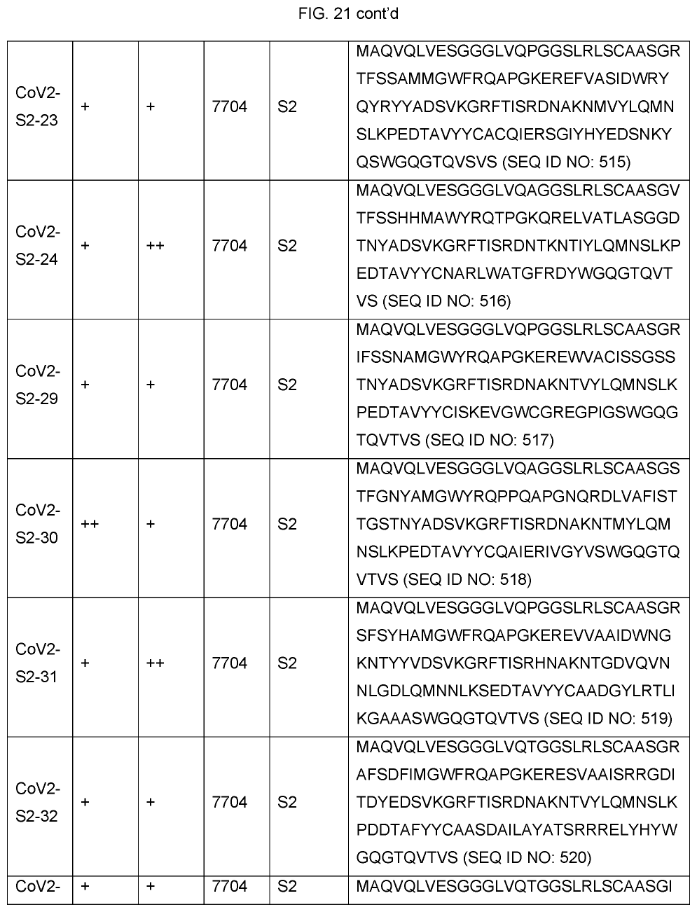

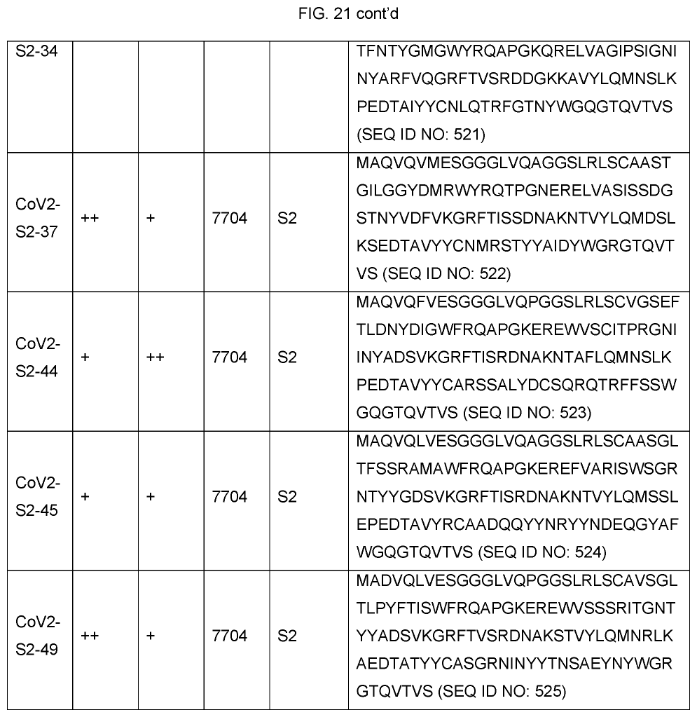

Combinations of S1-27 and S1-23 showed simple additive effects (FIG. 18A). These nanobodies belong to the same epitope bin (FIG. 8A); their additive effect is as expected for two nanobodies accessing the same site on S1-RBD, but e.g., in equal concentrations, effectively doubling the concentration of a single nanobody. The potential for synergy resides instead in nanobodies that bind to different epitopes, and can bind to spike monomers simultaneously. Therefore, combinations that bind to different epitopes were tested, first focusing on the RBD. Indeed, powerfully synergistic effects were observed between numerous nanobody pairs. For example, the combination of S1-23 and S1-1, which bind to different sites of the RBD, dramatically increased the potency of both nanobodies by 32 and 21 fold, respectively (FIG. 18E; FIG. 16). S1-RBD-15, which binds to a similar epitope as S1-1, shows corresponding synergy with S1-23, suggesting that synergy may be predictable based on epitope mapping (FIG. 18C). In this case, however, S1-RBD-15 had a greater influence on S1-23, promoting its potency by 300-fold. S1-RBD-15 showed a comparable synergy profile against S1-RBD-23, which binds to a different site on the RBD (FIG. 8A). It should not be taken for granted that simply binding to distinct epitopes on RBD simultaneously will always be sufficient to generate a strongly synergistic response. For example, S1-46 failed to show synergy with either S1-23 or S1-RBD-15 (FIGS. 18E, 18F). Indeed, synergy modeling indicates that S1-46 actually mildly antagonizes both S1-23 and S1-RBD-15 (FIG. 16).

S1-49, which binds to a different site on spike, substantially improved the neutralization potency of either S1-1 or S1-RBD-15 (FIGS. 18G, 18H). The synergy observed with S1-49 with S1-1 elicited a >1000-fold increase in potency.

Synergy between nanobodies targeting the S1-RBD and S2 were also tested, which revealed remarkable results. In this case, a dimer of S2-10 was used to increase its potency to be closer to that of the nanobodies to which it was paired. Interestingly, among all the nanobody pairs that were tested, the synergy was greatest with the S2-10-dimer, which showed >4000 fold increase in potency when combined with either S1-23 or S1-RBD-15 (FIGS. 18I, 18J). The strong synergy observed may also reflect the distinct mechanisms by which either the RBD-binders or S2-10 operate individually.

The repeated observation of strong synergistic effects between nanobodies is especially noteworthy, reflecting the unique properties of nanobodies that are not shared by, for example, human monoclonal antibodies. For example, human IgGs were structurally aligned with the S1-23 and S1-1 paratopes and found the same binding characteristics would not be predicted to act synergistically because they could not bind the same monomer simultaneously as they would clash with other RBDs on the spike trimer. In the case of S1-23 and S1-RBD-15 epitopes, structural alignment of IgGs with nanobody paratopes suggested a strong inter-IgG steric clash in addition to a clash with other RBDs in the down position.

Perspectives. The data presented here demonstrate the power of raising large and diverse repertoires of nanobodies against the entire ectodomain of SARS-CoV-2 spike to maximize the likelihood of generating potent reagents for prophylactics and therapeutics. Even if antibodies or nanobodies are resistant to the current variants, they will not necessarily be resistant to variants as they continually emerge. However, judicious choice of nanobody combinations that can synergize and have orthogonal and complementary neutralization mechanisms have the potential to result in potent and broadly neutralizing reagents that are resistant to viral escape. Collectively, this unique and readily modifiable repertoire has the potential to complement vaccines, drugs and single epitope reagents, and guard against single molecule failure in human trials even in the face of emerging variants. Most urgently, it paves the way to develop therapeutics for hospitalized patients with acute disease, and address the unmet needs of patients in the developing world, many of which will not see a COVID-19 vaccine before 2023 (Padma, 2021, Nature. 10.1038/d41586-021-01762-w).

Methods. Summary of Key Improvements to Nanobody Generation Pipeline. To maximize the purity of the serum HCAb sample, different binding conditions were explored to select for the tightest V_{H}H binders—a key step not generally available to display panning methods (Fridy et al., 2014, Nat Methods 11, 1253-1260). An additional HCAb purification step was also used to deplete VH IgG by incubation with immobilized Protein M, a mycoplasma protein specific for IgG light chain (Grover et al., 2014, Science 343, 656-661). To further enrich the V_{H}H sample for MS analysis and remove Fc, a digest with IdeS, a protease that cleaves the V_{H}H domain from the HCAb with higher specificity than conventionally used papain (von Pawel-Rammingen et al., 2002, EMBO J 21, 1607-1615.) was performed. Greater peptide coverage for LC-MS was attained by using complementary digestion with trypsin and chymotrypsin (Xiang et al., 2020, Science 370, 1479-1484), augmented by partial SDS-PAGE gel-based separation of different V_{H}Hs to reduce the V_{H}H complexity and to give more complete peptide coverage and candidate selection. PCR primers were redesigned to maximize coverage of V_{H}H sequences for the cDNA libraries. Also, to increase the reliability of the library, singletons were not considered as candidates and priority was given to sequences with high counts. Finally, the Llama-Magic software package (Fridy et al., 2014, Nat Methods 11, 1253-1260) was refined to include improved scoring functions, weighting the length, uniqueness, and quality of the MS data especially for complementarity-determining regions. This optimized protocol allowed us to identify 374 unique CDR3 sequences (from 847 unique V_{H}H candidates). Details are provided below.

Antigens. Recombinant Fc-tagged SARS-CoV-2 spike S1 and S2 proteins purified from HEK293 cells were used for llama immunization (The Native Antigen Company REC31806 and REC31807). For affinity isolation, binding screens, SPR analysis, and mass photometry, recombinant spike S1-His, untagged RBD, or S2-His proteins expressed in HEK293 (S1 and RBD), or insect cells (S2) were used (Sino Biological 40591-V08H, 40592-VNAH, and 40590-V08B). Native mass spectrometry (Olinares et al., 2016, Anal Chem 88, 2799-2807; Olinares et al., 2021, Structure 29, 186-195 ei86) was used to confirm the quality of these proteins, and were found to be glycosylated, with S1 and S2 observed to be heavily glycosylated (at least 10 kDa of attached glycans). RBD was observed to be monomeric, S1-His likely monomeric, and S2-His a mix of monomer and trimer.

Immunization and Isolation of V_{H}H Antibody Fractions. A pre-screening protocol was used to select llamas with naturally strong immune responses, as determined by activity against standard animal vaccines (Thompson et al., 2016, J Immunol Methods 430, 56-60). Two llamas, Marley (9-year-old male) and Rocky (5-year-old male) were immunized with recombinant SARS-CoV2 spike S1 and SARS-CoV2 spike S2 expressed in HEK293 cells as Fc fusion proteins. Llamas were injected subcutaneously with 0.25 mg of each antigen with CFA, then boosted with the same amounts with IFA at intervals of 14, 7, 21, and 10 days. Serum bleeds and bone marrow aspirates were obtained 9 days after the final injection. From the production serum bleeds, HCAb fractions of IgG were obtained by purification with immobilized Protein A and Protein G as previously described (Fridy et al., 2014, Nat Methods 11, 1253-1260). Residual light-chain containing IgG was removed from this fraction by incubating with 25 μl of 10 mg/ml Protein M-Sepharose per mg of HCAb (Grover et al., 2014, Science 343, 656-661). After a 30 min incubation, the HCAb flow-through was collected. 12 mg of this HCAb fraction was then incubated with Sepharose-conjugated recombinant SARS-CoV2 spike S1-His, RBD, or S2-His protein. This resin was washed with 1) 20 mM sodium phosphate, pH 7.4+500 mM NaCl; 2) 2 M MgCl₂ in 20 mM Tris, pH 7.5; 3) PBS+0.5% Triton X-100; and 4) PBS. The resin was then resuspended in a 200 μl solution of 2 U/μl IdeS enzyme (Genovis) in PBS, and digested for 3.5 hours at 37° C. on an orbital shaker. The resin was then washed with 1) PBS 2) PBS plus 0.1% Tween-20 3) PBS. Bound protein was eluted by incubating 10 min at 72° C. in 1×NuPAGE LDS sample buffer (Thermo Fisher). The samples were reduced with DTT and alkylated with iodoacetamide, then run on a 4-12% Bis-Tris gel. Bands at 15 kDa and 20 kDa corresponding to digested V_{H}H region were then cut out and prepared for MS.

RT-PCR and DNA Sequencing. Bone marrow aspirates were obtained from immunized llamas concurrent with production serum bleeds. Bone marrow plasma cells were isolated on a Ficoll gradient using Ficoll-Paque (Cytiva). RNA was isolated from 3-4×10⁷ cells using TRIzol reagent (Thermo Fisher), according to the manufacturer's instructions. cDNA was synthesized using SuperScript IV reverse transcriptase (Thermo Fisher). A PCR was then performed with V_{H}H IgG specific primers and Deep Vent polymerase (New England Biolabs). Forward primers 6N_CALL001 5′-NNNNNNGTCCTGGCTGCTCTTCTACAAGG-3′ (SEQ ID NO: 590) and 6N_CALL001B 5′-NNNNNNGTCCTGGCTGCTCTTTTACAAGG-3′ (SEQ ID NO: 591) target the leader sequence, (Conrath et al., 2001, Antimicrob Agents Chemother 45, 2807-2812) while reverse primers 6N_VHH_SH_rev 5′-NNNNNNCTGGGGTCTTCGCTGTGGTGC-3′ (SEQ ID NO: 592) and 6N_VHH_LH_rev 5′-NNNNNNGTGGTTGTGGTTTTGGTGTCTTGGG-3′ (SEQ ID NO: 593) target short and long hinge sequences at the 3′ side of V_{H}H. Primers included 6 random bases (N) to aid cluster identification. The 350-450 bp product of this reaction was gel purified, then ligated to Illumina adaptors before library preparation using Illumina kits, before MiSeq sequencing using 2×300 bp paired end reads.

Identification of Nanobodies by Mass Spectrometry. Trypsin (Roche) or chymotrypsin (Promega) solution were added to previously reduced, alkylated, diced, destained and dehydrated gel pieces at 1:4-3:1 enzyme to substrate mass ratios. Gel pieces were allowed to rehydrate with enzyme solution for 10 min on ice. 45 μl of digestion buffer (trypsin: 50 mM ammonium bicarbonate, 10% acetonitrile; chymotrypsin: 100 mM Tris pH 7.8, 10 mM CaCl₂) were then added, and samples were incubated for 6 h at 37° C. (trypsin) or 25° C. (chymotrypsin). Supernatant was then removed from gel pieces, and transferred to a new tube. 150 μl of a 1.67% FA, 67% ACN, 0.05% TFA solution were added to gel pieces, and shaken at 4° C. for 6 h. Supernatant was removed from gel pieces, transferred to the tube with previous supernatant, and evaporated in a speedvac until dry. Samples were resuspended in 5% formic acid, 0.1% TFA and cleaned on StageTips (Rappsilber, 2012, Expert Rev Proteomics 9, 485-487).

Samples were analyzed with a nano-LC 1200 (Thermo Fisher) using an EASYspray PepMap RSLC C18 3 micron, 100 Å, 75 μm by 15 cm column coupled to an Orbitrap Fusion Lumos Tribrid mass spectrometer (Thermo Fisher). An Active Background Ion Reduction Device (ABIRD, ESI Source Solutions) was used to reduce background. The Lumos was operated in data-dependent mode, and top intensity ions were fragmented by high-energy collisional dissociation (normalized collision energy 28). Ions with charge states 2-5 were selected for fragmentation. Orbitrap resolution was 120,000. The quadrupole isolation window was 1.4, and the MS/MS used a maximum injection time of 250 ms with 1 microscan.

The initial identification of nanobody sequences was performed as described (Fridy et al., 2014, Nat Methods 11, 1253-1260) using the program Llama-Magic (https://github.com/FenyoLab/llama-magic) with a few added features (including being able to deal with chymotryptic proteolysis and to rank V_{H}Hs by corresponding read counts in high-throughput sequencing data), where 23 MS datasets (concatenated from all MS acquisition data according to antigens, animal individuals, gel band positions and proteases) were independently searched. The results were filtered with criteria including read counts, uniqueness score, and quality and coverage of MS/MS fragments to generate a collection of high confidence nanobody sequences. A CDR3 network graph was created by connecting nodes (unique high confidence CDR3 sequences) by edges where a CDR3 pair has a Damerau-Levenshtein distance of no more than three, by using NetworkX 2.5 (https://networkx.org) and pyxDamerauLevenshtein (https://github.com/gfairchild/pyxDamerauLevenshtein). The diversity of nanobodies for screening was maximized by selecting CDR3 sequences from isolated components of the network graph, together with varying CDR3 lengths and animal individual origin.

Cloning and Purification of Nanobodies. Nanobody sequences were codon-optimized for expression in E. coli and synthesized as gene fragments (IDT), incorporating BamHl and XhoI restriction sites at 5′ and 3′ ends, respectively. Nanobody sequences were then subcloned into pET21-pelB using BamHl and XhoI restriction sites as previously described (Fridy et al., 2014, Nat Methods 11, 1253-1260). pelB-fused nanobodies were expressed and purified using Arctic Express (DE3) cells (Agilent) as previously described, using TALON metal affinity resin (Takara) (Fridy et al., 2014, Nat Methods 11, 1253-1260).

Nanobodies to be oligomerized were ordered from IDT as minigenes incorporating at the 5′ end a SalI site followed by codon optimized sequence for the linker GGGGSGGGGSGGGGSGGGGS (SEQ ID NO: 494) upstream of the start codon of the nanobody cDNA, and at the 3′ end of the nanobody the coding sequence a XhoI site was added. The minigene was cut with SalI and XhoI, the linker-nanobody insert was gel purified and ligated with the XhoI linearized recipient nanobody expression vector (pET21-pelB+nanobody). Restriction digests and sequencing was performed to identify 2×(dimer) and 3×(trimer) oligomers.

Nanobody Screening. To validate nanobody candidates, pelB-fused nanobodies were expressed in 50 ml cultures of Arctic Express (DE3) cells, and the periplasmic fractions were isolated by osmotic shock as previously described (Fridy et al., 2014, Nat Methods 11, 1253-1260). Spike S1-His, RBD, or S2-His proteins (Sino Biological 40591-V08H, 40592-VNAH, and 40590-V08B) were conjugated to cyanogen bromide-activated Sepharose 4 Fast Flow resin (Cytiva) according to the manufacturer's instructions, using 100 μg protein per mg of resin. Periplasm was incubated with 15 μl of the corresponding antigen-conjugated Sepharose for 30 min while rotating at room temperature. The resin was then transferred to a spin column and washed twice with buffer TBT-100 (20 mM HEPES pH 7.4, 100 mM NaCl, 110 mM KOAc, 2 mM MgCl₂, 0.1% Tween 20). Bound protein was eluted with 1.2×NuPAGE LDS sample buffer (Thermo Fisher) for 10 min at 72° C., then reduced with 50 mM DTT (10 min at 72° C.). Input and elution samples were separated by SDS-PAGE and Coomassie-stained bands were quantified using ImageJ software.

Surface Plasmon Resonance (SPR). K_{D}s were determined via surface plasmon resonance experiments. Measurements were either taken on a Proteon XPR36 Protein Interaction Array System (Bio-Rad) or a Biacore 8k (Cytiva). Recombinant spike S1, RBD and spike S2 was immobilized at 5 μg/ml, 5 μg/ml and 12.5 μg/ml respectively using the ProteOn Amine Coupling Kit (EDC/NHS coupling chemistry, Bio-Rad) according to the respective manufacturer's guidelines either on a ProteOn GLC sensor chip or a Series S CM5 sensor chip. All purified nanobodies in a final buffer containing 20 mM HEPES pH 7.4, 150 mM NaCl, 0.02% Tween, were prepared in 5-8 concentrations. For experiments performed on the Proteon XPR36, protein was then injected at 50 μl/min for 120 s, followed by a dissociation time of 600 s. Residual bound proteins were removed by regenerating the chip surface using glycine pH 3+1 M MgCl₂. Data was processed and analyzed using the ProteOn Manager software. For experiments performed on the Biacore 8k, protein was injected at 60 μl/min for 120 s, followed by a dissociation time of either 1200 s or 2400 s. Residual bound proteins were removed by regenerating the chip surface using glycine pH 2.5+1 M MgCl₂. Data was processed and analyzed using the Biacore Insight Evaluation software.

Differential Scanning Fluorimetry (DSF). Nanobody melting temperatures (T_{M}) were measured by differential scanning fluorimetry (DSF) using a CFX96 Real-Time PCR Detection System (Bio-Rad, Hercules, CA). A 96-well thin-wall hard-shell PCR plate (Bio-Rad) was set up with each well containing 10-40 pM of protein in 20 mM HEPES, 150 mM NaCl buffer (pH 7.4), 5×Sypro®Orange Protein Gel Stain (Sigma-Aldrich). Fluorescence variation was measured from 25° C. to 95° C. at a ramp rate of 0.5° C./5 s. Excitation was between 515-535 nm and emission was monitored between 560-580 nm. Tₘ was the transition midpoint value, calculated using the manufacturer's software (Niesen et al., 2007, Nat Protoc 2, 2212-2221).

Lyophilization. Nanobodies in 20 mM HEPES, 150 mM NaCl, pH 7.4 at concentrations between 0.33 mg/ml and 0.63 mg/ml were snap-frozen in liquid nitrogen and dried in a speed-vac to replicate Iyophilization conditions. Nanobodies were then reconstituted in _{dd}H2O and characterized using SPR and DSF.

Epitope Mapping of anti-RBD nanobodies. Biolayer interferometry for epitope binning. Epitope mapping studies were carried out using the Octet system (ForteBio, USA, Version 7) that measures biolayer interferometry (BLI). All steps were performed at 30° C. with shaking at 1300 rpm in a black 96-well plate containing 300 μl kinetics buffer (PBS; 0.2% BSA; 0.02% sodium azide) in each well. AMC-coated biosensors were loaded with mFc tagged RBD (SinoBio) at 40 μg/ml to reach >1 nm wavelength shift following binding and washing. The sensors were then reacted for 300 s with reference nanobodies and then transferred to kinetics buffer-containing wells for another 180 s. A new baseline was set, sensors were then reacted for 180 s with analyte nanobodies (association phase) and then transferred to buffer-containing wells for another 180 s (dissociation phase). Binding and dissociation were measured as changes over time in light interference after subtraction of parallel measurements from unloaded biosensors. Sensorgrams of analyte association/dissociation responses were analyzed using the Octet data analysis software 7.1 (Fortebio, USA, 2015). Analyte binding to mFc RBD was also measured in parallel to get response levels in the absence of the reference nanobodies.

Octet response values were used to compute a Pearson's Correlation Coefficient for pairwise combinations of nanobodies using Pandas (McKinney, 2010, Data Structures for Statistical Computing in Python 10.25080/Majora-92bf1922-00a) in Python 3.7.6 (https://www.python.org/). These coefficients were then used to hierarchically cluster the nanobodies and were visualized as a heatmap (Pedregosa et al., 2011, Journal of Machine Learning Research 12, 2825-2830).

The undirected unweighted network graph of Octet response values was constructed by treating each nanobody as a node, adding an edge to each measured pair of different nanobodies, and setting the maximum response value of a nanobody pair as an attribute to the edge, by using NetworkX 2.5 (https://networkx.org). The least responses of pair-wise nanobodies within all fully measured nanobody subsets were computed by iterating through all network cliques of size 2-14 by using NetworkX's “find_cliques” function, and taking the minimum value of edge attributes within each clique. Network coefficients (average shortest path length, average clustering coefficient and small-world coefficient sigma) were computed using NetworkX's “average_shortest_path_length”, “average_clustering” and “sigma” functions. Network visualization was created by using D3.js (https://d3js.org).

Mass Photometry. Select nanobodies were binned using mass photometry (MP). Experiments were performed on a Refeyn OneMP instrument (Refeyn Ltd). The instrument was calibrated with a mix of BSA (Sigma-Aldrich), thyroglobulin (Sigma-Aldrich) and beta-amylase (Sigma-Aldrich). Coverslips (Thorlabs) and gaskets (Grace Bio-Labs) were prepared by washing with 100% IPA followed by ddH₂O, repeated 3 times, followed by drying with HEPA filtered air. 12 μl of buffer was added to each well to focus the instrument after which 8 μl of protein solution was added and pipetted up and down to briefly mix after which movies/frame acquisition was promptly started. The final concentration in each experiment of recombinant spike S1 monomer (SinoBiological) and each nanobody was 30 nM and between 25-40 nM respectively. Movies were acquired for 60 s (6000 frames) using AcquireMP (version 2.3.0; Refeyn Ltd) using standard settings. All movies were processed, analyzed, and masses estimated by fitting a Gaussian distribution to the data using DiscoverMP (version 2.3.0; Refeyn Ltd).

Epitope Mapping of anti-S2 and non-RBD anti-S1 nanobodies. SPR was utilized to perform epitope binning experiments using a Biacore 8k (Cytiva) supplemented with the Biacore Insight Epitope Binning Extension. All nanobodies concentrations were ≥20×the concentration of their K_{D} for binning experiments, with the majority surpassing their K_{D} by 50×. For non-RBD anti-S1 nanobodies, experiments were performed utilizing either the tandem method or Dual-tandem method for epitope binning, whereas for anti-S2 nanobodies, only the tandem method was utilized. Series S CM5 sensor chips immobilized with spike S1 and spike S2 were used (see Surface Plasmon Resonance section above for full details). For the tandem method, nanobody “1” was injected at 10 μl/min for 240 s, followed by a brief wash, after which nanobody “2” was injected at 10 μl/min for 240 s and dissociated for 30 s. Residual bound proteins were removed by washing the chip surface four times with 10 mM Glycine pH 2+1 M MgCl₂ at 60 μl/min for 60 s. For the Dual-tandem method, nanobody “i” was injected at 10 μl/min for 120 s, followed by nanobody “2”, which was injected at 10 μl/min for 150 s and dissociated for 30 s. Residual bound proteins were removed by washing the chip surface three times with 10 mM Glycine pH 2+1 M MgCl₂ at 60 μl/min for 60 s. For the anti-S2 nanobody binning experiments, residual bound proteins were removed by washing the chip surface first with 0.1 M HCl at 60 μl/min for 60 s, followed by a second wash with 3 M MgCl₂ at 60 μl/min for 60 s. Data was processed and analyzed using the Biacore Insight Evaluation software utilizing the Epitope Binning Extension.

Cell Lines. Vero E6 cells (ATCC) were cultured at 30° C. in the presence of 5% CO2 in medium composed of high glucose Dulbecco's modified Eagle's medium (DMEM, Gibco) supplemented with 5% (v/v) heat inactivated fetal bovine serum (VWR). Cell line authentication was provided by the American Type Culture Collection. 293T/17 and 293T-hACE2 (Crawford et al., 2020, Viruses 12) cells (Life Technologies Cat #R70007, RRID:CVCL_6911) were cultured in DMEM (Gibco) supplemented with 10% FBS, penicillin/streptomycin, 10 mM HEPES, and with 0.1 mM MEM non-essential amino acids (Thermo Fisher). All experiments were performed with cells passaged less than 15 times. Cell cultures for these experiments were not tested for mycoplasma contamination.

Production of SARS-CoV-1, SARS-CoV-2, SARS-CoV-2 Variant Pseudotyped Lentiviral Reporter Particles. Pseudovirus stocks were prepared using a modified protocol published by (Crawford et al., 2020, Viruses 12; Qing et al., 2020, Methods Mol Biol 2099, 9-20). Briefly, pseudovirus stocks were prepared by cotransfecting 4.75 μg pHAGE-CMV-Luc2-IRES-ZsGreen-W (BEI catalog number NR-52516) (Crawford et al., 2020, Viruses 12), 3.75 μg psPAX and 1.5 μg spike containing plasmid using lipofectamine 3000 (Thermofisher). 4×10⁶ cells were plated 16-24 h prior to transfection. 60 h post transfection pseudovirus containing media was collected, cleared by centrifugation at 1000 g and filtered through a 0.45 μm syringe filter to clear debris. 1 ml aliquots were frozen at −80° C. Pseudovirus was titered by 3-fold serial dilution on 293T-hACE2 cells (Crawford et al., 2020, Viruses 12), treated with 2 μg/ml polybrene (Sigma). Infected cells were processed between 52-60 h by adding equal volume of Steady-Glo (Promega) and firefly luciferase signal was measured using the Biotek Model N4 with integration at 0.5 ms.

SARS-CoV-2 Pseudovirus Neutralization Assay. All periplasmic purified nanobodies were treated with Triton X-114 to remove any residual endotoxins so as to not have endotoxins contribute to the effective neutralization. 293-hACE2 cells were plated at 2500-4000 cells per well on 384 solid white TC treated plates. 3-fold serially diluted nanobodies (10 dilutions in total) were incubated with 40,000-60,000 RLU equivalents of pseudotyped SARS-CoV-2-Luc for 1 h at 37° C. Mock treatment, and a sham treatment with LaM2 nanobodies (Fridy et al., 2014, Nat Methods 11, 1253-1260) that do not bind to spike were included as negative controls while untreated wells were used to monitor background levels. The nanobody-pseudovirus mixtures were then added in quadruplicate to 293T-hACE2 cells along with 2 μg/ml polybrene (Sigma). Cells were incubated at 37° C. with 5% CO₂. Infected cells were processed between 52-60 h as described above. Data were processed using Prism7 (graphpad) Sigmoidal, 4PL, X is log(concentration) Least squares fit to calculate the log IC50 (half-maximal inhibitory concentration). All nanobodies were tested at least 2 times and with more than one pseudovirus preparation.

Nanobody Synergy. Experiments were performed as per the Pseudovirus Neutralization Assay. A robotic liquid handler was used to prepare 2D matrices of serial dilutions of two nanobodies and then mix these with SARS-CoV-2 pseudovirus for 1 h. After incubation with the virus, the mixture was overlaid on a monolayer of 293-hACE2 cells and left to incubate for 56 h. Luminescence was quantified as described above. Data was processed using synergy software (Wooten and Albert, 2020, Bioinformatics 37, 1473-1474).

SARS-CoV-2 Stocks and Titers. SARS-Related Coronavirus 2, Isolate USA-WA1/2020, NR-52281, was deposited by the Centers for Disease Control and Prevention and obtained through EEI Resources, NIAID, NIH. Viral stocks were propagated in Vero E6 cells. All experimental work involving live SARS-CoV-2 was performed at Seattle Children's Research Institute (SCRI) in compliance with SCRI guidelines for BioSafety Level 3 (BSL-3) containment. An initial inoculum was diluted in Opti-MEM (Gibco) at 1:1000, overlaid on a monolayer of Vero E6 and incubated for 90 min. Following the incubation the supernatant was removed and replaced with 2% (v/v) FBS in Opti-MEM medium. The cultures were inspected for cytopathic effects, which were prominent after 48 h of infection. After 72 h, infectious supernatants were collected, cleared of cellular debris by centrifugation and stored at −80° C. until use. Viral titers were determined by plaque assay using a liquid overlay and fixation-staining method, as described (Case et al., 2020, Virology 548, 39-48; Mendoza et al., 2020, Curr Protoc Microbiol 57, ecpmcl05). Briefly, serially diluted virus stocks were used to infect confluent monolayers of Vero E6 cells (1.2×10⁶ cells per well) cultured in six-well plates. After a 90 min incubation the virus was removed, and the cell monolayer overlaid with a medium composed of 3% (w/v) carboxymethylcellulose and 4% (v/v) FBS in phenyl-free Opti-MEM. 96 h post infection, the viscous carboxymethylcellulose medium was removed and the cells were washed once with Dulbecco's phosphate buffered saline (DPBS; Gibco) before being fixed with 4% (w/v) paraformaldehyde in DPBS. After a 30 min incubation the fixative was removed, and the cells were rinsed with DBPS before being stained with 1% (w/v) crystal violet in 20% (v/v) ethanol. Contrast was enhanced by successive washes with DPBS and clear plaques representing individual viral infections were visualized as spots lacking the stain. Plaques were enumerated by first identifying the dilution factor of the well containing 10-100 plaques. After counting the plaques, the average number of plaque forming units (pfus) from three experiments was used to determine the viral titer by dividing the average by the dilution factor and volume of virus delivered per well.

Focus Forming Reduction Assay with Authentic SARS-CoV-2. Nanobody neutralization of infectious SARS-CoV-2 was performed using a focus forming reduction assay. Briefly, eight three-fold serial dilutions of nanobodies were incubated with 7.5×10⁴ focus forming units of SARS-CoV-2 for one h at room temperature. The mixture was then added to a confluent monolayer of Vero E6 cells or 293-ACE2 (Crawford et al., 2020, Viruses 12) plated at 1.5×10⁵ cells per well and seeded in 48-well plates. 24 h post infection, the cells were washed once with DPBS, trypsinized with 0.05% trypsin (Gibco) and fixed for 30 min with 4% paraformaldehyde in DPBS. After fixation, the cells were permeabilized with 1% (w/v) Triton X-100 (Sigma Aldrich) for 30 min. After permeabilization, the cells were incubated with a blocking buffer (1% (w/v) bovine serum albumin (Calbiochem) and 0.5% (w/v) Triton X-100 in DBPS) for 60 min, and then stained with primary anti-spike CR3022 (Absolute Antibody) monoclonal antibodies (1:1000), and secondary anti-human IgG antibodies (1:2000) conjugated to Alexa fluor 488 (Invitrogen). Cells staining positive for spike were measured by flow cytometry on a Becton Dickinson BD LSR 11 Special Order System Flow Cytometer With HTS Sampler. The percentage of spike-positive cells from triplicate wells for each dilution was used to determine the half maximal inhibitory concentrations (IC50) using a parametric 1D Hill fitting algorithm with synergy (Wooten and Albert, 2020, Bioinformatics 37, 1473-1474). A mock treatment, sham treatment with LaM2 nanobodies (Fridy et al., 2014, Nat Methods 11, 1253-1260), and untreated cells were used as controls.

Crosslinking Mass Spectrometry. Nanobodies and antigens were incubated together at a 2×molar excess of nanobody at RT for 10 min in 20 mM HEPES pH 7.4 and 150 mM NaCl. Crosslinker was then added to a final concentration of 5 mM bissulfosuccinimidyl suberate (BS3) or 1 mM disuccinimidyl suberate (DSS), and samples were crosslinked for 30 min (RBD, NTD) or 18 min (ectodomain trimer) at RT. Reactions were quenched, reduced and alkylated, and run on an SDS-PAGE gel. The band corresponding to the crosslinked nanobody-antigen complex was then excised from the gel and subjected to in-gel digestion at 37° C. with trypsin (Roche, 1 μg, 4 h) or chymotrypsin (Roche, 0.5 μg, 1.5 h).

Peptides were extracted and analyzed with a nano-LC 1200 (Thermo Fisher) with an EASYspray PepMap RSLC C18 3 micron, 100 Å, 75 μm by 15 cm column coupled to an Orbitrap Fusion Lumos Tribrid mass spectrometer (Thermo Fisher). An Active Background Ion Reduction Device (ABIRD, ESI Source Solutions) was used to reduce background. The Lumos was operated in data-dependent mode, and ions were fragmented by high-energy collisional dissociation (normalized collision energy 28). Separate LC runs were used to analyze the +3 and the +4 through +7 charge states. Higher charge species were prioritized for selection for fragmentation when analyzing the 4-7 species. Orbitrap resolution was 30,000 for MS and 15,000 for MS/MS analyses. The quadrupole isolation window was 1.4, and the MS/MS used a maximum injection time of 800 ms with 4 microscans. Data were then searched by pLink 2.3 (Chen et al., 2019, Nat Commun 10, 3404) to identify cross-linked peptides. The mass accuracy in pLink was set to 10 p.p.m. for MS and 20 p.p.m. for MS/MS. Cysteine carbamidomethylation was included as a fixed modification and methionine oxidation as a variable modification. For trypsin, up to three missed-cleavages were permitted. For chymotrypsin, the enzyme setting was “non-specific.” Spectra were manually checked to ensure correct identifications of crosslinked peptides.
(xi) Closing Paragraphs. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Iie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32).

Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs.

Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y.. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 105 M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Ka of up to 10⁷ M⁻¹, up to 10⁶ M⁻¹, up to 10⁵ M⁻¹. Alternatively, affinity may be defined as an equilibrium dissociation constant (K_{D}) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a KD (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

In particular embodiments, KD can be characterized using BIAcore. For example, in particular embodiments, KD can be measured using surface plasmon resonance assays using a BIACORE®-2000 or a BIACORE®-3000 (BIAcore, Inc., Piscataway, N.J.) at 25° C. with immobilized antigen CM5 chips at 10 response units (RU). Briefly, carboxymethylated dextran biosensor chips (CM5, BIACORE, Inc.) can be activated with N-ethyl-N′-(3-dimethylaminopropyl)-carbodiimide hydrochloride (EDC) and N-hydroxysuccinimide (NHS) according to the supplier's instructions. Antigen can be diluted with 10 mM sodium acetate, pH 4.8, to 5 μg/ml (0.2 μM) before injection at a flow rate of 5 μl/minute to achieve y 10 response units (RU) of coupled protein. Following the injection of antigen, 1 M ethanolamine can be injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of 25 μl/min. Association rates (kₒₙ) and dissociation rates (k_{off}) can be calculated using a simple one-to-one Langmuir binding model (BIACORE® Evaluation Software version 3.2) by simultaneously fitting the association and dissociation sensorgrams. The equilibrium dissociation constant (K_{D}) can be calculated as the ratio k_{off}/kₒₙ. See, e.g., Chen et al., J. Mol. Biol. 293:865-881, 1999. If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; 17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

“Linked to” refers to a coupling that brings at least two components together in proximity to achieve a purpose. Protein linkages can be based on amino acid bonding. Linkages can also be based on ionic or covalent bonding reactions between components of a molecule or conjugate described herein.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A single-domain antibody comprising a set of complementarity determining regions (CDRs) comprising:
(i) a CDR1 having the sequence as set forth in SEQ ID NO: 117, a CDR2 having the sequence as set forth in SEQ ID NO: 118, and a CDRH3 having the sequence as set forth in SEQ ID NO: 119;
(ii) a CDR1 having the sequence as set forth in SEQ ID NO: 120, a CDR2 having the sequence as set forth in SEQ ID NO: 121, and a CDRH3 having the sequence as set forth in SEQ ID NO: 122;
(iii) a CDR1 having the sequence as set forth in SEQ ID NO: 123, a CDR2 having the sequence as set forth in SEQ ID NO: 124, and a CDRH3 having the sequence as set forth in SEQ ID NO: 125;
(iv) a CDR1 having the sequence as set forth in SEQ ID NO: 126, a CDR2 having the sequence as set forth in SEQ ID NO: 127, and a CDRH3 having the sequence as set forth in SEQ ID NO: 128;
(v) a CDR1 having the sequence as set forth in SEQ ID NO: 129, a CDR2 having the sequence as set forth in SEQ ID NO: 130, and a CDRH3 having the sequence as set forth in SEQ ID NO: 131;
(vi) a CDR1 having the sequence as set forth in SEQ ID NO: 132, a CDR2 having the sequence as set forth in SEQ ID NO: 133, and a CDRH3 having the sequence as set forth in SEQ ID NO: 134;
(vii) a CDR1 having the sequence as set forth in SEQ ID NO: 135, a CDR2 having the sequence as set forth in SEQ ID NO: 136, and a CDRH3 having the sequence as set forth in SEQ ID NO: 137;
(viii) a CDR1 having the sequence as set forth in SEQ ID NO: 138, a CDR2 having the sequence as set forth in SEQ ID NO: 139, and a CDRH3 having the sequence as set forth in SEQ ID NO: 140;
(ix) a CDR1 having the sequence as set forth in SEQ ID NO: 141, a CDR2 having the sequence as set forth in SEQ ID NO: 142, and a CDRH3 having the sequence as set forth in SEQ ID NO: 143;
(x) a CDR1 having the sequence as set forth in SEQ ID NO: 144, a CDR2 having the sequence as set forth in SEQ ID NO: 145, and a CDRH3 having the sequence as set forth
(xi) a CDR1 having the sequence as set forth in SEQ ID NO: 147, a CDR2 having the sequence as set forth in SEQ ID NO: 148, and a CDRH3 having the sequence as set forth in SEQ ID NO: 149;
(xii) a CDR1 having the sequence as set forth in SEQ ID NO: 150, a CDR2 having the sequence as set forth in SEQ ID NO: 151, and a CDRH3 having the sequence as set forth in SEQ ID NO: 152;
(xiii) a CDR1 having the sequence as set forth in SEQ ID NO: 153, a CDR2 having the sequence as set forth in SEQ ID NO: 154, and a CDRH3 having the sequence as set forth in SEQ ID NO: 155;
(xiv) a CDR1 having the sequence as set forth in SEQ ID NO: 156, a CDR2 having the sequence as set forth in SEQ ID NO: 157, and a CDRH3 having the sequence as set forth in SEQ ID NO: 158;
(xv) a CDR1 having the sequence as set forth in SEQ ID NO: 159, a CDR2 having the sequence as set forth in SEQ ID NO: 160, and a CDRH3 having the sequence as set forth in SEQ ID NO: 161;
(xvi) a CDR1 having the sequence as set forth in SEQ ID NO: 162, a CDR2 having the sequence as set forth in SEQ ID NO: 163, and a CDRH3 having the sequence as set forth in SEQ ID NO: 164;
(xvii) a CDR1 having the sequence as set forth in SEQ ID NO: 165, a CDR2 having the sequence as set forth in SEQ ID NO: 166, and a CDRH3 having the sequence as set forth in SEQ ID NO: 167;
(xviii) a CDR1 having the sequence as set forth in SEQ ID NO: 168, a CDR2 having the sequence as set forth in SEQ ID NO: 169, and a CDRH3 having the sequence as set forth in SEQ ID NO:170;
(xix) a CDR1 having the sequence as set forth in SEQ ID NO: 171, a CDR2 having the sequence as set forth in SEQ ID NO: 139, and a CDRH3 having the sequence as set forth in SEQ ID NO: 173;
(xx) a CDR1 having the sequence as set forth in SEQ ID NO: 174, a CDR2 having the sequence as set forth in SEQ ID NO: 175, and a CDRH3 having the sequence as set forth in SEQ ID NO: 176;
(xxi) a CDR1 having the sequence as set forth in SEQ ID NO: 177, a CDR2 having the sequence as set forth in SEQ ID NO: 178, and a CDRH3 having the sequence as set forth
(xxii) a CDR1 having the sequence as set forth in SEQ ID NO: 180, a CDR2 having the sequence as set forth in SEQ ID NO: 181, and a CDRH3 having the sequence as set forth in SEQ ID NO: 182;
(xxiii) a CDR1 having the sequence as set forth in SEQ ID NO: 183, a CDR2 having the sequence as set forth in SEQ ID NO: 184, and a CDRH3 having the sequence as set forth in SEQ ID NO: 185;
(xxiv) a CDR1 having the sequence as set forth in SEQ ID NO: 186, a CDR2 having the sequence as set forth in SEQ ID NO: 187, and a CDRH3 having the sequence as set forth in SEQ ID NO: 188;
(xxv) a CDR1 having the sequence as set forth in SEQ ID NO: 189, a CDR2 having the sequence as set forth in SEQ ID NO: 190, and a CDRH3 having the sequence as set forth in SEQ ID NO: 191;
(xxvi) a CDR1 having the sequence as set forth in SEQ ID NO: 192, a CDR2 having the sequence as set forth in SEQ ID NO: 193, and a CDRH3 having the sequence as set forth in SEQ ID NO: 194;
(xxvii) a CDR1 having the sequence as set forth in SEQ ID NO: 195, a CDR2 having the sequence as set forth in SEQ ID NO: 178, and a CDRH3 having the sequence as set forth in SEQ ID NO: 197;
(xxviii) a CDR1 having the sequence as set forth in SEQ ID NO: 198, a CDR2 having the sequence as set forth in SEQ ID NO: 160, and a CDRH3 having the sequence as set forth in SEQ ID NO: 200;
(xxix) a CDR1 having the sequence as set forth in SEQ ID NO: 201, a CDR2 having the sequence as set forth in SEQ ID NO: 202, and a CDRH3 having the sequence as set forth in SEQ ID NO: 203;
(xxx) a CDR1 having the sequence as set forth in SEQ ID NO: 204, a CDR2 having the sequence as set forth in SEQ ID NO: 205, and a CDRH3 having the sequence as set forth in SEQ ID NO: 206;
(xxxi) a CDR1 having the sequence as set forth in SEQ ID NO: 207, a CDR2 having the sequence as set forth in SEQ ID NO: 208, and a CDRH3 having the sequence as set forth in SEQ ID NO: 209;
(xxxii) a CDR1 having the sequence as set forth in SEQ ID NO: 210, a CDR2 having the sequence as set forth in SEQ ID NO: 211, and a CDRH3 having the sequence as set forth
(xxxiii) a CDR1 having the sequence as set forth in SEQ ID NO: 213, a CDR2 having the sequence as set forth in SEQ ID NO: 214, and a CDRH3 having the sequence as set forth in SEQ ID NO: 215;
(xxxiv) a CDR1 having the sequence as set forth in SEQ ID NO: 216, a CDR2 having the sequence as set forth in SEQ ID NO: 217, and a CDRH3 having the sequence as set forth in SEQ ID NO: 218;
(xxxv) a CDR1 having the sequence as set forth in SEQ ID NO: 120, a CDR2 having the sequence as set forth in SEQ ID NO: 220, and a CDRH3 having the sequence as set forth in SEQ ID NO: 221;
(xxxvi) a CDR1 having the sequence as set forth in SEQ ID NO: 222, a CDR2 having the sequence as set forth in SEQ ID NO: 223, and a CDRH3 having the sequence as set forth in SEQ ID NO: 224;
(xxxvii) a CDR1 having the sequence as set forth in SEQ ID NO: 225, a CDR2 having the sequence as set forth in SEQ ID NO: 226, and a CDRH3 having the sequence as set forth in SEQ ID NO: 227;
(xxxviii) a CDR1 having the sequence as set forth in SEQ ID NO: 228, a CDR2 having the sequence as set forth in SEQ ID NO: 229, and a CDRH3 having the sequence as set forth in SEQ ID NO: 230;
(xxxix) a CDR1 having the sequence as set forth in SEQ ID NO: 231, a CDR2 having the sequence as set forth in SEQ ID NO: 232, and a CDRH3 having the sequence as set forth in SEQ ID NO: 233;
(xI) a CDR1 having the sequence as set forth in SEQ ID NO: 234, a CDR2 having the sequence as set forth in SEQ ID NO: 235, and a CDRH3 having the sequence as set forth in SEQ ID NO: 236;
(xIi) a CDR1 having the sequence as set forth in SEQ ID NO: 237, a CDR2 having the sequence as set forth in SEQ ID NO: 238, and a CDRH3 having the sequence as set forth in SEQ ID NO: 239;
(xIii) a CDR1 having the sequence as set forth in SEQ ID NO: 240, a CDR2 having the sequence as set forth in SEQ ID NO: 241, and a CDRH3 having the sequence as set forth in SEQ ID NO: 242;
(xIiii) a CDR1 having the sequence as set forth in SEQ ID NO: 243, a CDR2 having the sequence as set forth in SEQ ID NO: 244, and a CDRH3 having the sequence as set forth
(xIiv) a CDR1 having the sequence as set forth in SEQ ID NO: 246, a CDR2 having the sequence as set forth in SEQ ID NO: 247, and a CDRH3 having the sequence as set forth in SEQ ID NO: 248;
(xIv) a CDR1 having the sequence as set forth in SEQ ID NO: 249, a CDR2 having the sequence as set forth in SEQ ID NO: 250, and a CDRH3 having the sequence as set forth in SEQ ID NO: 251;
(xIvi) a CDR1 having the sequence as set forth in SEQ ID NO: 252, a CDR2 having the sequence as set forth in SEQ ID NO: 253, and a CDRH3 having the sequence as set forth in SEQ ID NO: 254;
(xIvii) a CDR1 having the sequence as set forth in SEQ ID NO: 255, a CDR2 having the sequence as set forth in SEQ ID NO: 256, and a CDRH3 having the sequence as set forth in SEQ ID NO: 257;
(xIviii) a CDR1 having the sequence as set forth in SEQ ID NO: 258, a CDR2 having the sequence as set forth in SEQ ID NO: 259, and a CDRH3 having the sequence as set forth in SEQ ID NO: 260;
(xIix) a CDR1 having the sequence as set forth in SEQ ID NO: 261, a CDR2 having the sequence as set forth in SEQ ID NO: 262, and a CDRH3 having the sequence as set forth in SEQ ID NO: 263;
(I) a CDR1 having the sequence as set forth in SEQ ID NO: 264, a CDR2 having the sequence as set forth in SEQ ID NO: 265, and a CDRH3 having the sequence as set forth in SEQ ID NO: 266;
(Ii) a CDR1 having the sequence as set forth in SEQ ID NO: 129, a CDR2 having the sequence as set forth in SEQ ID NO: 268, and a CDRH3 having the sequence as set forth in SEQ ID NO: 269;
(Iii) a CDR1 having the sequence as set forth in SEQ ID NO: 270, a CDR2 having the sequence as set forth in SEQ ID NO: 271, and a CDRH3 having the sequence as set forth in SEQ ID NO: 272;
(Iiii) a CDR1 having the sequence as set forth in SEQ ID NO: 273, a CDR2 having the sequence as set forth in SEQ ID NO: 274, and a CDRH3 having the sequence as set forth in SEQ ID NO: 275;
(Iiv) a CDR1 having the sequence as set forth in SEQ ID NO: 276, a CDR2 having the sequence as set forth in SEQ ID NO: 277, and a CDRH3 having the sequence as set forth
(Iv) a CDR1 having the sequence as set forth in SEQ ID NO: 279, a CDR2 having the sequence as set forth in SEQ ID NO: 280, and a CDRH3 having the sequence as set forth in SEQ ID NO: 281;
(Ivi) a CDR1 having the sequence as set forth in SEQ ID NO: 282, a CDR2 having the sequence as set forth in SEQ ID NO: 283, and a CDRH3 having the sequence as set forth in SEQ ID NO: 284;
(Ivii) a CDR1 having the sequence as set forth in SEQ ID NO: 285, a CDR2 having the sequence as set forth in SEQ ID NO: 286, and a CDRH3 having the sequence as set forth in SEQ ID NO: 287;
(Iviii) a CDR1 having the sequence as set forth in SEQ ID NO: 288, a CDR2 having the sequence as set forth in SEQ ID NO: 289, and a CDRH3 having the sequence as set forth in SEQ ID NO: 290;
(Iix) a CDR1 having the sequence as set forth in SEQ ID NO: 246, a CDR2 having the sequence as set forth in SEQ ID NO: 292, and a CDRH3 having the sequence as set forth in SEQ ID NO: 293;
(Ix) a CDR1 having the sequence as set forth in SEQ ID NO: 294, a CDR2 having the sequence as set forth in SEQ ID NO: 295, and a CDRH3 having the sequence as set forth in SEQ ID NO: 296;
(Ixi) a CDR1 having the sequence as set forth in SEQ ID NO: 297, a CDR2 having the sequence as set forth in SEQ ID NO: 298, and a CDRH3 having the sequence as set forth in SEQ ID NO: 299;
(Ixii) a CDR1 having the sequence as set forth in SEQ ID NO: 300, a CDR2 having the sequence as set forth in SEQ ID NO: 301, and a CDRH3 having the sequence as set forth in SEQ ID NO: 302;
(Ixiii) a CDR1 having the sequence as set forth in SEQ ID NO: 303, a CDR2 having the sequence as set forth in SEQ ID NO: 304, and a CDRH3 having the sequence as set forth in SEQ ID NO: 305;
(Ixiv) a CDR1 having the sequence as set forth in SEQ ID NO: 306, a CDR2 having the sequence as set forth in SEQ ID NO: 307, and a CDRH3 having the sequence as set forth in SEQ ID NO: 308;
(Ixv) a CDR1 having the sequence as set forth in SEQ ID NO: 309, a CDR2 having the sequence as set forth in SEQ ID NO: 310, and a CDRH3 having the sequence as set forth
(Ixvi) a CDR1 having the sequence as set forth in SEQ ID NO: 312, a CDR2 having the sequence as set forth in SEQ ID NO: 313, and a CDRH3 having the sequence as set forth in SEQ ID NO:314;
(Ixvii) a CDR1 having the sequence as set forth in SEQ ID NO: 315, a CDR2 having the sequence as set forth in SEQ ID NO: 316, and a CDRH3 having the sequence as set forth in SEQ ID NO: 317;
(Ixviii) a CDR1 having the sequence as set forth in SEQ ID NO: 318, a CDR2 having the sequence as set forth in SEQ ID NO: 319, and a CDRH3 having the sequence as set forth in SEQ ID NO: 320;
(Ixix) a CDR1 having the sequence as set forth in SEQ ID NO: 321, a CDR2 having the sequence as set forth in SEQ ID NO: 322, and a CDRH3 having the sequence as set forth in SEQ ID NO: 323;
(Ixx) a CDR1 having the sequence as set forth in SEQ ID NO: 324, a CDR2 having the sequence as set forth in SEQ ID NO: 325, and a CDRH3 having the sequence as set forth in SEQ ID NO: 326;
(Ixxi) a CDR1 having the sequence as set forth in SEQ ID NO: 327, a CDR2 having the sequence as set forth in SEQ ID NO: 328, and a CDRH3 having the sequence as set forth in SEQ ID NO: 329;
(Ixxii) a CDR1 having the sequence as set forth in SEQ ID NO: 330, a CDR2 having the sequence as set forth in SEQ ID NO: 331, and a CDRH3 having the sequence as set forth in SEQ ID NO: 332;
(Ixxiii) a CDR1 having the sequence as set forth in SEQ ID NO: 270, a CDR2 having the sequence as set forth in SEQ ID NO: 193, and a CDRH3 having the sequence as set forth in SEQ ID NO: 335;
(Ixxiv) a CDR1 having the sequence as set forth in SEQ ID NO: 318, a CDR2 having the sequence as set forth in SEQ ID NO: 337, and a CDRH3 having the sequence as set forth in SEQ ID NO: 338;
(Ixxv) a CDR1 having the sequence as set forth in SEQ ID NO: 339, a CDR2 having the sequence as set forth in SEQ ID NO: 340, and a CDRH3 having the sequence as set forth in SEQ ID NO: 341;
(Ixxvi) a CDR1 having the sequence as set forth in SEQ ID NO: 282, a CDR2 having the sequence as set forth in SEQ ID NO: 343, and a CDRH3 having the sequence as set forth
(Ixxvii) a CDR1 having the sequence as set forth in SEQ ID NO: 339, a CDR2 having the sequence as set forth in SEQ ID NO: 346, and a CDRH3 having the sequence as set forth in SEQ ID NO: 347;
(Ixxviii) a CDR1 having the sequence as set forth in SEQ ID NO: 348, a CDR2 having the sequence as set forth in SEQ ID NO: 349, and a CDRH3 having the sequence as set forth in SEQ ID NO: 350;
(Ixxix) a CDR1 having the sequence as set forth in SEQ ID NO: 351, a CDR2 having the sequence as set forth in SEQ ID NO: 352, and a CDRH3 having the sequence as set forth in SEQ ID NO: 353;
(Ixxx) a CDR1 having the sequence as set forth in SEQ ID NO: 354, a CDR2 having the sequence as set forth in SEQ ID NO: 355, and a CDRH3 having the sequence as set forth in SEQ ID NO: 356;
(Ixxxi) a CDR1 having the sequence as set forth in SEQ ID NO: 357, a CDR2 having the sequence as set forth in SEQ ID NO: 358, and a CDRH3 having the sequence as set forth in SEQ ID NO: 359;
(Ixxxii) a CDR1 having the sequence as set forth in SEQ ID NO: 360, a CDR2 having the sequence as set forth in SEQ ID NO: 361, and a CDRH3 having the sequence as set forth in SEQ ID NO: 362;
(Ixxxiii) a CDR1 having the sequence as set forth in SEQ ID NO: 363, a CDR2 having the sequence as set forth in SEQ ID NO: 364, and a CDRH3 having the sequence as set forth in SEQ ID NO: 365;
(Ixxxiv) a CDR1 having the sequence as set forth in SEQ ID NO: 366, a CDR2 having the sequence as set forth in SEQ ID NO: 367, and a CDRH3 having the sequence as set forth in SEQ ID NO: 368;
(Ixxxv) a CDR1 having the sequence as set forth in SEQ ID NO: 369, a CDR2 having the sequence as set forth in SEQ ID NO: 370, and a CDRH3 having the sequence as set forth in SEQ ID NO: 371;
(Ixxxvi) a CDR1 having the sequence as set forth in SEQ ID NO: 372, a CDR2 having the sequence as set forth in SEQ ID NO: 373, and a CDRH3 having the sequence as set forth in SEQ ID NO: 374;
(Ixxxvii) a CDR1 having the sequence as set forth in SEQ ID NO: 375, a CDR2 having the sequence as set forth in SEQ ID NO: 376, and a CDRH3 having the sequence as set forth
(Ixxxviii) a CDR1 having the sequence as set forth in SEQ ID NO: 378, a CDR2 having the sequence as set forth in SEQ ID NO: 379, and a CDRH3 having the sequence as set forth in SEQ ID NO: 380;
(Ixxxix) a CDR1 having the sequence as set forth in SEQ ID NO: 381, a CDR2 having the sequence as set forth in SEQ ID NO: 382, and a CDRH3 having the sequence as set forth in SEQ ID NO: 383;
(xc) a CDR1 having the sequence as set forth in SEQ ID NO: 384, a CDR2 having the sequence as set forth in SEQ ID NO: 385, and a CDRH3 having the sequence as set forth in SEQ ID NO: 386;
(xci) a CDR1 having the sequence as set forth in SEQ ID NO: 387, a CDR2 having the sequence as set forth in SEQ ID NO: 388, and a CDRH3 having the sequence as set forth in SEQ ID NO: 389;
(xcii) a CDR1 having the sequence as set forth in SEQ ID NO: 390, a CDR2 having the sequence as set forth in SEQ ID NO: 391, and a CDRH3 having the sequence as set forth in SEQ ID NO: 392;
(xciii) a CDR1 having the sequence as set forth in SEQ ID NO: 387, a CDR2 having the sequence as set forth in SEQ ID NO: 394, and a CDRH3 having the sequence as set forth in SEQ ID NO: 395;
(xciv) a CDR1 having the sequence as set forth in SEQ ID NO: 396, a CDR2 having the sequence as set forth in SEQ ID NO: 397, and a CDRH3 having the sequence as set forth in SEQ ID NO: 398;
(xcv) a CDR1 having the sequence as set forth in SEQ ID NO: 399, a CDR2 having the sequence as set forth in SEQ ID NO: 400, and a CDRH3 having the sequence as set forth in SEQ ID NO: 401;
(xcvi) a CDR1 having the sequence as set forth in SEQ ID NO: 402, a CDR2 having the sequence as set forth in SEQ ID NO: 403, and a CDRH3 having the sequence as set forth in SEQ ID NO: 404;
(xcvii) a CDR1 having the sequence as set forth in SEQ ID NO: 396, a CDR2 having the sequence as set forth in SEQ ID NO: 406, and a CDRH3 having the sequence as set forth in SEQ ID NO: 407;
(xcviii) a CDR1 having the sequence as set forth in SEQ ID NO: 408, a CDR2 having the sequence as set forth in SEQ ID NO: 409, and a CDRH3 having the sequence as set forth
(xcix) a CDR1 having the sequence as set forth in SEQ ID NO: 411, a CDR2 having the sequence as set forth in SEQ ID NO: 412, and a CDRH3 having the sequence as set forth in SEQ ID NO: 413;
(c) a CDR1 having the sequence as set forth in SEQ ID NO: 414, a CDR2 having the sequence as set forth in SEQ ID NO: 415, and a CDRH3 having the sequence as set forth in SEQ ID NO: 416;
(ci) a CDR1 having the sequence as set forth in SEQ ID NO: 417, a CDR2 having the sequence as set forth in SEQ ID NO: 418, and a CDRH3 having the sequence as set forth in SEQ ID NO: 419;
(cii) a CDR1 having the sequence as set forth in SEQ ID NO: 420, a CDR2 having the sequence as set forth in SEQ ID NO: 421, and a CDRH3 having the sequence as set forth in SEQ ID NO: 422;
(ciii) a CDR1 having the sequence as set forth in SEQ ID NO: 423, a CDR2 having the sequence as set forth in SEQ ID NO: 424, and a CDRH3 having the sequence as set forth in SEQ ID NO: 425;
(civ) a CDR1 having the sequence as set forth in SEQ ID NO: 426, a CDR2 having the sequence as set forth in SEQ ID NO: 427, and a CDRH3 having the sequence as set forth in SEQ ID NO: 428;
(cv) a CDR1 having the sequence as set forth in SEQ ID NO: 429, a CDR2 having the sequence as set forth in SEQ ID NO: 430, and a CDRH3 having the sequence as set forth in SEQ ID NO: 431;
(cvi) a CDR1 having the sequence as set forth in SEQ ID NO: 432, a CDR2 having the sequence as set forth in SEQ ID NO: 433, and a CDRH3 having the sequence as set forth in SEQ ID NO: 434;
(cvii) a CDR1 having the sequence as set forth in SEQ ID NO: 435, a CDR2 having the sequence as set forth in SEQ ID NO: 436, and a CDRH3 having the sequence as set forth in SEQ ID NO: 437;
(cviii) a CDR1 having the sequence as set forth in SEQ ID NO: 438, a CDR2 having the sequence as set forth in SEQ ID NO: 439, and a CDRH3 having the sequence as set forth in SEQ ID NO: 440;
(cix) a CDR1 having the sequence as set forth in SEQ ID NO: 441, a CDR2 having the sequence as set forth in SEQ ID NO: 442, and a CDRH3 having the sequence as set forth
(cx) a CDR1 having the sequence as set forth in SEQ ID NO: 444, a CDR2 having the sequence as set forth in SEQ ID NO: 445, and a CDRH3 having the sequence as set forth in SEQ ID NO: 446;
(cxi) a CDR1 having the sequence as set forth in SEQ ID NO: 429, a CDR2 having the sequence as set forth in SEQ ID NO: 448, and a CDRH3 having the sequence as set forth in SEQ ID NO: 449;
(cxii) a CDR1 having the sequence as set forth in SEQ ID NO: 429, a CDR2 having the sequence as set forth in SEQ ID NO: 451, and a CDRH3 having the sequence as set forth in SEQ ID NO: 452;
(cxiii) a CDR1 having the sequence as set forth in SEQ ID NO: 453, a CDR2 having the sequence as set forth in SEQ ID NO: 454, and a CDRH3 having the sequence as set forth in SEQ ID NO: 455;
(cxiv) a CDR1 having the sequence as set forth in SEQ ID NO: 456, a CDR2 having the sequence as set forth in SEQ ID NO: 457, and a CDRH3 having the sequence as set forth in SEQ ID NO: 458;
(cxv) a CDR1 having the sequence as set forth in SEQ ID NO: 459, a CDR2 having the sequence as set forth in SEQ ID NO: 460, and a CDRH3 having the sequence as set forth in SEQ ID NO: 461; or
(cxvi) a CDR1 having the sequence as set forth in SEQ ID NO: 462, a CDR2 having the sequence as set forth in SEQ ID NO: 463, and a CDRH3 having the sequence as set forth in SEQ ID NO: 464,
each according to Martin numbering.

2. A single-domain antibody comprising
a sequence as set forth in: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, or SEQ ID NO: 525; or
a sequence having at least 90% sequence identity to a sequence as set forth in: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 509, SEQ ID NO: 510, SEQ ID NO: 511, SEQ ID NO: 512, SEQ ID NO: 513, SEQ ID NO: 514, SEQ ID NO: 515, SEQ ID NO: 516, SEQ ID NO: 517, SEQ ID NO: 518, SEQ ID NO: 519, SEQ ID NO: 520, SEQ ID NO: 521, SEQ ID NO: 522, SEQ ID NO: 523, SEQ ID NO: 524, or SEQ ID NO: 525.

3. The single-domain antibody of claim 2, with CDRs as predicted by Kabat, Chothia, Martin, Contact, IMGT, AHo, or North numbering schemes.

4. A fusion protein comprising at least two copies of the single-domain antibody of claim 1 or 2 wherein the at least two copies are joined by a protein linker.

5. The fusion protein of claim 4, wherein the protein linker is a Gly-Ser linker.

6. The fusion protein of claim 5, wherein the Gly-Ser linker is (GlyₓSer_{y})ₙ wherein x and y are independently an integer from 0 to 10 provided that x and y are not both 0 and wherein n is an integer of 1, 2, 3, 4, 5, 6, 7, 8, 9 or 10.

7. The fusion protein of claim 5, wherein the Gly-Ser linker is (Gly₄Ser)₄ (SEQ ID NO: 494).

8. The fusion protein of claim 4, comprising 2, 3, 4, 5, 6, 7, 8, 9, or 10 copies of the single-domain antibody of claim 1 or 2.

9. The fusion protein of claim 8, having two or three copies of S1-23, S1-49, S1-RBD-35, or S2-7.

10. A single-domain antibody of claim 1 or 2, wherein the single-domain antibody is multimerized.

11. The single-domain antibody of claim 10, wherein the multimerized single-domain antibody is a dimer, trimer, tetramer, pentamer, hexamer, or heptamer.

12. The single-domain antibody of claim 10, wherein the multimerized single-domain antibody is a dimer or trimer of S1-23, S1-49, S1-RBD-35, or S2-7.

13. A heavy chain only antibody (HcAb) comprising the single-domain antibody of claim 1 or 2 linked to an Fc region of an antibody.

14. The HcAb of claim 13, wherein the Fc region is an IgG Fc region, an IgA Fc region, an IgM Fc region, an IgD Fc region or an IgE Fc region.

15. The HcAb of claim 13, wherein the Fc region is an IgA Fc region, an IgM Fc region, or an IgG Fc region.

16. The HcAb of claim 13, wherein the Fc region is an IgM Fc region having the sequence as set forth in SEQ ID NOs: 470-480.

17. The HcAb of claim 13, wherein the Fc region is an IgA Fc region having the sequence as set forth in SEQ ID NOs: 466 and 467.

18. The HcAb of claim 13, wherein the Fc region is an IgG Fc region having the sequence as set forth in SEQ ID NOs: 465 and 587-589.

19. The HcAb of claim 13, wherein the Fc region comprises a multimerizing fragment of the IgM Fc region or a multimerizing fragment of the IgA Fc region.

20. The HcAb claim 19, wherein the multimerizing fragment of the IgM Fc region comprises the IgM tailpiece.

21. The HcAb of claim 19, wherein the multimerizing fragment of the IgM Fc region comprises the Cμ4 domain and the IgM tailpiece.

22. The HcAb of claim 19, wherein the multimerizing fragment of the IgM Fc region comprises the Cμ3 domain, the Cμ4 domain, and the IgM tailpiece.

23. The HcAb of claim 19, wherein the multimerizing fragment of the IgM Fc region comprises the Cμ2 domain, the Cμ3 domain, the Cμ4 domain, and the IgM tailpiece.

24. The HcAb of claim 19, wherein the multimerizing fragment of the IgM Fc region comprises the Cμ1 domain, the Cμ2 domain, the Cμ3 domain, the Cμ4 domain, and the IgM tailpiece.

25. The HcAb of claim 19, wherein the multimerizing fragment of the IgM Fc region has the sequence as set forth in SEQ ID NO: 669.

26. The HcAb of claim 19, wherein the multimerizing fragment of the IgA Fc region comprises the IgA tailpiece.

27. The HcAb of claim 26, wherein the IgA tailpiece has the sequence of residues 331-352 as set forth in SEQ ID NO: 644 or the sequence of residues 318-340 as set forth in SEQ ID NO: 645.

28. The HcAb of claim 19, wherein the multimerizing fragment of the IgA Fc region comprises the IgA CA3 domain and the IgA tailpiece.

29. The HcAb of claim 19, wherein the multimerizing fragment of the IgA Fc region comprises the IgA CA2 domain, the IgA CA3 domain, and the IgA tailpiece.

30. The HcAb of claim 19, wherein the multimerizing fragment of the IgA Fc region comprises the IgA CA1 domain, the IgA CA2 domain, the IgA CA3 domain, and the IgA tailpiece.

31. The HcAb of claim 13, wherein the Fc region comprises an IgG Fc region and a multimerizing fragment of the IgM Fc region or a multimerizing fragment of the IgA Fc region.

32. The HcAb of claim 31, wherein the multimerizing fragment of the IgM Fc region or the multimerizing fragment of the IgA Fc region is fused to the C-terminus of the IgG Fc region.

33. The HcAb of claim 31, wherein the IgG is IgG1, IgG2, IgG3 or IgG4.

34. The HcAb of claim 31, wherein the IgG Fc region and the IgM Fc region have the sequence as set forth in SEQ ID NO: 472.

35. The HcAb of claim 13, further comprising a J-chain.

36. The HcAb of claim 35, wherein the J-chain has the sequence as set forth in SEQ ID NOs: 482-488.

37. The HcAb of claim 35, wherein the binding domain is introduced into the J-chain.

38. The HcAb of claim 13, formatted into a multimer.

39. The HcAb of claim 39, wherein the multimer is an IgM multimer, an IgA multimer, or an IgG multimer.

40. The HcAb of claim 39, wherein the multimer is an IgA dimer or an IgM pentamer or hexamer.

41. The HcAb of claim 40, wherein the IgA dimer comprises the IgA tail piece.

42. The HcAb of claim 40, wherein the IgM pentamer or hexamer comprises the IgM tail piece.

43. The HcAb of claim 40, wherein the IgA dimer comprises a J chain.

44. The HcAb of claim 40, wherein the IgM pentamer comprises a J chain.

45. The HcAb of claim 40, wherein the IgM hexamer comprises a J chain.

46. The HcAb of claim 13, wherein the Fc region comprises a modification to produce an administration benefit.

47. The HcAb of claim 46, wherein the administration benefit comprises reduced susceptibility to proteolysis, reduced susceptibility to oxidation, altered binding affinity for forming protein complexes, altered binding affinities, reduced immunogenicity, and/or extended half-life.

48. The HcAb of claim 47, wherein the administration benefit is extended half-life and the modification comprises M252Y, T252L, T253S, S254T, T254F, T256E, T256N, E294delta, T307P, A379V, S383N, M428L, N434S, N434A, N434Y, and/or R435H.

49. The HcAb of claim 47, wherein the administration benefit is extended half-life and the modification is R435H; N434A; E294delta; M428L/N434S; M252Y/S254T/T256E; T252L/T253S/T254F; E294delta/T307P/N434Y; or T256N/A379V/S383N/N434Y.

50. A multi-specific binding molecule comprising at least two binding domains that bind distinct epitopes wherein at least one binding domain comprises a single-domain antibody of claim 1 or 2 or an HcAb of claim 13.

51. The multi-specific binding molecule of claim 50, wherein all binding domains of the multi-specific binding molecule bind a SARS-CoV-2 spike protein epitope.

52. The multi-specific binding molecule of claim 50, wherein all binding domains of the multi-specific binding molecule bind distinct SARS-CoV-2 spike protein epitopes.

53. The multi-specific binding molecule of claim 52, wherein the distinct SARS-CoV-2 spike protein epitopes are within the S1-non-RBD, RBD, or S2.

54. The multi-specific binding molecule of claim 50, wherein
at least one binding domain binds an S1-non-RBD epitope and at least one binding domain binds an S1-non-RBD epitope, an RBD epitope, or an S2 epitope,
at least one binding domain binds an RBD epitope and at least one binding domain binds an RBD epitope, an S1-non-RBD epitope, or an S2 epitope, or wherein
at least one binding domain binds an S2 epitope and at least one binding domain binds an S2 epitope, an RBD epitope, or an S1-non-RBD epitope.

55. The multi-specific binding molecule of claim 50, wherein at least one binding domain comprises S1-23.

56. The multi-specific binding molecule of claim 50, wherein at least one binding domain comprises S1-1, S1-27, S1-RBD-15, or S2-40.

57. The multi-specific binding molecule of claim 50, wherein at least one binding domain comprises S1-23 and at least one binding domain comprises S1-1, S1-27, S1-RBD-15, or S2-40.

58. The multi-specific binding molecule of claim 50, wherein at least one binding domain comprises S1-48 and at least one binding domain comprises S1-RBD-15.

59. The multi-specific binding molecule of claim 50, wherein the binding domains comprise a combination comprising S1-2 and S2-1; S1-3 and S2-2; S1-7 and S2-3; S1-9 and S2-4; S1-10 and S2-5; S1-11 and S2-6; S1-17 and S2-7; S1-24 and S2-9; S1-25 and S2-10; S1-30 and S2-11; S1-32 and S2-13; S1-41 and S2-14; S1-49 and S2-15; S1-50 and S2-18; S1-58 and S2-22; S1-60 and S2-26; S1-64 and S2-33; S1-65 and S2-35; S1-66 and S2-36; S1-24 and S2-39; S1-32 and S2-40; S1-60 and S2-42; S1-7 and S2-47; S1-11 and S2-57; S1-3 and S2-59; S1-66 and S2-62; S1-2 and S1-1; S1-3 and S1-4; S1-7 and S1-5; S1-9 and S1-6; S1-10 and S1-12; S1-11 and S1-14; S1-17 and S1-19; S1-24 and S1-20; S1-25 and S1-21; S1-30 and S1-23; S1-32 and S1-27; S1-41 and S1-28; S1-49 and S1-29; S1-50 and S1-31; S1-58 and S1-35; S1-60 and S1-36; S1-64 and S1-37; S1-65 and S1-38; S1-66 and S1-39; S1-2 and S1-46; S1-3 and S1-48; S1-7 and S1-51; S1-9 and S1-52; S1-10 and S1-53; S1-11 and S1-54; S1-17 and S1-55; S1-24 and S1-56; S1-25 and S1-61; S1-30 and S1-62; S1-32 and S1-63; S1-41 and S1-RBD-3; S1-49 and S1-RBD-4; S1-50 and S1-RBD-5; S1-58 and S1-RBD-6; S1-60 and S1-RBD-9; S1-64 and S1-RBD-10; S1-65 and S1-RBD-11; S1-66 and S1-RBD-12; S1-2 and S1-RBD-14; S1-3 and S1-RBD-15; S1-7 and S1-RBD-16; S1-9 and S1-RBD-18; S1-10 and S1-RBD-19; S1-11 and S1-RBD-20; S1-17 and S1-RBD-21; S1-24 and S1-RBD-22; S1-25 and S1-RBD-23; S1-30 and S1-RBD-24; S1-32 and S1-RBD-25; S1-41 and S1-RBD-26; S1-49 and S1-RBD-27; S1-50 and S1-RBD-28; S1-58 and S1-RBD-29; S1-60 and S1-RBD-30; S1-64 and S1-RBD-32; S1-65 and S1-RBD-34; S1-66 and S1-RBD-35; S1-2 and S1-RBD-36; S1-3 and S1-RBD-37; S1-7 and S1-RBD-38; S1-9 and S1-RBD-39; S1-10 and S1-RBD-40; S1-11 and S1-RBD-41; S1-17 and S1-RBD-43; S1-24 and S1-RBD-44; S1-25 and S1-RBD-45; S1-30 and S1-RBD-46; S1-32 and S1-RBD-47; S1-41 and S1-RBD-48; S1-49 and S1-RBD-49; S1-50 and S1-RBD-51; S2-1 and S1-1; S2-2 and S1-4; S2-3 and S1-5; S2-4 and S1-6; S2-5 and S1-12; S2-6 and S1-14; S2-7 and S1-19; S2- 9 and S1-20; S2-10 and S1-21; S2-11 and S1-23; S2-13 and S1-27; S2-14 and S1-28; S2-15 and S1-29; S2-18 and S1-31; S2-22 and S1-35; S2-26 and S1-36; S2-33 and S1-37; S2-35 and S1-38; S2-36 and S1-39; S2-39 and S1-46; S2-40 and S1-48; S2-42 and S1-51; S2-47 and S1-52; S2-57 and S1-53; S2-59 and S1-54; S2-62 and S1-55; S2-1 and S1-56; S2-2 and S1-61; S2-3 and S1-62; S2-4 and S1-63; S2-5 and S1-RBD-3; S2-6 and S1-RBD-4; S2-7 and S1-RBD-5; S2-9 and S1-RBD-6; S2-10 and S1-RBD-9; S2-11 and S1-RBD-10; S2-13 and S1-RBD-11; S2-14 and S1-RBD-12; S2-15 and S1-RBD-14; S2-18 and S1-RBD-15; S2-22 and S1-RBD-16; S2-26 and S1-RBD-18; S2-33 and S1-RBD-19; S2-35 and S1-RBD-20; S2-36 and S1-RBD-21; S2-39 and S1-RBD-22; S2-40 and S1-RBD-23; S2-42 and S1-RBD-24; S2-47 and S1-RBD-25; S2-57 and S1-RBD-26; S2-59 and S1-RBD-27; S2-62 and S1-RBD-28; S2-1 and S1-RBD-29; S2-2 and S1-RBD-30; S2-3 and S1-RBD-32; S2-4 and S1-RBD-34; S2-5 and S1-RBD-35; S2-6 and S1-RBD-36; S2-7 and S1-RBD-37; S2-9 and S1-RBD-38; S2-10 and S1-RBD-39; S2-11 and S1-RBD-40; S2-13 and S1-RBD-41; S2-14 and S1-RBD-43; S2-15 and S1-RBD-44; S2-18 and S1-RBD-45; S2-22 and S1-RBD-46; S2-26 and S1-RBD-47; S2-33 and S1-RBD-48; S2-35 and S1-RBD-49; S2-36 and S1-RBD-51.S1-2, S2-14 and S1-52; S1-3, S2-15 and S1-53; S1-7, S2-18 and S1-54; S1-9, S2-22 and S1-55; S1-10, S2-26 and S1-56; S1-11, S2-33 and S1-61; S1-17, S2-35 and S1-62; S1-24, S2-36 and S1-63; S1-25, S2-39 and S1-RBD-3; S1-30, S2-40 and S1-RBD-4; S1-32, S2-42 and S1-RBD-5; S1-41, S2-47 and S1-RBD-6; S1-49, S2-57 and S1-RBD-9; S1-50, S2-59 and S1-RBD-10; S1-58, S2-62 and S1-RBD-11; S1-60, S2-1 and S1-RBD-12; S1-64, S2-2 and S1-RBD-14; S1-65, S2-3 and S1-RBD-15; S1-66, S2-4 and S1-RBD-16; S1-2, S2-5 and S1-RBD-18; S1-3, S2-6 and S1-RBD-19; S1-7, S2-7 and S1-RBD-20; S1-9, S2-9 and S1-RBD-21; S1-10, S2-10 and S1-RBD-22; S1-11, S2-11 and S1-RBD-23; S1-17, S2-13 and S1-RBD-24; S1-24, S2-14 and S1-RBD-25; S1-25, S2-15 and S1-RBD-26; S1-30, S2-18 and S1-RBD-27; S1-32, S2-22 and S1-RBD-28; S1-41, S2-26 and S1-RBD-29; S1-49, S2-33 and S1-RBD-30; S1-50, S2-35 and S1-RBD-32; S1-58, S2-36 and S1-RBD-34; S1-60, S2-39 and S1-RBD-35; S1-64, S2-40 and S1-RBD-36; S1-65, S2-42 and S1-RBD-37; S1-66, S2-47 and S1-RBD-38; S1-2, S2-57 and S1-RBD-39; S1-3, S2-59 and S1-RBD-40; S1-7, S2-62 and S1-RBD-41; S1-9, S2-1 and S1-RBD-43; S1-10, S2-2 and S1-RBD-44; S1-11, S2-3 and S1-RBD-45; S1-17, S2-4 and S1-RBD-46; S1-24, S2-5 and S1-RBD-47; S1-25, S2-6 and S1-RBD-48; S1-30, S2-7 and S1-RBD-49; S1-32, S2-9 and S1-RBD-51; S1-41, S2-10 and S1-1; S1-49, S2-11 and S1-4; S1-50, S2-13 and S1-20; S1-58, S2-14 and S1-21; S1-60, S2-15 and S1-23; S1-64, S2-18 and S1-27; S1-65, S2-1 and S1-28; S1-66, S2-2 and S1-29; S1-2, S2-3 and S1-31; S1-3, S2-4 and S1-35; S1-7, S2-5 and S1-36; S1-9, S2-6 and S1-37; S1-10, S2-7 and S1-38; S1-11, S2-9 and S1-39; S1-17, S2-10 and S1-46; S1-24, S2-11 and S1-48; or S1-25, S2-13 and S1-51.

60. The multi-specific binding molecule of claim 50, wherein a subset of binding domains of the multi-specific binding molecule bind a secondary respiratory virus.

61. The multi-specific binding molecule of claim 60, wherein the secondary respiratory virus comprises a human adenovirus, human boca virus (HBoV), human coronavirus other than SARS-CoV-2, influenza virus, human parainfluenza virus, or human rhinovirus.

62. The multi-specific binding molecule of claim 60, wherein the binding domain that binds the secondary respiratory virus is derived from 8C4, 5Hx-I, 5Hx-2, 5Hx-3, 5Hx-4, 5Hx-5, 5.100K-1, 5PB-1, 5Fb-1, 1E11, EPR23305-44, 47D11, CR3022, CDC2-A2, G2, 5F9, FIB-H1, JC57-13, 32D6, CH65, CR9114, MAb 22/1, MAb70/I, MAb 110/1, MAb 264/2, MAb W18/1, MAb 14/3, MAb 24/4, MAb 47/8, MAb 198/2, MAb 215/2, H2/6A5, H3/4C4, H2/6C4, H2/4B3, H9/B20, H2/4B1, CA6261, 6F12, CR9114, PEG-1, CR8033, CR8071, 113/2, 124/4, 128/2, 134/1, 146/1, 152/2, 160/1, 162/1, 195/3, 206/2, 238/4, 280/2, PAR2, or TCN-711.

63. The multi-specific binding molecule of claim 50, wherein the multi-specific binding molecule comprises an immune cell engaging molecule.

64. The multi-specific binding molecule of claim 63, wherein the immune cell engaging molecule activates a B cell, T cell, natural killer (NK) cell, or macrophage.

65. The multi-specific binding molecule of claim 63, wherein a binding domain of the immune cell engaging molecule binds CD2, CD3, CD7, CD8, CD27, CD28, CD30, CD40, CD83, 4-1BB (CD137), OX40, lymphocyte function-associated antigen-1 (LFA-1), LIGHT, NKG2C, or B7-H3.

66. The multi-specific binding molecule of claim 65, wherein the binding domain binds CD3 and is derived from the OKT3 antibody or TR66.

67. The multi-specific binding molecule of claim 65, wherein the binding domain binds CD28 and is derived from CD80, CD86, the 9D7 antibody, 9.3 antibody, KOLT-2 antibody, 15E8 antibody, 248.23.2 antibody, or EX5.3D10 antibody.

68. The multi-specific binding molecule of claim 65, wherein the binding domain binds 4-1 BB and comprises: a light chain variable domain comprising a CDRL1 sequence as set forth in SEQ ID NO: 504, a CDRL2 sequence as set forth in SEQ ID NO: 505, and a CDRL3 sequence as set forth in SEQ ID NO: 506; and a heavy chain variable domain comprising a CDRH1 sequence as set forth in SEQ ID NO: 507, a CDRH2 sequence of INH, and a CDRH3 sequence as set forth in SEQ ID NO: 508.

69. The multi-specific binding molecule of claim 65, wherein the binding domain binds CD8 and is derived from the OKT8 antibody or SK1 antibody.

70. The multi-specific binding molecule of claim 63, wherein a binding domain of the immune cell engaging molecule binds an NK cell receptor.

71. The multi-specific binding molecule of claim 70, wherein the binding domain binds:
NKG2D and is derived from the 5C6 antibody or 1D11 antibody;
KIR2DL4 and is derived from mAb 33;
NKp44 and is derived from the P44-8 antibody;
CD8 and is derived from the OKT8 antibody or the SK1 antibody; or
CD16 and is derived from the 3G8 antibody.

72. The multi-specific binding molecule of claim 50, wherein a binding domain of the multi-specific binding molecule binds human albumin.

73. The multi-specific binding molecule of claim 72, wherein a binding domain comprises a single domain antibody of claim 1 and one binding domain comprises a single domain antibody that binds human albumin.

74. The multi-specific binding molecule of claim 72, wherein the binding domain that binds albumin comprises CA645.

75. The multi-specific binding molecule of claim 72, wherein the binding domain that binds albumin has the sequence as set forth in SEQ ID NO: 594 or SEQ ID NO: 595.

76. The multi-specific binding molecule of claim 75, wherein the binding domain that binds albumin comprises a sequence with at least 95% sequence identity to SEQ ID NO: 594 or SEQ ID NO: 595.

77. The multi-specific binding molecule of claim 75, wherein the binding domain that binds albumin comprises a sequence with 98% sequence identity to SEQ ID NO: 594 or SEQ ID NO: 595.

78. A conjugate comprising a single-domain antibody of claim 1 or 2 or an HcAb of claim 13 linked to an immunotoxin, a drug, a radioisotope, a detectable label, or a microparticle.

79. The conjugate of claim 78, comprising an immunotoxin.

80. The conjugate of claim 79, wherein the immunotoxin comprises a plant toxin or bacterial toxin.

81. The conjugate of claim 80, wherein the plant toxin comprises ricin, abrin, mistletoe lectin, modeccin, pokeweed antiviral protein, saporin, Bryodin 1, bouganin, or gelonin.

82. The conjugate of claim 80, wherein the bacterial toxin comprises diphtheria toxin, or Pseudomonas exotoxin.

83. The conjugate of claim 78, comprising a drug.

84. The conjugate of claim 83, wherein the drug comprises a cytotoxic drug.

85. The conjugate of claim 84, wherein the cytotoxic drug comprises actinomycin D, auristatin, camptothecin, colchicin, daunorubicin, dihydroxy dolastatin, doxorubicin, duocarmycin, emetine, etoposide, gramicidin D, glucocorticoids, maytansinoid mithramycin, mitomycin, nemorubicin, propranolol, puromycin, taxane, taxol, tetracaine, trichothecene, vinblastine, vinca alkaloid, or vincristine.

86. The conjugate of claim 78, comprising a radioisotope.

87. The conjugate of claim 86, wherein the radioisotope comprises ²²⁵Ac, ²²⁷Th, ²²⁸Ac, ¹²⁴Am, ²¹¹As, ¹⁹⁴Au, ⁷Be, ²⁴⁵Bk, ⁷⁶Br, ¹¹C, ²⁵⁴Cf, ²⁴²Cm ⁵¹Cr, ⁶⁷Cu, ¹⁵³Dy, ¹⁷¹Er, ²⁵⁰Es, ¹⁴⁷Eu, ⁵²Fe, ²⁵¹Fm, ⁶⁶Ga, ¹⁴⁶Gd, ⁶⁸Ge, ¹⁷⁰Hf, ¹⁹³Hg, ¹³¹I, ¹⁸⁵Ir, ⁴²K, ⁷⁹Kr, ¹³²La, ²⁶²Lr, ¹⁶⁹Lu, ²⁶⁰Md, ⁵²Mn, ⁹⁰Mo, ²⁴Na, ⁹⁵Nb, ¹³⁸Nd, ⁵⁷Ni, ¹⁵O, ¹⁸²Os, ³²P, ²⁰¹Pb, ¹⁰¹Pd, ¹⁴³Pr, ¹⁹¹Pt, ²⁴³Pu, ²²⁵Ra, ⁸¹Rb, ¹⁸⁸Re, ¹⁰⁵Rh, ²¹¹Rn, ¹⁰³Ru, ³⁵S, ⁴⁴Sc, ⁷²Se, ¹⁵³Sm, ¹²⁵Sn, ⁹¹Sr, ¹⁷³Ta, ¹⁵⁴Tb, ¹²⁷Te, ²³⁴Th, ⁴⁵Ti, ¹⁶⁶Tₘ, ²³⁰U, ⁴⁸V, ¹⁷⁸W, ¹²⁵Xe, ⁸⁶Y, ¹⁶⁹Yb, ⁶⁵Zn, ⁹⁵Zr, or ⁹⁷Zr.

88. The conjugate of claim 78, comprising a detectable label.

89. The conjugate of claim 88, wherein the detectable label comprises a chemiluminescent label, a spectral colorimetric label, an affinity tag, an enzymatic label, or a fluorescent label.

90. The conjugate of claim 78, comprising a microparticle.

91. The conjugate of claim 90, wherein the microparticle comprises a latex bead, a polystyrene bead, a fluorescent bead, or a colored bead.

92. A chimeric antigen receptor (CAR) that, when expressed by a cell, comprises an extracellular component linked to an intracellular component by a transmembrane domain, wherein the extracellular component comprises a single-domain antibody of claim 1 or 2.

93. The CAR of claim 92, wherein the single-domain antibody of claim 1 or 2 comprises S1-1, S1-6, S1-23, S1-27, S1-37, S1-39, S1-48, S1-49, S1-RBD-5, S1-RBD-6, S1-RBD-9, S1-RBD-11, S1-RBD-15, S1-RBD-21, S1-RBD-22, S1-RBD-35, S2-7, S2-10, or S2-40.

94. The CAR of claim 92, wherein the intracellular component comprises an effector domain comprising: 4-1BB (CD137), CD3γ, CD3δ, CD3ε, CD3ζ, CD27, CD28, DAP10, ICOS, LAG3, NKG2D, NOTCH1, OX40, ROR2, SLAMF1, TCRα, TCRβ, TRIM, Wnt, Zap70, or a combination thereof.

95. The CAR of claim 92, wherein the transmembrane domain comprises a transmembrane region of: the α, β or ζ chain of a T-cell receptor; CD28; CD27; CD3; CD45; CD4; CD5; CD8; CD9; CD16; CD22; CD33; CD37; CD64; CD80; CD86; CD134; CD137; CD154; or a combination thereof.

96. The CAR of claim 92, wherein the CAR further comprises a spacer region.

97. A cell genetically modified to express a single-domain antibody of claim 1 or 2, an HcAb of claim 13, or the CAR of claim 92.

98. The cell of claim 97, wherein the cell is an immune cell.

99. The cell of claim 98, wherein the immune cell is a T cell, B cell, natural killer cell, or macrophage.

100. The cell of claim 97, wherein the cell is a bacterial cell, an insect cell, or a yeast cell.

101. A composition comprising a single-domain antibody of claim 1 or 2, an HcAb of claim 13, or a cell of claim 97 for administration to a subject.

102. The composition of claim 101, further comprising a pharmaceutically acceptable carrier.

103. The composition of claim 101, wherein the single-domain antibody of claim 1 or 2 comprises S1-1, S1-6, S1-23, S1-27, S1-37, S1-39, S1-48, S1-49, S1-RBD-5, S1-RBD-6, S1-RBD-9, S1-RBD-11, S1-RBD-15, S1-RBD-21, S1-RBD-22, S1-RBD-35, S2-7, S2-10, or S2-40.

104. The composition of claim 101, comprising a fusion protein having two or three copies of S1-23, S1-49, S1-RBD-35, or S2-7.

105. The composition of claim 101, comprising a multimerized dimer or trimer of S1-23, S1-49, S1-RBD-35, or S2-7

106. The composition of claim 101, wherein the composition comprises at least two single-domain antibodies of claim 1 or 2.

107. The composition of claim 106, wherein the at least two single-domain antibodies bind distinct epitopes.

108. The composition of claim 106, wherein
one of the at least two single-domain antibodies binds an S1-non-RBD epitope and at least one of the at least two single-domain antibodies binds an S1-non-RBD epitope, an RBD epitope, or an S2 epitope,
one of the at least two single-domain antibodies binds an RBD epitope and at least one of the at least two single-domain antibodies binds an RBD epitope, an S1-non-RBD epitope, or an S2 epitope, or wherein
one of the at least two single-domain antibodies binds an S2 epitope and at least one of the at least two single-domain antibodies binds an S2 epitope, an RBD epitope, or an S1-non-RBD epitope.

109. The composition of claim 106, wherein the at least two single-domain antibodies comprise S1-23 and S1-1, S1-27, S1-RBD-15, or S2-40.

110. The composition of claim 106, wherein at least one single-domain antibody comprises S1-23 and at least one single-domain antibody comprises S1-1, S1-27, S1-RBD-15, or S2-40.

111. The composition of claim 106, wherein at least one single-domain antibody comprises S1-48 and at least one single-domain antibody comprises S1-RBD-15.

112. The composition of claim 106, comprising S1-2 and S2-1; S1-3 and S2-2; S1-7 and S2-3; S1-9 and S2-4; S1-10 and S2-5; S1-11 and S2-6; S1-17 and S2-7; S1-24 and S2-9; S1-25 and S2-10; S1-30 and S2-11; S1-32 and S2-13; S1-41 and S2-14; S1-49 and S2-15; S1-50 and S2-18; S1-58 and S2-22; S1-60 and S2-26; S1-64 and S2-33; S1-65 and S2-35; S1-66 and S2-36; S1-24 and S2-39; S1-32 and S2-40; S1-60 and S2-42; S1-7 and S2-47; S1-11 and S2-57; S1-3 and S2-59; S1-66 and S2-62; S1-2 and S1-1; S1-3 and S1-4; S1-7 and S1-5; S1-9 and S1-6; S1- 10 and S1-12; S1-11 and S1-14; S1-17 and S1-19; S1-24 and S1-20; S1-25 and S1-21; S1-30 and S1-23; S1-32 and S1-27; S1-41 and S1-28; S1-49 and S1-29; S1-50 and S1-31; S1-58 and S1-35; S1-60 and S1-36; S1-64 and S1-37; S1-65 and S1-38; S1-66 and S1-39; S1-2 and S1-46; S1-3 and S1-48; S1-7 and S1-51; S1-9 and S1-52; S1-10 and S1-53; S1-11 and S1-54; S1-17 and S1-55; S1-24 and S1-56; S1-25 and S1-61; S1-30 and S1-62; S1-32 and S1-63; S1-41 and S1-RBD-3; S1-49 and S1-RBD-4; S1-50 and S1-RBD-5; S1-58 and S1-RBD-6; S1-60 and S1-RBD-9; S1-64 and S1-RBD-10; S1-65 and S1-RBD-11; S1-66 and S1-RBD-12; S1-2 and S1-RBD-14; S1-3 and S1-RBD-15; S1-7 and S1-RBD-16; S1-9 and S1-RBD-18; S1-10 and S1-RBD-19; S1-11 and S1-RBD-20; S1-17 and S1-RBD-21; S1-24 and S1-RBD-22; S1-25 and S1-RBD-23; S1-30 and S1-RBD-24; S1-32 and S1-RBD-25; S1-41 and S1-RBD-26; S1-49 and S1-RBD-27; S1-50 and S1-RBD-28; S1-58 and S1-RBD-29; S1-60 and S1-RBD-30; S1-64 and S1-RBD-32; S1-65 and S1-RBD-34; S1-66 and S1-RBD-35; S1-2 and S1-RBD-36; S1-3 and S1-RBD-37; S1-7 and S1-RBD-38; S1-9 and S1-RBD-39; S1-10 and S1-RBD-40; S1-11 and S1-RBD-41; S1-17 and S1-RBD-43; S1-24 and S1-RBD-44; S1-25 and S1-RBD-45; S1-30 and S1-RBD-46; S1-32 and S1-RBD-47; S1-41 and S1-RBD-48; S1-49 and S1-RBD-49; S1-50 and S1-RBD-51; S2-1 and S1-1; S2-2 and S1-4; S2-3 and S1-5; S2-4 and S1-6; S2-5 and S1-12; S2-6 and S1-14; S2-7 and S1-19; S2-9 and S1-20; S2-10 and S1-21; S2-11 and S1-23; S2-13 and S1-27; S2-14 and S1-28; S2-15 and S1-29; S2-18 and S1-31; S2-22 and S1-35; S2-26 and S1-36; S2-33 and S1-37; S2-35 and S1-38; S2-36 and S1-39; S2-39 and S1-46; S2-40 and S1-48; S2-42 and S1-51; S2-47 and S1-52; S2-57 and S1-53; S2-59 and S1-54; S2-62 and S1-55; S2-1 and S1-56; S2-2 and S1-61; S2-3 and S1-62; S2-4 and S1-63; S2-5 and S1-RBD-3; S2-6 and S1-RBD-4; S2-7 and S1-RBD-5; S2-9 and S1-RBD-6; S2-10 and S1-RBD-9; S2-11 and S1-RBD-10; S2-13 and S1-RBD-11; S2-14 and S1-RBD-12; S2-15 and S1-RBD-14; S2-18 and S1-RBD-15; S2-22 and S1-RBD-16; S2-26 and S1-RBD-18; S2-33 and S1-RBD-19; S2-35 and S1-RBD-20; S2-36 and S1-RBD-21; S2-39 and S1-RBD-22; S2-40 and S1-RBD-23; S2-42 and S1-RBD-24; S2-47 and S1-RBD-25; S2-57 and S1-RBD-26; S2-59 and S1-RBD-27; S2-62 and S1-RBD-28; S2-1 and S1-RBD-29; S2-2 and S1-RBD-30; S2-3 and S1-RBD-32; S2-4 and S1-RBD-34; S2-5 and S1-RBD-35; S2-6 and S1-RBD-36; S2-7 and S1-RBD-37; S2-9 and S1-RBD-38; S2-10 and S1-RBD-39; S2-11 and S1-RBD-40; S2-13 and S1-RBD-41; S2-14 and S1-RBD-43; S2-15 and S1-RBD-44; S2-18 and S1-RBD-45; S2-22 and S1-RBD-46; S2-26 and S1-RBD-47; S2-33 and S1-RBD-48; S2-35 and S1-RBD-49; S2-36 and S1-RBD-51.S1-2, S2-14 and S1-52; S1-3, S2-15 and S1-53; S1-7, S2-18 and S1-54; S1-9, S2-22 and S1-55; S1-10, S2-26 and S1-56; S1-11, S2-33 and S1-61; S1-17, S2-35 and S1-62; S1-24, S2-36 and S1-63; S1-25, S2-39 and S1-RBD-3; S1-30, S2-40 and S1-RBD-4; S1-32, S2-42 and S1-RBD-5; S1-41, S2-47 and S1-RBD-6; S1-49, S2-57 and S1-RBD-9; S1-50, S2-59 and S1-RBD-10; S1-58, S2-62 and S1-RBD-11; S1-60, S2-1 and S1-RBD-12; S1-64, S2-2 and S1-RBD-14; S1-65, S2-3 and S1-RBD-15; S1-66, S2-4 and S1-RBD-16; S1-2, S2-5 and S1-RBD-18; S1-3, S2-6 and S1-RBD-19; S1-7, S2-7 and S1-RBD-20; S1-9, S2-9 and S1-RBD-21; S1-10, S2-10 and S1-RBD-22; S1-11, S2-11 and S1-RBD-23; S1-17, S2-13 and S1-RBD-24; S1-24, S2-14 and S1-RBD-25; S1-25, S2-15 and S1-RBD-26; S1-30, S2-18 and S1-RBD-27; S1-32, S2-22 and S1-RBD-28; S1-41, S2-26 and S1-RBD-29; S1-49, S2-33 and S1-RBD-30; S1-50, S2-35 and S1-RBD-32; S1-58, S2-36 and S1-RBD-34; S1-60, S2-39 and S1-RBD-35; S1-64, S2-40 and S1-RBD-36; S1-65, S2-42 and S1-RBD-37; S1-66, S2-47 and S1-RBD-38; S1-2, S2-57 and S1-RBD-39; S1-3, S2-59 and S1-RBD-40; S1-7, S2-62 and S1-RBD-41; S1-9, S2-1 and S1-RBD-43; S1-10, S2-2 and S1-RBD-44; S1-11, S2-3 and S1-RBD-45; S1-17, S2-4 and S1-RBD-46; S1-24, S2-5 and S1-RBD-47; S1-25, S2-6 and S1-RBD-48; S1-30, S2-7 and S1-RBD-49; S1-32, S2-9 and S1-RBD-51; S1-41, S2-10 and S1-1; S1-49, S2-11 and S1-4; S1-50, S2-13 and S1-20; S1-58, S2-14 and S1-21; S1-60, S2-15 and S1-23; S1-64, S2-18 and S1-27; S1-65, S2-1 and S1-28; S1-66, S2-2 and S1-29; S1-2, S2-3 and S1-31; S1-3, S2-4 and S1-35; S1-7, S2-5 and S1-36; S1-9, S2-6 and S1-37; S1-10, S2-7 and S1-38; S1-11, S2-9 and S1-39; S1-17, S2-10 and S1-46; S1-24, S2-11 and S1-48; or S1-25, S2-13 and S1-51.

113. The composition of claim 101, wherein the composition comprises HcAb wherein HcAb within the composition comprises at least two different single-domain antibodies of claim 1 or 2.

114. The composition of claim 101, wherein the composition comprises cells of claim 63, wherein the cells within the composition express at least two different single-domain antibodies of claim 1 or 2 or HcAb wherein expressed HcAb comprise at least two different single-domain antibodies of claim 1 or 2.

115. The composition of claim 101, formulated for pulmonary administration.

116. The composition of claim 101, freeze-dried without a cryoprotectant.

117. A method of treating a subject in need thereof comprising administering a therapeutically effective amount of the composition of claim 101 thereby treating the subject in need thereof.

118. The method of claim 117, wherein the therapeutically effective amount provides a prophylactic or a therapeutic treatment against a viral infection.

119. The method of claim 118, wherein the therapeutically effective amount provides a prophylactic or a therapeutic treatment against a coronavirus infection.

120. The method of claim 118, wherein the therapeutically effective amount provides a prophylactic or a therapeutic treatment against a SARS coronavirus infection.

121. The method of claim 118, wherein the therapeutically effective amount provides a prophylactic or a therapeutic treatment against a SARS-CoV-2 or a SARS-CoV-1 coronavirus infection.

122. The method of claim 117, wherein the therapeutically effective amount provides an anti-infection effect against SARS-CoV-2.

123. The method of claim 122, wherein the anti-infection effect against SARS-CoV-2 increases lung capacity, increases ability to taste, increases ability to smell, decreases infection-induced blood clotting, decreases gastrointestinal distress, decreases headaches, and/or decreases “COVID-toe”.

124. The method of claim 117, wherein the administering is through inhalation.


SEQUENCE LISTING (595 sequences)

SEQ ID NO: 1 - protein, 122 aa, Artificial Sequence
  Variable Heavy Domain of S1-1
MAQVQLVESGGGLVQAGGSLRLSCAASGMPFSSYAMGWFRQAPGKEREFVAAVNWNGISTYLADSVKGRFTASRDNAKNTAYLQMNSLKPEDTAVYYCAADSKGWSEWGMDYWGKGTLVTVS

SEQ ID NO: 2 - protein, 119 aa, Artificial Sequence
  Variable Heavy Domain of S1-2
MAQTQLEESGGGLVQAGGSLRLSCAASGSIIRPNVMAWYRQGPGKQRELVASIHSSGTKNYADSVKGRFTISRDNAKNTVYLQMNSLKPEDTAVYYCATRSTWATQPDYWGQGTQVTVS

SEQ ID NO: 3 - protein, 115 aa, Artificial Sequence
  Variable Heavy Domain of S1-3
MGQVQLVESGGGLVQAGGSLRLSCAASGSTFSNTGLGWYRQAPGKQREWVASISPSGSTSYLDSVRGRFIFSRDNAKNIAYLQMNSLKPEDTAVYYCNTLPRSGSWGQGTQVTVS

SEQ ID NO: 4 - protein, 126 aa, Artificial Sequence
  Variable Heavy Domain of S1-4
MAQVQLVESGGGLVQAGGSLRLSCAAAGRTFSSYFMAWFRQAPGKEREFVAAVDWSGGDTLYTDSVKGRFTISRDNGANTVYLQMNNLKREDTAVYYCAADRGYSLYYYQAREYEYWGQGTQVTVS

SEQ ID NO: 5 - protein, 128 aa, Artificial Sequence
  Variable Heavy Domain of S1-5
MAQVQLVESGGGLVQPGGSLRLSCAASGFTLGYYAIGWFRQAPGKEREGVSCIGSSDGSTYYADSVKGRFTISRDNAKNTVYLQINSLKPEDTAVYYCATKGPPQGVLRCSISGRYDFWGQGTQVTVS

SEQ ID NO: 6 - protein, 127 aa, Artificial Sequence
  Variable Heavy Domain of S1-6
MAQVQLVESGGGLVQAGDSLRLSCVVSGRTSSFFSMAWFRQAPGKEREFVAVIRGSGGVTFYSESVKGRFTISRDNAKNTVSLQMSSLKPEDTAVYYCAGSDFGDHGPLSGRTDFSSWGQGTQVTVS

SEQ ID NO: 7 - protein, 127 aa, Artificial Sequence
  Variable Heavy Domain of S1-7
MAQVQLAESGGGLVQVGDSLRLSCAASGRTYTSSMYGMAWFRQAPGKEREFVAVIGWNTYYADPVKGRFTISRDNAKNIVYLQMNNLKPEDTAVYYCASRKAAGLASGYPSPSYYDYWGQGTQVTVS

SEQ ID NO: 8 - protein, 124 aa, Artificial Sequence
  Variable Heavy Domain of S1-9
MAQVRLVESGGGLVQPGGSLRLSCVVSGVSGSAFRFNRLGWYRQAPGKERELVASISSGGSTNYADSVKGRFTISRDNAKSTVSLQMNSLKPEDTAVYYCNIRADFFSSTRTGDWGQGTQVTVS

SEQ ID NO: 9 - protein, 123 aa, Artificial Sequence
  Variable Heavy Domain of S1-10
MAQVQLVESGGGLVQAGGSLRLSCAASGRTFSSAGMAWFRQAPGKEREFVATSNWNGGDTDYADSVKGRFTISRDNAKNTVYLQMNNLKPEDTAVYYCAAHLQKYGGDYYARIWGQGTQVTVS

SEQ ID NO: 10 - protein, 127 aa, Artificial Sequence
  Variable Heavy Domain of S1-11
MAEVQLVESGGGLVQFGGSLRLSCAASGFTLSPYDIGWFRQAPGKEREGVSCISVTGGTTDYTDSVKGRFTISRDHARNTVYLQMNNLKPEDTAVYYCAAHTASFCRGDYSASVARYWGQGTQVTVS

SEQ ID NO: 11 - protein, 125 aa, Artificial Sequence
  Variable Heavy Domain of S1-12
MAQVQLVESGGGLVQAGGSLRLSCAASGRTFSNYAMGWFRQAPGKEREFVVAISWKSGLITAANFLEGRFTISRDNAKNTMYLQMNNLKPEDAAVYYCAVDPNNRRGTLTSYYDHWGQGTQVTVS

SEQ ID NO: 12 - protein, 124 aa, Artificial Sequence
  Variable Heavy Domain of S1-14
MAQVQLVESGGGLVQAGGSLRLSCAASGSTFSRLSMGWYRQVPGKQRELVARILPLGGPYYRDFVQGRFTISRDNVKNMLYLQMNSLKPEDTAVYYCNRAPFGTAWDGPDNYDYWGQGTQVTVS

SEQ ID NO: 13 - protein, 127 aa, Artificial Sequence
  Variable Heavy Domain of S1-17
MAQVQLVESGGGLVQAGGSLRLSCPASGLASTYSAKGWFRQAPGKEREFVAAINRSGGITSYADSVKGRFTISRDDAKNTVYLQMNNLKPEDTAVYYCAAKVTLSDDTYNQPGNYPSWGQGTQVTVS

SEQ ID NO: 14 - protein, 119 aa, Artificial Sequence
  Variable Heavy Domain of S1-19
MAQVQLVESGGGLVRAGGSLRLSCAASVSTFSSYAMGWYRQAPGNQRELVAGISPDGSTNYADSVKGRFTISRDNAKNTLVLQMNSLKSEDTAVYYCRIFLPEIPGRGSWGQGTQVTVS

SEQ ID NO: 15 - protein, 128 aa, Artificial Sequence
  Variable Heavy Domain of S1-20
MAQVQLVESGGGLVQPGGSLRLSCAVSGFPLDFYAIGWFRQASGKEREWVSCISRRDDYTSYVDSVKGRFTISRDNAENTVYLQMNSLKLEDTAVYYCAGVRTSSGTVCQAYPRWYDTWGQGTQVTVS

SEQ ID NO: 16 - protein, 117 aa, Artificial Sequence
  Variable Heavy Domain of S1-21
MAQVQLVESGGGLVQAGGSLRLSCAVSGIAVRASTMGWYRQAPGKQRELVATINIGGSTNYADSVKGRFTIARDNAENTAYLQMNSLKPEDTAVYYCKAQYYTGYDYWGRGTQVTVS

SEQ ID NO: 17 - protein, 120 aa, Artificial Sequence
  Variable Heavy Domain of S1-23
MAQVQLVESGGGLVQAGGSLRLSCVASGRTSRPYRMGWFRQAPGKEREFVAAIFWSGSHTLYEDSVKGRFTISTDNAQNTVYLQMNSLKPEDTAVYYCAARNQATGNYDVWGQGTQVTVS

SEQ ID NO: 18 - protein, 126 aa, Artificial Sequence
  Variable Heavy Domain of S1-24
MAQVQPVESGGGLVQAGGSLRLSCVASGSTTTNYHMGWYRQTPGEQRELVAAINAGGITNYADSVKGRFTISRDNAKNTMYLQMNNLRFEDTAVYYCNIGGGWDYRNSYYIPRVDSWGQGTQVTVS

SEQ ID NO: 19 - protein, 124 aa, Artificial Sequence
  Variable Heavy Domain of S1-25
MAQVRLVESGGGLVQPGGSLRLSCVVSGVSGSGFRFNRLGWYRQAPGKERELVASISSGGSTNYADSVKGRFTISRDNAKNSVSLQMNSLKPEDTAVYYCNIRADFFTSTRTGDWGQGTQVTVS

SEQ ID NO: 20 - protein, 120 aa, Artificial Sequence
  Variable Heavy Domain of S1-27
MAQVQLVESGGGLAQPGGSLRLSCAASAGTFGSSRMGWFRQAPGKEREFVAAIMPSGSHTNYADSVKGRFTISRDNAKDTIYLQMNSLKPEDTAVYYCGARDPSSYTYDRWGQGTQVTVS

SEQ ID NO: 21 - protein, 129 aa, Artificial Sequence
  Variable Heavy Domain of S1-28
MAQVQLVESGGGLVQAGGSLKLSCAVSGRTLSSYVMGWFRQAPGKEREFVAAIRWNGGSTFYADSVQGRLTISRDNAKNMVYLQMDSLKPEDTAAYYCAASPRTMYYASYYYTRTSYDYWGQGTQVTVS

SEQ ID NO: 22 - protein, 129 aa, Artificial Sequence
  Variable Heavy Domain of S1-29
MAQVQLVESGGGQAQAGDSLRLSCAASGDTFSSAPMGWFRQAPGKERELIAGISWSALNTYYADSVKGRFTISREDAKNTVYLQMNSLKQEDTAVYHCAARTRSHFYSEVYRRASDYDFWGQGTQVTVS

SEQ ID NO: 23 - protein, 127 aa, Artificial Sequence
  Variable Heavy Domain of S1-30
MAQVPLVESGGGLVQAGGSLRLSCTTSGSTFSTSHMGWYRQAPGKQRELVASISTEGTTEYADFVKGRFTISRNNAQNTVYLEMASLKPEDTAVYHCNRGGWRYYGDGSHYIPDFRFWGQGTQVTVS

SEQ ID NO: 24 - protein, 118 aa, Artificial Sequence
  Variable Heavy Domain of S1-31
MAQVQLVESGGGLVQPGGSLRLSCAASGSISSIIYRGWFRQVPGKEGEFVASIDTAGSTTYADSVKGRFTISRDNAKNTLYLQMNSLKPEDTAVYYCKYVTWAPRVEYRGQGTQVTVS

SEQ ID NO: 25 - protein, 127 aa, Artificial Sequence
  Variable Heavy Domain of S1-32
MAQVQLVESGGGLVQAGGSLRLSCTASGVAFSLYHMAWFRQAPGKEREFVTAINWNGGIADYVDSVKGRFTISRDNAKNTVYLQMNSLKPEDTAVYYCAAKLGAYYTTYRKANEYQYWGQGTQVTVS

SEQ ID NO: 26 - protein, 126 aa, Artificial Sequence
  Variable Heavy Domain of S1-35
MAQVHLIESGGGLVQAGGSLRLSCVASGRTFSSLVMAWFRQAPGKEREFVAFIEWSGAETYYTDSVKGRFTISRDNAKNTVILQMNSVKPEDTGVYYCAADKGYSLYYYRESDYEYWGQGTRVTVS

SEQ ID NO: 27 - protein, 129 aa, Artificial Sequence
  Variable Heavy Domain of S1-36
MAQVQLVESGGGLVQAGGSLRVSCAASERILSSYVMGWFRQAPGKEREFVAAIRWNGGSTFYADSVKGRLTISRDNAKNTVYLQMNSLKPEDTAVYYCAASPRTMYLASYYYHRTSYDYWGQGTQVTVS

SEQ ID NO: 28 - protein, 128 aa, Artificial Sequence
  Variable Heavy Domain of S1-37
MAQVQLVESGGGLVQPGGSLRLSCVVSGFPLDFFAIGWFRQASGKEREWVSCISRRDDYTSYVDSVNGRFTISRDNAENTVYLQMNSLKLEDTAVYYCAGVRTSSDTVCQNYPRWYLTWGQGTQVTVS

SEQ ID NO: 29 - protein, 123 aa, Artificial Sequence
  Variable Heavy Domain of S1-38
MAQVQLVESGGGLVQAGGSLRLSCAASGRTFSYAIMGWFRQAPGKDREFVGWMAWSGPAKYYADSVKGRFSISRDNAKNTVYLQMNSLKPEDTAVYYCAASKEITTMTTRYDYWGQGTQVTVS

SEQ ID NO: 30 - protein, 127 aa, Artificial Sequence
  Variable Heavy Domain of S1-39
MAQVQLVESGGGLVQAGGSLRLSCAASGRTFGSYAMGWFRQAPGKERECVSAINWSGDHTYYADSVKGRFTISRDNAKNTVYLQMNSLKPEDTAVYYCAARGPNEWGLPREDLFYDYWGQGTQVTVS

SEQ ID NO: 31 - protein, 127 aa, Artificial Sequence
  Variable Heavy Domain of S1-41
MAQVPLVESGGGLVQAGGSLRLSCTTSGSTFSTYHMGWYRQAPGKQRELVAAINSEGSTYYPDSVKGRFTISRDNAQNTVYLEMASLKPEDTAVYHCNRGGWRYYGDGSRYIPDFRSWGQGTQVTVS

SEQ ID NO: 32 - protein, 128 aa, Artificial Sequence
  Variable Heavy Domain of S1-46
MAQVQLVESGGGLVQAGGSLRVSCVISGHQFSDYVIGWFRQAPGKEREFVGAIWWSDDITYYEDSVKGRFTVSRDNAKNTVYLQMNSLKPEDTAVYHCAVRFRPVSTYQVRSTDDYGYWGQGTQVTVS

SEQ ID NO: 33 - protein, 131 aa, Artificial Sequence
  Variable Heavy Domain of S1-48
MAQVQLVESGGGLVQPGGSLRLSCAASGITLDYYAIGWFRQAPGKEREGVSLISSSDGSTYYADSVKGRFTISRDNAKNTVYLQMNSLKPEDTAVYYCATGSLTRYGSSWHVPFGSRSYENWGQGTQVTVS

SEQ ID NO: 34 - protein, 122 aa, Artificial Sequence
  Variable Heavy Domain of S1-49
MAQIQMQLVESGGGLVQAGGSLRLSCTASGSIFSIGAMSWYRQAPGKQRELVAQISSGGITNYADSVKGRFTISRDNAKNTVYLQMNTLKPEDTAVYYCAADETTPWGATRGWGQGTQVTVS

SEQ ID NO: 35 - protein, 119 aa, Artificial Sequence
  Variable Heavy Domain of S1-50
MAQTQLEESGGGLVQAGGSLRLSCAASGSIIRPNVMAWYRQGPGKQRELVASMHSSGTKNYAESVKGRFTISRDNAKNTVYLQMNSLKPEDTAVYYCATRSTWAGQPDYWGQGTQVTVS

SEQ ID NO: 36 - protein, 127 aa, Artificial Sequence
  Variable Heavy Domain of S1-51
MAQVQLVESGGGLVQAGGSLRLSCAASGFSFSNYYMVWFRQAPGKEREFVAAITTWLSNTYYTDSVKDRFTISRDNAKKMLYLQMNSLKPEDTALYYCAAEQSGRDWRRTSPSPYAYWGQGTQVTVS

SEQ ID NO: 37 - protein, 121 aa, Artificial Sequence
  Variable Heavy Domain of S1-52
MAQVQLVESGGGLVQAGGSLRLSCAASGSIFSLNAKAWYRQAPGSQRELVARITGSGTITNYADSVKGRFTISRDNVKNTVTLQMNSLRPEDTAIYICAAAPGGLWTGPAYWGQGTQVTVS

SEQ ID NO: 38 - protein, 123 aa, Artificial Sequence
  Variable Heavy Domain of S1-53
MAQVQLVESGGGLVQAGDSLRLSCAASGYTITNYWMAWFRQAPGKERESVATITYGAGMKYYADSVKGRFTISRDNTKNTVYLQMESLKPEDTAVYYCRSPQRPYSGFGSDDYWGQGTQVTVS

SEQ ID NO: 39 - protein, 125 aa, Artificial Sequence
  Variable Heavy Domain of S1-54
MAQVQLVESGGGLVQAGGSLRLSCAASGIIFSKYAVAWYRQAPGKQRELVATINHSGDTKYADSVKGRFTISRDNAKSTLYLQMNSLKPEDTAVYYCNIGIWIASAHSPDGRADHWGRGTQVTVS

SEQ ID NO: 40 - protein, 125 aa, Artificial Sequence
  Variable Heavy Domain of S1-55
MAQVQVVESGGGLVQAGGSLRLSCAVSGRTFGMRAMGWFRQAPGKEREFVAVISTSGGDTSYADSVKGRFTISRDNAKNTVYLQMNTLKPEDTAVYYCAVPRIGAWSRSPRDYDIWGQGTQVTVS

SEQ ID NO: 41 - protein, 122 aa, Artificial Sequence
  Variable Heavy Domain of S1-56
MAQVQLVESGGGLVQPGGSLRLSCAASGSIFSIDAMGWFRQAPGKEREPVAHIRNTGTTAYADSVKGRFTISRDNTKNTVYLQMNSLKPEDTAVYYCNELKDFLGGVIPLNSWGQGTQVTVS

SEQ ID NO: 42 - protein, 126 aa, Artificial Sequence
  Variable Heavy Domain of S1-58
MAQVQLVESGGGLVQAGGSLRLSCAASGSTFSAYSMGWYRQAPGKQRELVAAISSGGSTNYADSVKGRFTISRDNAKNTVYLQMNSLKPEDTAVYYCNTVGWDYRYDYPITGRNDYWGQGTQVTVS

SEQ ID NO: 43 - protein, 122 aa, Artificial Sequence
  Variable Heavy Domain of S1-60
MAQVQLVESGGGLVQAGGSLRLSCAASGSTFSTNAMGWYRQAPGKERELVAEITTDGTTRYADSVKGRFTISRDNAKNTVYLQMNSLKSEDTAVYYCNTVTRFLGGLLRPDYWGQGTQVTVS

SEQ ID NO: 44 - protein, 128 aa, Artificial Sequence
  Variable Heavy Domain of S1-61
MAQVQLIESGGGLVQAGGSLRLSCAASGRTFSSYAMGWLRQAPGEEREFVAAISRRGMDTYYADSVKGRFTISRDNAKNTVYLQMNSLKPEDTAVYYCARKMVAPIFVVVTTRDEYDYWGQGTQVTVS

SEQ ID NO: 45 - protein, 124 aa, Artificial Sequence
  Variable Heavy Domain of S1-62
MAQVQLVESGGGLVQAGGSLRLSCAPSGRTSNTYPMAWFRQAPGKEREFVAAITRSVPNTYYADSVKGRFTISRDIAKNTMYLQMNSLKPEDTAVYYCAGSSNVYLLTRDEYDYWGQGTQVTVS

SEQ ID NO: 46 - protein, 122 aa, Artificial Sequence
  Variable Heavy Domain of S1-63
MAQVQLVESGGGLVQFGGSLRLSCAASGIIFSINAMGWYRQAPGKERESVALIMNNGNTYYENSVKGRFSISRDNAKNTVYLQMDSLKPEDTAVYYCNAVAYRDYDRKRYDYWGQGTQVTVS

SEQ ID NO: 47 - protein, 121 aa, Artificial Sequence
  Variable Heavy Domain of S1-64
MAQVQLVESGGGLVQPGGSLRLSCAASGSIFSVNSMGWYRQAPGKERELVATITSHGITNYADSVKGRFTISRDNAKDTVYLQMNSLKPEDTAVYFCHREDTGLWNSKDTYWGQGTQVTVS

SEQ ID NO: 48 - protein, 121 aa, Artificial Sequence
  Variable Heavy Domain of S1-65
MAQVQLVESGGGLVQPGGSLRLSCAASGSIFNVNSMGWYRQAPGKERELVATITTRGITNYADSVKGRFTISRGNAKDTVHLQMNSLKPEDTAVYYCNREDTGLWNSKDVYWGQGTQVTVS

SEQ ID NO: 49 - protein, 120 aa, Artificial Sequence
  Variable Heavy Domain of S1-66
MAHVQLVESGGGLVQAGGSLRLSCVASTRTFMGWFRQAPGKERAFVAAISWNGVSITYADSVAGRFTISRDNAKNTVLLQMNSLKPEDTAVYYCAAADVVAGTRWTDYNYWGQGTQVTVS

SEQ ID NO: 50 - protein, 129 aa, Artificial Sequence
  Variable Heavy Domain of S1-RBD-3
MAEVQLVESGGGLVQAGGSLRLSCAASGFTLDTTAIGWFRQAPGKEREGVSCISTSGERTYYADSVKGRFTISRDNAKNMVYLQMNNLKPEDTAVYSCAAKYASFQYLPVGLPWSMYDVRGQGTQVTVS

SEQ ID NO: 51 - protein, 128 aa, Artificial Sequence
  Variable Heavy Domain of S1-RBD-4
MAEVQLVESGGGLVQAGGSLRLSCAASEFTLGYYAIGWFRQAPGKEREGVSFISRHGSTTYYTKSVRGRFTISRDNSENTVYLQMDGLKPEDTAVYYCAASKTTAWDTIRVSAIEYDYWGQGTQVTVS

SEQ ID NO: 52 - protein, 126 aa, Artificial Sequence
  Variable Heavy Domain of S1-RBD-5
MAQVQLVESGGGLVQAGGSLRLSCAASGRTFSSLVMGWFRQAPGKEREFVAFIEWNGGDTYYTDAVKGRFTISRDNAKNTVFLQMNSLKPEDTAVYYCAADRAYSLYYYRVSEYQYWGQGTRVTVS

SEQ ID NO: 53 - protein, 127 aa, Artificial Sequence
  Variable Heavy Domain of S1-RBD-6
MGQVQVAESGGGLVQAGDSLRLSCAASGRTFTTNAMGWFRQAPGKEREFVAAISWSTGTTYYSDPVKGRFTISRDNAKNTVYLQMNSLKPGDTAVYYCTLRSAFNAYAWTTERAYDYWGQGTQVTVS

SEQ ID NO: 54 - protein, 126 aa, Artificial Sequence
  Variable Heavy Domain of S1-RBD-9
MAQVQLVESGGGSVQAGGSLRLSCAASARTFSNYVVAWFRQAPGKEREFVALITWSGADRYYGDSVKGRFTISRDDAKNTVYLQMNSLKPEDTADYYCAADRGSSYHYAESKNYDYWGQGTQVTVS

SEQ ID NO: 55 - protein, 126 aa, Artificial Sequence
  Variable Heavy Domain of S1-RBD-10
MAQVQLVESGGGLVQAGGSLRLSCAVSGRTFRNYAMGWFRQAPGKEREFVAVITATDDITYYADSVKGRFTISRDNAKNTVYLQMNSLKPEDTAVYYCAADYGDSYYYTRRTEYGYWGQGTQVTVS

SEQ ID NO: 56 - protein, 119 aa, Artificial Sequence
  Variable Heavy Domain of S1-RBD-11
MAQVQVVESGGGLVQAGGSLRLSCVASGSTDSNYVMGWYRQTAGKQREWVASINSGGETRSVDSVKGRFTISGDNAKNTVYLQMNSLKPEDTAVYYCFYERLAWDPSTSWGQGTQVTVS

SEQ ID NO: 57 - protein, 126 aa, Artificial Sequence
  Variable Heavy Domain of S1-RBD-12
MAQVQLVESGGGLVQPGGSLRLSCAASGSILSINDMGWYRQAPGKERELVAFITSGGSTNYAESVKGRFTVSRDNAKHAVYLQMNSLKPEDTAVYYCNADIRESGFVYGMNAWGKYWGKGTLVTVS

SEQ ID NO: 58 - protein, 130 aa, Artificial Sequence
  Variable Heavy Domain of S1-RBD-14
MAQVQLVESGGGLVQAGGSVRLSCAASGRSFSINPMGWFRQAPGKEREFVAAISWSGSKTVYVDSVKGRFSISRDNAKNTVYLQMNSLEPEDTAVYHCAVASRGPVYGANYVPGPHEYEYWGQGTQVTVS

SEQ ID NO: 59 - protein, 127 aa, Artificial Sequence
  Variable Heavy Domain of S1-RBD-15
MAQVQLVESGGGLVQAGGSLRLSCAASGRTFSSYAMGWFRQAPGKEREFVASINWNGGNTYYADFVKGRFTISRDNAKNTVYLQMNSLKPEDTAVYYCAATGPNEYGLPREDLFYDYWGQGTQVTVS

SEQ ID NO: 60 - protein, 127 aa, Artificial Sequence
  Variable Heavy Domain of S1-RBD-16
MAQVQLVESGGGLVQSGGSLRLSCVASGRGFDRYPMGWFRQAPGKEREFVGGINWNGANTYYADSVKGRFTISRDNTKNTVYLQMNSLKPEDTALYYCAADQHYMGSPPPKEYEYDYWGQGTQVTVS

SEQ ID NO: 61 - protein, 115 aa, Artificial Sequence
  Variable Heavy Domain of S1-RBD-18
MAQVQLVESGGGLVQAGGSLRLSCTASGITFSGYVMAWYRQAPGKQRELVGWISHGGIKDYADSVKGRFTISRDNPKNTVYLQMNSLKPEDTAVYYCNRKDAQSSWGQGTQVTVS

SEQ ID NO: 62 - protein, 129 aa, Artificial Sequence
  Variable Heavy Domain of S1-RBD-19
MAEVQLVESGGGLVQPGGSLRLSCVASGFSFSSYGMGWFRQAPGKEREFVATISGTGGNTAYSDSVKDRFTISRDNAKNTVYLEMNSLKPEDTAVYYCAAPPSWYTRRYINFAPSEVRHWGQGTQVTVS

SEQ ID NO: 63 - protein, 128 aa, Artificial Sequence
  Variable Heavy Domain of S1-RBD-20
MAQVQLVESGGGLVQAGDSRRLSCAVSGRTFSSGAMAWFRQAPGEERDFVAVIRWSGSTYYADSVKDRFTISTDNVKNTVFLQMNNLAPEDTAVYYCAATDRTHYDIVEYTRDHFYSNWGQGTQVTVS

SEQ ID NO: 64 - protein, 124 aa, Artificial Sequence
  Variable Heavy Domain of S1-RBD-21
MAQVQLVESGGGSVQAGGSLRLSCAASGRAFSDYGMGWFRQAPGKEREFVATIDSTGSNTYYADSVKGRFTISRVNAENTVYLQMNSLKPEDTAVYYCAADRFGGGRMEYRYEYWGQGTQVTVS

SEQ ID NO: 65 - protein, 126 aa, Artificial Sequence
  Variable Heavy Domain of S1-RBD-22
MAQVQLAESGGGLVQAGDSLRLSCVASGRTFSAYTLAWFRLAPGKEREFVAHIDWSGTETNYADSNKGRFTISRDNALNTVYLQMTGLKPEDTAVYYCAADRAYSLMYYDTREYEYWGQGTQVTVS

SEQ ID NO: 66 - protein, 122 aa, Artificial Sequence
  Variable Heavy Domain of S1-RBD-23
MAQVHLVESGGDLVQPGGSLRLSCVASGSGFENNAITWYRQAPGKERELVSGITSGGSTNYADSVKGRFTISRDNAKNTVYLEMNSLKPEDTAVYLCQAVAWDSRRRSVVAFWGQGTQVTVS

SEQ ID NO: 67 - protein, 125 aa, Artificial Sequence
  Variable Heavy Domain of S1-RBD-24
MAQVQLVESGGGLVQAGGSLRLSCVVSGHTFGSLTMAWFRQAPGKEREFVAGTDWSGINYADSVKGRFTISRDSTTNTLFLQMNSLKPEDTAVYYCAFDRAASLYPLRDQADYMYWGQGTQVTVS

SEQ ID NO: 68 - protein, 128 aa, Artificial Sequence
  Variable Heavy Domain of S1-RBD-25
MAEVQLVESGGGLVQPGGSLKLSCAASGFTLDYYTIGWFRQAPGKEREGVSYISSSGSNTYYEDSVKGRFTISRDNAKSTVYLQMNSLKPEDTAVYYCAARRVWQETIVVRGPEHYEYWGQGTQVTVS

SEQ ID NO: 69 - protein, 125 aa, Artificial Sequence
  Variable Heavy Domain of S1-RBD-26
MAQVQLVESGGGLVQPGGSLRLSCAISGRTFGGYGVGWFRQAPGKEREFVAGITSTSFGTSYADAVKGRFTISRDNAKNTVTLQMNSLKFEDTAVYYCAASVLSPPPRTNSAYAYWGQGIQVTVS

SEQ ID NO: 70 - protein, 124 aa, Artificial Sequence
  Variable Heavy Domain of S1-RBD-27
MAQVHVVESGGGLVQAGGSLRLSCAASGRTFGIYNMGWFRQAPGKEREFVAAITGDASDTYYADSVKGRFTISRQNAKNTVFLQMDNLKPEDTAVYYCAATGAITRATMWKPNFWGQGTQVTVS

SEQ ID NO: 71 - protein, 125 aa, Artificial Sequence
  Variable Heavy Domain of S1-RBD-28
MAQVQLAESGGGLVQAGGSLRLSCAASGRTISTDAMGWFRQAPGKEREFVAISWRSDDTYYADNVKGRFTMSRDNAKNTVYLQMNNLKPEDTAVYYCVADQGRSYYYTRESEYNYWGQGTQVTVS

SEQ ID NO: 72 - protein, 127 aa, Artificial Sequence
  Variable Heavy Domain of S1-RBD-29
MAQVQLVESGGGLVQDGGSLRLSCAASGLASIVFNMGWFRQAPGKEREFVAGILGSGQSTYYGNSVKGRFTISRDSAKNTVYLQMNSLKPEDTAVYYCAAGGRLDLVNTTGGYEPTYWGQGTQVTVS

SEQ ID NO: 73 - protein, 126 aa, Artificial Sequence
  Variable Heavy Domain of S1-RBD-30
MAQVQLVESGGGLVQAGGSLRLSCAASGRTFSSLVMGWFRQAPGKEREFVAFIEWSGAETYYTDSVKGRFTISRDNAKNTVFLQMNSLKPEDTAVYYCAADAAYSLYYYRVSEYEYWGQGTRVTVS

SEQ ID NO: 74 - protein, 128 aa, Artificial Sequence
  Variable Heavy Domain of S1-RBD-32
MAEVQLVESGGGLVQPGGSLKLSCAASGFTLDYYTIGWFRQAPGKEREGVSYISSSGSNSYYVDSVKGRFTISRDNAKSTVYLQMNSLKPEDTAVYYCAARRVWQPTIVVSGPEHYDYWGQGTQVTVS

SEQ ID NO: 75 - protein, 132 aa, Artificial Sequence
  Variable Heavy Domain of S1-RBD-34
MAQVQLVESGGGLVQAGDSLRLSCVASGRTFSTTFMGWFRQAPGKERDFVAAINWNGGSTRYADSVKGRFTISRDNAKNMVYLQLNSLKPEDTAVYYCAADPSDGRVVGYNYVPVSTHEYDYWGQGTQVTVS

SEQ ID NO: 76 - protein, 119 aa, Artificial Sequence
  Variable Heavy Domain of S1-RBD-35
MTQVQVVESGGGLVQAGGSLRLSCVASGSTDSNYVMGWYRQASGKQREWVASINSGGDTKSVDSVKGRFTISIDNAKNTMYLQMNSLKPEDTAVYYCFYEKLAWDPSTTWGQGTQVTVS

SEQ ID NO: 77 - protein, 132 aa, Artificial Sequence
  Variable Heavy Domain of S1-RBD-36
MAQVQLVESGGGLVQAGDSLRLSCVASGRTFSTTFMGWFRQAPGKEREFVAAINWSGGSTRYADSVKGRFTISRDNAKNTVYLQLNSLKPEDTAVYYCAADPSDGRVFGVNYVPVSTHEYDYWGQGTQVTVS

SEQ ID NO: 78 - protein, 118 aa, Artificial Sequence
  Variable Heavy Domain of S1-RBD-37
MAQVQLVESGGGLVQAGGSLRLSCGASGDSFSFRWYGMNWYRQGPGKPREWVAGISSAGSTNYADSVKGRFTISRDNAKNTVYLQMNSLKPEDTAVYYCYSATQERDYWGQGTQVTVS

SEQ ID NO: 79 - protein, 116 aa, Artificial Sequence
  Variable Heavy Domain of S1-RBD-38
MAQVQLVESGGGLVQAGGSLRLSCAASGSTFDNYPVTWYRQAPGKQREFVASIRSGHITNYANSVKGRFTMSKDNAKNTVYLEMNNLKPEDTAVYYCYTVDAQSDYWGQGTQVTVS

SEQ ID NO: 80 - protein, 117 aa, Artificial Sequence
  Variable Heavy Domain of S1-RBD-39
MAQVQLVESGGGRVQAGGSLRLSCAVSGSTFSSDVMAWYRQPPGKQREWVASFSSSGSGNYAASVKGRFTISRENAKNTVYLQMNSLKPEDTGVYYCHAAHVNGGDSWGQGTQVTVS

SEQ ID NO: 81 - protein, 116 aa, Artificial Sequence
  Variable Heavy Domain of S1-RBD-40
MAQVQLVESGGGLVQAGGSLRLSCAASGITFSNYGMVWWRQAPGKQREFVASISSGRDTNYADFVKGRFTISRDNAKNTVHLQMNSLKPEDTAVYYCYTVTDRSDYWGQGTQVTVS

SEQ ID NO: 82 - protein, 118 aa, Artificial Sequence
  Variable Heavy Domain of S1-RBD-41
MAQVQLVESGGGLVQAGGSLRLSCAASGITFSRSAVGWHRQAPGKPREWVAAISNSGTTSYTDPMKGRVTISRDNAKNTVYLQMINLKPEDTAVYYCYIADGLLGGDYWGQGTQVTVS

SEQ ID NO: 83 - protein, 117 aa, Artificial Sequence
  Variable Heavy Domain of S1-RBD-43
MAQVQLVESGGGLVQAGGSLRLSCAASGSTFSSDVITWYRQPPGKQREWVASIQSSGSRNYAASVKGRFTISRENAKNTVYLQMNSLKPEDTGVYYCHAGHVTGGDSWGQGTQVTVS

SEQ ID NO: 84 - protein, 116 aa, Artificial Sequence
  Variable Heavy Domain of S1-RBD-44
MAQVQLVESGGGLVQAGGSLRLSCAASSSTVSNSPMDWFRQAPGKQREFVATVQSGGNASYSYSVKGRFTISRDNAKKMVYLQMNSLKPEDTAVYYCHAADDRSDYWGQGTQVTVS

SEQ ID NO: 85 - protein, 116 aa, Artificial Sequence
  Variable Heavy Domain of S1-RBD-45
MAQVQLVESGGGLVQAGGSLRLSCLASGITWMPYGMEWYRQAPGKQRVFVATISSNGRTNYAASVMGRFTISRDNAKNTVYLQMNSLKPEDTAVYYCHGATALSDYWGQGTQVTVS

SEQ ID NO: 86 - protein, 128 aa, Artificial Sequence
  Variable Heavy Domain of S1-RBD-46
MAEVQLVESGGGLVQAGGSLRLSCAASESTLGSYAIGWFRQAPGKEREGVSFISRDGSTTFYTSSVRGRFTISRDNTKNMVYLQMDGLKPEDTAVYYCAASKITAWDTIIVSAIEYTYWGQGTQVTVS

SEQ ID NO: 87 - protein, 115 aa, Artificial Sequence
  Variable Heavy Domain of S1-RBD-47
MAQVQLVESGGGLAQTGGSLRLSCAASGISFSTYDMNWFRQTPGKQQEWVAGIGTLNSPRYANAVKGRFTISRDDAKNTVYLQMNSLKPEDTAVYYCNTASDRDYWGQGTQVTVS

SEQ ID NO: 88 - protein, 121 aa, Artificial Sequence
  Variable Heavy Domain of S1-RBD-48
MAQVQLVESGGGWVQAGGSLRLSCSASGRTFDNYNIGFIEWFRQAPGKERTFVATISRSGDITNYAGSVAGRFTISRDNNKNTVYLQMNSLKPEDTAVYYCHMSMTTGGDYWGQGTQVTVS

SEQ ID NO: 89 - protein, 117 aa, Artificial Sequence
  Variable Heavy Domain of S1-RBD-49
MAQVQLVESGGGLVQAGGSLRLSCTSSASTFSIGAMGWFRQAPGKQREFVATITSSGTTSYAGSVKGRFTISRENAKNSVYLQMNTLKPEDTAVYYCYANNLHGGDYWGQGTQVTVS

SEQ ID NO: 90 - protein, 120 aa, Artificial Sequence
  Variable Heavy Domain of S1-RBD-51
MAQVQLVESGGGLVQAGGSLRLSCVASGRTLSTYRMGWFRQAPGKERKFVAAIFWSGGHTVYEDSVKGRFTISTDNAQNTVYLQMNSLKPEDTAAYYCAARNPATGNYDYWGQGTQITVS

SEQ ID NO: 91 - protein, 118 aa, Artificial Sequence
  Variable Heavy Domain of S2-1
MAQVQVVESGGGLVQAGGSLRLSCAASTLVLGGYDMRWYRQVPGNGRELVASISNGGSTDYADFVKGRFTISSDVAKNTVYLQMDSLKSEDTAVYYCNMRSTYYGIDYWGRGTQVTVS

SEQ ID NO: 92 - protein, 117 aa, Artificial Sequence
  Variable Heavy Domain of S2-2
MAQVRLVESGGGLAQAGGSLRLSCAASGLQFSRITMAWYRQAPGKQRELVATLSTRGTTNYPDSVKGRFTISRDNAKNMVYLQMNSLKPEDTAVYYCNTGPGYKTDYWGQGTQVTVS

SEQ ID NO: 93 - protein, 118 aa, Artificial Sequence
  Variable Heavy Domain of S2-3
MAQILVTQSGGGLVQTGGSLRLSCAASTLVLGGYDMRWYRQAPGKERELVASITNGGDTRYVDSVKGRFTISSDNAKNAVYLQMDSLKSEDTAVYYCNMHSTYYGRDFWGQGTQVTVS

SEQ ID NO: 94 - protein, 127 aa, Artificial Sequence
  Variable Heavy Domain of S2-4
MTQVQLVESGGGLVQAGDSLRLSCVSSEPTFSSRTMGWFRQAPGKERDNVATIPWWRGALPYYKDSVKGRFTISRDSAKNTMYLQMNSLKPEDTAVYYCAASKTWSPSTGVATEYEFRGQGTQVTVS

SEQ ID NO: 95 - protein, 129 aa, Artificial Sequence
  Variable Heavy Domain of S2-5
MAQVQLVESGGGLVQAGGSLRLSCGASGLTFKNYAMGWFRQPPGKEREFVATINSIGGPANYANSVKGRFTISRDNAKNTVYLQMNSLKPEDTAVYYCAAERTPSTLLAGSSNPSRVDVWGQGTQVTVS

SEQ ID NO: 96 - protein, 118 aa, Artificial Sequence
  Variable Heavy Domain of S2-6
MTQVQLVESGGGLVQAGGSLRLSCAASAGIFGGSTMGWYRQAPGEQREMVATISNSGSANYADSVKGRFTISRDHAKRTVYLQMDSLKPEDTAVYYCNKHSIYYGRDYWGQGTQVTVS

SEQ ID NO: 97 - protein, 127 aa, Artificial Sequence
  Variable Heavy Domain of S2-7
MTQVQLVESGGGLVQAGDSLRLSCVASEPTFSSRTMGWFRQAPGKERESVAAIPWWKGALPYYKDSVKGRFTISRDSAKNTMYLQMNSLKPEDTAVYYCAASKTWSPYTDAATELEFRGQGTQVTVS

SEQ ID NO: 98 - protein, 117 aa, Artificial Sequence
  Variable Heavy Domain of S2-9
MLQVQLVESGGGLVQAGGSLRLSCAASGSTFSTYGVAWYRQAPGKQRELVASINSWGWINYADSVKGRFTISRDNAKNTVSLQMNSLKPEDTAVYTCNSQNSRGDNYWGQGTQVTVS

SEQ ID NO: 99 - protein, 127 aa, Artificial Sequence
  Variable Heavy Domain of S2-10
MAQVQLVESGGGLVQTGGSLRLSCAASGRIVSSSAMSWFRQAPGKEREFVAVIASSNSHTYYADAVKGRFTISRDNAKNTVYLQMNSLKPEDTAIYYCAARSHLTIAAWERLKTYDDWGQGTQVTVS

SEQ ID NO: 100 - protein, 128 aa, Artificial Sequence
  Variable Heavy Domain of S2-11
MAQVQLVESGGGLVQPGGSLRLSCAASGRTFSGATMGWFRQAPGKEREFVASILWRYEYTNYANSVKGRFTISADKAKNTVYLQMNSLKPEDTAVYYCACAIERSGIYYHEDSNKYQYWGQGTQVSVS

SEQ ID NO: 101 - protein, 124 aa, Artificial Sequence
  Variable Heavy Domain of S2-13
MAQVQLVESGGGLVQPGGSLRLSCAASGFTMTYYTMTWFRQAPGKEREWVSSSRITGNTYYTDSVKGRFTISRDNAKSTVYLQMNSLKPEDTATYYCASGRNINYYTNSAQYDYWGRGTQVTVS

SEQ ID NO: 102 - protein, 127 aa, Artificial Sequence
  Variable Heavy Domain of S2-14
MAQVQLVESGGGLVQTGGSLRLSCAPSGRTFSPYAMGWFRQAPGKEREFVSGIGWYSGTTYYADSVKGRFTISSDIIKNTLTLQMNSLKPEDTAVYYCAASKDRYGNMDPNGVRYDYWGQGTQVTVS

SEQ ID NO: 103 - protein, 133 aa, Artificial Sequence
  Variable Heavy Domain of S2-15
MAQVQLVESGGGLVQAGGSLRLSCAASGRTFSWYNMAWFRQAPGKEREFVAGISLTDISRSGGNTYYADSVKGRFTISRDNAKNTVYMQMDSLKPEDTAVYYCAADDRRMPAAIMTSVRDYVYRGQGTQVTVS

SEQ ID NO: 104 - protein, 126 aa, Artificial Sequence
  Variable Heavy Domain of S2-18
MTQVQLVESGGGLVQAGDSLRLSCVASESTFSSRTMGWFRQAPGKERDFVAAIPWWIGALPNYKDSVKGRFTISRDSAKNTMYLQMNSLKPEDTAVYYCAASKTWSPRTDVTELEFRGQGTQVTVS

SEQ ID NO: 105 - protein, 128 aa, Artificial Sequence
  Variable Heavy Domain of S2-22
MAQVHLAESGGGLVQAGGSLRLSCAASGRTFSWYNLAWFRQTPGKEREFVAGIGRIGGNIYYADSVKGRFTISEDNAKNTVYLKMDSLKPEDTAVYHCAADDRRMPAAIMTSARDYVYWGQGTQVTVS

SEQ ID NO: 106 - protein, 127 aa, Artificial Sequence
  Variable Heavy Domain of S2-26
MAQVQLVESGGGLVQAGGSLRLSCAVSGGTFSRYSMGWFRQAPGKEREFVAAISSNDKATYYKDSVKDRFTISRDNAKNTVYLHMNTLKPEDTAVYYCAADPVLKMTPDIMASRYVYWGQGTQVTVS

SEQ ID NO: 107 - protein, 117 aa, Artificial Sequence
  Variable Heavy Domain of S2-33
MAQVQLLESGGGLVQPGGSLRLSCVVSGSIANMRVMKWYRQAPGKEREWVGTMFNPGPTMYRDSVKGRFTISRDNAKNTVNLQMNSLKVEDTAVYYCNVVDPRNYEYWGQGTQVTVS

SEQ ID NO: 108 - protein, 117 aa, Artificial Sequence
  Variable Heavy Domain of S2-35
MAQVQLVESGGGLVQPGGSLRLSCAASFRISSVEVLGWYRQTPGKERELVATLTRAGTTNYIDAVKGRFTISRDNAKNMLYLQMNSLKPEDTAVYYCNVRDLIGQNFWGQGTHVTVS

SEQ ID NO: 109 - protein, 121 aa, Artificial Sequence
  Variable Heavy Domain of S2-36
MAQVQLVESGGGLVQAGGSLRLSCAASGRIATNYVMDWYRQAPGKQQELVASISNDGSTNYADSVKGRFTISRDNAKNTLYLQMNSLKPEDTAVYLCHLKHWWFGFAKYDYWGQGTQVTVS

SEQ ID NO: 110 - protein, 114 aa, Artificial Sequence
  Variable Heavy Domain of S2-39
MAQVQLVESGGDLVQAGESLRLSCAASGIIISRYAMDWYRQAPGKQREFVAEISSGGTTNYADSVKGRFTIFRDNAKNTMYLQMNSLKPEDTAVYYCKVSYTTYSGQGTRVTVS

SEQ ID NO: 111 - protein, 128 aa, Artificial Sequence
  Variable Heavy Domain of S2-40
MAQVQLVESGGGLVQAGGSLRLSCAASGRTFSWYNLAWFRQAPGKEREFVAGISRIAGNTYYADSVKGRFTISEDNAKNTVYMQMDSLKPEDTAVYHCAADDRWMPAATMTSVRDYVYRGQGTQVTVS

SEQ ID NO: 112 - protein, 128 aa, Artificial Sequence
  Variable Heavy Domain of S2-42
MAQVQLVESGGGLVQAGGSLRLSCAASGRTFSWYNLAWFRQAPGKEFEFVGGISRTAGNTYYADSVKGRFTISEDNAKNTVYIQMDSLKPEDTAVYHCAADDRRMAAATMTSVRDYVYRGQGTQVTVS

SEQ ID NO: 113 - protein, 114 aa, Artificial Sequence
  Variable Heavy Domain of S2-47
MAQVRVEESGGDLVQAGGSLRLSCTTSAILNTWPMGWYRQAPGKQRELVAQITSGGTTNYADSVKGRFTISRDGRQNIVYLQMNRLKPEDTAVYYCNLRADRDYWGQGTQVTVS

SEQ ID NO: 114 - protein, 125 aa, Artificial Sequence
  Variable Heavy Domain of S2-58
MAQVQLVESGGGLVQAGGSLRLSCVASGRIFSSYAMAWFRQAPGKEREFVATITWSGKLTMYQDSVKGRFTISKDNPKNTVYLQMNTLKPEDTALYYCAAHEKGWKQVSPDDVKYWGQGTQVTVS

SEQ ID NO: 115 - protein, 126 aa, Artificial Sequence
  Variable Heavy Domain of S2-59
MAQVQLVESGGGQAQAGGSLRLSCAASGRTISNYNMGWFRQAPGKEREFVAAAKWSDINESYADSVKGRFTISRDNAKNTVYLQMNSLKPEDTAVYYCAAAGGYYSDNIRQTDYNYWGQGTQVTVS

SEQ ID NO: 116 - protein, 128 aa, Artificial Sequence
  Variable Heavy Domain of S2-62
MAQVQLVESGGGLVQPGGSLRLSCAASGRTFSSAMMGWFRQAPGKEREFVASIDWRYQYRYYADSVKGRFTISRDNAKNTVYLQMNSLKPDDTAVYYCACQIQRDGIYYYEDSSKYQYWGQGTQVSVS

SEQ ID NO: 117 - protein, 9 aa, Artificial Sequence
  CDR1 of S1-1
MPFSSYAMG

SEQ ID NO: 118 - protein, 10 aa, Artificial Sequence
  CDR2 of S1-1
AVNWNGISTY

SEQ ID NO: 119 - protein, 12 aa, Artificial Sequence
  CDR3 of S1-1
DSKGWSEWGMDY

SEQ ID NO: 120 - protein, 9 aa, Artificial Sequence
  CDR1 of S1-2 and S1-50
SIIRPNVMA

SEQ ID NO: 121 - protein, 9 aa, Artificial Sequence
  CDR2 of S1-2
SIHSSGTKN

SEQ ID NO: 122 - protein, 10 aa, Artificial Sequence
  CDR3 of S1-2
RSTWATQPDY

SEQ ID NO: 123 - protein, 9 aa, Artificial Sequence
  CDR1 of S1-3
STFSNTGLG

SEQ ID NO: 124 - protein, 9 aa, Artificial Sequence
  CDR2 of S1-3
SISPSGSTS

SEQ ID NO: 125 - protein, 6 aa, Artificial Sequence
  CDR3 of S1-3
LPRSGS

SEQ ID NO: 126 - protein, 9 aa, Artificial Sequence
  CDR1 of S1-4
RTFSSYFMA

SEQ ID NO: 127 - protein, 10 aa, Artificial Sequence
  CDR2 of S1-4
AVDWSGGDTL

SEQ ID NO: 128 - protein, 16 aa, Artificial Sequence
  CDR3 of S1-4
DRGYSLYYYQAREYEY

SEQ ID NO: 129 - protein, 9 aa, Artificial Sequence
  CDR1 of S1-5 and S1-RBD-4
FTLGYYAIG

SEQ ID NO: 130 - protein, 10 aa, Artificial Sequence
  CDR2 of S1-5
CIGSSDGSTY

SEQ ID NO: 131 - protein, 18 aa, Artificial Sequence
  CDR3 of S1-5
KGPPQGVLRCSISGRYDF

SEQ ID NO: 132 - protein, 9 aa, Artificial Sequence
  CDR1 of S1-6
RTSSFFSMA

SEQ ID NO: 133 - protein, 10 aa, Artificial Sequence
  CDR2 of S1-6
VIRGSGGVTF

SEQ ID NO: 134 - protein, 17 aa, Artificial Sequence
  CDR3 of S1-6
SDFGDHGPLSGRTDFSS

SEQ ID NO: 135 - protein, 11 aa, Artificial Sequence
  CDR1 of S1-7
RTYTSSMYGMA

SEQ ID NO: 136 - protein, 7 aa, Artificial Sequence
  CDR2 of S1-7
VIGWNTY

SEQ ID NO: 137 - protein, 18 aa, Artificial Sequence
  CDR3 of S1-7
RKAAGLASGYPSPSYYDY

SEQ ID NO: 138 - protein, 12 aa, Artificial Sequence
  CDR1 of S1-9
VSGSAFRFNRLG

SEQ ID NO: 139 - protein, 9 aa, Artificial Sequence
  CDR2 of S1-9 and S1-25
SISSGGSTN

SEQ ID NO: 140 - protein, 12 aa, Artificial Sequence
  CDR3 of S1-9
RADFFSSTRTGD

SEQ ID NO: 141 - protein, 9 aa, Artificial Sequence
  CDR1 of S1-10
RTFSSAGMA

SEQ ID NO: 142 - protein, 10 aa, Artificial Sequence
  CDR2 of S1-10
TSNWNGGDTD

SEQ ID NO: 143 - protein, 13 aa, Artificial Sequence
  CDR3 of S1-10
HLQKYGGDYYARI

SEQ ID NO: 144 - protein, 9 aa, Artificial Sequence
  CDR1 of S1-11
FTLSPYDIG

SEQ ID NO: 145 - protein, 10 aa, Artificial Sequence
  CDR2 of S1-11
CISVTGGTTD

SEQ ID NO: 146 - protein, 17 aa, Artificial Sequence
  CDR3 of S1-11
HTASFCRGDYSASVARY

SEQ ID NO: 147 - protein, 9 aa, Artificial Sequence
  CDR1 of S1-12
RTFSNYAMG

SEQ ID NO: 148 - protein, 10 aa, Artificial Sequence
  CDR2 of S1-12
AISWKSGLIT

SEQ ID NO: 149 - protein, 15 aa, Artificial Sequence
  CDR3 of S1-12
DPNNRRGTLTSYYDH

SEQ ID NO: 150 - protein, 9 aa, Artificial Sequence
  CDR1 of S1-14
STFSRLSMG

SEQ ID NO: 151 - protein, 9 aa, Artificial Sequence
  CDR2 of S1-14
RILPLGGPY

SEQ ID NO: 152 - protein, 15 aa, Artificial Sequence
  CDR3 of S1-14
APFGTAWDGPDNYDY

SEQ ID NO: 153 - protein, 9 aa, Artificial Sequence
  CDR1 of S1-17
LASTYSAKG

SEQ ID NO: 154 - protein, 10 aa, Artificial Sequence
  CDR2 of S1-17
AINRSGGITS

SEQ ID NO: 155 - protein, 17 aa, Artificial Sequence
  CDR3 of S1-17
KVTLSDDTYNQPGNYPS

SEQ ID NO: 156 - protein, 9 aa, Artificial Sequence
  CDR1 of S1-19
STFSSYAMG

SEQ ID NO: 157 - protein, 9 aa, Artificial Sequence
  CDR2 of S1-19
GISPDGSTN

SEQ ID NO: 158 - protein, 10 aa, Artificial Sequence
  CDR3 of S1-19
FLPEIPGRGS

SEQ ID NO: 159 - protein, 9 aa, Artificial Sequence
  CDR1 of S1-20
FPLDFYAIG

SEQ ID NO: 160 - protein, 10 aa, Artificial Sequence
  CDR2 of S1-20 and S1-37
CISRRDDYTS

SEQ ID NO: 161 - protein, 18 aa, Artificial Sequence
  CDR3 of S1-20
VRTSSGTVCQAYPRWYDT

SEQ ID NO: 162 - protein, 9 aa, Artificial Sequence
  CDR1 of S1-21
IAVRASTMG

SEQ ID NO: 163 - protein, 9 aa, Artificial Sequence
  CDR2 of S1-21
TINIGGSTN

SEQ ID NO: 164 - protein, 8 aa, Artificial Sequence
  CDR3 of S1-21
QYYTGYDY

SEQ ID NO: 165 - protein, 9 aa, Artificial Sequence
  CDR1 of S1-23
RTSRPYRMG

SEQ ID NO: 166 - protein, 10 aa, Artificial Sequence
  CDR2 of S1-23
AIFWSGSHTL

SEQ ID NO: 167 - protein, 10 aa, Artificial Sequence
  CDR3 of S1-23
RNQATGNYDV

SEQ ID NO: 168 - protein, 9 aa, Artificial Sequence
  CDR1 of S1-24
STTTNYHMG

SEQ ID NO: 169 - protein, 9 aa, Artificial Sequence
  CDR2 of S1-24
AINAGGITN

SEQ ID NO: 170 - protein, 17 aa, Artificial Sequence
  CDR3 of S1-24
GGGWDYRNSYYIPRVDS

SEQ ID NO: 171 - protein, 12 aa, Artificial Sequence
  CDR1 of S1-25
VSGSGFRFNRLG

SEQ ID NO: 172 - skipped

SEQ ID NO: 173 - protein, 12 aa, Artificial Sequence
  CDR3 of S1-25
RADFFTSTRTGD

SEQ ID NO: 174 - protein, 9 aa, Artificial Sequence
  CDR1 of S1-27
GTFGSSRMG

SEQ ID NO: 175 - protein, 10 aa, Artificial Sequence
  CDR2 of S1-27
AIMPSGSHTN

SEQ ID NO: 176 - protein, 10 aa, Artificial Sequence
  CDR3 of S1-27
RDPSSYTYDR

SEQ ID NO: 177 - protein, 9 aa, Artificial Sequence
  CDR1 of S1-28
RTLSSYVMG

SEQ ID NO: 178 - protein, 10 aa, Artificial Sequence
  CDR2 of S1-28 and S1-36
AIRWNGGSTF

SEQ ID NO: 179 - protein, 19 aa, Artificial Sequence
  CDR3 of S1-28
SPRTMYYASYYYTRTSYDY

SEQ ID NO: 180 - protein, 9 aa, Artificial Sequence
  CDR1 of S1-29
DTFSSAPMG

SEQ ID NO: 181 - protein, 10 aa, Artificial Sequence
  CDR2 of S1-29
GISWSALNTY

SEQ ID NO: 182 - protein, 19 aa, Artificial Sequence
  CDR3 of S1-29
RTRSHFYSEVYRRASDYDF

SEQ ID NO: 183 - protein, 9 aa, Artificial Sequence
  CDR1 of S1-30
STFSTSHMG

SEQ ID NO: 184 - protein, 9 aa, Artificial Sequence
  CDR2 of S1-30
SISTEGTTE

SEQ ID NO: 185 - protein, 18 aa, Artificial Sequence
  CDR3 of S1-30
GGWRYYGDGSHYIPDFRF

SEQ ID NO: 186 - protein, 9 aa, Artificial Sequence
  CDR1 of S1-31
SISSIIYRG

SEQ ID NO: 187 - protein, 9 aa, Artificial Sequence
  CDR2 of S1-31
SIDTAGSTT

SEQ ID NO: 188 - protein, 9 aa, Artificial Sequence
  CDR3 of S1-31
VTWAPRVEY

SEQ ID NO: 189 - protein, 9 aa, Artificial Sequence
  CDR1 of S1-32
VAFSLYHMA

SEQ ID NO: 190 - protein, 10 aa, Artificial Sequence
  CDR2 of S1-32
AINWNGGIAD

SEQ ID NO: 191 - protein, 17 aa, Artificial Sequence
  CDR3 of S1-32
KLGAYYTTYRKANEYQY

SEQ ID NO: 192 - protein, 9 aa, Artificial Sequence
  CDR1 of S1-35
RTFSSLVMA

SEQ ID NO: 193 - protein, 10 aa, Artificial Sequence
  CDR2 of S1-36 and S1-RBD-30
FIEWSGAETY

SEQ ID NO: 194 - protein, 16 aa, Artificial Sequence
  CDR3 of S1-35
DKGYSLYYYRESDYEY

SEQ ID NO: 195 - protein, 9 aa, Artificial Sequence
  CDR1 of S1-36
RILSSYVMG

SEQ ID NO: 196 - skipped

SEQ ID NO: 197 - protein, 19 aa, Artificial Sequence
  CDR3 of S1-36
SPRTMYLASYYYHRTSYDY

SEQ ID NO: 198 - protein, 9 aa, Artificial Sequence
  CDR1 of S1-37
FPLDFFAIG

SEQ ID NO: 199 - skipped

SEQ ID NO: 200 - protein, 18 aa, Artificial Sequence
  CDR3 of S1-37
VRTSSDTVCQNYPRWYLT

SEQ ID NO: 201 - protein, 9 aa, Artificial Sequence
  CDR1 of S1-38
RTFSYAIMG

SEQ ID NO: 202 - protein, 10 aa, Artificial Sequence
  CDR2 of S1-38
WMAWSGPAKY

SEQ ID NO: 203 - protein, 13 aa, Artificial Sequence
  CDR3 of S1-38
SKEITTMTTRYDY

SEQ ID NO: 204 - protein, 9 aa, Artificial Sequence
  CDR1 of S1-39
RTFGSYAMG

SEQ ID NO: 205 - protein, 10 aa, Artificial Sequence
  CDR2 of S1-39
AINWSGDHTY

SEQ ID NO: 206 - protein, 17 aa, Artificial Sequence
  CDR3 of S1-39
RGPNEWGLPREDLFYDY

SEQ ID NO: 207 - protein, 9 aa, Artificial Sequence
  CDR1 of S1-41
STFSTYHMG

SEQ ID NO: 208 - protein, 9 aa, Artificial Sequence
  CDR2 of S1-41
AINSEGSTY

SEQ ID NO: 209 - protein, 18 aa, Artificial Sequence
  CDR3 of S1-41
GGWRYYGDGSRYIPDFRS

SEQ ID NO: 210 - protein, 9 aa, Artificial Sequence
  CDR1 of S1-46
HQFSDYVIG

SEQ ID NO: 211 - protein, 10 aa, Artificial Sequence
  CDR2 of S1-46
AIWWSDDITY

SEQ ID NO: 212 - protein, 18 aa, Artificial Sequence
  CDR3 of S1-46
RFRPVSTYQVRSTDDYGY

SEQ ID NO: 213 - protein, 9 aa, Artificial Sequence
  CDR1 of S1-48
ITLDYYAIG

SEQ ID NO: 214 - protein, 10 aa, Artificial Sequence
  CDR2 of S1-48
LISSSDGSTY

SEQ ID NO: 215 - protein, 21 aa, Artificial Sequence
  CDR3 of S1-48
GSLTRYGSSWHVPFGSRSYEN

SEQ ID NO: 216 - protein, 9 aa, Artificial Sequence
  CDR1 of S1-49
SIFSIGAMS

SEQ ID NO: 217 - protein, 9 aa, Artificial Sequence
  CDR2 of S1-49
QISSGGITN

SEQ ID NO: 218 - protein, 11 aa, Artificial Sequence
  CDR3 of S1-49
DETTPWGATRG

SEQ ID NO: 219 - skipped

SEQ ID NO: 220 - protein, 9 aa, Artificial Sequence
  CDR2 of S1-50
SMHSSGTKN

SEQ ID NO: 221 - protein, 10 aa, Artificial Sequence
  CDR3 of S1-50
RSTWAGQPDY

SEQ ID NO: 222 - protein, 9 aa, Artificial Sequence
  CDR1 of S1-51
FSFSNYYMV

SEQ ID NO: 223 - protein, 10 aa, Artificial Sequence
  CDR2 of S1-51
AITTWLSNTY

SEQ ID NO: 224 - protein, 17 aa, Artificial Sequence
  CDR3 of S1-51
EQSGRDWRRTSPSPYAY

SEQ ID NO: 225 - protein, 9 aa, Artificial Sequence
  CDR1 of S1-52
SIFSLNAKA

SEQ ID NO: 226 - protein, 10 aa, Artificial Sequence
  CDR2 of S1-52
RITGSGTITN

SEQ ID NO: 227 - protein, 11 aa, Artificial Sequence
  CDR3 of S1-52
APGGLWTGPAY

SEQ ID NO: 228 - protein, 9 aa, Artificial Sequence
  CDR1 of S1-53
YTITNYWMA

SEQ ID NO: 229 - protein, 10 aa, Artificial Sequence
  CDR2 of S1-53
TITYGAGMKY

SEQ ID NO: 230 - protein, 13 aa, Artificial Sequence
  CDR3 of S1-53
PQRPYSGFGSDDY

SEQ ID NO: 231 - protein, 9 aa, Artificial Sequence
  CDR1 of S1-54
IIFSKYAVA

SEQ ID NO: 232 - protein, 9 aa, Artificial Sequence
  CDR2 of S1-54
TINHSGDTK

SEQ ID NO: 233 - protein, 16 aa, Artificial Sequence
  CDR3 of S1-54
GIWIASAHSPDGRADH

SEQ ID NO: 234 - protein, 9 aa, Artificial Sequence
  CDR1 of S1-55
RTFGMRAMG

SEQ ID NO: 235 - protein, 10 aa, Artificial Sequence
  CDR2 of S1-55
VISTSGGDTS

SEQ ID NO: 236 - protein, 15 aa, Artificial Sequence
  CDR3 of S1-55
PRIGAWSRSPRDYDI

SEQ ID NO: 237 - protein, 9 aa, Artificial Sequence
  CDR1 of S1-56
SIFSIDAMG

SEQ ID NO: 238 - protein, 9 aa, Artificial Sequence
  CDR2 of S1-56
HIRNTGTTA

SEQ ID NO: 239 - protein, 13 aa, Artificial Sequence
  CDR3 of S1-56
LKDFLGGVIPLNS

SEQ ID NO: 240 - protein, 9 aa, Artificial Sequence
  CDR1 of S1-58
STFSAYSMG

SEQ ID NO: 241 - protein, 9 aa, Artificial Sequence
  CDR2 of S1-58
AISSGGSTN

SEQ ID NO: 242 - protein, 17 aa, Artificial Sequence
  CDR3 of S1-58
VGWDYRYDYPITGRNDY

SEQ ID NO: 243 - protein, 9 aa, Artificial Sequence
  CDR1 of S1-60
STFSTNAMG

SEQ ID NO: 244 - protein, 9 aa, Artificial Sequence
  CDR2 of S1-60
EITTDGTTR

SEQ ID NO: 245 - protein, 13 aa, Artificial Sequence
  CDR3 of S1-60
VTRFLGGLLRPDY

SEQ ID NO: 246 - protein, 9 aa, Artificial Sequence
  CDR1 of S1-61 and S1-RBD-15
RTFSSYAMG

SEQ ID NO: 247 - protein, 10 aa, Artificial Sequence
  CDR2 of S1-61
AISRRGMDTY

SEQ ID NO: 248 - protein, 18 aa, Artificial Sequence
  CDR3 of S1-61
KMVAPIFVVVTTRDEYDY

SEQ ID NO: 249 - protein, 9 aa, Artificial Sequence
  CDR1 of S1-62
RTSNTYPMA

SEQ ID NO: 250 - protein, 10 aa, Artificial Sequence
  CDR2 of S1-62
AITRSVPNTY

SEQ ID NO: 251 - protein, 14 aa, Artificial Sequence
  CDR3 of S1-62
SSNVYLLTRDEYDY

SEQ ID NO: 252 - protein, 9 aa, Artificial Sequence
  CDR1 of S1-63
IIFSINAMG

SEQ ID NO: 253 - protein, 9 aa, Artificial Sequence
  CDR2 of S1-63
LIMNNGNTY

SEQ ID NO: 254 - protein, 13 aa, Artificial Sequence
  CDR3 of S1-63
VAYRDYDRKRYDY

SEQ ID NO: 255 - protein, 9 aa, Artificial Sequence
  CDR1 of S1-64
SIFSVNSMG

SEQ ID NO: 256 - protein, 9 aa, Artificial Sequence
  CDR2 of S1-64
TITSHGITN

SEQ ID NO: 257 - protein, 12 aa, Artificial Sequence
  CDR3 of S1-64
EDTGLWNSKDTY

SEQ ID NO: 258 - protein, 9 aa, Artificial Sequence
  CDR1 of S1-65
SIFNVNSMG

SEQ ID NO: 259 - protein, 9 aa, Artificial Sequence
  CDR2 of S1-65
TITTRGITN

SEQ ID NO: 260 - protein, 12 aa, Artificial Sequence
  CDR3 of S1-65
EDTGLWNSKDVY

SEQ ID NO: 261 - protein, 5 aa, Artificial Sequence
  CDR1 of S1-66
RTFMG

SEQ ID NO: 262 - protein, 10 aa, Artificial Sequence
  CDR2 of S1-66
AISWNGVSIT

SEQ ID NO: 263 - protein, 14 aa, Artificial Sequence
  CDR3 of S1-66
ADVVAGTRWTDYNY

SEQ ID NO: 264 - protein, 9 aa, Artificial Sequence
  CDR1 of S1-RBD-3
FTLDTTAIG

SEQ ID NO: 265 - protein, 10 aa, Artificial Sequence
  CDR2 of S1-RBD-3
CISTSGERTY

SEQ ID NO: 266 - protein, 19 aa, Artificial Sequence
  CDR3 of S1-RBD-3
KYASFQYLPVGLPWSMYDV

SEQ ID NO: 267 - skipped

SEQ ID NO: 268 - protein, 10 aa, Artificial Sequence
  CDR2 of S1-RBD-4
FISRHGSTTY

SEQ ID NO: 269 - protein, 18 aa, Artificial Sequence
  CDR3 of S1-RBD-4
SKTTAWDTIRVSAIEYDY

SEQ ID NO: 270 - protein, 9 aa, Artificial Sequence
  CDR1 of S1-RBD-5 and S1-RBD-30
RTFSSLVMG

SEQ ID NO: 271 - protein, 10 aa, Artificial Sequence
  CDR2 of S1-RBD-5
FIEWNGGDTY

SEQ ID NO: 272 - protein, 16 aa, Artificial Sequence
  CDR3 of S1-RBD-5
DRAYSLYYYRVSEYQY

SEQ ID NO: 273 - protein, 9 aa, Artificial Sequence
  CDR1 of S1-RBD-6
RTFTTNAMG

SEQ ID NO: 274 - protein, 10 aa, Artificial Sequence
  CDR2 of S1-RBD-6
AISWSTGTTY

SEQ ID NO: 275 - protein, 17 aa, Artificial Sequence
  CDR3 of S1-RBD-6
RSAFNAYAWTTERAYDY

SEQ ID NO: 276 - protein, 9 aa, Artificial Sequence
  CDR1 of S1-RBD-9
RTFSNYVVA

SEQ ID NO: 277 - protein, 10 aa, Artificial Sequence
  CDR2 of S1-RBD-9
LITWSGADRY

SEQ ID NO: 278 - protein, 16 aa, Artificial Sequence
  CDR3 of S1-RBD-9
DRGSSYHYAESKNYDY

SEQ ID NO: 279 - protein, 9 aa, Artificial Sequence
  CDR1 of S1-RBD-10
RTFRNYAMG

SEQ ID NO: 280 - protein, 10 aa, Artificial Sequence
  CDR2 of S1-RBD-10
VITATDDITY

SEQ ID NO: 281 - protein, 16 aa, Artificial Sequence
  CDR3 of S1-RBD-10
DYGDSYYYTRRTEYGY

SEQ ID NO: 282 - protein, 9 aa, Artificial Sequence
  CDR1 of S1-RBD-11 and S1-RBD-35
STDSNYVMG

SEQ ID NO: 283 - protein, 9 aa, Artificial Sequence
  CDR2 of S1-RBD-11
SINSGGETR

SEQ ID NO: 284 - protein, 10 aa, Artificial Sequence
  CDR3 of S1-RBD-11
ERLAWDPSTS

SEQ ID NO: 285 - protein, 9 aa, Artificial Sequence
  CDR1 of S1-RBD-12
SILSINDMG

SEQ ID NO: 286 - protein, 9 aa, Artificial Sequence
  CDR2 of S1-RBD-12
FITSGGSTN

SEQ ID NO: 287 - protein, 17 aa, Artificial Sequence
  CDR3 of S1-RBD-12
DIRESGFVYGMNAWGKY

SEQ ID NO: 288 - protein, 9 aa, Artificial Sequence
  CDR1 of S1-RBD-14
RSFSINPMG

SEQ ID NO: 289 - protein, 10 aa, Artificial Sequence
  CDR2 of S1-RBD-14
AISWSGSKTV

SEQ ID NO: 290 - protein, 20 aa, Artificial Sequence
  CDR3 of S1-RBD-14
ASRGPVYGANYVPGPHEYEY

SEQ ID NO: 291 - skipped

SEQ ID NO: 292 - protein, 10 aa, Artificial Sequence
  CDR2 of S1-RBD-15
SINWNGGNTY

SEQ ID NO: 293 - protein, 17 aa, Artificial Sequence
  CDR3 of S1-RBD-15
TGPNEYGLPREDLFYDY

SEQ ID NO: 294 - protein, 9 aa, Artificial Sequence
  CDR1 of S1-RBD-16
RGFDRYPMG

SEQ ID NO: 295 - protein, 10 aa, Artificial Sequence
  CDR2 of S1-RBD-16
GINWNGANTY

SEQ ID NO: 296 - protein, 17 aa, Artificial Sequence
  CDR3 of S1-RBD-16
DQHYMGSPPPKEYEYDY

SEQ ID NO: 297 - protein, 9 aa, Artificial Sequence
  CDR1 of S1-RBD-18
ITFSGYVMA

SEQ ID NO: 298 - protein, 9 aa, Artificial Sequence
  CDR2 of S1-RBD-18
WISHGGIKD

SEQ ID NO: 299 - protein, 6 aa, Artificial Sequence
  CDR3 of S1-RBD-18
KDAQSS

SEQ ID NO: 300 - protein, 9 aa, Artificial Sequence
  CDR1 of S1-RBD-19
FSFSSYGMG

SEQ ID NO: 301 - protein, 10 aa, Artificial Sequence
  CDR2 of S1-RBD-19
TISGTGGNTA

SEQ ID NO: 302 - protein, 19 aa, Artificial Sequence
  CDR3 of S1-RBD-19
PPSWYTRRYINFAPSEVRH

SEQ ID NO: 303 - protein, 9 aa, Artificial Sequence
  CDR1 of S1-RBD-20
RTFSSGAMA

SEQ ID NO: 304 - protein, 9 aa, Artificial Sequence
  CDR2 of S1-RBD-20
VIRWSGSTY

SEQ ID NO: 305 - protein, 19 aa, Artificial Sequence
  CDR3 of S1-RBD-20
TDRTHYDIVEYTRDHFYSN

SEQ ID NO: 306 - protein, 9 aa, Artificial Sequence
  CDR1 of S1-RBD-21
RAFSDYGMG

SEQ ID NO: 307 - protein, 10 aa, Artificial Sequence
  CDR2 of S1-RBD-21
TIDSTGSNTY

SEQ ID NO: 308 - protein, 14 aa, Artificial Sequence
  CDR3 of S1-RBD-21
DRFGGGRMEYRYEY

SEQ ID NO: 309 - protein, 9 aa, Artificial Sequence
  CDR1 of S1-RBD-22
RTFSAYTLA

SEQ ID NO: 310 - protein, 10 aa, Artificial Sequence
  CDR2 of S1-RBD-22
HIDWSGTETN

SEQ ID NO: 311 - protein, 16 aa, Artificial Sequence
  CDR3 of S1-RBD-22
DRAYSLMYYDTREYEY

SEQ ID NO: 312 - protein, 9 aa, Artificial Sequence
  CDR1 of S1-RBD-23
SGFENNAIT

SEQ ID NO: 313 - protein, 9 aa, Artificial Sequence
  CDR2 of S1-RBD-23
GITSGGSTN

SEQ ID NO: 314 - protein, 13 aa, Artificial Sequence
  CDR3 of S1-RBD-23
VAWDSRRRSVVAF

SEQ ID NO: 315 - protein, 9 aa, Artificial Sequence
  CDR1 of S1-RBD-24
HTFGSLTMA

SEQ ID NO: 316 - protein, 8 aa, Artificial Sequence
  CDR2 of S1-RBD-24
GTDWSGIN

SEQ ID NO: 317 - protein, 17 aa, Artificial Sequence
  CDR3 of S1-RBD-24
DRAASLYPLRDQADYMY

SEQ ID NO: 318 - protein, 9 aa, Artificial Sequence
  CDR1 of S1-RBD-25 and S1-RBD-32
FTLDYYTIG

SEQ ID NO: 319 - protein, 10 aa, Artificial Sequence
  CDR2 of S1-RBD-25
YISSSGSNTY

SEQ ID NO: 320 - protein, 18 aa, Artificial Sequence
  CDR3 of S1-RBD-25
RRVWQETIVVRGPEHYEY

SEQ ID NO: 321 - protein, 9 aa, Artificial Sequence
  CDR1 of S1-RBD-26
RTFGGYGVG

SEQ ID NO: 322 - protein, 10 aa, Artificial Sequence
  CDR2 of S1-RBD-26
GITSTSFGTS

SEQ ID NO: 323 - protein, 15 aa, Artificial Sequence
  CDR3 of S1-RBD-26
SVLSPPPRTNSAYAY

SEQ ID NO: 324 - protein, 9 aa, Artificial Sequence
  CDR1 of S1-RBD-27
RTFGIYNMG

SEQ ID NO: 325 - protein, 10 aa, Artificial Sequence
  CDR2 of S1-RBD-27
AITGDASDTY

SEQ ID NO: 326 - protein, 14 aa, Artificial Sequence
  CDR3 of S1-RBD-27
TGAITRATMWKPNF

SEQ ID NO: 327 - protein, 9 aa, Artificial Sequence
  CDR1 of S1-RBD-28
RTISTDAMG

SEQ ID NO: 328 - protein, 9 aa, Artificial Sequence
  CDR2 of S1-RBD-28
ISWRSDDTY

SEQ ID NO: 329 - protein, 16 aa, Artificial Sequence
  CDR3 of S1-RBD-28
DQGRSYYYTRESEYNY

SEQ ID NO: 330 - protein, 9 aa, Artificial Sequence
  CDR1 of S1-RBD-29
LASIVFNMG

SEQ ID NO: 331 - protein, 10 aa, Artificial Sequence
  CDR2 of S1-RBD-29
GILGSGQSTY

SEQ ID NO: 332 - protein, 17 aa, Artificial Sequence
  CDR3 of S1-RBD-29
GGRLDLVNTTGGYEPTY

SEQ ID NO: 333-334 - skipped (2 entries)

SEQ ID NO: 335 - protein, 16 aa, Artificial Sequence
  CDR3 of S1-RBD-30
DAAYSLYYYRVSEYEY

SEQ ID NO: 336 - skipped

SEQ ID NO: 337 - protein, 10 aa, Artificial Sequence
  CDR2 of S1-RBD-32
YISSSGSNSY

SEQ ID NO: 338 - protein, 18 aa, Artificial Sequence
  CDR3 of S1-RBD-32
RRVWQPTIVVSGPEHYDY

SEQ ID NO: 339 - protein, 9 aa, Artificial Sequence
  CDR1 of S1-RBD-34 and S1-RBD-36
RTFSTTFMG

SEQ ID NO: 340 - protein, 10 aa, Artificial Sequence
  CDR2 of S1-RBD-34
AINWNGGSTR

SEQ ID NO: 341 - protein, 22 aa, Artificial Sequence
  CDR3 of S1-RBD-34
DPSDGRVVGYNYVPVSTHEYDY

SEQ ID NO: 342 - skipped

SEQ ID NO: 343 - protein, 9 aa, Artificial Sequence
  CDR2 of S1-RBD-35
SINSGGDTK

SEQ ID NO: 344 - protein, 10 aa, Artificial Sequence
  CDR3 of S1-RBD-35
EKLAWDPSTT

SEQ ID NO: 345 - skipped

SEQ ID NO: 346 - protein, 10 aa, Artificial Sequence
  CDR2 of S1-RBD-36
AINWSGGSTR

SEQ ID NO: 347 - protein, 22 aa, Artificial Sequence
  CDR3 of S1-RBD-36
DPSDGRVFGVNYVPVSTHEYDY

SEQ ID NO: 348 - protein, 11 aa, Artificial Sequence
  CDR1 of S1-RBD-37
DSFSFRWYGMN

SEQ ID NO: 349 - protein, 9 aa, Artificial Sequence
  CDR2 of S1-RBD-37
GISSAGSTN

SEQ ID NO: 350 - protein, 7 aa, Artificial Sequence
  CDR3 of S1-RBD-37
ATQERDY

SEQ ID NO: 351 - protein, 9 aa, Artificial Sequence
  CDR1 of S1-RBD-38
STFDNYPVT

SEQ ID NO: 352 - protein, 9 aa, Artificial Sequence
  CDR2 of S1-RBD-38
SIRSGHITN

SEQ ID NO: 353 - protein, 7 aa, Artificial Sequence
  CDR3 of S1-RBD-38
VDAQSDY

SEQ ID NO: 354 - protein, 9 aa, Artificial Sequence
  CDR1 of S1-RBD-39
STFSSDVMA

SEQ ID NO: 355 - protein, 9 aa, Artificial Sequence
  CDR2 of S1-RBD-39
SFSSSGSGN

SEQ ID NO: 356 - protein, 8 aa, Artificial Sequence
  CDR3 of S1-RBD-39
AHVNGGDS

SEQ ID NO: 357 - protein, 9 aa, Artificial Sequence
  CDR1 of S1-RBD-40
ITFSNYGMV

SEQ ID NO: 358 - protein, 9 aa, Artificial Sequence
  CDR2 of S1-RBD-40
SISSGRDTN

SEQ ID NO: 359 - protein, 7 aa, Artificial Sequence
  CDR3 of S1-RBD-40
VTDRSDY

SEQ ID NO: 360 - protein, 9 aa, Artificial Sequence
  CDR1 of S1-RBD-41
ITFSRSAVG

SEQ ID NO: 361 - protein, 9 aa, Artificial Sequence
  CDR2 of S1-RBD-41
AISNSGTTS

SEQ ID NO: 362 - protein, 9 aa, Artificial Sequence
  CDR3 of S1-RBD-41
ADGLLGGDY

SEQ ID NO: 363 - protein, 9 aa, Artificial Sequence
  CDR1 of S1-RBD-43
STFSSDVIT

SEQ ID NO: 364 - protein, 9 aa, Artificial Sequence
  CDR2 of S1-RBD-43
SIQSSGSRN

SEQ ID NO: 365 - protein, 8 aa, Artificial Sequence
  CDR3 of S1-RBD-43
GHVTGGDS

SEQ ID NO: 366 - protein, 9 aa, Artificial Sequence
  CDR1 of S1-RBD-44
STVSNSPMD

SEQ ID NO: 367 - protein, 9 aa, Artificial Sequence
  CDR2 of S1-RBD-44
TVQSGGNAS

SEQ ID NO: 368 - protein, 7 aa, Artificial Sequence
  CDR3 of S1-RBD-44
ADDRSDY

SEQ ID NO: 369 - protein, 9 aa, Artificial Sequence
  CDR1 of S1-RBD-45
ITWMPYGME

SEQ ID NO: 370 - protein, 9 aa, Artificial Sequence
  CDR2 of S1-RBD-45
TISSNGRTN

SEQ ID NO: 371 - protein, 7 aa, Artificial Sequence
  CDR3 of S1-RBD-45
ATALSDY

SEQ ID NO: 372 - protein, 9 aa, Artificial Sequence
  CDR1 of S1-RBD-46
STLGSYAIG

SEQ ID NO: 373 - protein, 10 aa, Artificial Sequence
  CDR2 of S1-RBD-46
FISRDGSTTF

SEQ ID NO: 374 - protein, 18 aa, Artificial Sequence
  CDR3 of S1-RBD-46
SKITAWDTIIVSAIEYTY

SEQ ID NO: 375 - protein, 9 aa, Artificial Sequence
  CDR1 of S1-RBD-47
ISFSTYDMN

SEQ ID NO: 376 - protein, 9 aa, Artificial Sequence
  CDR2 of S1-RBD-47
GIGTLNSPR

SEQ ID NO: 377 - protein, 6 aa, Artificial Sequence
  CDR3 of S1-RBD-47
ASDRDY

SEQ ID NO: 378 - protein, 12 aa, Artificial Sequence
  CDR1 of S1-RBD-48
RTFDNYNIGFIE

SEQ ID NO: 379 - protein, 10 aa, Artificial Sequence
  CDR2 of S1-RBD-48
TISRSGDITN

SEQ ID NO: 380 - protein, 8 aa, Artificial Sequence
  CDR3 of S1-RBD-48
SMTTGGDY

SEQ ID NO: 381 - protein, 9 aa, Artificial Sequence
  CDR1 of S1-RBD-49
STFSIGAMG

SEQ ID NO: 382 - protein, 9 aa, Artificial Sequence
  CDR2 of S1-RBD-49
TITSSGTTS

SEQ ID NO: 383 - protein, 8 aa, Artificial Sequence
  CDR3 of S1-RBD-49
NNLHGGDY

SEQ ID NO: 384 - protein, 9 aa, Artificial Sequence
  CDR1 of S1-RBD-51
RTLSTYRMG

SEQ ID NO: 385 - protein, 10 aa, Artificial Sequence
  CDR2 of S1-RBD-51
AIFWSGGHTV

SEQ ID NO: 386 - protein, 10 aa, Artificial Sequence
  CDR3 of S1-RBD-51
RNPATGNYDY

SEQ ID NO: 387 - protein, 9 aa, Artificial Sequence
  CDR1 of S2-1 and S2-3
LVLGGYDMR

SEQ ID NO: 388 - protein, 9 aa, Artificial Sequence
  CDR2 of S2-1
SISNGGSTD

SEQ ID NO: 389 - protein, 9 aa, Artificial Sequence
  CDR3 of S2-1
RSTYYGIDY

SEQ ID NO: 390 - protein, 9 aa, Artificial Sequence
  CDR1 of S2-2
LQFSRITMA

SEQ ID NO: 391 - protein, 9 aa, Artificial Sequence
  CDR2 of S2-2
TLSTRGTTN

SEQ ID NO: 392 - protein, 8 aa, Artificial Sequence
  CDR3 of S2-2
GPGYKTDY

SEQ ID NO: 393 - skipped

SEQ ID NO: 394 - protein, 9 aa, Artificial Sequence
  CDR2 of S2-3
SITNGGDTR

SEQ ID NO: 395 - protein, 9 aa, Artificial Sequence
  CDR3 of S2-3
HSTYYGRDF

SEQ ID NO: 396 - protein, 9 aa, Artificial Sequence
  CDR1 of S2-4 and S2-7
PTFSSRTMG

SEQ ID NO: 397 - protein, 11 aa, Artificial Sequence
  CDR2 of S2-4
TIPWWRGALPY

SEQ ID NO: 398 - protein, 16 aa, Artificial Sequence
  CDR3 of S2-4
SKTWSPSTGVATEYEF

SEQ ID NO: 399 - protein, 9 aa, Artificial Sequence
  CDR1 of S2-5
LTFKNYAMG

SEQ ID NO: 400 - protein, 10 aa, Artificial Sequence
  CDR2 of S2-5
TINSIGGPAN

SEQ ID NO: 401 - protein, 19 aa, Artificial Sequence
  CDR3 of S2-5
ERTPSTLLAGSSNPSRVDV

SEQ ID NO: 402 - protein, 9 aa, Artificial Sequence
  CDR1 of S2-6
GIFGGSTMG

SEQ ID NO: 403 - protein, 9 aa, Artificial Sequence
  CDR2 of S2-6
TISNSGSAN

SEQ ID NO: 404 - protein, 9 aa, Artificial Sequence
  CDR3 of S2-6
HSIYYGRDY

SEQ ID NO: 405 - skipped

SEQ ID NO: 406 - protein, 11 aa, Artificial Sequence
  CDR2 of S2-7
AIPWWKGALPY

SEQ ID NO: 407 - protein, 16 aa, Artificial Sequence
  CDR3 of S2-7
SKTWSPYTDAATELEF

SEQ ID NO: 408 - protein, 9 aa, Artificial Sequence
  CDR1 of S2-9
STFSTYGVA

SEQ ID NO: 409 - protein, 9 aa, Artificial Sequence
  CDR2 of S2-9
SINSWGWIN

SEQ ID NO: 410 - protein, 8 aa, Artificial Sequence
  CDR3 of S2-9
QNSRGDNY

SEQ ID NO: 411 - protein, 9 aa, Artificial Sequence
  CDR1 of S2-10
RIVSSSAMS

SEQ ID NO: 412 - protein, 10 aa, Artificial Sequence
  CDR2 of S2-10
VIASSNSHTY

SEQ ID NO: 413 - protein, 17 aa, Artificial Sequence
  CDR3 of S2-10
RSHLTIAAWERLKTYDD

SEQ ID NO: 414 - protein, 9 aa, Artificial Sequence
  CDR1 of S2-11
RTFSGATMG

SEQ ID NO: 415 - protein, 10 aa, Artificial Sequence
  CDR2 of S2-11
SILWRYEYTN

SEQ ID NO: 416 - protein, 18 aa, Artificial Sequence
  CDR3 of S2-11
AIERSGIYYHEDSNKYQY

SEQ ID NO: 417 - protein, 9 aa, Artificial Sequence
  CDR1 of S2-13
FTMTYYTMT

SEQ ID NO: 418 - protein, 9 aa, Artificial Sequence
  CDR2 of S2-13
SSRITGNTY

SEQ ID NO: 419 - protein, 15 aa, Artificial Sequence
  CDR3 of S2-13
GRNINYYTNSAQYDY

SEQ ID NO: 420 - protein, 9 aa, Artificial Sequence
  CDR1 of S2-14
RTFSPYAMG

SEQ ID NO: 421 - protein, 10 aa, Artificial Sequence
  CDR2 of S2-14
GIGWYSGTTY

SEQ ID NO: 422 - protein, 17 aa, Artificial Sequence
  CDR3 of S2-14
SKDRYGNMDPNGVRYDY

SEQ ID NO: 423 - protein, 9 aa, Artificial Sequence
  CDR1 of S2-15
RTFSWYNMA

SEQ ID NO: 424 - protein, 15 aa, Artificial Sequence
  CDR2 of S2-15
GISLTDISRSGGNTY

SEQ ID NO: 425 - protein, 18 aa, Artificial Sequence
  CDR3 of S2-15
DDRRMPAAIMTSVRDYVY

SEQ ID NO: 426 - protein, 9 aa, Artificial Sequence
  CDR1 of S2-18
STFSSRTMG

SEQ ID NO: 427 - protein, 11 aa, Artificial Sequence
  CDR2 of S2-18
AIPWWIGALPN

SEQ ID NO: 428 - protein, 15 aa, Artificial Sequence
  CDR3 of S2-18
SKTWSPRTDVTELEF

SEQ ID NO: 429 - protein, 9 aa, Artificial Sequence
  CDR1 of S2-22, S2-40, and S2-42
RTFSWYNLA

SEQ ID NO: 430 - protein, 10 aa, Artificial Sequence
  CDR2 of S2-22
GIGRIGGNIY

SEQ ID NO: 431 - protein, 18 aa, Artificial Sequence
  CDR3 of S2-22
DDRRMPAAIMTSARDYVY

SEQ ID NO: 432 - protein, 9 aa, Artificial Sequence
  CDR1 of S2-26
GTFSRYSMG

SEQ ID NO: 433 - protein, 10 aa, Artificial Sequence
  CDR2 of S2-26
AISSNDKATY

SEQ ID NO: 434 - protein, 17 aa, Artificial Sequence
  CDR3 of S2-26
DPVLKMTPDIMASRYVY

SEQ ID NO: 435 - protein, 9 aa, Artificial Sequence
  CDR1 of S2-33
SIANMRVMK

SEQ ID NO: 436 - protein, 9 aa, Artificial Sequence
  CDR2 of S2-33
TMFNPGPTM

SEQ ID NO: 437 - protein, 8 aa, Artificial Sequence
  CDR3 of S2-33
VDPRNYEY

SEQ ID NO: 438 - protein, 9 aa, Artificial Sequence
  CDR1 of S2-35
RISSVEVLG

SEQ ID NO: 439 - protein, 9 aa, Artificial Sequence
  CDR2 of S2-35
TLTRAGTTN

SEQ ID NO: 440 - protein, 8 aa, Artificial Sequence
  CDR3 of S2-35
RDLIGQNF

SEQ ID NO: 441 - protein, 9 aa, Artificial Sequence
  CDR1 of S2-36
RIATNYVMD

SEQ ID NO: 442 - protein, 9 aa, Artificial Sequence
  CDR2 of S2-36
SISNDGSTN

SEQ ID NO: 443 - protein, 12 aa, Artificial Sequence
  CDR3 of S2-36
KHWWFGFAKYDY

SEQ ID NO: 444 - protein, 9 aa, Artificial Sequence
  CDR1 of S2-39
IIISRYAMD

SEQ ID NO: 445 - protein, 9 aa, Artificial Sequence
  CDR2 of S2-39
EISSGGTTN

SEQ ID NO: 446 - protein, 5 aa, Artificial Sequence
  CDR3 of S2-39
SYTTY

SEQ ID NO: 447 - skipped

SEQ ID NO: 448 - protein, 10 aa, Artificial Sequence
  CDR2 of S2-40
GISRIAGNTY

SEQ ID NO: 449 - protein, 18 aa, Artificial Sequence
  CDR3 of S2-40
DDRWMPAATMTSVRDYVY

SEQ ID NO: 450 - skipped

SEQ ID NO: 451 - protein, 10 aa, Artificial Sequence
  CDR2 of S2-42
GISRTAGNTY

SEQ ID NO: 452 - protein, 18 aa, Artificial Sequence
  CDR3 of S2-42
DDRRMAAATMTSVRDYVY

SEQ ID NO: 453 - protein, 8 aa, Artificial Sequence
  CDR1 of S2-47
ILNTWPMG

SEQ ID NO: 454 - protein, 9 aa, Artificial Sequence
  CDR2 of S2-47
QITSGGTTN

SEQ ID NO: 455 - protein, 6 aa, Artificial Sequence
  CDR3 of S2-47
RADRDY

SEQ ID NO: 456 - protein, 9 aa, Artificial Sequence
  CDR1 of S2-57
RIFSSYAMA

SEQ ID NO: 457 - protein, 10 aa, Artificial Sequence
  CDR2 of S2-57
TITWSGKLTM

SEQ ID NO: 458 - protein, 15 aa, Artificial Sequence
  CDR3 of S2-57
HEKGWKQVSPDDVKY

SEQ ID NO: 459 - protein, 9 aa, Artificial Sequence
  CDR1 of S2-59
RTISNYNMG

SEQ ID NO: 460 - protein, 10 aa, Artificial Sequence
  CDR2 of S2-59
AAKWSDINES

SEQ ID NO: 461 - protein, 16 aa, Artificial Sequence
  CDR3 of S2-59
AGGYYSDNIRQTDYNY

SEQ ID NO: 462 - protein, 9 aa, Artificial Sequence
  CDR1 of S2-62
RTFSSAMMG

SEQ ID NO: 463 - protein, 10 aa, Artificial Sequence
  CDR2 of S2-62
SIDWRYQYRY

SEQ ID NO: 464 - protein, 18 aa, Artificial Sequence
  CDR3 of S2-62
QIQRDGIYYYEDSSKYQY

SEQ ID NO: 465 - protein, 225 aa, Homo sapiens
THTCPPCPAPEFFGGPSVFFFPPKPKDTFMISRTPEVTCVVVDVSHEDPEVKFNWYVDGVEVHNAKTKPREEQYNSTYRVVSVETVFHQDWENGKEYKCKVSNKAFPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 466 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 467 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 468 - protein, 764 aa, Artificial Sequence
  exemplary precursor secretory component
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 469 - protein, 585 aa, Artificial Sequence
  exemplary mature secretory component
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR

SEQ ID NO: 470 - protein, 349 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 471 - protein, 18 aa, Homo sapiens
VEPKSSDKTHTCPPCPAP

SEQ ID NO: 472 - protein, 255 aa, Homo sapiens
VEPKSSDKTHTCPPCPAPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 473 - protein, 453 aa, Homo sapiens [sequence omitted]

SEQ ID NO: 474 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 475 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLASSLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 476 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVAEEEWNTGETYTCVVAHEALPNRVTERTVDKSTGKPTLYNVSLVMSDTAGTCY

SEQ ID NO: 477 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSAEEWNTGETYTCVVAHEALPNRVTERTVDKSTGKPTLYNVSLVMSDTAGTCY

SEQ ID NO: 478 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEAEWNTGETYTCVVAHEALPNRVTERTVDKSTGKPTLYNVSLVMSDTAGTCY

SEQ ID NO: 479 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLAESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 480 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRASATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 481 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 482 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 483 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 484 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCSTYDRNKCYTAVVPLVYGGETKMVETALTPDACYPD

SEQ ID NO: 485 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCRTYDRNKCYTAVVPLVYGGETKMVETALTPDACYPD

SEQ ID NO: 486 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYAYDRNKCYTAVVPLVYGGETKMVETALTPDACYPD

SEQ ID NO: 487 - protein, 137 aa, Homo sapiens
QEDERIVLVDNKCKCARITSRIIRSSEDPNEDIVERNIRIIVPLNNREAISDPTSPLRTRFVYHLSDLCKKCDPTEVELDNQIVTATQSNICDEDSATETCYTYDRNKCYTAVVPLVYGGETKMVETALTPDACYPD

SEQ ID NO: 488 - protein, 136 aa, Homo sapiens
EDERIVLVDNKCKCARITSRIIRSSEDPNEDIVERNIRIIVPLNNRENIADPTSPLRTRFVYHLSDLCKKCDPTEVELDNQIVTATQSNICDEDSATETCYTYDRNKCYTAVVPLVYGGETKMVETALTPDACYPD

SEQ ID NO: 489 - protein, 5 aa, Artificial Sequence
  GlySer linker
  MISC_FEATURE (1)..(5): (GlyGlyGlyGlySer) can be repeated n times wherein n is an integer of 1, 2, 3, 4, 5, 6, 7, 8, 9 or 10
GGGGS

SEQ ID NO: 490 - protein, 200 aa, Artificial Sequence
  GlySer linker
  MISC_FEATURE (2)..(10): Gly can be present or absent
  MISC_FEATURE (12)..(20): Ser can be present or absent
  MISC_FEATURE (21)..(200): GlySer sequence can be present or absent
GGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSSGGGGGGGGGGSSSSSSSSSS

SEQ ID NO: 491 - protein, 90 aa, Artificial Sequence
  GlySer linker
  MISC_FEATURE (5)..(40): (GlyGlyGlySer) can be present or absent
  MISC_FEATURE (46)..(90): (GlyGlyGlyGlySer) can be present or absent
GGGSGGGSGGGSGGGSGGGSGGGSGGGSGGGSGGGSGGGSGGGGSGGGGSGGGGSGGGGSGGGGSGGGGSGGGGSGGGGSGGGGSGGGGS

SEQ ID NO: 492 - protein, 70 aa, Artificial Sequence
  GlySer linker
  MISC_FEATURE (5)..(37): (GlyGlyGlySer) can be present or absent
  MISC_FEATURE (41)..(70): (GlyGlySer) can be present or absent
GGGSGGGSGGGSGGGSGGGSGGGSGGGSGGGSGGGSGGGSGGSGGSGGSGGSGGSGGSGGSGGSGGSGGS

SEQ ID NO: 493 - protein, 45 aa, Artificial Sequence
  GlySer linker
  MISC_FEATURE (5)..(40): (GlyGlyGlySer) can be present or absent
GGGSGGGSGGGSGGGSGGGSGGGSGGGSGGGSGGGSGGGSGGGGS

SEQ ID NO: 494 - protein, 20 aa, Artificial Sequence
  GlySer linker
GGGGSGGGGSGGGGSGGGGS

SEQ ID NO: 495 - protein, 15 aa, Artificial Sequence
  GlySer linker
GGGGSGGGGSGGGGS

SEQ ID NO: 496 - protein, 10 aa, Artificial Sequence
  GlySer linker
GGGGSGGGGS

SEQ ID NO: 497 - protein, 5 aa, Artificial Sequence
  GlySer linker
GGGGS

SEQ ID NO: 498 - protein, 8 aa, Artificial Sequence
  GlySer linker
GGGSGGGS

SEQ ID NO: 499 - protein, 4 aa, Artificial Sequence
  GlySer linker
GGGS

SEQ ID NO: 500 - protein, 6 aa, Artificial Sequence
  GlySer linker
GGSGGS

SEQ ID NO: 501 - protein, 11 aa, Artificial Sequence
  GlySer linker
GGSGGGSGGSG

SEQ ID NO: 502 - protein, 10 aa, Artificial Sequence
  GlySer linker
GGSGGGSGSG

SEQ ID NO: 503 - protein, 8 aa, Artificial Sequence
  GlySer linker
GGSGGGSG

SEQ ID NO: 504 - protein, 7 aa, Artificial Sequence
  anti-4-1BB CDRL1
RASQSVS

SEQ ID NO: 505 - protein, 6 aa, Artificial Sequence
  anti-4-1BB CDRL2
ASNRAT

SEQ ID NO: 506 - protein, 10 aa, Artificial Sequence
  anti-4-1BB CDRL3
QRSNWPPALT

SEQ ID NO: 507 - protein, 4 aa, Artificial Sequence
  anti-4-1BB CDRH1
YYWS

SEQ ID NO: 508 - protein, 12 aa, Artificial Sequence
  anti-4-1BB CDRH3
YGPGNYDWYFDL

SEQ ID NO: 509 - protein, 128 aa, Artificial Sequence
  single-domain antibody CoV2-S1-13
MAEVQLVESGGGLVQPGGSLKLSCAASGFTLDDYTIGWFRQAPGKEREGVSYISSSGSNSYYEDSVKGRFTISRDNAKSTVYLQMNSLKPEDTAVYYCAARRVWQQTIVVRGPVHYEYWGHGTQVTVS

SEQ ID NO: 510 - protein, 124 aa, Artificial Sequence
  single-domain antibody CoV2-S1-18
MAQVRLVESGGGLVQPGGSLRLSCVVSGVSGSAFRFNRLGWYRQPPGKERELVASISSGGSTNYADSVKGRFTISRDNAKSTVSLQMNSLKPEDTAVYYCNIRADFFSSGRTGDWGQGTQVTVS

SEQ ID NO: 511 - protein, 129 aa, Artificial Sequence
  single-domain antibody CoV2-S1-8
MAQVQLVESGGGLVQAGGSLRLSCAASGRVLSSYVMGWFRQAPGKEREFVAAIRWNGGSTFYADSVKGRLTISRDNAKNTVYLQMNSLKPEDTAAYYCAASPRTMYDATYYYSRTSYDYWGQGTQVTVS

SEQ ID NO: 512 - protein, 126 aa, Artificial Sequence
  single-domain antibody CoV2-S1-RBD-13
MAEVQLVESGGGLVQAGGSLRLSCAAPTLTLSSHTIGWFRQAPGKEREGVSYIPMTGLPIYCRDSVKGRFTISTDNAKNTVYLQMDSLKPEDTAVYYCAALLGVRSRMCPESVYTYWGQGTQVTVS

SEQ ID NO: 513 - protein, 127 aa, Artificial Sequence
  single-domain antibody CoV2-S2-12
MTQVQLVESGGGLVQAGGSLRLSCVASEPTFSSRTMGWFRQAPGKERDFVAAMPWWKGALPNYKDSVKGRFTISIDSAKNTMYLQMNSLKPEDTAVYYCAASKTWSPRTEVATEHEFRGQGTQVTVS

SEQ ID NO: 514 - protein, 126 aa, Artificial Sequence
  single-domain antibody CoV2-S2-20
MAQVQLVESGGGLVQPGGSLRLSCAASGFTLTYYTISWFRQAPGKEREWVSSSRISGNTYYTDSVKGRFTISRDNAKSVVYLQMNSLKPEDTATYYCASGRNIPYDTYTNPTKYDYWGRGTQVTVS

SEQ ID NO: 515 - protein, 128 aa, Artificial Sequence
  single-domain antibody CoV2-S2-23
MAQVQLVESGGGLVQPGGSLRLSCAASGRTFSSAMMGWFRQAPGKEREFVASIDWRYQYRYYADSVKGRFTISRDNAKNMVYLQMNSLKPEDTAVYYCACQIERSGIYHYEDSNKYQSWGQGTQVSVS

SEQ ID NO: 516 - protein, 119 aa, Artificial Sequence
  single-domain antibody CoV2-S2-24
MAQVQLVESGGGLVQAGGSLRLSCAASGVTFSSHHMAWYRQTPGKQRELVATLASGGDTNYADSVKGRFTISRDNTKNTIYLQMNSLKPEDTAVYYCNARLWATGFRDYWGQGTQVTVS

SEQ ID NO: 517 - protein, 123 aa, Artificial Sequence
  single-domain antibody CoV2-S2-29
MAQVQLVESGGGLVQPGGSLRLSCAASGRIFSSNAMGWYRQAPGKEREWVACISSGSSTNYADSVKGRFTISRDNAKNTVYLQMNSLKPEDTAVYYCISKEVGWCGREGPIGSWGQGTQVTVS

SEQ ID NO: 518 - protein, 121 aa, Artificial Sequence
  single-domain antibody CoV2-S2-30
MAQVQLVESGGGLVQAGGSLRLSCAASGSTFGNYAMGWYRQPPQAPGNQRDLVAFISTTGSTNYADSVKGRFTISRDNAKNTMYLQMNSLKPEDTAVYYCQAIERIVGYVSWGQGTQVTVS

SEQ ID NO: 519 - protein, 132 aa, Artificial Sequence
  single-domain antibody CoV2-S2-31
MAQVQLVESGGGLVQPGGSLRLSCAASGRSFSYHAMGWFRQAPGKEREVVAAIDWNGKNTYYVDSVKGRFTISRHNAKNTGDVQVNNLGDLQMNNLKSEDTAVYYCAADGYLRTLIKGAAASWGQGTQVTVS

SEQ ID NO: 520 - protein, 117 aa, Artificial Sequence; identical to SEQ ID NO: 107
  single-domain antibody CoV2-S2-32

SEQ ID NO: 521 - protein, 117 aa, Artificial Sequence
  single-domain antibody CoV2-S2-34
MAQVQLVESGGGLVQTGGSLRLSCAASGITFNTYGMGWYRQAPGKQRELVAGIPSIGNINYARFVQGRFTVSRDDGKKAVYLQMNSLKPEDTAIYYCNLQTRFGTNYWGQGTQVTVS

SEQ ID NO: 522 - protein, 118 aa, Artificial Sequence
  single-domain antibody CoV2-S2-37
MAQVQVMESGGGLVQAGGSLRLSCAASTGILGGYDMRWYRQTPGNERELVASISSDGSTNYVDFVKGRFTISSDNAKNTVYLQMDSLKSEDTAVYYCNMRSTYYAIDYWGRGTQVTVS

SEQ ID NO: 523 - protein, 127 aa, Artificial Sequence
  single-domain antibody CoV2-S2-44
MAQVQFVESGGGLVQPGGSLRLSCVGSEFTLDNYDIGWFRQAPGKEREWVSCITPRGNIINYADSVKGRFTISRDNAKNTAFLQMNSLKPEDTAVYYCARSSALYDCSQRQTRFFSSWGQGTQVTVS

SEQ ID NO: 524 - protein, 127 aa, Artificial Sequence
  single-domain antibody CoV2-S2-45
MAQVQLVESGGGLVQAGGSLRLSCAASGLTFSSRAMAWFRQAPGKEREFVARISWSGRNTYYGDSVKGRFTISRDNAKNTVYLQMSSLEPEDTAVYRCAADQQYYNRYYNDEQGYAFWGQGTQVTVS

SEQ ID NO: 525 - protein, 124 aa, Artificial Sequence
  single-domain antibody CoV2-S2-49
MADVQLVESGGGLVQPGGSLRLSCAVSGLTLPYFTISWFRQAPGKEREWVSSSRITGNTYYADSVKGRFTVSRDNAKSTVYLQMNRLKAEDTATYYCASGRNINYYTNSAEYNYWGRGTQVTVS

SEQ ID NO: 526 - protein, 6 aa, Artificial Sequence
  His tag
HHHHHH

SEQ ID NO: 527 - protein, 7 aa, Artificial Sequence
  Flag tag
DYKDDDD

SEQ ID NO: 528 - protein, 8 aa, Artificial Sequence
  Xpress tag
DLYDDDDK

SEQ ID NO: 529 - protein, 15 aa, Artificial Sequence
  Avi tag
GLNDIFEAQKIEWHE

SEQ ID NO: 530 - protein, 26 aa, Artificial Sequence
  Calmodulin tag
KRRWKKNFIAVSAANRFKKISSSGAL

SEQ ID NO: 531 - protein, 6 aa, Artificial Sequence
  Polyglutamate tag
EEEEEE

SEQ ID NO: 532 - protein, 9 aa, Artificial Sequence
  HA tag
YPYDVPDYA

SEQ ID NO: 533 - protein, 10 aa, Artificial Sequence
  Myc tag
EQKLISEEDL

SEQ ID NO: 534 - protein, 8 aa, Artificial Sequence
  Strep tag
WRHPQFGG

SEQ ID NO: 535 - protein, 8 aa, Artificial Sequence
  STREP tag II
WSHPQFEK

SEQ ID NO: 536 - protein, 13 aa, Artificial Sequence
  Softag 1
SLAELLNAGLGGS

SEQ ID NO: 537 - protein, 8 aa, Artificial Sequence
  Softag 3
TQDPSRVG

SEQ ID NO: 538 - protein, 14 aa, Artificial Sequence
  V5 tag
GKPIPNPLLGLDST

SEQ ID NO: 539-543 - skipped (5 entries)

SEQ ID NO: 544 - protein, 39 aa, Artificial Sequence
  leucine zipper domain of Jun
RIARLEEKVKTLKAQNSELASTANMLREQVAQLKQKVMN

SEQ ID NO: 545 - protein, 39 aa, Artificial Sequence
  dimerization domain of Fos
LTDTLQAETDQLEDKKSALQTEIANLLKEKEKLEFILAA

SEQ ID NO: 546 - protein, 59 aa, Artificial Sequence
  dimerization domain of the SH2B adapter protein
WREFCESHARAAALDFARRFRLYLASHPQYAGPGAEAAFSRRFAELFLQHFEAEVARAS

SEQ ID NO: 547 - protein, 55 aa, Artificial Sequence
  SH3 domain of IB1
THRAIFRFVPRHEDELELEVDDPLLVELQAEDYWYEAYNMRTGARGVFPAYYAIE

SEQ ID NO: 548 - protein, 101 aa, Homo sapiens
LGEVHRFHVTVAPGTKLESGPATLHLCNDVLVLARDIPPAVTGQWKLSDLRRYGAVPSGFIFEGGTRCGYWAGVFFLSSAEGEQISFLFDCIVRGISPTKG

SEQ ID NO: 549 - protein, 115 aa, Artificial Sequence
  PDZ-like domain of SATB1
DCKEEHAEFVLVRKDMLFNQLIEMALLSLGYSHSSAAQAKGLIQVGKWNPVPLSYVTDAPDATVADMLQDVYHVVTLKIQLHSCPKLEDLPPEQWSHTTVRNALKDLLKDMNQSS

SEQ ID NO: 550 - protein, 427 aa, Artificial Sequence
  WD40 repeats of APAF
CAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGSQLFSYAAFSDSNIITVVVDTALYIAKQNSPVVEVWDKKTEKLCGLIDCVHFLREVMVKETKIFSFSNDFTIQKLIETRTNKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 551 - protein, 75 aa, Artificial Sequence
  PAS motif of the dioxin receptor
DQELKHLILEAADGFLFIVSCETGRVVYVSDSVTPVLNQQQSEWFGSTLYDQVHPDDVDKLREQLSTSENALTGR

SEQ ID NO: 552 - protein, 33 aa, Artificial Sequence
  EF hand motif of parvalbumin
LSAKETKMLMAAGDKDGDGKIGVDEFSTLVAES

SEQ ID NO: 553 - protein, 44 aa, Artificial Sequence
  dimerization and docking domain 1
SHIQIPPGLTELLQGYTVEVLRQQPPDLVEFAVEYFTRLREARA

SEQ ID NO: 554 - protein, 45 aa, Artificial Sequence
  dimerization and docking domain 2
CGHIQIPPGLTELLQGYTVEVLRQQPPDLVEFAVEYFTRLREARA

SEQ ID NO: 555 - protein, 17 aa, Artificial Sequence
  anchoring domain 1
QIEYLAKQIVDNAIQQA

SEQ ID NO: 556 - protein, 21 aa, Artificial Sequence
  anchoring domain 2
CGQIEYLAKQIVDNAIQQAGC

SEQ ID NO: 557 - protein, 106 aa, Artificial Sequence
  FK-binding protein 12 (FKBP12)
MGVQVETISPGDGRTFPKRGQTCWHYTGMLEDGKKFDSSRDRNPFKFMLGKQEVIRGWEEGVAQMSVGQRAKLTISPDYAYGATGHPGIIPPHATLVFDVELLKLE

SEQ ID NO: 558 - protein, 100 aa, Artificial Sequence
  FKBP12-Rapamycin Binding (FRB) domain
MASRILWHEMWHEGLEEASRLYFGERNVKGMFEVLEPLHAMMERGPQTLKETSFNQAYGRDLMEAQEWCRKYMKSGNVKDLTQAWDLYYHVFRRISKLES

SEQ ID NO: 559 - protein, 29 aa, Artificial Sequence
  S1-1 peptide
DNAKNTAYLQMNSLKPEDTAVYYCAADSK

SEQ ID NO: 560 - protein, 7 aa, Artificial Sequence
  S1-1, S1-23, and S1-RBD-15 peptide
QAPGKER

SEQ ID NO: 561 - protein, 9 aa, Artificial Sequence
  S1-1 peptide
TASRDNAKN

SEQ ID NO: 562 - protein, 9 aa, Artificial Sequence
  S1-1 peptide
LADSVKGRF

SEQ ID NO: 563 - protein, 12 aa, Artificial Sequence
  S1-1 peptide
TASRDNAKNTAY

SEQ ID NO: 564 - protein, 14 aa, Artificial Sequence
  S1-1 peptide
LQMNSLKPEDTAVY

SEQ ID NO: 565 - protein, 9 aa, Artificial Sequence
  S1-1 peptide
YCAADSKGW

SEQ ID NO: 566 - protein, 8 aa, Artificial Sequence
  S1-23 peptide
EDSVKGRF

SEQ ID NO: 567 - protein, 10 aa, Artificial Sequence
  S1-23 peptide
RQAPGKEREF

SEQ ID NO: 568 - protein, 8 aa, Artificial Sequence
  S1-23 peptide
QMNSLKPE

SEQ ID NO: 569 - protein, 12 aa, Artificial Sequence
  S1-23 peptide
MNSLKPEDTAVY

SEQ ID NO: 570 - protein, 8 aa, Artificial Sequence
  S1-23 peptide
KPEDTAVY

SEQ ID NO: 571 - protein, 12 aa, Artificial Sequence
  S1-RBD-15 peptide
TISRDNAKNTVY

SEQ ID NO: 572 - protein, 9 aa, Artificial Sequence
  S1-RBD-15 peptide
TISRDNAKN

SEQ ID NO: 573 - protein, 16 aa, Artificial Sequence
  RBD peptide
QIAPGQTGKIADYNYK

SEQ ID NO: 574 - protein, 9 aa, Artificial Sequence
  RBD peptide
KSNLKPFER

SEQ ID NO: 575 - protein, 8 aa, Artificial Sequence
  RBD peptide
SNLKPFER

SEQ ID NO: 576 - protein, 12 aa, Artificial Sequence
  RBD peptide
GVSPTKLNDLCF

SEQ ID NO: 577 - protein, 8 aa, Artificial Sequence
  RBD peptide
RKSNLKPF

SEQ ID NO: 578 - protein, 7 aa, Artificial Sequence
  RBD peptide
SKVGGNY

SEQ ID NO: 579 - protein, 13 aa, Artificial Sequence
  RBD peptide
NSNNLDSKVGGNY

SEQ ID NO: 580 - protein, 21 aa, Artificial Sequence
  RBD peptide
VIRGDEVRQIAPGQTGKIADY

SEQ ID NO: 581 - protein, 7 aa, Artificial Sequence
  RBD peptide
GVSPTKL

SEQ ID NO: 582 - protein, 8 aa, Artificial Sequence
  RBD peptide
RKSNLKPF

SEQ ID NO: 583 - protein, 11 aa, Artificial Sequence
  RBD peptide
APGQTGKIADY

SEQ ID NO: 584 - protein, 13 aa, Artificial Sequence
  RBD peptide
KVGGNYNYLYRLF

SEQ ID NO: 585 - protein, 16 aa, Artificial Sequence
  RBD peptide
EVRQIAPGQTGKIADY

SEQ ID NO: 586 - protein, 12 aa, Artificial Sequence
  RBD peptide
NRKRISNCVADY

SEQ ID NO: 587 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 588 - protein, 218 aa, Homo sapiens
PAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 589 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 590 - DNA, 29 bp, Artificial Sequence
  Forward primer 6N_CALL001
  misc_feature (1)..(6): n is a, c, g, or t
nnnnnngtcctggctgctcttctacaagg

SEQ ID NO: 591 - DNA, 29 bp, Artificial Sequence
  Forward primer 6N_CALL001B
  misc_feature (1)..(6): n is a, c, g, or t
nnnnnngtcctggctgctcttttacaagg

SEQ ID NO: 592 - DNA, 27 bp, Artificial Sequence
  Reverse primer 6N_VHH_SH_rev
  misc_feature (1)..(6): n is a, c, g, or t
nnnnnnctggggtcttcgctgtggtgc

SEQ ID NO: 593 - DNA, 31 bp, Artificial Sequence
  Reverse primer 6N_VHH_LH_rev
  misc_feature (1)..(6): n is a, c, g, or t
nnnnnngtggttgtggttttggtgtcttggg

SEQ ID NO: 594 - protein, 52 aa, Artificial Sequence
  Albumin-binding domain
DITGAALLEAKEAAINELKQYGISDYYVTLINKAKTVEGVNALKAEILSALP

SEQ ID NO: 595 - protein, 45 aa, Artificial Sequence
  Albumin-binding domain
LKEAKEKAIEELKKAGITSDYYFDLINKAKTVEGVNALKDEILKA